(19) United States
(12) Patent Application Publication
(10) Pub. No.: US 2013/0028942 A1
(43) Pub. Date: Jan. 31, 2013

(54) AEROSOLIZED NITRITE AND NITRIC OXIDE-DONATING COMPOUNDS AND USES THEREOF

(71) Applicant: AIRES PHARMACEUTICALS, INC., San Diego, CA (US)

(72) Inventors: MARK W. SURBER, SAN DIEGO, CA (US); GARY T. ELLIOTT, SAN DIEGO, CA (US)

(73) Assignee: AIRES PHARMACEUTICALS, INC., SAN DIEGO, CA (US)

(21) Appl. No.: 13/626,445

(22) Filed: Sep. 25, 2012

Related U.S. Application Data
(62) Division of application No. 12/343,920, filed on Dec. 24, 2008.
(60) Provisional application No. 61/017,126, filed on Dec. 27, 2007.
(60) Provisional application No. 61/104,548, filed on Oct. 10, 2008.

(51) Int. Cl. A61K 33/00; A61P 9/12; A61P 9/10; A61K 9/14

(52) U.S. Cl. 424/400; 424/718

(57) ABSTRACT

Disclosed herein are formulations of nitrite, nitrite salt, or nitrite- or nitric oxide-producing compounds suitable for aerosolization and use of such formulations for aerosol administration of nitrite, nitrite salt, or nitrite- or nitric oxide-donating compounds for the treatment of pulmonary arterial hypertension, intra-nasal or pulmonary bacterial infections, or to treat or prevent ischemic reperfusion injury of the heart, brain and organs involved in transplantation. In particular, inhaled nitrite, nitrite salt, or nitrite- or nitric oxide-donating compound specifically formulated and delivered to the respiratory tract for the indications is described. Compositions include all formulations, kits, and device combinations described herein. Methods include inhalation procedures and manufacturing processes for production and use of the compositions described.

### CROSS REFERENCE TO RELATED APPLICATIONS

This application claims the benefit of U.S. Provisional Patent Application No. 61/017,126, filed Dec. 27, 2007, and U.S. Provisional Patent Application No. 61/104,548, filed Oct. 10, 2008, which are incorporated herein by reference in their entireties.

### BACKGROUND

1. Technical Field

The present invention relates in its several embodiments to liquid and dry powder formulations for therapeutic delivery of nitric oxide-producing compositions such as nitrite anions (NO₂⁻) to desired anatomical sites, for treatment and/or prophylaxis of a variety of respiratory, pulmonary, vascular and cardiovascular conditions.

2. Description of the Related Art

In a number of undesirable respiratory, pulmonary, vascular and cardiovascular conditions such as pulmonary arterial hypertension, ischemia-reperfusion injury and other conditions, harmful decreases in local pH and/or oxygen tension within tissues produce deleterious consequences such as vasoconstriction, induction of cellular apoptosis or necrosis, inflammation, tissue damage from reactive free radicals, and other clinical detriments. In conditions such as pulmonary arterial hypertension or ischemia/reperfusion injury as may accompany stroke, myocardial infarction or damage to vascularized transplantation tissues, physiological responses characterized by nitric oxide (NO) production have been observed beneficially to promote, inn a affected region, vasodilation, inhibition of inappropriate cellular proliferation and/or blockade of hematopoietic or inflammatory cell infiltration, adhesion or aggregation. Therapeutic strategies exploiting such NO effects in these and other indications (e.g., microbial infection) have been contemplated, with highly variable results.

Nitrite anion (“nitrite”, NO₂⁻) forms following nitric oxide (NO) oxidation and is present in the plasma (0.3-1.0 μM) and tissue (1-20 μM). Both tissue and plasma nitrite may be reduced to NO during hypoxia and acidosis. For instance, at low tissue pH and/or low oxygen tension, nitrite anion may be reduced to NO by acid reduction or enzymatic action (from enzymes such as xanthine oxidoreductase). However, at pH levels and oxygen tensions that are considered within the normal physiological range, nitrite anion is considered an inert metabolic end-product of NO oxidation and has limited biological activity. It has recently been demonstrated that near-physiological levels of nitrite are reduced to NO by reaction with deoxyhemoglobin along the physiological oxygen gradient; a chemical reaction having a rate that is oxygen- and pH-dependent, and that potentially contributes to hypoxic vasodilation. From these observations, it is believed that hypoxia- and/or pH-dependent NO production from nitrite may have physiologic benefit to diseased tissue. For example, beneficial nitrite conversion to NO is associated with acute or chronic vasodilation, and/or with complete or partial inhibition or reversal of detrimental vascular remodeling, in clinical indications such as pulmonary arterial hypertension (PAH), and ischemia/reperfusion (I/R) injury in heart, brain, liver, lung and other tissues, following infarction, stroke and/or transplantation.

Although clinical benefits derived from nitrite-dependent NO production have been described for several vascular and other diseases, effective delivery of NO benefits to desired tissues has been hindered by physicochemical factors. In particular, the instability of NO, its occurrence as a gas, and its short biological half-life in view of physiological degradative pathways have presented obstacles to obtaining sustained foci of significant NO concentrations at afflicted anatomical sites. (Hunter et al., 2004) Specific examples of indications for which there remains a need for effective NO delivery to tissues include those described in the following paragraphs.

Pulmonary Aterial Hypertension (See, e.g., Rubin L J et al., 2006; Gladwin et al., 2006; and Hunter et al., 2004). Most patients with pulmonary arterial hypertension (PAH) present in the clinic with exertional dyspnea, which is indicative of an inability to increase pulmonary blood flow with exercise. Exertional chest pain, syncope, and edema are indications of more severely impaired right heart function. Prognosis for patients with PAH, although improved with the advent of modern therapies, is still dire, with a median life expectancy of approximately 2.5 years following diagnosis. Establishing the diagnosis of PAH, which is frequently delayed, is often made by echocardiography, which demonstrates evidence of right ventricular volume and pressure overload. Pulmonary artery pressure can be estimated during echocardiography using Doppler techniques. Many patients ultimately undergo cardiac catheterization to support a definitive diagnosis.

Although specific triggers for the development of PAH remain unknown, a number of mechanisms have been proposed, and many have translated into targeted therapies for PAH. Currently available therapies for PAH, including prostanoids, endothelin receptor antagonists (ETA), and phosphodiesterase-5 (PDE5) inhibitors, have led to significantly improved quality of life and survival for many patients. However, the route and frequency of administration of the prostanoids, the hepatotoxicity of the ETA receptor antagonists, and concerns about the efficacy of both the ETA receptor antagonists and PDE5 inhibitors suggest that many patients with PAH could benefit from other effective therapies that would offer currently unavailable advantages such as ease of administration, greater time intervals between dosing and a favorable toxicity profile.

Observations of NO-induced hypoxic vasodilation suggest a role in this process for nitrite as an in vivo NO precursor. Diminished expression of the enzymes responsible for synthesis of nitric oxide (NO) and loss of NO signaling via disruption of normal vascular endothelium are also proposed to play a role in the development of PAH. Because loss of arterial vasodilatory capacity and capacitance, vascular lumenal narrowing and occlusion of pulmonary arteries have been attributed at least in part to a nitric oxide deficiency in PAH patients, development of a therapeutic strategy that attempts to reconstitute NO signaling is attractive. (Rubin L J et al., 2006; Gladwin et al., 2006; Hunter et al., 2004) Despite such proposals, delivery of therapeutically effective amounts of NO or an in vivo NO precursor that is both rapid and sustained over time remains an elusive goal.

Nitric oxide is normally produced from endothelial NO synthase under normoxic states and participates in the regulation of basal blood vessel tone and vascular homeostasis (antiplatelet activity, modulation of oxidative/nitrosative stress and inflammation, endothelial and smooth muscle proliferation and adhesion molecule expression). NO as a paracrine signaling molecule diffuses from the endothelium to vicinal smooth muscle, binds avidly to the heme of soluble guanylyl cyclase (which produces cyclic guanosine monophosphate), activates cyclic guanosine monophosphate dependent protein kinases, and ultimately produces smooth muscle relaxation.

Artery-to-venous formation of iron-nitrosyl-hemoglobin (HbFeII-NO) was observed during nitrite infusions into the brachial artery of humans. An analysis of the iron-nitrosyl-hemoglobin levels during all experimental conditions (rest, L-NMMA co-infusion, and exercise) revealed a striking inverse correlation with oxyhemoglobin saturation, i.e., as hemoglobin deoxygenated more NO was formed. These physiological observations were consistent with a reaction between nitrite anion and deoxyhemoglobin to form NO:

NO₂⁻+HbFeII(deoxyhemoglobin)+H⁺→NO(nitric oxide)+HbFeIII+OH

The reaction requires deoxyhemoglobin and a proton, providing oxygen and pH sensor chemistry, respectively, and generates the potent vasodilator NO.

Much of the formed NO is then captured as iron nitrosyl-hemoglobin (HbFeII-NO) on vicinal hemes, thus constituting a depot for NO production in venous blood:

NO+HbFeII(deoxyhemoglobin)→HbFeII-NO(iron-nitrosyl-hemoglobin)

Potential use of nitrite anion as a therapy for PAH has been considered. For example, in patients with New York Heart Association (NYHA) Class III-IV PAH (as defined by Rich S. ed. Executive Summary from the World Symposium on Primary Pulmonary Hypertension, 1998, Evian, France) the limited cardiac output resulting from right ventricular failure leads to abnormally low mixed venous oxygen content. In a study of subjects undergoing atrial septostomy, the mean mixed venous oxygen saturation was 45.1±5.0% and the mixed venous partial pressure of oxygen was 24.4±1.9 mmHg, despite therapy with prostanoids, bosentan, or diuretics (Kurzyna et al., 2007). Delivery of nitrite to the pulmonary circulation has the theoretical advantage of maximizing local NO production due to the peak reductase activity around this oxygen saturation. The resulting pulmonary vasodilation may also result in improved oxygen uptake by the lungs, oxygen delivery to the tissues, and higher mixed venous oxygen content under steady state conditions. Under conditions of higher metabolic demand, as occurs with exercise, the increased peripheral oxygen uptake will result, in a lower mixed venous oxygen content, and a shift toward maximal reductase activity and enhanced NO generation from administered nitrite. Despite such apparent advantages of pulmonary nitrite delivery for PAH, current efforts have been disappointing for a variety of reasons, including poor NO stability and difficulties in achieving sustained localized NO generation.

Published studies have shown that decreased levels of NO also stimulate vascular remodeling (Ozaki et al., 2001; Chou et al., 2005; Yamashita et al., 2007). To this end, decreased NO inhibits a pro-apoptotic kinase (ASK1) which normally functions as a signal in vascular hypertrophy and neointimal thickening. It appears that these adverse events occur in response to low nitric oxide levels whereby ASK1 is inhibited and the pro-apoptotic effect is lost. Under conditions of normal basal NO generation, ASK1 pro-apoptotic activity is maintained and these adverse events do not occur (Yamashita et al., 2007). To further illustrate the importance of NO in maintaining healthy vessel morphology, stimulation of endogenous NO synthesized by endothelial nitric oxide synthase (eNOS) has been shown to prevent chronic hypoxia-induced remodeling of pulmonary vasculature. Taken together, it appears that elevated levels of NO provide a protective mechanism against detrimental pulmonary vascular remodeling (Ozaki et al., 2001).

Ischemic Reperfusion Injury: Coronary Heart Disease (See, e.g., Yellon D. M. and Hausenloy, 2007; Duranski et al., 2005). Coronary heart disease is the leading cause of death worldwide, and 3.8 million men and 3.4 million women die of the disease each year. After an acute myocardial infarction, early and successful myocardial reperfusion with the use of thrombolytic therapy or primary percutaneous coronary intervention (PCI) is the most effective strategy for reducing the size of a myocardial infarct and improving the clinical outcome. The process of restoring blood flow to the ischemic myocardium, however, can induce injury. This phenomenon, termed myocardial reperfusion injury, can paradoxically reduce the beneficial effects of myocardial reperfusion.

The potentially detrimental form of myocardial reperfusion injury, termed lethal reperfusion injury, is defined as myocardial injury caused by the restoration of coronary blood flow after an ischemic episode. The injury culminates in the death of cardiac myocytes that were viable immediately before myocardial reperfusion. This type of myocardial injury, which by itself can induce cardiomyocyte death and increase infarct size, may in part explain why, despite optimal myocardial reperfusion, the rate of death after an acute myocardial infarction approaches 10%, and the incidence of cardiac failure after an acute myocardial infarction is almost 25%.

Reperfusion of ischemic tissues provides oxygen and metabolic substrates necessary for the recovery and survival of reversibly injured cells, but reperfusion itself actually results in the acceleration of cellular necrosis. Ischemic reperfusion (I/R) injury is characterized by the formation of oxygen radicals upon reintroduction of molecular oxygen to ischemic tissues, resulting in widespread lipid and protein oxidative modifications, mitochondrial injury, and tissue apoptosis and necrosis. In addition, after reperfusion of ischemic tissues, blood flow may not return uniformly to all portions of the ischemic tissues, a phenomenon that has been termed the “no-reflow” phenomenon. Reductions in blood flow after reperfusion are thought to contribute to cellular injury and necrosis. The sudden re-introduction of blood into ischemic tissue also results in massive tissue disruption, enzyme release, reductions in high energy phosphate stores, mitochondrial injury, and necrosis. Furthermore, it has also been suggested that I/R injury is characterized by an inappropriate inflammatory response in the microcirculation, resulting in leukocyte-endothelial cell interactions that are mediated by the upregulation of both leukocyte and endothelial cell adhesion molecules. Intensive research efforts have been focused on the amelioration of various pathophysiological components of I/R injury to limit the extent of tissue injury and necrosis.

Studies in animal models of acute myocardial infarction suggest that lethal reperfusion injury accounts for up to 50% of the final size of a myocardial infarct, and in these models a number of strategies have been shown to ameliorate lethal reperfusion injury. Yet, the translation of these beneficial effects into the clinical setting has been disappointing. Nevertheless, recent demonstrations (e.g., Lefer et al., 1993) suggest that nitric oxide produced by nitric oxide donors such as nitrite and nitrite salts, as well as other NO donors such as SPM-5185 and SPM-5267, may limit ischemic preperfusion injury to the myocardium. Despite recognition of the potential benefits theoretically afforded by localized increases in bioavailable NO, actually achieving such increases has remained a challenging and elusive goal.

Although NO, NO donors, and NO synthase activation or transgenic overexpression have been shown to exert protective effects to counter reperfusion injury in a number of reported experimental model systems, contrary evidence accumulated using other experimental models points to harmful consequences of excessive NO in this process. Evaluation of these studies suggests that variations in dosage and duration of NO exposure can have significant effects, resulting in a narrow therapeutic safety window for NO in I/R pathophysiology. An additional constraint is that NO formation from NO synthase requires oxygen as a substrate, the availability of which is limited during ischemia. By as yet uncharacterized physiological regulatory processes, nitrite may thus be selectively reduced to NO in tissues with low oxygen tension.

For instance, the coincidence of low pH and NO is known to maintain herne proteins in a reduced and liganded state, to limit free iron- and heme-mediated oxidative chemistry, to transiently inhibit mitochondrial respiration (including inhibition of mitochondrial cytochrome C oxidase), and to modulate apoptotic effectors. One or more of these mechanisms may therefore contribute to cytotoxicity that is observed following severe ischemia. Evaluation of nitrite therapy in controlled murine models of myocardial I/R injury, for example, provided evidence for a protective effect of nitrite against cellular necrosis and apoptosis, mediated by a hypoxia-dependent bioconversion of nitrite to NO and nitrosated or nitrosylated proteins (Duranski et al., 2005).

Ischemic Reperfusion Injury: Stroke (See, e.g., Jung et al., 2006). Recent insight into the basic mechanism involved in ischemic stroke indicates that endothelial dysfunctions along with the oxidative stress and inflammation represent a key step in the cerebral ischemias/reperfusion (I/R) injury. Nitric oxide (NO) is primarily known for an endothelial survival factor maintaining the endothelia integrity and a vasodilator regulating the blood flow. In addition to its major role, as a potentially protective agent, NO can improve neuronal survival, inhibit platelet aggregation and neutrophil adhesion, and scavenge reactive free radicals, thus reducing the ischemic injury. However, a concomitant surge in production of superoxide and NO after reperfusion may lead to formation of peroxynitrite, a powerful oxidant. So far, evidence indicates that NO may be linked both to protective and toxic effects after I/R, depending on the level, the location, the source, and the environment.

NO synthase (NOS) is a dominant physiological source of NO. However, the enzymatic activity of NOS requires oxygen and is blocked under hypoxia. Therefore, alternative pathways for hypoxic release of NO have high physiological relevance. The agents that liberate NO have been recognized as potentially important for therapeutic purposes, especially in ischemic disorders. A variety of structurally different NO precursors and NO donors have been shown to limit infarct size by improving blood flow in the penumbra areas and reducing the oxidative stress in an NO-dependent fashion. Recent work supports the application of nitrite as a precursor from which NO can be formed for treatment of ischemic disorders. The nitrite anion is reduced to form NO as a result of reduction by deoxyhemoglobin, myoglobin, tissue heme proteins, and non enzymatic disproportionating. The NO formation from nitrite and, in parallel, the vasodilatory effect, are increased under conditions of acidosis, hypoxia, and tissue I/R. This improved understanding of the biochemical conversion of nitrite to NO has resulted in a great deal of interest in the potential beneficial effects of nitrite therapy in animal models of ischemia, despite recognized challenges associated with regulating local levels of this highly unstable mediator, as also noted above.

The ischemic cerebral environment might allow for the acidic and hypoxic reduction of nitrite to NO. In rat models of cerebral ischemic reperfusion injury, evaluation of nitrite therapy compared to control therapies provided evidence that nitrite exerts a profound neuroprotective effect with antioxidant properties in the ischemic brains. Nitrite, as a precursor from which NO can be formed under appropriate conditions, may therefore represent a novel therapeutic agent in the setting of acute stroke.

Ischemic Reperfusion Injury: Lung Transplant (See, e.g., de Perrot et al. 2003; and Esme et al., 2006). Since 1983, lung transplantation has enjoyed increasing success and has become the mainstay of therapy for most end-stage lung diseases. Despite refinements in lung preservation and improvements in surgical techniques and perioperative care, ischemia reperfusion-induced lung injury remains a significant cause of early morbidity and mortality after lung transplantation. The syndrome typically occurs within the first 72 hours after transplantation and is characterized by nonspecific alveolar damage, lung edema, and hypoxemia. The clinical spectrum can range from mild hypoxemia associated with few infiltrates on chest X-ray to a picture similar to full-blown acute respiratory distress syndrome requiring positive pressure ventilation, pharmacologic therapy, and occasionally extracorporeal membrane oxygenation. A number of terms have been used to describe this syndrome, but ischemia-reperfusion injury is most commonly used, with primary graft failure attributed to the most severe form of injury that frequently leads to death or prolonged mechanical ventilation beyond 72 hours. In addition to significant morbidity and mortality in the early postoperative period, severe ischemia-reperfusion injury can also be associated with an increased risk of acute rejection that may lead to graft dysfunction in the long term.

Primary graft failure is the end-result of a series of clinical insults occurring from the time of brain death to the time of lung reperfusion after transplantation. Ischemia-reperfusion injury has been identified as the main cause of primary graft failure. However, other injuries that occur in the donor before the retrieval procedure can contribute to and amplify the lesions of ischemia and reperfusion. Attention of lung transplant physicians has therefore been focused on selective assessment of donor lungs, effective techniques for lung preservation, and careful management of transplanted lungs after reperfusion to reduce the severity of ischemia-reperfusion injury and the incidence of primary graft failure. Donor lung assessment is an attempt to select lungs that will be able to handle a period of several hours of ischemia without significant impairment in their function after reperfusion. Unfortunately, currently only 10 to 30% of donor lungs are judged suitable for transplantation.

Lungs that have been selected for transplantation are generally flushed with a preservation solution and hypothermically preserved to decrease their metabolic rate and energy requirement until implantation in the recipient. The period of cold ischemic storage is kept as short as possible and usually ranges from about four to eight hours, according to the location of the donor. Although hypothermia is essential for organ storage, it is associated with a series of events such as oxidative stress, sodium pump inactivation, intracellular calcium overload, iron release, and induction of cell death that may induce upregulation of certain molecules on cell surface membranes and the release of proinflammatory mediators that will eventually activate passenger (donor) and recipient leukocytes after reperfusion. Prolonged ischemia may also result in a “no-reflow phenomenon” demonstrated by significant microvascular damage leading to persistent blood flow obstruction and subsequent ischemia despite reperfusion.

Over the past decade, numerous studies have been performed to optimize the technique of lung preservation. A new preservation solution, which combines a low potassium concentration and dextran, has also been developed specifically for the lungs. Several strategies for the prevention and treatment of ischemia/reperfusion-induced lung injury have been introduced into clinical practice and have translated into a reduction in the incidence of severe ischemia reperfusion injury from approximately 30% to 15% or less.

The ischemic lung transplant environment might be permissive for the acidic and hypoxic reduction of nitrite to NO, discussed above. For example, inclusion of the nitric oxide donor nitroglycerin during flush perfusion and reperfusion periods in an ischemic rabbit lung model coincided with the appearance of a protective effect on lung function against reperfusion injury during in situ normothermic ischemic lung model therapy (Emse et al, 2006).

Ischemic Reperfusion Injury: Kidney Transplant (See, e.g., Neto et al., 2004). Ischemic reperfusion (I/R) injury of the kidney graft has been considered one of the major deleterious factors of successful renal transplantation. In the immediate posttransplant period, I/R injury can cause an increased risk of delayed or primary nonfunction of transplanted grafts, and complicates posttransplant recipient management, associating with high morbidity and mortality. In addition, in clinical and experimental studies, I/R injury has been identified as a key risk factor in a predisposition to the early appearance of chronic allograft nephropathy and short graft life, in part, by accelerating alloantigen-specific immune reactions. Because of the current shortage of organs for transplantation, the donor pool has been expanded with the use of marginal donors (e.g., old donors, non-heart-beating donors, grafts with prolonged cold storage), and grafts from these donors have a higher incidence of severe cold I/R injury.

I/R injury in the kidney has complex sequelae, resulting in pathophysiological features of persistent intrarenal vasoconstriction, injury of microvascular endothelial cells and tubular epithelial cells, and activation of inflammatory cascades, it is instigated by the lack of oxygen during cold preservation and ATP depletion, followed by an alteration in intracellular calcium and sodium concentrations and activation of cytotoxic enzymes (e.g., proteases, phospholipases, etc.). Subsequent warm reperfusion of kidney grafts initiates a rapid increase in the generation of reactive oxygen species, which further promotes cell damage and activates inflammatory cascades. Vascular endothelial cell injury and upregulation of adhesion molecules are also implicated during renal I/R injury and result in vasoconstriction, platelet activation, and increased leukocyte extravasation, which subsequently lead to further inflammatory injury.

Ischemic Reperfusion Injury: Liver Transplant (See, e.g., Lang et al., 2007). Liver ischemia with consequent reperfusion results in a multitude of cellular, humoral, and biochemical events leading to hepatocellular injury and liver dysfunction. Hepatic ischemia/reperfusion (I/R) injury is a significant complication in liver transplantation that can predispose patients to a profound reperfusion syndrome, resulting in primary graft nonfunction and initial poor function of the graft. In addition, increased susceptibility of marginal livers to IR injury limits the number that are available for transplantation. Pharmacological approaches to curtailing the perturbations of liver I/R during allograft transplantation have generally been unsuccessful due in large part to the complex mechanisms involved. Experimental studies of hepatic I/R injury indicate roles for infiltrating polymorphonuclear cells (PMNs) and T cells, activation of Kupffer cells and endothelial cells, and formation of ROS/reactive nitrogen species (ROS/RNS). This complexity arises in part from the involvement of different mediators and cell types at temporally distinct stages of the injury response, and from the nature of the experimental model studied (species, age, sex, etc.). Irrespective of the precise mechanisms involved, increased inflammation and cytotoxicity are key components in hepatocellular dysfunction during the pathogenesis of liver I/R injury and provide targets for therapeutic intervention.

Recently, it was suggested that decreased hepatic enzymatic production of NO from eNOS (also known as NOS3) within 1 hour of reperfusion in humans undergoing orthotopic liver transplantation contributes to the I/R-dependent injury observed. Moreover, studies in mice have shown that administration of NO-donors or overexpression of hepatic eNOS inhibits I/R injury in the liver. NO-mediated protection in I/R injury can occur via multiple mechanisms, including cytoprotection, anti-inflammatory effects, modulation of mitochondrial respiration, antioxidant effects, and maintenance of vasomotor tone at the presinusoidal site within the hepatic sinusoid. However, NO can also contribute to i/R injury via formation of secondary RNS, including peroxyntrite.

Inhaled nitric oxide gas (iNO) has been used clinically for nearly two decades for the treatment of reduced oxygen tensions and reduced pulmonary artery pressures in patients suffering from inflammatory-mediated lung injury, and to assist in enhancing flow in ventricular assist devices. Unfortunately, its use in adults has met with limited success, as the clinical evidence does not support its administration as a first-line therapeutic agent for pulmonary related diseases. Traditional thinking has been that as iNO crosses the alveolar-capillary membrane, it is rendered inactive by rapid reactions with oxy- or deoxyhemoglobin in the red blood cell. However, seminal studies by Kubes et al. dismissed this concept, demonstrating that iNO possesses extrapulmonary bioactivity in the mesenteric vasculature by preventing neutrophil adhesion a feline model of I/R injury. These concepts have been extended to show that iNO inhibits myocardial I/R injury in mice, inhibits myocardial injury in patients undergoing cardiopulmonary bypass, improves forearm blood flow in healthy volunteers, and inhibits I/R dependent inflammatory injury in patients undergoing knee surgery.

How iNO mediates extrapulmonary effects remains unclear, with the general hypothesis being that iNO forms a relatively stable, NO-containing intermediate in the circulation, which then mediates systemic effects either directly or after being recycled to NO. Recent evidence in a feline model of I/R suggests that the intermediate may be plasma S-nitrosothiols (SNO) (e.g., S-nitrosoalbumin), whereas studies in humans and mice indicate nitrite as a possible mediator. Direct administration of nitrite has conferred protection against hepatic and myocardial I/R injury in murine models, possibly as an effect of biological mechanisms described above for nitrite reduction to NO under ischemic conditions. It should be noted that other NO-containing candidates in the circulation that are relatively labile under biological conditions may also be formed upon NO inhalation (via nitrosylation or S-nitrosation reactions). These include SNO in the red blood cell, ferrous nitrosylhemoglobin (HbNO), and C- or N-nitrosamines (referred to as XNO). Patients receiving iNO had improved hepatic function after transplantation, which was associated with inhibition of hepatic cell death, with little effect on PMN accumulation. In addition, measurement of different NO derivatives in these patients suggested that the beneficial effects of iNO may occur via increasing circulating levels of nitrite.

Despite such accumulating evidence of NO roles in a number of clinically relevant contexts such as PAH, I/R injury, transplantation, vital organ dysfunction and others, clearly there remains a need for improved compositions and methods for the effective delivery of appropriate sources of NO to appropriate tissue sites in appropriate quantities and for appropriate periods of time. The presently disclosed invention embodiments address this need and provide other related advantages.

### BRIEF SUMMARY

According to a certain embodiment of the present invention, there is provided a nitrite compound formulation composition for pulmonary delivery, comprising (a) a nitrite compound aqueous solution having a pH greater than 7.0; and (b) an acidic excipient aqueous solution, wherein upon admixture of (a) and (b) to form a nitrite compound formulation: (i) the nitrite compound is present at a concentration of from about 0.667 mg NO₂⁻/mL to about 100 mg NO₂⁻/mL, (ii) the nitrite compound formulation has a pH of from about 4.7 to about 6.5, and (iii) nitric oxide bubbles are not visually detectable for at least 15, 30, 45 or 60 minutes following admixture. In a further embodiment upon admixture of (a) and (b) the nitrite compound is present at a molar ratio relative to the acidic excipient that exceeds 150:1, 200:1 or 250:1.

In other embodiments there is provided a nitrite compound formulation composition for pulmonary delivery, comprising: (a) a nitrite compound aqueous solution having a pH greater than 7.0; and (b) an acidic excipient aqueous solution, wherein upon admixture of (a) and (b) to form a nitrite compound formulation: (i) the nitrite compound is present at a concentration of from about 0.667 mg NO₂⁻/mL to about 100 mg NO₂⁻/mL, (ii) the nitrite compound formulation has a pH of from about 4.7 to about 6.5, and (iii) the nitrite compound is present at a molar ratio relative to the acidic excipient that exceeds 150:1, 200:1 or 250:1. In certain further embodiments, upon nebulization (e.g., vibrating-mesh nebulization) of the nitrite compound formulation to form an aerosol comprising liquid particles of about 0.1 to 5.0 microns volumetric mean diameter, the aerosol comprises from 12 parts per billion to 1800 parts per billion nitric oxide. In certain other further embodiments, within 15 minutes after admixture, nebulization of the nitrite compound formulation by a nebulizer (e.g., vibrating-mesh nebulizer) is not detectably impaired relative to nebulization by the nebulizer of the nitrite compound aqueous solution. In certain other further embodiments the nitrite compound formulation composition further comprises a taste-masking agent, which in certain still further embodiments comprises sodium saccharin.

In other embodiments there is provided a nitrite compound formulation for pulmonary delivery, comprising an aqueous solution having a pH of from about 4.7 to about 6.5, the solution comprising: (a) a nitrite compound at a concentration of from about 0.667 mg NO₂⁻/mL to about 100 mg NO₂⁻/mL; and (b) citric acid at a concentration of from about 0.021 mM to about 3.2 mM. In certain embodiments, upon nebulization (e.g., vibrating-mesh nebulization) of the nitrite compound formulation to form an aerosol comprising liquid particles of about 0.1 to 5.0 microns volumetric mean diameter, the aerosol comprises from 12 parts per billion to 1800 parts per billion nitric oxide. In certain further embodiments the nitrite compound formulation comprises a taste-masking agent, which in certain still further embodiments comprises sodium saccharin.

According to certain embodiments there is provided a nitrite compound formulation for pulmonary delivery, comprising an aqueous solution having a pH of from about 4.7 to about 6.5, the solution comprising: (a) a nitrite compound of a concentration of from about 0.667 mg NO₂⁻/mL to about 100 mg NO₂⁻/mL, (b) a buffer that has a pKa between 5.1 and 6.8 and that is present at a concentration sufficient to maintain a pH from about 4.7 to about 6.5 for a time period of at least one hour at 23° C.; and (c) a taste-masking agent. In certain embodiments, the nitrite compound formulation upon nebulization (e.g., vibrating-mesh nebulization) of the nitrite compound formulation to form an aerosol comprising liquid particles of about 0.1 to about 5.0 microns volumetric mean diameter, the aerosol comprises from 12 parts per billion to 1800 parts per billion nitric oxide. In certain other embodiments the buffer is selected from malate, pyridine, piperazine, succinate, histidine, maleate, bis-Tris, pyrophosphate, PIPES, ACES, histidine, MES, cacodylic acid, H₂CO₃/NaHCO₃ and N-(2-Acetamido)-2-iminodiacetic acid (ADA).

In another embodiment there is provided a nitrite compound formulation for pulmonary delivery, comprising: an aqueous solution having a pH of from about 4.7 to about 6.5 and an osmolality of from about 100 to about 3600 mOsmol/kg, the solution comprising: (i) a nitrite compound at a concentration of from about 0.667 mg NO₂⁻/mL to about 100 mg NO₂⁻/mL; and (ii) a pH buffer having a pKa between 5.1 and 6.8, wherein upon nebulization (e.g., vibrating-mesh nebulization), the nitrite compound formulation forms an aerosol that comprises liquid particles of about 0.1 to about 5.0 microns volumetric mean diameter, the aerosol comprising from 12 parts per billion to 1800 parts per billion nitric oxide. In certain further embodiments the nitrite compound formulation is selected from: (a) the nitrite compound formulation which further comprises a taste-masking agent, (b) the nitrite compound formulation in which the nitrite compound concentration is at least 16.7 mg NO₂⁻/mL, the formulation further comprising a taste-masking agent, (c) the nitrite compound formulation in which the osmolality is less than about 650 mOsmol/kg and the nitrite compound is present at a molar concentration relative to the pH buffer that exceeds 150:1, 200:1, 250:1, 300:1, 400:1 or 500:1, and (d) the nitrite compound formulation in which the osmolality is less than about 1000 mOsmol/kg [50 mg NO₂⁻/mL] and the nitrite compound is present at a molar concentration relative to the pH buffer that exceeds 150:1, 200:1, 250:1, 300:1, 400:1 or 500:1, the formulation further comprising a taste-masking agent. In certain still further embodiments the taste-masking agent comprises sodium saccharin. In certain other embodiments the pH buffer is selected from malate, pyridine, piperazine, succinate, histidine, maleate, Bis-Tris, pyrophosphate, PIPES, ACES, histidine, MES, cacodylic acid, H₂CO₃/NaHCO₃ and N-(2-Acetamido)-2-iminodiacetic acid (ADA).

In certain embodiments, the nitrite formulations of the invention have low iron concentrations with the proportion of iron to nitrite being less than 1:1 weight/weight. In other related embodiments the nitrite formulations contain only trace amounts of iron.

There is also provided according to certain embodiments a nitrite compound formulation for pulmonary delivery, comprising: an aqueous solution having a pH of from about 4.7 to about 6.5 and an osmolality of from about 100 to about 3600 mOsmol/kg, the solution comprising: (i) a nitrite compound at a concentration of from about 0.667 mg NO₂⁻/mL [14.5 mM] to about 100 mg NO₂⁻/mL [2.174 M]; and (ii) citric acid, wherein upon nebulization (e.g., vibrating-mesh nebulization) of the nitrite compound formulation to form an aerosol comprising liquid particles of about 0.1 to about 5.0 microns volumetric mean diameter, the aerosol comprises from 12 parts per billion to 1800 parts per billion nitric oxide. According to certain further embodiments the nitrite compound formulation is selected from: (a) the nitrite compound formulation which further comprises a taste-masking agent, (b) the nitrite compound formulation in which the nitrite compound concentration is at least 16.7 mg NO₂⁻/mL [362.5 mM], the formulation further comprising a taste-masking agent, (c) the nitrite compound formulation in which the osmolality is less than about 650 mOsmol/kg and the nitrite compound is present at a molar concentration relative to the pH buffer that exceeds 150:1, 200:1, 250:1, 300:1, 400:1 or 500:1, and (d) the nitrite compound formulation in which the osmolality is less than about 1000 mOsmol/kg and the nitrite compound is present at a molar concentration relative to the pH buffer that exceeds 150:1, 200:1, 250:1, 300:1, 400:1 or 500:1, the formulation further comprising a taste-masking agent. In certain further embodiments the taste-masking agent comprises sodium saccharin.

Certain embodiments also provide a nitrite compound formulation for pulmonary delivery, comprising: an aqueous solution having a pH of from about 4.7 to about 685, the solution comprising sodium nitrite; sodium saccharin; and citric acid, wherein: (i) sodium nitrite is present in the solution, relative to sodium saccharin, at a molar ratio of from about 1.3×10³:1 to about 4.4×10³:1, and (ii) sodium nitrite is present in the solution, relative to citric acid, at a molar ratio of from about 2.0×10²:1 to about 6.9×10²:1. In a further embodiment, upon nebulization (e.g., vibrating-mesh nebulization) of the formulation to form an aerosol comprising liquid particles of about 0.1 to about 5 microns volumetric mean diameter, the aerosol comprises from 12 parts per billion to 1800 parts per billion nitric oxide.

In another embodiment there is provided a nebulized liquid particle of about 0.1 to 5 microns volumetric mean diameter that is formed by a method comprising: (1) admixing (a) a nitrite compound aqueous solution having a pH greater than 7.0, and (b) an acidic excipient aqueous solution, to form a nitrite compound formulation; and (2) nebulizing, within about 15-30 minutes of said step of admixing, the nitrite compound formulation of (1) in at least one of a vibrating-mesh nebulizer and a jet nebulizer to obtain an aerosol that comprises said nebulized liquid particle, wherein: (i) the nitrite compound is present in the nitrite compound formulation at a concentration of from about 0.667 mg NO₂⁻/mL [14.5 mM] to about 100 mg NO₂⁻/mL [2174 M], (ii) the nitrite compound formulation has a pH of from about 4.7 to about 6.5, and (iii) the aerosol comprises from 12 parts per billion to 1800 parts per billion nitric oxide. In a further embodiment, the nebulized liquid particle is selected from (a) the particle that is formed by the method wherein step (1) further comprises admixing a taste-masking agent such that the nitrite compound formulation comprises said taste-masking agent, and (b) the particle that is formed by the method wherein step (1) further comprises admixing a taste-masking agent such that the nitrite compound formulation comprises said taste-masking agent, wherein the nitrite compound concentration in the nitrite compound formulation is at least 16.7 mg NO₂⁻/mL [362.5 mM]. In a further embodiment the taste-masking agent comprises sodium saccharin.

There is also provided in another embodiment a nebulized liquid particle of about 0.1 to 5 microns volumetric mean diameter, comprising an aqueous solution having a pH of from about 4.7 to about 6.5, the solution comprising (a) a nitrite compound at a concentration of from about 0.667 mg NO₂⁻/mL to about 100 mg NO₂⁻/mL; and (b) citric acid at a concentration of from about 0.021 mM to about 3.2 mM, wherein the nebulized liquid particle is present in an aerosol that comprises from 12 parts per billion to 1800 parts per billion nitric oxide. In another embodiment there is provided a nebulized liquid particle of about 0.1 to 5 microns volumetric mean diameter, comprising an aqueous solution having a pH of from about 47 to about 6.5, the solution comprising: (a) a nitrite compound at a concentration of from about 0.667 mg NO₂⁻/mL to about 150 mg NO₂⁻/mL; (b) a buffer that has a pKa between 5.1 and 6.8 and that is present at a concentration sufficient to maintain a pH from about 4.7 to about 6.5 for a time period of at least one hour at 23° C., wherein the nebulized liquid particle is present in an aerosol that comprises between 12 parts per billion and 1800 parts per billion nitric oxide. In certain further embodiments the buffer is selected from malate, pyridine, piperazine, succinate, histidine, maleate, Bis-Tris, pyrophosphate, PIPES, ACES, histidine, MES, cacodylic acid, H₂CO₃/NaHCO₃ and N-(2-Acetamido)-2-iminodiacetic acid (ADA).

In another embodiment there is provided a nebulized liquid particle of about 0.1 to about 5 microns volumetric mean diameter, comprising an aqueous solution having a pH of from about 4.7 to about 6.5 and an osmolality of from about 100 to about 3600 mOsmol/kg, the solution comprising (i) a nitrite compound at a concentration of from about 0.667 mg NO₂⁻/mL to about 100 mg NO₂⁻/mL; and (ii) a pH buffer having a pKa between 5.1 and 6.8, wherein the nebulized liquid particle is present in an aerosol that comprises from 12 parts per billion to 1800 parts per billion nitric oxide. In certain embodiments the buffer is selected from malate, pyridine, piperazine, succinate, histidine, maleate, Bis-Tris, pyrophosphate, PIPES, ACES, histidine, MES, cacodylic acid, H₂CO₃/NaHCO₃ and N-(2-Acetamido)-2-iminodiacetic acid (ADA).

In certain other embodiments there is provided a nebulized liquid particle of about 0.1 to 5 microns volumetric mean diameter, comprising an aqueous solution having a pH of from about 4.7 to about 6.5 and an osmolality of from about 100 to about 3600 mOsmol/kg, the solution comprising (i) a nitrite compound at a concentration of from about 0.667 mg NO₂⁻/mL to about 100 mg NO₂⁻/mL; and (ii) citric acid, wherein the nebulized liquid particle is present in an aerosol that comprises from 12 parts per billion to 1800 parts per billion nitric oxide, in certain further embodiments of the above described nebulized liquid particles, the particle is selected from (a) the nebulized liquid particle which further comprises a taste-masking agent, (b) the nebulized liquid particle in which the nitrite compound concentration is at least 16.7 mg NO₂⁻/mL, the liquid particle further comprising a taste-masking agent, (c) the particle comprising the nitrite compound formulation in which the osmolality is less than about 650 mOsmol/kg and the nitrite compound is present at a molar concentration relative to the pH buffer that exceeds 150:1, 200:1, 250:1, 300:1, 400:1 or 500:1, and (d) the particle comprising the nitrite compound formulation in which the osmolality is less than about 1000 mOsmol/kg and the nitrite compound is present at a molar concentration relative to the pH buffer that exceeds 150:1, 200:1, 250:1, 300:1, 400:1 or 500:1, the formulation further comprising a taste-masking agent. In a further embodiment the taste-masking agent comprises sodium saccharin.

According to certain other embodiments there is provided a nebulized liquid particle of about 0.1 to about 5 microns volumetric mean diameter, comprising an aqueous solution having a pH of from about 4.7 to about 6.5, the solution comprising sodium nitrite; sodium saccharin; and citric acid, wherein (i) sodium nitrite is present in the solution, relative to sodium saccharin, at a molar ratio of from about 1.3×10³:1 to about 4.4×10³:1, (ii) sodium nitrite is present in the solution, relative to citric acid, at a molar ratio of from about 2.0×10²:1 to about 6.9×10²:1, and (iii) the nebulized liquid particle is present in an aerosol that comprises from 12 parts per billion to 1800 parts per billion nitric oxide.

In certain other embodiments there is provided a method of delivering a nitrite compound to a pulmonary bed, comprising administering by inhalation one or a plurality of nebulized liquid particles as described above. In certain embodiments the one or a plurality of nebulized liquid particles is selected from (a) the nebulized liquid particle which further comprises a taste-masking agent, (b) the nebulized liquid particle in which the nitrite compound concentration is at least 16.7 mg NO₂⁻/mL, the liquid particle further comprising a taste-masking agent, (c) the nebulized liquid particle which comprises a nitrite compound formulation in which the osmolality is less than about 650 mOsmol/kg and the nitrite compound is present at a molar concentration relative to the pH buffer that exceeds 150:1, 200:1, 250:1, 300:1, 400:1 or 500:1, and (d) the nebulized liquid particle which comprises a nitrite compound formulation in which the osmolality is less than about 1000 mOsmol/kg and the nitrite compound is present at a molar concentration relative to the pH buffer that exceeds 150:1, 200:1, 250:1, 300:1, 400:1 or 500:1, the formulation further comprising a taste-masking agent. In certain further embodiments the taste-masking agent comprises sodium saccharin.

In another embodiment there is provided a method for delivering a therapeutically effective amount of a nitrite compound to a pulmonary bed, comprising (a) admixing (i) a nitrite compound aqueous solution having a pH greater than 7.0, and (ii) an acidic excipient aqueous solution, to form a nitrite compound formulation, wherein (1) the nitrite compound is present at a concentration of from about 0.667 mg NO₂⁻/mL to about 100 mg NO₂⁻/mL, and (2) the nitrite compound formulation has a pH of from about 47 to about 6.5; (b) nebulizing, within a time period of less than 6, 5, 4, 3, 2, 1, 0.75, 0.5, or 0.25 hour after said step of admixing, the nitrite compound formulation of (a) to form an aerosol comprising liquid particles of about 0.1 to about 5 microns volumetric mean diameter, wherein said aerosol comprises from 12 parts per billion to 1800 parts per billion nitric oxide; and (c) administering by inhalation the aerosolized suspension of (b), and thereby delivering a therapeutically effective amount of a nitrite compound to a pulmonary bed. In one embodiment the method comprises a peak period of nitrite compound delivery to the pulmonary bed of at least 60 minutes following inhalation. In another embodiment the method comprises a peak period of nitrite compound delivery to the pulmonary bed of at least 35 minutes following the step of admixing.

In another embodiment there is provided a nitrite compound formulation for pulmonary delivery, comprising an aqueous solution having a pH of from about 417 to about 6.5, the solution comprising sodium nitrite and citric acid, wherein sodium nitrite is present in the solution, relative to citric acid, at a molar ratio of from about 2.0×10²:1 to about 6.9×10²:1. In another embodiment there is provided a nitrite compound formulation for pulmonary delivery, comprising an aqueous solution having a pH of from about 4.7 to about 6.5, the solution comprising sodium nitrite and sodium saccharin, wherein sodium nitrite is present in the solution, relative to sodium saccharin, at a molar ratio of from about 1.3×10³:1 to about 4.4×10³:1. In certain further embodiments, upon nebulization (e.g., vibrating-mesh nebulization) into liquid particles of about 0.1 to about 5 microns volumetric mean diameter, the nitrite compound formulation produces an aerosol that comprises from 12 parts per billion to 1800 parts per billion nitric oxide. In certain other embodiments, the nitrite compound formulation is selected from (a) the nitrite compound formulation which further comprises a taste-masking agent, (b) the nitrite compound formulation in which the nitrite compound concentration is at least 16.7 mg NO₂⁻/mL, the formulation further comprising a taste-masking agent, (c) the nitrite compound formulation in which the osmolality is less than about 650 mOsmol/kg and the nitrite compound is present at a molar concentration relative to the pH buffer that exceeds 150:1, 200:1, 250:1, 300:1, 400:1 or 500:1, and (d) the nitrite compound formulation in which the osmolality is less than about 1000 mOsmol/kg [50 mg NO₂⁻/mL] and the nitrite compound is present at a molar concentration relative to the pH buffer that exceeds 150:1, 200:1, 250:1, 300:1, 400:1 or 500:1, the formulation further comprising a taste-masking agent, which in certain still further embodiments comprises sodium saccharin.

In another embodiment there is provided a nebulized liquid particle of about 0.1 to about 5 microns volumetric mean diameter, comprising an aqueous solution having a pH of from about 4.7 to about 6.5, the solution comprising sodium nitrite and citric acid, wherein (i) sodium nitrite is present in the solution, relative to citric acid, at a molar ratio of from about 2.0×10²:1 to about 6.9×10²:1, and (ii) the nebulized liquid particle is present in an aerosol that comprises from 12 parts per billion to 1800 parts per billion nitric oxide. In another embodiment there is provided a nebulized liquid particle of about 0.1 to 5 microns volumetric mean diameter, comprising an aqueous solution having a pH of from about 4.7 to about 6.5, the solution comprising sodium nitrite and sodium saccharin, wherein (i) sodium nitrite is present in the solution, relative to sodium saccharin, at a molar ratio of from about 1.3×10³:1 to about 4.4×10³:1, and (ii) the nebulized liquid particle is present in an aerosol that comprises from 12 parts per billion to 1800 parts per billion nitric oxide.

Another embodiment as disclosed herein provides a nitrite compound formulation composition for pulmonary delivery, comprising (a) sodium nitrite dissolved in a liquid solution at a concentration of at least 50 mg/mL; and (b) a taste-masking agent. In another embodiment there is provided a nitrite compound formulation composition for pulmonary delivery, comprising (a) sodium nitrite dissolved in a liquid solution at a concentration of at least 25 mg/mL; (b) an acidic excipient dissolved in the liquid solution; and (c) a taste-masking agent. In certain embodiments the acidic excipient comprises citric acid at a molar ratio relative to sodium nitrite of 1:150, 1:200 or 1:250. In certain embodiments the taste-masking agent comprises sodium saccharin.

According to certain preferred embodiments of the nitrite compound formulation composition disclosed herein, pulmonary delivery is by inhalation. According to certain preferred embodiments of the nitrite compound formulation disclosed herein, pulmonary delivery is by inhalation. According to certain preferred embodiments of the nebulized liquid particle disclosed herein, the nebulized liquid particle is for pulmonary delivery by inhalation.

According to a certain embodiment of the present invention, there is provided a nitrite compound formulation composition for pulmonary delivery, comprising (a) a nitrite compound aqueous solution having a pH of from about 7.0 to about 9.0; and (b) a taste-masking excipient, wherein the nitrite compound formulation has the following characteristics: (i) the nitrite compound is present at a concentration of from about 0.667 mg NO₂⁻/mL to about 100 mg NO₂⁻/mL, (ii) the nitrite compound formulation has a pH of from about 7.0 to about 9.0, and (iii) the nitrite compound formulation contains a taste-masking excipient, wherein the molar ratio of nitrite relative to the taste-masking agent exceeds 10:1, 100:1, 1000:1, 2000:1, 4000:1, 8000:1, or 10000:1.

In another embodiment, there is provided a nitrite compound formulation for pulmonary delivery, comprising: an aqueous solution having a pH of from about 7.0 to about 9.0 and an osmolality of from about 100 to about 3600 mOsmol/kg, wherein the solution comprises: (i) a nitrite compound at a concentration of from about 0.667 mg NO₂⁻/mL to about 100 mg NO₂⁻/mL; and (ii) a pH buffer having a pKa between about 7.0 and 9.0, wherein upon nebulization (e.g., vibrating-mesh nebulization), the nitrite compound formulation forms an aerosol that comprises liquid particles of about 0.1 to about 5.0 microns volumetric mean diameter. In further embodiments, the nitrite compound formulation is selected from: (a) the nitrite compound formulation which further comprises a taste-masking agent, (b) the nitrite compound formulation in which the nitrite compound concentration is at least 16.7 mg NO₂⁻/mL, and the formulation further comprises a taste-masking agent, (c) the nitrite compound formulation in which the osmolality is less than about 650 mOsmol/kg and the nitrite compound is present at a molar concentration relative to the pH buffer that exceeds 10:1, 75:1, 150:1, 200:1, 250:1, 300:1, 400:1, 500:1 or 1000:1, (d) the nitrite compound formulation in which the osmolality is less than about 1200 mOsmol/kg [50 mg NO₂⁻/mL] wherein the nitrite compound is present at a molar concentration relative to the pH buffer that exceeds 10:1, 75:1, 150:1, 200:1, 250:1, 300:1, 400:1, 500:1 or 1000:1, and (e) the nitrite compound formulation in which the osmolality is less than about 2400 mOsmol/kg [100 mg NO₂⁻/mL] and the nitrite compound is present at a molar concentration relative to the pH buffer that exceeds 10:1, 75:1, 150:1, 200:1, 250:1, 300:1, 400:1, 500:1 or 1000:1, wherein the formulation further comprises a taste-masking agent. In certain still further embodiments, the taste-masking agent comprises sodium saccharin. In certain other embodiments, the pH buffer is selected from one or more of 2-amino-2-methyl-1,3-propanediol, N-(2-acetamido)-2-aminoethanesulfonic acid (ACES), N-(2-ametamino)iminodiacetic acid (ADA), N-(1,1-dimethyl-2-hydroxyethyl)-3-amino-2-hydroxypropane-sulfonic acid (AMPSO), N,N-Bis(2-hydroxyethyl)-2-aminoethanesulfonic acid (BES), N,N-Bis(2-hydroxyethyl)glycine (BICINE), Bis(2-hydroxytheyl(amino-tris(hydroxymethyl)methane (BIS-TRIS), 1,3-Bis[tris(hydroxymethyl)methylamino]propane (BIS-TRIS Propane), 2-(cyclohexylamino)ethanesulfonic acid (CHES), 3-(N,N-Bis[2-hydroxyethyl]amino)-2-hydroxypropanesulfonic acid (DIPSO), N-(2-hydroxyethyl)piperazine-N′-(3-propanesulfonic acid) (EPPS), Diglycine, N-(2-hydroxyethyl)piperazine-N′-(4-butanesulfonic acid) (HEPBS), N-(2-hydroxyethyl)piperazine-N′-(2-ethanesulfonic acid) (HEPES), 4-morpholinepropanesulfonic acid (MOPS), beta-hydroxy-4-morpholinepropanesulfonic acid (MOPSO), piperazine-N,N′-bis(2-ethanesulfonic acid) (PIPES), piperazine-N,N′-bis(2-hydroxypropanesulfonic acid) (POPSO), Sodium phosphate dibasic, Sodium phosphate monobasic, Potassium phosphate dibasic, Potassium phosphate monobasic, [(2-hydroxy-1,1-bis(hydroxymethyl)ethyl)amino]-1-propane-sulfonic acid (TAPS), 2-hydroxy-3-[tris(hydroxymethyl)methylamino]-propanesulfonic acid (TAPSO), N-[tris(hydroxymethyl)methyl]-2-aminoethanesulfonic acid (TES), Tricine, 2-amino-2-(hydroxymethyl)-1,3-propanediol, where each has a selected pKa between 6.5 and 9.3.

Certain embodiments also provide a nitrite compound formulation for pulmonary delivery, comprising: an aqueous solution having a pH of from about 7.0 to about 9.0, the solution comprising sodium nitrite; and sodium saccharin, wherein sodium saccharin is present at a concentration selected from: (i) about 0.1 mM to about 2.0 mM, or (ii) about 0.1 mM to about 5.0 mM. In a further embodiment, upon nebulization (e.g., vibrating-mesh nebulization) of the formulation, the formulation forms an aerosol comprising liquid particles of about 0.1 to about 5 microns volumetric mean diameter.

In certain embodiments there is provided a pharmaceutically acceptable nitrite compound formulation composition for pulmonary delivery, comprising (a) a nitrite compound aqueous solution having a final pH greater than 7.0, but less than 9.0; containing (i) the nitrite compound at a concentration of from about 0.667 mg NO₂⁻/mL to about 100 mg NO₂⁻/mL; (ii) a taste-masking agent; and (iii) a pH buffering agent. In certain embodiments the taste-masking agent is sodium saccharin. In certain embodiments the sodium saccharin is at a concentration of 0.1 mM to 2.0 mM. In certain embodiments the pH buffering agent has a pKa from about 6.5 to about 9.3 and is present at a concentration sufficient to maintain a pH from about 7.0 to about 9.0. In certain embodiments the pH buffering agent is sodium phosphate. In certain embodiments the sodium phosphate is at a concentration from about 0.1 mM to about 5.0 mM. In certain embodiments upon nebulization (e.g., vibrating-mesh nebulization) of the nitrite compound formulation composition, the composition forms an aerosol comprising liquid particles of about 0.1 to 5.0 microns volumetric mean diameter. In certain embodiments the pH buffering agent comprises one or more agents selected from 2-amino-2-methyl-1,3-propanediol, ACES, ADA, AMPSO, BES, BICINE, BIS-TRIS, BIS-TRIS Propane, CHES, DIPSO, EPPS, Diglycine, HEPBS, HEPES, MOPS, MOPSO, PIPES, POPSO, sodium phosphate dibasic, sodium phosphate monobasic, potassium phosphate dibasic, potassium phosphate monobasic, TAPS, TAPSO, TES, Tricine, and TRIZMA.

In certain embodiments there is provided a pharmaceutically acceptable nitrite compound formulation for pulmonary delivery, comprising an aqueous solution having a final pH of from about 7.0 to about 9.0 and an osmolality of from about 100 to about 3600 mOsmol/kg, the solution comprising (i) a nitrite compound at a concentration of from about 0.667 mg NO₂⁻/mL to about 100 mg NO₂⁻/mL; (ii) a taste-masking agent; and (iii) a pH buffer having a pKa between 6.5 and 9.3, wherein upon nebulization (e.g., vibrating-mesh nebulization), the nitrite compound formulation forms an aerosol that comprises liquid particles of about 0.1 to about 5.0 microns volumetric mean diameter. In certain embodiments the osmolality is selected from (a) the osmolality that is less than about 300 mOsmol/kg (b) the osmolality that is less than about 600 mOsmol/kg (c) the osmolality that is less than about 1200 mOsmol/kg; (d) the osmolality that is less than about 2400 mOsmol/kg; and (e) the osmolality that is less than about 3000 mOsmol/kg. In certain embodiments the taste-masking agent comprises sodium saccharin. In certain embodiments the pH buffer is one or more of 2-amino-2-methyl-1,3-propanediol, ACES, ADA, AMPSO, BES, BICINE, BIS-TRIS, BIS-TRIS Propane, CHES, DIPSO, EPPS, Diglycine, HEPBS, HEPES, MOPS, MOPSO, PIPES, POPSO, sodium phosphate dibasic, Sodium phosphate monobasic, potassium phosphate dibasic, potassium phosphate monobasic, TAPS, TAPSO, TES, Tricine, and TRIZMA.

In certain embodiments there is provided a pharmaceutically acceptable nitrite compound formulation for pulmonary delivery, comprising (i) a nitrite compound at a concentration of from about 0.667 mg NO₂⁻/mL to about 100 mg NO₂⁻/mL; (ii) a taste-masking agent; and (iii) a pH buffer having a pKa between 6.5 and 9.3, wherein upon nebulization (e.g., vibrating-mesh nebulization), the nitrite compound formulation forms an aerosol that comprises liquid particles of about 0.1 to about 5.0 microns volumetric mean diameter, in certain embodiments the formulation has an osmolality selected from (a) the osmolality that is less than about 300 mOsmol/kg (b) the osmolality that is less than about 600 mOsmol/kg (c) the osmolality that is less than about 1200 mOsmol/kg; (d) the osmolality that is less than about 2400 mOsmol/kg; and (e) the osmolality that is less than about 3000 mOsmol/kg. In certain embodiments the taste-masking agent comprises sodium saccharin. In certain embodiments the pH buffer is sodium phosphate.

In certain embodiments there is provided a pharmaceutically acceptable nitrite compound formulation for pulmonary delivery, comprising (i) an aqueous solution having a final pH of from about 7.0 to about 9.0; (ii) sodium nitrite at a concentration of from about 0.667 mg NO₂⁻/mL to about 100 mg NO₂⁻/mL; (iii) sodium saccharin at a concentration of from about 0.1 mM to about 2.0 mM; and (iv) sodium phosphate at a concentration of from about 0.1 mM to about 5.0 mM. In certain embodiments upon nebulization (e.g., vibrating-mesh nebulization) of the formulation, an aerosol comprising liquid particles of about 0.1 to about 5 microns volumetric mean diameter is formed. In certain embodiments there is provided a pharmaceutically acceptable nebulized liquid particle of about 0.1 to 5 microns volumetric mean diameter that is formed by a method comprising (1) nebulizing a nitrite compound formulation in at least one of a vibrating-mesh nebulizer and a jet nebulizer to obtain an aerosol that comprises said nebulized liquid particle, wherein the nitrite compound formulation comprises (i) a nitrite compound at a concentration of from about 0.667 mg NO₂⁻/mL to about 100 mg NO₂⁻/mL; (ii) a taste-masking agent; and (iii) a pH buffer having a pKa between 6.5 and 9.3. In certain embodiments the nebulized nitrite compound formulation has an osmolality of from about 100 to about 3000 mOsmol/kg. In certain embodiments the nebulized nitrite compound formulation has an osmolality selected from (a) the osmolality that is less than about 300 mOsmol/kg (b) the osmolality that is less than about 600 mOsmol/kg (c) the osmolality that is less than about 1200 mOsmol/kg; (d) the osmolality that is less than about 2400 mOsmol/kg; and (e) the osmolality that is less than about 3000 mOsmol/kg. In certain embodiments the taste-masking agent comprises sodium saccharin. In certain embodiments the pH buffer is at least one (i.e., one or more) agent selected from the group consisting of 2-amino-2-m-ethyl-1,3-propanediol, ACES, ADA, AMPSO, BES, BICINE, BIS-TRIS, BIS-TRIS Propane, CHES, DIPSO, EPPS, Diglycine, HEPBS, HEPES, MOPS, MOPSO, PIPES, POPSO, sodium phosphate dibasic, sodium phosphate monobasic, potassium phosphate dibasic, potassium phosphate monobasic, TAPS, TAPSO, TES, Tricine, and TRIZMA. In certain embodiments the pH buffer is sodium phosphate.

In certain embodiments there is provided a method for delivering a therapeutically effective amount of a pharmaceutically acceptable nitrite compound to a pulmonary bed in a subject in need of such delivery, comprising (a) nebulizing a nitrite compound formulation that comprises an aqueous solution having a pH of from about 7.0 to about 9.0, wherein the solution comprises (i) sodium nitrite from about 0.667 mg NO₂⁻/mL to about 100 mg NO₂⁻/mL; (ii) sodium saccharin from about 0.1 mM to about 2.0 mM; and (iii) sodium phosphate from about 0.1 mM to about 5.0 mM to form an aerosol comprising liquid particles of about 0.1 to about 5 microns volumetric mean diameter; and (b) administering by inhalation the aerosol of (a) and thereby delivering a therapeutically effective amount of the nitrite compound to the pulmonary bed. In certain embodiments administering comprises administering for a peak period of nitrite compound delivery to the pulmonary bed within 60 minutes following initiation of inhalation. In certain embodiments administering comprises administering for a peak period of nitrite compound delivery to the pulmonary bed within 35 minutes following initiation of inhalation. In certain embodiments administering comprises administering for a peak period of nitrite compound delivery to the pulmonary bed within 25 minutes following initiation of inhalation. In certain embodiments administering comprises administering for a peak period of nitrite compound delivery to the pulmonary bed within 15 minutes following initiation of inhalation. In certain embodiments administering comprises administering for a peak period of nitrite compound delivery to the pulmonary bed within 10 minutes following initiation of inhalation, in certain embodiments administering comprises administering for a peak period of nitrite compound delivery to the pulmonary bed within 5 minutes following initiation of inhalation.

In certain embodiments there is provided a pharmaceutically acceptable nitrite compound formulation composition for pulmonary delivery, comprising (a) sodium nitrite dissolved in a liquid solution at a concentration of at least 90 mg/mL, the solution having a final pH of from about 7.0 to about 9.0; (b) sodium saccharin at a concentration of from about 0.1 mM to about 2.0 mM; and (c) sodium phosphate at a concentration of from about 0.1 mM to about 5.0 mM.

In certain embodiments there is provided a pharmaceutically acceptable nitrite compound formulation composition for pulmonary delivery, comprising (a) a liquid solution that comprises sodium nitrite dissolved at a concentration of at least 70 mg/mL, the solution having a final pH of from about 7.0 to about 9.0; (b) sodium saccharin at a concentration of from about 0.1 mM to about 2.0 mM; and (c) sodium phosphate at a concentration of from about 0.1 mM to about 5.0 mM.

In certain embodiments there is provided a pharmaceutically acceptable nitrite compound formulation composition for pulmonary delivery, comprising (a) a liquid solution that comprises sodium nitrite dissolved at a concentration of at least 50 mg/mL, the solution having a final pH of from about 7.0 to about 9.0; (b) sodium saccharin at a concentration, of from about 0.1 mM to about 2.0 mM; and (c) sodium phosphate at a concentration from about 0.1 mM to about 5.0 mM.

In certain embodiments there is provided a pharmaceutically acceptable nitrite compound formulation composition for pulmonary delivery, comprising (a) a liquid solution that comprises sodium nitrite dissolved at a concentration of at least 30 mg/mL, the solution having a final pH of from about 7.0 to about 9.0; (b) sodium saccharin at a concentration of from about 0.1 mM to about 2.0 mM; and (c) sodium phosphate at a concentration of from about 0.1 mM to about 5.0 mM.

In certain embodiments there is provided a pharmaceutically acceptable nitrite compound formulation composition for pulmonary delivery, comprising (a) a liquid solution that comprises sodium nitrite dissolved at a concentration of at least 20 mg mL, the solution having a final pH of from about 7.0 to about 9.0; (b) sodium saccharin at a concentration of from about 0.1 mM to about 2.0 mM; and (c) sodium phosphate at a concentration of from about 0.1 mM to about 5.0 mM.

In certain embodiments there is provided a pharmaceutically acceptable nitrite compound formulation composition for pulmonary delivery, comprising (a) a liquid solution that comprises sodium nitrite dissolved at a concentration of at least 10 mg/mL, the solution having a final pH of from about 7.0 to about 9.0; (b) sodium saccharin at a concentration of from about 0.1 mM to about 2.0 mM; and (c) sodium phosphate at a concentration of from about 0.1 mM to about 5.0 mM.

In certain embodiments there is provided a pharmaceutically acceptable nitrite compound formulation composition for pulmonary delivery, comprising (a) a liquid solution that comprises sodium nitrite dissolved at a concentration of at least 5 mg/mL or at least 1 mg/mL, the solution having a final pH of from about 7.0 to about 9.0; (b) sodium saccharin at a concentration of from about 0.1 mM to about 2.0 mM; and (c) sodium phosphate at a concentration of from about 0.1 mM to about 5.0 mM.

In certain further embodiments of any of the above described nitrite compound formulation compositions, pulmonary delivery is by inhalation. In certain further embodiments the above described nitrite compound formulations, or the above described nebulized liquid particles, are for pulmonary delivery by inhalation.

In certain embodiments there is provided a method of treating pulmonary arterial hypertension or ischemic reperfusion injury comprising administering to a subject in need thereof a therapeutically effective dose of a nitrite compound formulation composition as described herein, or of a nitrite compound formulation as also described herein. In certain embodiments the ischemic reperfusion injury is associated with coronary heart disease, stroke, or transplant. In certain embodiments the pulmonary arterial hypertension (PAH) is Group I PAH, Group II pulmonary hypertension (pulmonary venous hypertension), Group III pulmonary hypertension (pulmonary hypertension associated with lung diseases and/or hypoxeria, Group IV pulmonary hypertension (pulmonary hypertension due to chronic thrombotic and/or embolic disease, or Group V pulmonary hypertension, including, histiocytosis X, lymphangiomatosis, and/or other pathology causing compression of pulmonary vessels.

In certain embodiments there is provided a kit, comprising (a) a pharmaceutically acceptable nitrite formulation, said formulation comprising a nitrite compound aqueous solution having a final pH greater than 7.0, but less than 9.0 and containing (i) the nitrite compound at a concentration of from about 0.667 mg NO₂⁻/mL to about 100 mg NO₂⁻/mL; (ii) a taste-masking agent; and (iii) a pH buffering agent; and (b) a nebulizer adapted to aerosolize the nitrite formulation of (a). In certain embodiments the taste-masking agent is sodium saccharin. In certain embodiments the pH buffer is sodium phosphate.

According to certain embodiments there is provided a method of treating pulmonary arterial hypertension or ischemic reperfusion injury comprising administering, via inhalation using a nebulizer, to a subject in need thereof a therapeutically effective dose of a nitrite liquid compound formulation composition wherein the nebulizer delivers to the subject an inhaled aerosol containing about 0.25 to 90 mg sodium nitrite, in particles of less than 5 microns volumetric mean. In another embodiment there is provided an aerosolizing device loaded with a liquid sodium nitirite formulation so that the device contains about 1 to about 360 mg sodium nitrite wherein said device delivers to the subject an aerosol containing about 0.25 to 90 mg sodium nitrite in particles of less than 5 microns volumetric mean diameter, in another embodiment there is provided an aerosolizing device loaded with a liquid sodium nitirite formulation so that the device contains about 0.36 to about 129 mg sodium nitrite wherein said device delivers to the subject an aerosol containing about 0.25 to 90 mg sodium nitrite in particles of less than 5 microns volumetric mean diameter.

In another embodiment there is provided a method of treating pulmonary arterial hypertension or ischemic reperfusion injury comprising administering, via inhalation using a dry powder inhaler, to a subject in need thereof a therapeutically effective dose of a dry powder nitrite compound formulation composition wherein the dry powder inhaler delivers to the subject an aerosol containing about 0.18 to 18 mg sodium nitrite in particles of less than 5 microns volumetric mean diameter. In another embodiment there is provided a dry powder inhaler for single or multiple dosing loaded with a dry powder sodium nitrite formulation so that the dry powder inhaler contains about 0.35 mg to about 35 mg per inhalation breath of sodium nitrite wherein said dry powder inhaler delivers to the subject an aerosol containing about 0.18 mg to about 18 mg sodium nitirite in particles of less than 5 microns mean diameter per inhalation breath. In certain further embodiments of the above described methods, the administration of the sodium nitrite results in about 0.1 μM to about 10 μM peak plasma nitrite. In certain further embodiments of the above described aerosolizing device or dry powder inhaler, the delivery results in about 0.1 μM to about 10 μM peak plasma nitrite. In certain further embodiments of the above described nitrite compound formulation composition, the nitrite is sodium nitrite. In certain further embodiments of the above described nitrite compound formulation composition, pulmonary delivery is by inhalation.

These and other aspects of the invention will be evident upon reference to the following detailed description and attached drawings. All of the U.S. patents, U.S. patent application publications, U.S. patent applications, foreign patents, foreign patent applications and non-patent publications referred to in this specification and/or listed in the Application Data Sheet, are incorporated herein by reference in their entireties, as if each was incorporated individually. Aspects of the invention can be modified, if necessary, to employ concepts of the various patents, applications and publications to provide yet further embodiments of the invention.

### BRIEF DESCRIPTION OF THE SEVERAL VIEWS OF THE DRAWINGS

FIG. 1 shows effects of inhaled sodium nitrite on PAP and nitric oxide production. Isolated rabbit lungs were cannulated in the pulmonary artery and perfused with buffer containing ˜12% hematocrit. Lungs were ventilated as described by Weissmann et al 2001, and pulmonary/arterial pressures were monitored by pressure transducers. After system stabilization, hypoxic maneuvers were induced by lowering the oxygen content to 3% over 15 minute periods which resulted in increased PAP. The effect of sodium nitrite prepared in either phosphate buffer (PB) or citric acid (CA)/phosphate buffer (both at pH 5.5, n=5/6 per group) was then administered via nebulization during the second hypoxic challenge. FIG. 1, Left panel: sodium nitrite in both buffer systems significantly decreased PAP (over 50%) compared with pre-drug hypoxic challenge (p<0.05). FIG. 1, Right panel: expired nitric oxide was significantly increased by both sodium nitrite preparations compared to control, but sodium nitrite prepared in citric acid produced significantly more nitric oxide prepared in phosphate buffer only (p<0.05). *Indicates significant difference from control, **indicates significant difference from nitrite in phosphate buffer.

FIG. 2 shows sustained-effect of inhaled sodium nitrite on PAP. Isolated rabbit lungs were cannulated in the pulmonary artery and perfused as described in FIG. 1. After system stabilization, hypoxic maneuvers were induced by lowering the oxygen content to 3% over 15 minute periods which resulted in increased PAP. The effect of sodium nitrite prepared in phosphate buffer was then administered via nebulization during the third hypoxic challenge. The sustained effect is measured as a function of time to return to the same level of hypoxia-induced PAP as that measured prior to dosing. Half life is calculated as ˜10 min, with a sustained effect being ≧60 min.

FIG. 3 shows a dose-dependent relaxation of isolated rat aortic ring in the presence of increasing concentrations of Sildenafil. The isolated rat aortic ring model tests whether a drug solution reduces the phenylephrine-induced pre-contractions of aortic rings. Briefly, rat aorta was excised and cleansed of fat and adhering tissue. Vessels were then cut into individual ring segments (2-3 mm in width) and suspended from a force-displacement transducer in a tissue bath. Ring segments were bathed in a bicarbonate-buffered, Krebs-Henseleit (KH) solution of the following composition (mM): NaCl 118; KCl 4.6; NaHCO₃ 2.72; KH₂PO₄ 1.2; MgSO₄ 1.2; CaCl₂ 1.75; Na₂EDTA 0.03, and glucose 11.1. A passive load of 2 grams was applied to all ring segments and maintained at this level throughout the experiments. At the beginning of each experiment, indomethacin-treated ring segments were depolarized with KCl (70 mM) to determine the maximal contractile capacity of the vessel. Rings were then washed extensively and allowed to equilibrate. For subsequent experiments, vessels were submaximally contracted (50% of KCl response) with phenylephrine (PE) (3×10⁻⁸-10⁻⁷ M).

FIG. 4 shows a dose-dependent relaxation of isolated rat aortic ring in the presence of increasing concentrations of sodium nitrite (solid circles) and an additive effect of sodium nitrite in the presence of Sildenafil (at ˜50% the effective dose measured in FIG. 3). Briefly, 50 nM sildenafil was chosen for the sodium nitrite potentiation experiments as this afforded approximately a 50% reduction in phenylephrine-induced aortic constriction. For sodium nitrite potentiation measurements, aortic rings were first exposed to sildenafil at 50 nM to partially reduce aortic ring constriction. After equilibration, increasing amounts of sodium nitrite (500 nM-50 μM) were added to the buffer with tension measurements recorded after each addition.

### DETAILED DESCRIPTION

The present invention provides, in several embodiments as herein disclosed, compositions and methods for nitrite compound formulations that offer unprecedented advantages with respect to localized delivery of nitrite anion in a manner that permits both rapid and sustained availability of therapeutically useful nitric oxide (NO) and or nitrite levels to one or more desired tissues.

In certain preferred embodiments, and as described in greater detail below, delivery of the nitrite compound formulation is to the respiratory tract tissues in mammalian subjects, for example, via the respiratory airways to pulmonary beds (e.g., aiveolar capillary beds) in human patients. According to certain particularly preferred embodiments, delivery to pulmonary beds is achieved by inhalation therapy of a nitrite compound formulation as described herein.

These and related embodiments will usefully provide therapeutic and/or prophylactic benefit, by making therapeutically effective NO and or nitrite available to a desired tissue promptly upon administration, while with the same administration event also offering time periods of surprisingly sustained duration during which locally delivered nitrite anion is converted into bioavailable NO, for a prolonged therapeutic effect.

The compositions and methods disclosed herein provide for such rapid and sustained localized delivery of a nitrite compound and its product, NO, to a wide variety of tissues. Contemplated are embodiments for the treatment of numerous clinically significant conditions including ischemia-reperfusion injury and pulmonary arterial hypertension and other conditions, as may pertain, for example, in stroke, heart attack or other cardiovascular disease, transplantation (e.g. lung, liver, kidney, heart, etc.) or vascular grafts, and/or other conditions for which rapid and sustained bioavailable NO therapy may be indicated.

Various embodiments thus provide compositions and methods for optimal prophylactic and therapeutic activity in prevention and treatment of pulmonary hypertension in human and/or veterinary subjects using aerosol administration, and through the delivery of high-concentration, sustained-release active drug exposure directly to the affected tissue. Specifically, and in certain preferred embodiments, concentrated doses are delivered of a nitrite compound, which includes nitrite anion (NO₂⁻) or any nitrite salt, for example, sodium nitrite, potassium nitrite or magnesium nitrite.

Without wishing to be bound by theory, according to certain of these and related embodiments as described in greater detail herein, a nitrite compound (e.g., nitrite anion (NO₂⁻) or any nitrite salt, for example, sodium nitrite, potassium nitrite or magnesium nitrite) is provided in a nitrite compound formulation having components that are selected to permit gradual reduction of the nitrite compound to yield bioavailable nitric oxide, in a manner that provides for continua and sustained NO generation in vivo, and by a formulation that does not result in rapid loss from the formulation of substantial amounts of NO as an evolved gas. Instead, the embodiments disclosed herein derive from the discovery that regulation of the solution parameters of nitrite compound concentration and pH can result in a nitrite compound formulation in which NO is slowly generated and remains dissolved in solution. Additionally, regulation of pH and of total solute concentration in the formulation, as shown herein by selection of appropriate nitrite formulation components, is believed to result in a desirably sustained release of bioavailable NO following in vivo administration of the formulation. Moreover, nitrite itself may itself be responsible for some or all of the therapeutic effects described herein.

Further according to non-limiting theory, by advantageously retaining NO as a liquid-dissolved solute instead of losing gaseous NO to the gas-phase environment, certain nitrite compound formulations disclosed herein permit inhalation delivery to pulmonary beds of higher NO concentrations, and which higher NO concentrations are sustained at the pulmonary beds for longer time periods without the need for commensurately prolonged inhalation administration events, than was previously believed possible. This inhalation delivery may also include the use of nebulizer devices that generate aerosol mists having controlled liquid particle sizes such as vibrating-mesh nebulizers, which would not be capable of delivering nitrite solutions in which are present NO gas bubbles caused by high levels of nitrite-to-NO conversion. As such, it is disclosed herein for the first time that significant benefits derive from selecting a nitrite compound formulation, as provided herein, the components of which do not permit generation of more NO gas than can be retained in the dissolved state by the aqueous formulation solution.

According to certain related embodiments, regulation of the total amount of dissolved solutes in a nitrite compound formulation is believed, according to non-limiting theory, to result in aqueous nitrite compound formulations having therapeutically beneficial properties, including the properties of nebulized liquid particles formed from aqueous solutions of such formulations. Additionally, and as disclosed herein, it has been discovered that within the parameters provided herein as pertain to nitrite compound concentration, pH, and total solute concentration, tolerability of formulations at or near the upper portion of the total solute concentration range can be increased by inclusion of a taste-masking agent as provided herein.

In certain such embodiments, for example, a nitrite compound formulation that comprises sodium nitrite dissolved in aqueous solution (pH from about 4.7 to about 6.5) at a concentration of at least 25 mg/mL, or at least 50 mg/mL, or a nitrite compound at a concentration of from about 14.5 mM nitrite anion to 2.174 M nitrite anion in an aqueous solution having total osmolality from about 100 to 3600 mOsmol/kg, may further comprise a taste-masking agent thereby to become tolerable for inhalation administration (i.e., to overcome undesirable taste or irritative properties that would otherwise preclude effective therapeutic administration). Hence and as described in greater detail herein, regulation of formulation conditions with respect to one or more of (i) solution pH, (ii) molar ratio of nitrite compound to acidic excipient or pH buffer, (iii) rate of nitrite anion reduction to NO such that NO is retained in solution and is not evolved as visible bubbles, (iv) molar ratio of nitrite anion to taste-masking agent, and (v) total osmolality of the formulation, provides certain therapeutic and other advantages.

As noted above, in certain preferred embodiments, a nitrite compound comprises nitrite anion (NO₂⁻) or any nitrite salt thereof, for example, sodium nitrite, potassium nitrite or magnesium nitrite, or the like. Other embodiments contemplate agents selected from other nitrite- or nitric oxide-donating compounds. By non-limiting example, nitrite (NO₂⁻), nitrate (NO₃⁻), nitrous acid (HNO₂), nitrogen dioxide (NO₂ gas), nitrite-donating compounds, nitric oxide-donating compounds, nitric oxide (NO gas) itself, or salts thereof may serve as prodrugs, sustained-release or active substances in the presently disclosed formulations and compositions and may be delivered, under conditions and for a time sufficient to produce maximum concentrations (e.g., without appreciable loss by the nitrite compound formulation to the environment, prior to administration, of NO formed therein as evolved NO gas, which loss may be less than about 40%, 30%, 20%, 15%, 10%, 5%, 3%, 2% or 1% of total NO present in the nitrite compound formulation within the first 15 minutes of its preparation) of sustained-release or active drug, to the respiratory tract (including pulmonary beds), and other non-oral and non-nasal topical compartments including, but not limited to the skin, rectum, vagina, urethra, urinary bladder, eye, and ear. As disclosed herein, certain particularly preferred embodiments relate to ad ministration, via oral and/or nasal inhalation, of a nitrite compound to the lower respiratory tract, in other words, to the lungs or pulmonary compartment (e.g., respiratory bronchioles, alveolar ducts, and/or alveoli), as may be effected by such “pulmonary delivery” to provide effective amounts of the nitrite compound to the pulmonary compartment and/or to other tissues and organs as may be reached via the circulatory system subsequent to such pulmonary delivery of the nitrite compound to the pulmonary vasculature.

Because different drug products are known to have varying efficacies depending on the dose, form, concentration and delivery profile, certain presently disclosed embodiments provide specific formulation and delivery parameters that produce anti-hypertensive, vasodilatory, arteriodilatory, and/or vasculature-remodeling results that are prophylactic or therapeutically significant. These and related embodiments thus preferably include a nitrite compound such as nitrite anion or a salt thereof, e.g., sodium nitrite. As noted above, however, the invention is not intended to be so limited and may relate, according to particularly preferred embodiments, to nitrite anion or a salt thereof such as sodium nitrite, potassium nitrite or magnesium nitrite. Other contemplated embodiments may relate to another agent selected from nitrite- or nitric oxide-donating compounds such as those disclosed herein.

Certain embodiments contemplate a nitrite compound as provided herein (e.g., nitrite anion or a nitrite salt thereof, such as sodium nitrite, potassium nitrite or magnesium nitrite), or alternatively, an agent selected from nitrite- or NO-donating compounds, formulated to permit mist, gas-liquid suspension or liquid nebulized, dry powder and/or metered-dose aerosol administration to supply effective concentrations conferring desired anti-hypertensive, vasodilatory, arteriodilatory, or vasculature-remodeling benefits, for instance, to treat patients with pulmonary hypertension and/or to prevent deleterious vascular remodeling. These and related applications are also contemplated for use in the ischemic environment of diseased pulmonary tissue and associated vasculature. According to non-limiting theory, the relevant disease-associated hypoxic environment will enhance the reduction of nitrite anion or nitrite salt (or nitrite- or nitric oxide-donating compound) to nitric oxide. The nitrite compound formulations and methods described herein may be used with commercially available inhalation devices, or with other devices for aerosol therapeutic product administration.

Certain embodiments provide compositions and methods for optimal prophylactic and therapeutic activity in prevention and treatment of ischemic reperfusion injury of the heart in human and/or veterinary subjects, using aerosol administration (e.g., inhalation) during reperfusion of the heart following or during an ischemic episode as may accompany, for example, a myocardial infarction, a coronary arterial catheterization or a heart transplant. Such embodiments provide for direct and high concentration delivery of the nitrite compound (e.g., nitrite anion or a salt thereof) as a source of sustained-release NO to provide maximum NO levels directly to the pulmonary vasculature immediately upstream of the left atrium and hence, to the coronary arterial system with interlumenal atrial and ventricular exposure.

Because different drug products are known to vary in efficacy depending on the dose, form, concentration and delivery profile, the presently disclosed embodiments provide specific formulation and delivery parameters that produce protection against acute ischemic reperfusion injury and against ischemic reperfusion injury following myocardial infarction or other cardiac ischemic event, such as that created during coronary arterial catheterization.

Certain other embodiments contemplate a nitrite compound (e.g., nitrite anion or nitrite salts), or alternatively, an agent selected from nitrite- or NO-donating compounds, formulated to permit mist, gas-liquid suspension or liquid nebulized, dry powder and/or metered-dose aerosol administration to supply effective concentrations conferring desired blood levels entering the left atrium and coronary arteries to treat and/or prevent ischemic myocardial reperfusion injury. These and related embodiments are contemplated for use in the ischemic environment of diseased myocardium and associated vasculature, or of a manipulated coronary arterial system during such events as catheterization. According to non-limiting theory, the disease-associated hypoxic environment will enhance the reduction of nitrite anion or nitrite salt (or nitrite- or nitric oxide-donating compound) to nitric oxide. The nitrite compound formulations and methods described herein may be used with commercially available inhalation devices, or with other devices for aerosol therapeutic product administration.

Various other embodiments provide compositions and methods for optimal prophylactic and therapeutic activity in prevention and treatment of ischemic reperfusion injury of the brain in human and/or veterinary subjects using aerosol administration during reperfusion of the brain following or during an ischermic episode such as, by way of non-limiting example, an infarction or carotid arterial catheterization. Such exposure provides for direct and high concentration delivery of a nitrite compound as provided herein according to preferred embodiments (e.g., nitrite anion or a salt thereof, such as sodium nitrite, potassium nitrite or magnesium nitrite) or, in other embodiments, of agents selected from other nitrite- or nitric oxide-donating compounds.

As non-limiting examples, in preferred embodiments a nitrite compound such as nitrite anion (NO₂⁻) or a salt thereof (e.g., sodium nitrite, potassium nitrite, magnesium nitrite), or alternatively and in other distinct embodiments, a nitrite- or nitric oxide-donating agent such as nitrate (NO₃⁻) or a salt thereof, nitrous acid (HNO₂), nitrogen dioxide (NO₂ gas), nitric oxide (NO gas) itself, or another nitrite-donating or nitric oxide-donating compound, may serve as a sustained-release or active substance, and may be delivered to produce maximum concentrations of sustained-release or active drug directly to the pulmonary vasculature immediately upstream of the left atrium, left ventrical and hence, carotid arterial system. Because different drug products are known to have varying efficacies depending on the dose, form, concentration and delivery profile, the embodiments described herein provide specific formulation and delivery parameters that confer protection against acute ischemic reperfusion injury and against I/R injury following stroke or other cerebral ischemic event.

Nitrite compounds as provided herein in preferred embodiments (e.g., nitrite anion (NO₂⁻) or a salt thereof), or in distinct embodiments, other nitrite- or nitric oxide-donating agents, may be formulated for liquid nebulized, dry powder and/or metered-dose aerosol administration at suitable dosages to provide desired pulmonary concentrations. From such concentrations sufficient blood levels may be achieved of the nitrite compound (or other agent) in the left atrium of the heart and entering the carotid arteries, as may beneficially treat and/or prevent ischemic reperfusion injury in the brain, such as may follow stroke or infarct, or as may follow carotid arterial catheterization. According to these and related embodiments, it is predicted by way of non-limiting theory that the associated disease-induced hypoxic environment will enhance the reduction of the nitrite compound (or of the nitrite- or nitric oxide-donating agent), to produce nitric oxide. The nitrite compound formulations and methods described herein may be used with commercially available inhalation devices, or with other devices for aerosol therapeutic product administration.

According to other embodiments there are provided compositions and methods for optimal prophylactic and therapeutic activity in prevention and treatment of ischemic reperfusion injury prior to, during or following lung transplantation in human and/or veterinary subjects. For such embodiments, nitrite compounds as provided herein in preferred embodiments (e.g., nitrite anion or salts thereof such as sodium nitrite, potassium nitrite, magnesium nitrite), or other nitrite- or nitric oxide-donating agents, are introduced using aerosol administration, or perfusion and/or washing the donor lung prior to or during transplantation. Such exposure provides for direct and high concentration delivery of the nitrite compound or other nitrite- or NO-donating agent, as may be selected from nitrate (NO₃⁻) or a salt thereof, nitrous acid (HNO₂), nitrogen dioxide (NO₂ gas), or other compound. Maximum concentrations of the nitrite compound or other nitrite- or NO-donating agent provide sustained-release and/or active drug directly to the epithelial surface of the lung and pulmonary vasculature.

Because different drug products are known to have varying efficacies depending on the dose, form, concentration and delivery profile, the embodiments described herein provide specific formulation and delivery parameters that confer protection against ischemic reperfusion injury prior to and during lung transplantation acutely and following lung transplant. Nitrite compounds as provided herein in preferred embodiments, or other nitrite- or nitric oxide-donating agents (such as those disclosed herein), may be formulated for liquid nebulized, dry powder and/or metered-dose aerosol administration at suitable dosages to provide desired pulmonary concentrations that are sufficient to be absorbed directly from the pulmonary epithelial surface into the pulmonary vasculature, as may beneficially treat and/or prevent ischemic reperfusion injury prior to and during lung transplantation. According to these and related embodiments, it is predicted by way of non-limiting theory that the ischemic environment of the donor lung (during the transplant process) will enhance the reduction of the nitrite (compound (e.g., nitrite anion or salt thereof), or of the nitrite- or nitric oxide-donating compound, to produce nitric oxide. The nitrite compound formulations and methods described herein may be used with commercially available inhalation devices, or with other devices for aerosol therapeutic product administration.

Certain other embodiments provide compositions and methods for optimal prophylactic and therapeutic activity in prevention and treatment of ischemic reperfusion injury prior to or during heart transplantation in human and/or veterinary subjects using perfusion and/or washing of the donor heart prior to or during transplantation. Such exposure provides for direct and high concentration delivery of a nitrite compound as provided herein according to preferred embodiments (e.g., nitrite anion or a salt thereof, such as sodium nitrite, potassium nitrite or magnesium nitrite) or, in other embodiments, of agents selected from other nitrite- or nitric oxide-donating compounds, including as non-limiting examples nitrite, nitrate (NO₃⁻) and salts thereof, nitrous acid (HNO₂), nitrogen dioxide (NO₂ gas), nitrite-donating compounds, nitric oxide-donating compounds, and nitric oxide NO gas) itself. These compounds may serve as sustained-release or active substance, and may be delivered to produce maximum concentrations of sustained-release or active drug directly to the epithelial surface of the lung and coronary vasculature. Because different drug products are known to have varying efficacies depending on the dose, form, concentration and delivery profile, the embodiments described herein provide specific formulation and delivery parameters that confer protection against ischemic reperfusion injury prior to or during heart transplantation.

Nitrite compounds as provided herein according to preferred embodiments (e.g., nitrite anion and salts thereof), or, alternatively, nitrite- or nitric oxide-donating agents, may be formulated for liquid perfusion or for washing the donor heart at desired concentrations for sufficient myocardial vascular or tissue levels of the nitrite compound or other agent to be attained, to treat and/or prevent ischemic reperfusion injury prior to and during heart transplantation. According to these and related embodiments, it is predicted by way of non-limiting theory that the ischemia-derived hypoxic environment within the donor heart will enhance the reduction of nitrite anion, nitrite salt, or nitrite- or nitric oxide-donating compound, to produce nitric oxide. These nitrite compound formulations and methods described herein may be used with commercially available inhalation devices, or with other devices for aerosol therapeutic product administration.

Certain other embodiments provide compositions and methods for optimal prophylactic and therapeutic activity in prevention and treatment of ischemic reperfusion injury prior to, during or following kidney transplantation in human and/or veterinary subjects using aerosol administration and/or perfusion and/or washing the donor kidney prior to or during transplantion. Such exposure provides for direct and high concentration delivery of a nitrite compound as provided herein according to preferred embodiments (e.g., nitrite anion or a salt thereof, such as sodium nitrite, magnesium nitrite, potassium nitrite, etc.) or, in other embodiments, of an agent selected from nitrite- or nitric oxide-donating compound. As non-limiting examples, a nitrite compound such as sodium nitrite or, alternatively, nitrate or a salt thereof, nitrous acid, nitrogen dioxide (NO₂ gas), or nitric oxide (NO gas) itself, may serve as a sustained-release or active substance. These compounds may be delivered to produce maximum concentrations of sustained-release or active drug directly to the vasculature, to obtain sufficient blood concentrations for treating or preventing ischemic reperfusion injury during and following kidney transplantation.

Because different drug products are known to have varying efficacies depending on the dose, form, concentration and delivery profile, the embodiments described herein provide specific formulation and delivery parameters that confer protection against ischemic reperfusion injury during and following kidney transplantation. Nitrite compounds as provided herein in preferred embodiments, or alternatively, other nitrite- or NO-donating agents as disclosed herein, may be formulated for liquid nebulized, dry powder and/or metered-dose aerosol administration to provide desired pulmonary concentrations for sufficient blood levels of the nitrite compound or other agent to be attained in blood entering the left atrium as may beneficially treat and/or prevent ischemic reperfusion injury during and following kidney transplantation. According to these and related embodiments, it is predicted by way of non-limiting theory that the ischemia-induced hypoxic environment of the donor kidney (during the transplant process) will enhance the reduction of (in the case of nitrite compounds) nitrite anion, nitrite salt, or (alternatively in the case of other agents disclosed herein) nitrite- or nitric oxide-donating compound, to produce nitric oxide. The nitrite compound formulations and methods described herein may be used with commercially available inhalation devices, or with other devices for aerosol therapeutic product administration.

Certain other embodiments provide compositions and methods for optimal prophylactic and therapeutic activity in prevention and treatment of ischermic reperfusion injury prior to, during or following liver transplantation in human and/or veterinary subjects. For such embodiments, nitrite compounds as provided herein in preferred embodiments (e.g., nitrite anion or salts thereof, such as sodium nitrite) or, alternatively and in other embodiments, other nitrite- or nitric oxide-donating agents, are introduced using aerosol administration, or perfusion and/or washing the donor liver prior to or during transplantion. Such exposure provides for direct and high concentration delivery of (in preferred embodiments) the nitrite compound or (in other embodiments) of other nitrite- or nitric oxide-donating agents, which compound or agents may serve as a sustained-release or active substance, and may be delivered to produce maximum concentrations of sustained-release or active drug directly to the vasculature to obtain sufficient blood concentrations to treat or prevent ischemic reperfusion injury during or following liver transplantation. Because different drug products are known to vary in efficacy depending on the dose, form, concentration and delivery profile, the embodiments described herein provide specific formulation and delivery parameters that confer protection against ischemic reperfusion injury during or following liver transplantation. Nitrite compounds as provided herein in preferred embodiments (e.g., nitrite anion and salts thereof, such as sodium nitrite), or in other embodiments nitrite- or NO-donating agents, may be formulated for liquid nebulized, dry powder and/or metered-dose aerosol administration at suitable doses to provide desired pulmonary concentrations for sufficient blood levels of the nitrite compound or other agent to be attained upon entering the left atrium, as may beneficially treat and/or prevent ischemic reperfusion injury during or following liver transplantation. According to these and related embodiments, it is predicted by way of non-limiting theory that the ischemia-induced hypoxic environment in the donor liver (during the liver transplant process) will enhance the reduction of nitrite anion, nitrite salt, or nitrite- or nitric oxide-donating compound, to produce nitric oxide. The nitrite compound formulations and methods described herein may be used with commercially available inhalation devices, or with other devices for aerosol therapeutic product administration.

Certain other embodiments provide compositions and methods for optimal prophylactic activity in prevention of ischemic reperfusion injury prior to or during organ (by non-limiting example, liver, lung, kidney, heart) transplantation in human and/or veterinary subjects using flush perfusion and/or reperfusion of the organ prior to or during transplantation. For such embodiments, nitrite compounds as provided herein in preferred embodiments (e.g., nitrite anion or salts thereof such as sodium nitrite), or in alternative embodiments, a nitrite- or nitric oxide-donating agent such as those disclosed herein, may act as a sustained-release or active substance that is delivered directly to the epithelial surface or vasculature of the organ being transplanted at a desired maximum concentration of drug, or that may instead be so directly delivered but titrated to achieve a desired concentration of drug.

Because different drug products are known to vary in efficacy depending on the dose, form, concentration and delivery profile, these and related embodiments provide specific formulation and delivery parameters that confer protection against ischemic reperfusion injury prior to and during transplantation. Nitrite compounds as provided herein in preferred embodiments, or other nitrite- or NO-donating compounds, may be formulated for washing, perfusing or reperfusion following liquid or dry powder (inhalation) administration to achieve desired concentrations to reduce (e.g., decrease in a statistically significant manner, such as relative to an appropriate control treatment) or prevent ischemic reperfusion injury prior to and during organ transplantation.

In still other embodiments there are provided compositions and methods for the treatment of respiratory tract infections (including infections of the upper respiratory tract, respiratory tract airways, and pulmonary compartment) in human and/or veterinary subjects, featuring optimized nitrite compound antimicrobial activity (or nitrite- or NO-donor agent antimicrobial activity) that may be achieved by aerosol administration, and through the delivery of high drug concentrations directly to the affected tissue. In certain preferred embodiments a nitrite compound as provided herein (e.g., nitrite anion or a salt thereof, such as sodium nitrite) is delivered, and in certain other embodiments another nitrite- or nitric oxide-donating agent, as disclosed herein, may be delivered. The nitrite compound, or nitrite- or NO-donating agent, may serve as a sustained-release or active substance upon delivery, under conditions and for a time sufficient as described herein, to produce maximum concentrations (e.g., without appreciable toss by the nitrite compound formulation to the environment, prior to administration, of NO formed therein as evolved NO gas, which loss may be less than about 40%, 30%, 20%, 15%, 10%, 5%, 3%, 2% or 1% of total NO present in the nitrite compound formulation within the first 15 minutes of its preparation) of active drug to the respiratory, pulmonary, and/or other non-oral topical compartments including, but not limited to the skin, rectum, vagina, urethra, urinary bladder, eye, and ear.

Because different drug products (e.g., nitrite compounds as provided herein, or other nitrite- or NO-donating agents as described herein) are known to produce different antimicrobial effects depending on the dose, form, concentration and delivery profile, these embodiments relate to specific formulation and delivery parameters to obtain therapeutically significant antimicrobial results, for instance, by providing bioavailable NO at higher concentrations and for sustained time periods of longer duration than have previously been realized. Nitrite compounds as provided herein in preferred embodiments, or other nitrite- or NO-donating compounds, may be formulated for liquid nebulized, dry powder and/or metered-dose aerosol administration at dosages to achieve desired concentrations according to antimicrobial criteria as will be familiar to those skilled in the art (e.g., detectable effect on microbial infection, viability, colonization or growth at a tissue site, as can be determined according to existing routine methodologies) to treat patients with distinct bacterial infections. The nitrite compound formulations and methods described herein (and the other nitrite- and NO-donor agent formulations and methods) may be used with commercially available inhalation devices, or with other devices for aerosol therapeutic product administration.

Aerosol administration directly to one or more desired regions of the respiratory tract, which includes the upper respiratory tract (e.g., nasal, sinus, and pharyngeal compartments), the respiratory airways (e.g., laryngeal, tracheal, and bronchia, compartments) and the lungs or pulmonary compartments (e.g. respiratory bronchioles, alveolar ducts, alveoli), may be effected (e.g., “pulmonary delivery”) in certain preferred embodiments through intra-nasal or oral inhalation to obtain high and titrated concentration of drug, pro-drug active or sustained-release delivery to a site of respiratory pathology. Aerosol administration such as by intra-nasal or oral inhalation may also be used to provide drug, pro-drug active or sustained-release delivery through the pulmonary vasculature (e.g., further to pulmonary delivery) to reach other tissues or organs, by non-limiting example, the heart, brain, liver and/or kidney, with decreased risk of extra-respiratory toxicity associated with non-respiratory routes of drug delivery. Accordingly, because the efficacy of a particular nitrite compound (e.g., nitrite anion or a salt thereof, such as sodium nitrite), or of another nitrite- or nitric oxide-donating compound therapeutic composition, may vary depending on the formulation and delivery parameters, certain embodiments described herein reflect re-formulations of compositions and novel delivery methods for recognized active drug compounds. Other embodiments contemplate topical pathologies and/or infections that may also benefit from the discoveries described herein, for example, through direct exposure of a nitrite compound formulation as provided herein, or of other or nitrite- or nitric oxide-donating compounds, to infected skin, rectum, vagina, urethra, urinary bladder, eye, and/or ear.

In addition to the clinical and pharmacological criteria according to which any composition intended for therapeutic administration (such as the herein described nitrite compound formulations) may be characterized, those familiar with the art will be aware of a number of physicochemical factors unique to a given drug composition. These include, but are not limited to aqueous solubility, viscosity, partitioning coefficient (Log P), predicted stability in various formulations, osmolality, surface tension, pH, pKa, pK_{b}, dissolution rate, sputum permeability, sputum binding/inactivation, taste, throat irritability and acute tolerability.

Other factors to consider when selecting the particular product form include physical chemistry of the formulation (e.g., a nitrite compound formulation), the intended disease indication(s) for which the formulation is to be used, clinical acceptance, and patient compliance. As non-limiting examples, a desired nitrite compound formulation for aerosol delivery (e.g., by oral and/or intra-nasal inhalation of a mist such as a nebulized suspension of liquid particles), and/or a desired nitrite- or nitric oxide-donating compound formulation for aerosol delivery, may be provided in the form of a simple liquid such as an aqueous liquid (e.g., soluble nitrite compound with non-encapsulating soluble excipients/salts), a complex liquid such as an aqueous liquid (e.g., nitrite compound encapsulated or complexed with soluble excipients such as lipids, liposomes, cyclodextrins, microencapsulations, and emulsions), a complex suspension (e.g., nitrite compound as a low-solubility, stable nanosuspension alone, as co-crystal/co-precipitate complexes, and/or as mixtures with low solubility lipids such as solid-lipid nanoparticles), a dry powder (e.g., dry powder nitrite compound alone or in co-crystal/co-precipitate/spray-dried complex or mixture with low solubility excipients/salts or readily soluble blends such as lactose), or an organic soluble or organic suspension solution, for packaging and administration using an inhalation device such as a metered-dose inhalation device.

Selection of a particular nitrite compound formulation or nitrite compound formulation composition as provided herein according to certain preferred embodiments may be influenced by the desired product packaging. Factors to be considered in selecting packaging may include, for example, intrinsic product stability, whether the formulation may be subject to lyophilization, device selection (e.g., liquid nebulizer, dry-powder inhaler, meter-dose inhaler) and/or packaging form (e.g., simple liquid or complex liquid formulation, whether provided in a vial as a liquid or as a lyophilisate to be dissolved prior to or upon insertion into the device; complex suspension formulation whether provided in a vial as a liquid or as a lyophilisate, and with or without a soluble salt/excipient component to be dissolved prior to or upon insertion into the device, or separate packaging of liquid and solid components; dry powder formulations in a vial, capsule or blister pack; and other formulations packaged as readily soluble or low-solubility solid agents in separate containers alone or together with readily soluble or low-solubility solid agents.)

One or more separately packaged agents may be manufactured in such a way as to be mixed prior to or upon insertion into the delivery device). Accordingly, certain preferred embodiments relate to a nitrite compound formulation composition for pulmonary delivery that comprises a first solution which is provided as a nitrite compound aqueous solution having a pH greater than 7.0; and a second solution which is provided as an acidic excipient aqueous solution, wherein the first solution and the second solution are admixed to form a nitrite compound formulation, prior to administration by oral inhalation or by intra-nasal inhalation, for example as an aerosol such as a nebulized mist. According to certain such embodiments, upon admixture of the first and second solutions to form the nitrite compound formulation, the nitrite compound is present at a concentration of from about 14.5 mM (0.667 mg/mL) to about 2.174 M (100 mg/mL) nitrite anion, the nitrite compound formulation has a pH of from about 4.7 to about 6.5, and nitric oxide bubbles are not visually detectable for at least 15, 30, 45 or 60 minutes following admixture, and/or the nitrite compound is present at a molar ratio relative to the acidic, excipient that exceeds 150:1, 200:1 or 250:1.

In some embodiments, the present invention relates to the aerosol and/or topical delivery of a nitrite compound (e.g., nitrite anion or a salt thereof, such as sodium nitrite (NaNO₂), potassium nitrite (KNO₂) or magnesium nitrite (Mg(NO₂)₂), or calcium nitrite (Ca(NO₂)₂) or lithium nitrite (LiNO₂). These and related embodiments contemplate respiratory tract delivery and in particular pulmonary delivery (e.g., to alveoli, alveolar ducts and/or bronchioles), with certain such embodiments additionally or alternatively contemplating extrapulmonary exposure such as by absorption in the pulmonary compartment into the pulmonary vasculature as may be useful in methods, not limited to prophylaxis and/or therapy against ischemic reperfusion injury in the heart, brain, transplanted lung, transplanted liver, transplanted kidney and other organs. For example, pulmonary delivery via inhalation and subsequent absorption into the circulatory system via pulmonary vascular beds may beneficially place nitrite anions immediately upstream of the coronary and carotid arterial systems, and upstream of the liver and kidneys, for direct access to these organs as disease sites or potential disease sites. Sodium nitrite and magnesium nitrite have favorable solubility characteristics with magnesium nitrite and calcium nitrite in addition offering favorable stoichiometric characteristics.

Any of these nitrite salts (e.g., sodium nitrite, magnesium nitrite, potassium nitrite, calcium nitrite, lithium nitrite) alone or in combination, thereby permit dosing of clinically-desirable nitrite anion and/or (further to reduction of nitrite to NO) nitric oxide levels by aerosol (e.g., through liquid nebulization, dry powder dispersion or meter-dose administration) or topically (e.g., aqueous suspension, oily preparation or the like or as a drip, spray, suppository, salve, or an ointment or the like), and can be used in methods for acute or prophylactic treatment of a subject having pulmonary hypertension, e.g., pulmonary arterial hypertension (PAH), or of a subject at risk for having pulmonary hypertension, or to counteract I/R injury such as in the organs noted above, or for treatment of an acute microbial infection (e.g., bacteria, fungal, parasitic, etc.) or prophylaxis against such infection. Clinical criteria for determining when a subject has or is at risk for having PAH, or when ischemic reperfusion injury has transpired in the heart, brain, transplanted lung, transplanted liver, or transplanted kidney, or when a microbial infection is present, are known to the art. Pulmonary delivery via inhalation permits direct and titrated dosing directly to the clinically-desired site with reduced systemic exposure. According to certain contemplated embodiments, the stoichiometric advantage of magnesium nitrite or calcium nitrite may be exploited for maximal administration of nitrite compound per inhaled breath of aerosolized nitrite compound formulation, e.g., as a nebulized liquid mist or as a dry powder formulation.

In a preferred embodiment, the method treats or serves as prophylaxis against pulmonary hypertension by administering a nitrite compound formulation as an aerosol (e.g., a suspension of liquid particles in air or another gas) containing liquid-dissolved nitrite anion, or a nitrite salt thereof (e.g., NaNO₂), to a subject having or suspected to have pulmonary hypertension. Pulmonary hypertension includes those conditions within the Group I-V Classification as defined by the Third World Health Conference on Pulmonary Hypertension, 2003, Venice. As defined, these groups are Group I pulmonary hypertension (pulmonary arterial hypertension (PAH)), Group II pulmonary hypertension (pulmonary venous hypertension), Group III pulmonary hypertension (pulmonary hypertension associated with lung diseases and/or hypoxemia, Group IV pulmonary hypertension (pulmonary hypertension due to chronic thrombotic and/or embolic disease, and Group V pulmonary hypertension (miscellaneous, including, but not limited to sarcoidosis, histiocytosis X, lymph angiomatosis, and other pathology causing compression of pulmonary vessels). These and related embodiments also include the sub-categories of Group I pulmonary hypertension, which may, for example, include further classification as defined by Rich S. ed. Executive Summary from the World Symposium on Primary Pulmonary Hypertension, 1998, Evian, France. As defined therein, this further classification of Group I pulmonary hypertension includes Class I PAH (no limitation of usual physical activity), Class II PAH (slight limitation of activity), Class III PAH (marked limitation in physical activity), and Class IV PAH (inability to perform any physical activity).

In a preferred embodiment, the method treats or serves as prophylaxis against pulmonary hypertension by co-administering in a separate formulation or together in a fixed-combination liquid nebulizable, dry powder or metered-dose formulation aerosol nitrite anion or salt thereof, (or in distinct embodiments, a nitrite- or nitric oxide-donating compound) with a second or third substance, by non-limiting example, sildenafil, epoprostinol, treprostinil, iloprost, bosentan, sitaxsentan, ambrisentan, heparin, heparinoids, ancrod, other thrombolytics, aspirin, dipyridamole, ticlopidine, clopidogrel, warfarin, digitalis and nimodipine to a subject having or suspected to have pulmonary hypertension.

In a preferred embodiment, the method treats or serves as prophylaxis against ischemic reperfusion injury of the heart following an ischemic episode by administering a liquid nebulized, dry powder or metered-dose aerosol nitrite anion or salt thereof (or in distinct embodiments a nitrite- or nitric oxide-containing compound) formulation to a subject having or suspected to have myocardial ischemia, an infarction or as prophylaxis during coronary arterial catheterization.

In a preferred embodiment, the method treats or serves as prophylaxis against ischemic reperfusion injury of the brain following an ischemic episode by administering a liquid nebulized, dry powder or metered-dose aerosol nitrite anion or a salt thereof (or in distinct embodiments a nitrite- or nitric oxide-containing compound) formulation to a subject having or suspected to have cerebral ischemia, an infarction (or stroke) or as prophylaxis during carotid arterial catheterization.

In a preferred embodiment, the method treats or serves as prophylaxis against ischemic reperfusion injury of the lung prior to or following transplantation by administering a nitrite anion or a salt thereof (or in distinct embodiments a nitrite- or nitric oxide-donating compound) formulation as a flushate (prior to or during transplantation) or as a liquid nebulized, dry powder or metered-dose aerosol (post-transplantation) to a subject having a pulmonary transplant.

In a preferred embodiment, the method treats or serves as prophylaxis against ischemic reperfusion injury of the kidney prior to or following transplantation by administering a nitrite anion or a salt thereof (or in distinct embodiments a nitrite- or nitric oxide-donating compound) formulation as a flushate (prior to or during transplantation) or as a liquid nebulized, dry powder or metered-dose aerosol (post-transplantation) to a subject having a kidney transplant.

In a preferred embodiment, the method treats or serves as prophylaxis against ischemic reperfusion injury of the liver prior to or following transplantation by administering a nitrite anion or a salt thereof, (or in distinct embodiments a nitrite- or nitric oxide-donating compound) formulation as a flushate (prior to or during transplantation) or as a liquid nebulized, dry powder or metered-dose aerosol (post-transplantation) to a subject having a liver transplant.

In a preferred embodiment, the method treats or serves as prophylaxis against ischemic reperfusion injury of the heart prior to or following transplantation by administering a nitrite anion or a salt thereof (or in distinct embodiments a nitrite- or nitric oxide-donating compound) formulation as a flushate (prior to during transplantation) or as a liquid nebulized, dry powder or metered-dose aerosol (post-transplantation) to a subject having a heart transplant.

In a preferred embodiment, the method treats or serves as prophylaxis against ischemic reperfusion injury of the heart and/or brain following an ischemic episode by co-administering in a separate formulation or together in a fixed-combination a liquid nebulizable, dry powder or metered-dose formulation for aerosol of a nitrite anion or salt thereof (or in distinct embodiments a nitrite- or nitric oxide-donating compound) with a second or third substance, by non-limiting example, sildenafil, trimetazidine, allopurinol, edaravone, diltiazem, cariporide, eniporide, MCC-135, anti-CD18 antibody, anti-CD11 antibody, P-selectin antagonist, pexelizumab, adenosine, nicorandil, intravenous magnesium, heparin, heparinoids, ancrod, other thrombolytics, aspirin, dipyridamole, ticlopidine, clopidogrel, digitalis, warfarin, and nimodipine to a subject having or suspected to have myocardial or cerebral ischemia, an infarction or as prophylaxis during coronary or carotid arterial catheterization.

In a preferred embodiment, the method treats or serves as prophylaxis against ischemic reperfusion injury of the heart and/or brain following an ischemic episode by administering combination therapy (which may, for example, be performed/administered separately or in a fixed-combination) comprising cardio- and/or cerebral-protective therapy with a liquid nebulized, dry powder or metered-dose aerosol formulation of a nitrite anion or salt thereof (or in distinct embodiments a nitrite- or nitric oxide-donating compound) to a subject having or suspected of having myocardial and/or cerebral ischemia, and/or an infarction, or as prophylaxis during coronary or carotid arterial catheterization. Such combination cardio- and/or cerebral-protective therapy may, by non-limiting example, include administering one or more of ischemic preconditioning, atrial natriuretic peptide, a protein kinase C-delta inhibitor, glucagon-like peptide 1, darbepoetin alfa, atorvastatin, and cyclosporin.

In a preferred embodiment, the method flushes, reperfuses with, treats or serves as prophylaxis against ischemic reperfusion injury prior to, during or following kidney, lung and/or liver transplantation by co-administering in a separate formulation or together in a fixed-combination liquid nebulizable, dry powder or metered-dose formulation for aerosol a nitrite anion or salt thereof (or in distinct embodiments a nitrite- or nitric oxide-donating compound) with a second or third substance, by non-limiting example, sildenafil, trimetazidine, allopurinol, edaravone, diltiazem, cariporide, eniporide, MCC-135, anti-CD18 antibody, anti-CD11 antibody, P-selectin antagonist, pexelizumab, adenosine, nicorandil, intravenous magnesium, heparin, heparinoids, ancrod, other thrombolytics, aspirin, dipyridamole, ticlopidine, clopidogrel, digitalis, warfarin, and nimodipine to an organ being prepared for transplant or to a subject having received a transplant.

In a preferred embodiment, the method flushes, reperfuses with, treats or serves as prophylaxis against ischemic reperfusion injury prior to, during or following kidney, lung and/or liver transplantation by administering agents known to be cardio- or cerebral-protective agents or procedures in combination (performed/administered separately or in a fixed-combination) with a liquid nebulized, dry powder or metered-dose aerosol nitrite anion or salt thereof (or in distinct embodiments a nitrite- or nitric oxide-donating compound) to an organ being prepared for transplant, during transplant or to a subject having received a transplant. Such cardio- or cerebral-protective therapy, by non-limiting example include ischemic preconditioning, atrial natriuretic peptide, protein kinase C-delta inhibitor, glucagon-like peptide 1, darbepoetin alfa, atorvastatin, and cyclosporin.

In another preferred embodiment, the method treats a bacterial or other microbial (e.g., fungal, parasitic, viral, etc.) infection in a subject using concentrated liquid nebulized, dry powder or metered-dose aerosol nitrite anion or salt thereof (or in distinct embodiments a nitrite- or nitric oxide-donating compound) formulation administered to a subject infected, predisposed to or suspected of having an infection by pathogenic or opportunistic bacteria (or other microbial species) in the lungs.

The therapeutic method may also include a diagnostic step, such as identifying a subject with or suspected of having pulmonary hypertension, in some embodiments, the method further classification into Class I-IV Group I PAH. In some embodiments, the delivered amount of aerosol nitrite anion or salt thereof (or in distinct embodiments a nitrite- or nitric oxide-donating compound) formulation is sufficient to provide acute, sub-acute, or chronic symptomatic relief or stimulate reversal of vasculature remodeling and subsequent increase in survival and/or improved quality of life.

The therapeutic method may also include a diagnostic step, such as identifying a subject with or suspected of having an ischemic event, by non-limiting example in the brain (such as in the case of stroke), or heart (such as in the case of myocardial infarction), or preceding, during or following pulmonary, liver or kidney transplant. In some embodiments, the delivered amount of liquid nebulized, dry powder or metered-dose aerosol nitrite or salt thereof (or in distinct embodiments a nitrite- or nitric oxide-donating compound) formulation is sufficient to prevent reperfusion injury or provide protection prior to, during or following liver, kidney, heart or lung transplant and subsequent increase in survival and/or improved quality of life.

The therapeutic method may also include a diagnostic step, such as identifying a patient infected with a particular pathogenic bacteria, opportunistic bacteria, or antimicrobial-resistant bacteria. In some embodiments, the method further includes identifying a patient as colonized with bacteria that are capable of developing resistance to one or more antimicrobial agents. In some embodiments, the delivered amount of liquid nebulized, dry powder or metered-dose aerosol nitrite anion or salt thereof (or in distinct embodiments, a nitrite- or nitric oxide-donating compound) is sufficient to have an antimicrobial effect upon otherwise antimicrobial-resistant bacteria, and/or overcome, circumvent or prevent resistance development to other antimicrobial agents.

In another embodiment, the delivered amount of liquid nebulized, dry powder or metered-dose aerosol nitrite anion or salt thereof (or in distinct embodiments a nitrite- or nitric oxide-donating compound) is sufficient to overcome pre-existing antimicrobial resistance or prevent further resistance of an organism.

In another embodiment, the delivered amount of aerosol nitrite anion or salt thereof (or in distinct embodiments a nitrite- or nitric oxide-donating compound) is sufficient to reduce the pre-existing antimicrobial resistant infecting bacterial population to levels enabling re-introduction of previously ineffective antimicrobial agents. Such an embodiment may include pre-cursor, concurrent or subsequent therapy of liquid nebulized, dry powder or metered-dose aerosol nitrite anion or salt thereof (or in distinct embodiments a nitrite- or nitric oxide-donating compound) formulation with one or more antimicrobial agents. Without limitation, co-administered or subsequently administered antimicrobial agents may include: aerosol tobramycin and/or other aminoglycoside such as amikacin, aerosol aztreonam and/or other beta or mono-bactam, aerosol ciprofloxacin, aerosol levofloxacin and/or other aerosol, oral or parenteral fluoroquinolones, aerosol azithromycin and/or other macrolides or ketolides, tetracycline and/or other tetracyclines, quinupristin and/or other streptogramins, linezolid and/or other oxazolidinones, vancomycin and/or other glycopeptides, and chloramphenicol and/or other phenicols, and colisitin and/or other polymyxins.

In another embodiment, liquid nebulized, dry powder or metered-dose aerosol nitrite anion or salt thereof (or in distinct embodiments a nitrite- or nitric oxide-donating compound) may be prepared in a fixed-combination with antimicrobial agents which may include: tobramycin and/or other aminoglycoside such as amikacin, aztreonam and/or other beta or mono-bactam, ciprofloxacin, levofloxacin and/or other, fluoroquinolones, azithromycin and/or other macrolides or ketolides, tetracycline and/or other tetracyclines, quinupristin and/or other streptogramins, linezolid and/or other oxazolidinones, vancomycin and/or other glycopeptides, and chloramphenicol and/or other phenicols, and colisitin and/or other polymyxins.

In some embodiments of the methods described above, the bacteria may be gram-negative bacteria such as Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella doysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parpertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Campylobacter fetus, Camphylobacter jejuni, Campylobacter coli, Borrelia burgdorferi, Vibrio cholerae, Vibrio parahaemolyticus, Legionella pneumophila, Listeria monacytogenes, Neisseria gonorrhoeae, Neisseria meningitidis, Kingella, Moraxella, Gardnerella vaginalis, Bacteroides fragilis, Bacteroides distasonis, Bacteroides thetaiotaomicron, Bacteroides uniformis, Bacteroides eggerthii, and Bacteroides splanchnicus. In some embodiments of the methods described above, the bacteria are gram-negative anaerobic bacteria, by non-limiting example these include Bacteroides fragilis, Bacteroides distasonis, Bacteroides 3452A homology group, Bacteroides vulgatus, Bacteroides ovalus, Bacteroides thetaiaomicron, Bacteroides uniformis, Bacteroides eggerthii, and Bacteroides splanchnicus, In some embodiments of the methods described above, the bacteria are gram-positive bacteria, by non-limiting example these include: Corynebacterium diphtheriae, Corynebacterium ulcerans, Streptococcus pneumoniae, Streptococcus agalactiae, Streptococcus pyogenes, Streptococcus milleri; Streptococcus (Group G); Streptococcus (Group C/F); Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, and Staphylococcus saccharolyticus. In some embodiments of the methods described above, the bacteria are gram-positive anaerobic bacteria, by non-limiting example these include Clostridium difficile, Clostridium perfringens, Clostridium tetini, and Clostridium botulinum. In some embodiments of the methods described above, the bacteria are acid-fast bacteria, by non-limiting example these include Mycobacterium tuberculosis, Mycobacterium avium, Mycobacterium intracellulare, and Mycobacterium leprae. In some embodiments of the methods described above, the bacteria are atypical bacteria, by non-limiting example these include Chlamydia pneumoniae and Mycoplasma pneumoniae.

In another embodiment, a method is provided for prophylactic treatment of a subject, including administering to a subject, susceptible to microbial infection or a chronic carrier of an asymptomatic or low symptomatic microbial infection, a nitrite anion or salt thereof (or in distinct embodiments a nitrite- or nitric oxide-donating compound) formulation to achieve a minimal inhibitory concentration of nitrite anion or salt thereof (or in distinct embodiments nitrite- or nitric oxide-donating compound) at a site of potential or current infection following liquid nebulized, dry powder or metered-dose aerosol administration. In one embodiment, the method further comprises identifying a subject as a subject at risk of a bacterial infection or at risk for an exacerbation of an infection.

In another embodiment, a method is provided for acute, chronic or prophylactic treatment of a patient through liquid nebulized, dry powder or metered-dose aerosol administration of a nitrite compound (e.g., nitrite anion or a salt thereof, such as sodium nitrite) formulation, or in certain distinct embodiments of a nitrite- or nitric oxide-donating compound formulation, to produce and maintain threshold drug concentrations in the blood and/or lung, which may be measured as drug levels in epithelial lining fluid (ELF), sputum, lung tissue, bronchial lavage fluid (BAL), or by deconvolution of blood concentrations through pharmacokinetic analysis. One embodiment includes the use of aerosol administration, delivering high or titrated concentration drug exposure directly to the affected tissue for treatment of pulmonary hypertension in animals and humans, in one such embodiment, the peak plasma levels achieved following aerosol administration to the lung will be between 0.01 and 1000 micromolar nitrite, in another preferred embodiment, the peak plasma levels following such an administration would be 0.1-100 micromolar nitrite, in another preferred embodiment, the peak plasma levels following such an administration would be 0.5-75 micromolar nitrite, in a most preferred embodiment, the peak plasma levels following inhalation administration to the lung would be 1-50 micromolar nitrite and in other preferred embodiments the peak plasma levels may be 0.1-10 micromolar nitrite.

In another embodiment, a method is provided for acute, chronic or prophylactic treatment of a patient through liquid nebulized, dry powder or metered-dose aerosol administration of nitrite anion or a salt thereof (e.g., sodium nitrite, potassium nitrite, magnesium nitrite) (or in distinct embodiments a nitrite- or nitric oxide-donating compound) formulation to produce threshold drug concentrations in the blood and/or lung, which may be measured as drug levels in epithelial lining fluid (ELF), sputum, lung tissue, bronchial lavage fluid (BAL), or by deconvolution of blood concentrations through pharmacokinetic analysis that absorb to the pulmonary vasculature producing drug levels sufficient for extra-pulmonary therapeutics or prophylaxis. One embodiment includes the use of aerosol administration, delivering high concentration drug exposure in the vasculature for treatment and/or prophylaxis of, but not limited to ischemic reperfusion injury or the heart and/or brain and tissues such as the lung, kidney, liver and heart prior to, during and following transplantation. In one such embodiment, the peak plasma levels achieved following aerosol administration to the lung will be between 0.01 and 1000 micromolar nitrite, in another preferred embodiment, the peak plasma levels following such an administration may be 0.1-100 micromolar nitrite, in another preferred embodiment, the peak plasma levels following such an administration may be 0.5-75 micromolar nitrite, in certain preferred embodiments, the peak plasma levels following inhalation administration to the lung may be 1-50 micromolar nitrite and in other preferred embodiments the peak plasma levels may be 0.1-10 micromolar nitrite. Flushing solutions may vary outside these preferred embodiments.

In another embodiment, a method is provided for prophylactic treatment of an organ (by non-limiting example liver, kidney, lung and heart) prior to and during transplantation to reduce or eliminate the possibility of developing injury following reperfusion. To this end, a flushate of nitrite anion or a salt thereof (or in distinct embodiments of a nitrite- or nitric oxide-donating compound) formulation is prepared such that upon washing, perfusing or reperfusion the to-be-transplanted or in-process of being transplanted organ is exposed to wash solution or plasma levels with peak plasma and/or wash levels of 0.1-100 micromolar nitrite, in another preferred embodiment using nitrite anion or a salt thereof, the peak plasma and/or wash levels contain 0.5-75 micromolar nitrite, in a most preferred embodiment using nitrite anion or a salt thereof, the peak plasma and/or wash levels contain 1-50 micromolar nitrite and in other preferred embodiments the peak plasma and/or wash levels may contain 0.1-10 micromolar nitrite. Flushing solutions may vary outside these preferred embodiments.

In another embodiment, a method is provided for acute, chronic or prophylactic treatment of a patient through liquid nebulized, dry powder or metered-dose aerosol administration of nitrite anion or a salt thereof (or in distinct embodiments a nitrite- or nitric oxide-donating compound) formulation to produce and maintain threshold drug concentrations in the plasma and/or lung, which may be measured as drug levels in epithelial lining fluid (ELF), sputum, lung tissue, bronchial lavage fluid (BAL), or by deconvolution of blood concentrations through pharmacokinetic analysis. One embodiment includes the use of aerosol administration, delivering high concentration drug exposure directly to the affected tissue for treatment of bacterial infections in animals and humans. In one such embodiment, the lung epithelial lining fluid or sputum levels achieved following aerosol administration to the lung will be between 1 and 100 millimolar nitrite, in another preferred embodiment, the peak plasma levels following such an administration would be 1-50 millimolar nitrite.

In another embodiment, a method is provided for acute or prophylactic treatment of a patient through non-oral or non-nasal topical administration of nitrite anion or a salt thereof (or in distinct embodiments a nitrite- or nitric oxide-donating compound) formulation to produce and maintain threshold drug concentrations at the site of infection or at risk of infection. One embodiment includes the use of aerosol administration, delivering high concentration drug exposure directly to the affected tissue for treatment or prevention of bacterial infections in skin, rectal, vaginal, urethral, ocular, and auricular tissues. For example according to these and related embodiments, the term aerosol may include a spray, mist, or other nucleated liquid or dry powder form.

In another embodiment, a method is provided for administering a nitrite anion or salt thereof for in distinct embodiments a nitrite- or nitric oxide-donating compound) formulation by inhalation, wherein the inhaled liquid aerosol (e.g., following liquid nebulization or metered-dose administration) or dry powder aerosol has a mean particle size from about 1 micron to 10 microns mass median aerodynamic diameter and a particle size geometric standard deviation of less than or equal to about 3 microns. In another embodiment, the particle size is 2 microns to about 5 microns mass median aerodynamic diameter and a particle size geometric standard deviation of less than or equal to about 3 microns. In one embodiment, the particle size geometric standard deviation is less than or equal to about 2 microns. In certain related and preferred embodiments there is provided one or a plurality of liquid particles of about 0.1 to 5.0 microns volumetric mean diameter, the particle comprising a nitrite compound formulation as described herein.

In some embodiments of the methods described above, nitrite anion or a salt thereof (or in distinct embodiments a nitrite- or nitric oxide-donating compound) remains at the therapeutically effective concentration at the site of pulmonary hypertension pathology, suspected pulmonary pathology, and/or site of pulmonary absorption into the pulmonary vasculature for at least about 1 minute, at least about a 5 minute period, at least about a 10 min period, at least about a 20 min period, at least about a 30 min period, at least about a 1 hour period, at least a 2 hour period, at least about a 4 hour period, at least an 8 hour period, at least a 12 hour period, at least a 24 hour period, at least a 48 hour period, at least a 72 hour period, or at least one week. The effective nitrite anion or salt thereof (or in distinct embodiments nitrite- or nitric oxide-donating compound) concentration is sufficient to cause a therapeutic effect and the effect may be localized or broad-acting to or from the site of hypertensive pathology.

In some embodiments of the methods described above, the nitrite anion or a salt thereof (or in distinct embodiments nitrite- or nitric oxide-donating compound) following inhalation administration remains at the therapeutically effective concentration at the site of ischemic, potential reperfusion injury site, by non-limiting example, heart, brain, transplanted lung, transplanted kidney and/or transplanted liver for at least about 1 minute, at least about a 5 minute period, at least about a 10 min period, at least about a 20 min period, at least about a 30 min period, at least about a 1 hour period, at least a 2 hour period, at least about a 4 hour period, at least an 8 hour period, at least a 12 hour period, at least a 24 hour period, at least a 48 hour period, at least a 72 hour period, or at least one week. The effective nitrite anion or salt thereof (or in distinct embodiments nitrite- or nitric oxide-donating compound) concentration is sufficient to cause a therapeutic effect and the effect may be localized or broad-acting to or from the site of potential ischemic reperfusion injury.

In another embodiment, a method is provided for prophylactic treatment of an organ (by non-limiting example liver, kidney, lung and heart) prior to and during transplantation to reduce or eliminate the possibility of developing injury following reperfusion. To this end, a flushate of nitrite anion or a salt thereof (or in distinct embodiments a nitrite- or nitric oxide-donating compound) formulation is prepared such that upon washing, perfusing or reperfusion the to-be-transplanted or in-process of being transplanted organ is exposed to wash solution or plasma levels with peak and/or sustained levels of nitrite anion at the site of ischemic, potential reperfusion injury site, by non-limiting example, heart, brain, transplanted lung, transplanted heart, transplanted kidney and/or transplanted liver for at least about 1 minute, at least about a 5 minute period, at least about a 10 min period, at least about a 20 min period, at least about a 30 min period, at least about a 1 hour period, at least a 2 hour period, at least about a 4 hour period, at least an 8 hour period, at least a 12 hour period, at least a 24 hour period, at least a 48 hour period, at least a 72 hour period, or at least one week. The effective nitrite anion or salt thereof (or in distinct embodiments nitrite- or nitric oxide-donating compound) concentration is sufficient to cause a therapeutic effect and the effect may be localized or broad-acting to or from the site of potential ischemic reperfusion injury.

In some embodiments of the methods described above, the nitrite anion or a salt thereof (or in distinct embodiments a nitrite- or nitric oxide-donating compound) remains at the minimal anti-bacterial inhibitory concentration at the site of infection, suspected infection, or pre-disposed infection for at least about a 5 minute period, at least about a 10 min period, at least about a 20 min period, at least about a 30 min period, at least about a 1 hour period, at least a 2 hour period, at least about a 4 hour period, at least an 8 hour period, at least a 12 hour period, at least a 24 hour period, at least a 48 hour period, at least a 72 hour period, or at least one week. The effective nitrite anion or salt thereof (or in distinct embodiments nitrite- or nitric oxide-donating compound) minimal inhibitory concentration (MIC) is sufficient to cause a therapeutic effect and the effect may be localized to the site of infection, in some embodiments, one or more nitrite anion or salt thereof (or in distinct embodiments nitrite- or nitric oxide-donating compound) formulation administrations achieve an ELF, BAL, and/or sputum nitrite anion (or in distinct embodiments nitrite- or nitric oxide-donating compound) concentrations of at least 1-fold to 5000-fold the infecting or potentially infecting organisms MIC, including all integral values therein such as 2-fold, 4-fold, 8-fold, 16-fold, 32-fold, 64-fold, 128-fold, 256-fold, 512-fold, 1028-fold, 2056-fold, and 4112-fold the microbials MIC.

In some embodiments, such as a pulmonary site, the nitrite anion or salt thereof (or in distinct embodiments the nitrite- or nitric oxide-donating compound) formulation is administered in one or more administrations so as to achieve a respirable delivered dose daily of nitrite anion (or in distinct embodiments of other nitrite or nitric oxide-donating compound) of at least about 0.5 mg to about 100 mg, including all integral values therein such as 1, 2, 4, 6, 10, 15, 20, 25, 30, 35, 40, 45, 50, 60, 70, 80, and 90 milligrams. Similarly, the nitrite anion or salt thereof (or in distinct embodiments, nitrite- or nitric oxide-donating compound) formulation is administered in one or more administrations so as to achieve a respirable delivered dose daily of nitrite anion (or in distinct embodiments of other nitric oxide-donating compound) of at least about 100 to about 300 mg including all integral values therein, such as 110, 120, 130, 140, 150, 175, 200, and 250 mg. The nitrite anion or salt thereof (or in distinct embodiments nitrite- or nitric oxide-donating compound) formulation is administered in the described respirable delivered dose in less than 20 minutes, less than 10 minutes, less than 7 minutes, less than 5 minutes, in less than 3 minutes, in less than 2 minutes, in 10 inhalation breaths, 8 inhalation breaths, 6 inhalation breaths, 4 inhalation breaths, 3 inhalation breaths, 2 inhalation breaths or 1 inhalation breath.

As also noted elsewhere, herein, in preferred embodiments the nitrite compound for use in a nitrite compound formulation as described herein comprises nitrite anion (NO₂⁻) or a salt thereof, for example, in particularly preferred embodiments sodium nitrite, potassium nitrite, or magnesium nitrite, and in other preferred embodiments the nitrite salt may be calcium nitrite, silver nitrite or lithium nitrite.

According to certain other distinct embodiments of the compositions and methods described herein, the nitrite- or nitric oxide-donating compound is one or more of the (compounds selected from the group consisting of nitrate, nitrogen dioxide, nitric oxide (gas) itself, nitrous acid, arginine, nitrosothiols, nitroglycerine, glutamine, lysine, asparagine, amyl nitrite, nitric oxide-donating aspirin, NG-nitro-L-arginine methylester, nitroprusside, nitrosobenzene, nitrosyl chloride, O-nitrosoethanol, ethyl nitrite, ethyl nitrate, S-nitrosoglutathione, Ruthenium(III) nitrosyl chloride, Nitrosyl tetrafluoroborate, Potassium pentachloronitrosylruthenate(II), Ruthenium(III) nitrosyl nitrate, 1-Nitroso-2-naphthol, 1-Nitroso-2-naphthol-3,6-disulfonic acid, 2-Methyl-2-nitrosopropane, 2-Nitroso-1-naphthol, 3-(3-Hydroxy-4-nitroso-N-propylanilino)propanesulfonic acid, 3-Hydroxy-4-nitroso-2,7-naphthalenedisulfonic acid, 6-Nitroso-1,2-benzopyrone, Cupferron, N-Benzyl-N-nitroso-p-toluenesulfonamide, N,N-Dimethyl-4-nitrosoaniline, N-Nitroso-N-ethylbutylamine, N-Nitroso-N-ethylurea, N-Nitroso-N-methylbutylamine, N-Nitroso-N-methylurea, N-Nitrosodiphenylamine, S-Nitroso-N-acetyl-DL-penicillamine, 1,3,5-Tri-tert-butyl-2-nitrosobenzene, 4-Hydroxy-3-nitroso-1-naphthalenesulfonic acid, Diazald®, N,N-Diethyl-4-nitrosoaniline, N-Nitrosodiphenylamine, N-Nitrosodiphenylamine, N-Nitrosodiphenyamine solution, Dephostatin, Diazald®-N-methyl, PAPA NONOate, 6-Amino-1-methyl-5-nitrosouracil, Diazald®-N-methyl-N-methyl, 1,3-difluoro-2-nitroso-benzene, 1,8-dihydroxy-2-nitroso-3,6-naphthalenedisulfonic acid, copper complex, 1-ethyl-3-nitroso-2-phenylindole, 1-ethyl-3-nitroso-piperazine, 17-alpha-chloro-17-beta-nitroso-5-alpha-androstane 2,6-diamino-5-nitroso-4-pyrimidinol, 2-nitro-1-nitroso-1-phenylcyclohexane, 2-nitroso-1,2-dihydroharmine, 2-nitroso-1-naphthol-3,6-disulfonic acid, 2-nitroso-4,7,7-trimethyl-2-azabicyclo(2.2.1)heptan-3-one, 2-tert-butyl-6-methyl-4-nitroso-phenol, 3,5-dimethyl-4-nitroso-1H-pyrazole-3-alpha-chloro-3-beta-nitroso-5-alpha-cholestane, 3-alpha-chloro-3-eta-nitroso-5-alpha-cholestane, 3-chloro-3-nitroso-5-beta-cholestane, 3-nitro-1-nitroso-1-octylguanidine, 3-nitroso-1-oxa-3-azaspiro(4,5)decan-2-one, 3-nitroso-2,4,6-triacetamidopyridine, 3-nitroso-2-phenylimidazo[1,2-A]pyrimidine, 4-alpha-chloro-4-beta, nitroso-5-alpha-cholestane, 4-hydroxy-3-nitroso-1-naphthalene-sulfonic acid, 4-hydroxy-3-nitroso-1-naphthalene-sulfonic acid, 5-(3,5-di-tert-butylphenyl)-3-nitroso-2-oxazolidinone, 5-nitroso-quinolin-8-ol, 6-amino-5-nitroso-2-thiouracil, 7-alpha-chloro-7-beta-nitroso-5-alpha-cholestane, 7-methyl-3-nitroso-2-phenylimidazo[1,2-A]pyridine, diethyl-(n3-nitroso-phenyl)-amine, N-(2-ethoxy-Ph)-2-(1-nitroso-3-oxo-1,2,3,4-tetrahydroquinozalin-2-yl)-acetamine-(4-bromo-phenyl)-5-nitroso-pyrimidine-2,4,6-triamine, N-methyl-N-nitroso-3-tetrahydrothiophenamine, 1,1-dioxide, N—(N′-methyl-N′-nitroso-amino-methyl)benzamide, N-nitroso-N-(2-pyridyl)-3-(trifluoromethyl)aniline, N-nitroso-N-(trimethylsilylmethyl)-P-toluenesulfonamine, S-(9-nitrosos-9H-purin-6-yl)-2-chloroethylthiocarbamate, 2-Nitrosotoluene, 4-Nitrosodiphenylamine, N-Nitrosodiethylamine, Nitrosobenzene, Semustine, Butyl nitrite, Dicyclohexylamine nitrite, Dicyclohexylammonium nitrite, Ethyl nitrite, Isoamyl nitrite, Isobutyl nitrite, Isopentyl nitrite, tert-Butyl nitrite, Tetrabutylammonium nitrite, Bis(triphenylphosphoranylidene)ammonium nitrite, 2-Ethylhexyl nitrate, isobutyl nitrate, and isopropyl nitrate.

In some embodiments of the methods described herein, a composition as provided herein such as a nitrite compound or a nitrite compound formulation or a liquid particle comprising a nitrite compound or a plurality of nebulized liquid particles that comprise a nitrite compound formulation or that comprise an aqueous solution which comprises a nitrite compound may be administered or delivered to a subject, wherein the subject is a human. In some related embodiments the subject is a human with pulmonary hypertension or a human requiring reperfusion therapy or prophylaxis following a cerebral ischemic episode such as a stroke or during carotid arterial catheterization or a human requiring reperfusion therapy or prophylaxis following a cardiac ischemic episode such as a myocardial infarction or during coronary arterial catheterization or a human requiring a lung, liver, kidney or heart transplant where in reperfusion therapy or prophylaxis is desired or a human requiring antimicrobial (e.g., antibacterial, ant-fungal, anti-parasitic, anti-viral, etc.) therapy or a human with cystic fibrosis or a human with pneumonia, a chronic obstructive pulmonary disease, or sinusitis. In certain further non-limiting embodiments the human subject has or is suspected of having one or more of Group I-V pulmonary hypertension.

In certain other related embodiments of the methods described herein, the human subject as provided herein (e.g., a subject as described in the preceding paragraph) may be mechanically ventilated, and in certain further such embodiments, aerosol administration is performed, for example, using an in-line device such as a liquid nebulizer (by non-limiting example, the Aerogen Aeroneb Pro, Aerogen, Inc., Galway, Ireland) or similar adaptor with a device for liquid nebulization. Aerosol administration may also be performed using an in-line adaptor for dry powder or metered-dose aerosol generation and delivery.

In certain embodiments disclosed herein, a pharmaceutical composition is provided that comprises a simple liquid (e.g., aqueous) solution of a nitrite anion or salt thereof, such as sodium nitrite, potassium nitrite or magnesium nitrite. Certain other distinct embodiments provide a pharmaceutical composition that comprises a simple liquid (e.g., aqueous) solution of a nitrite- or nitric oxide-donating compound formulation (e.g., a soluble nitrite- or nitric oxide-donating compound with non-encapsulating water soluble excipients) as described herein and having an osmolality (which as known in the art refers to the number of moles of solute dissolved in one kilogram of solvent and may be represented as osmolality (Osm) or osmoles per kilogram (Osmol/kg)) from about 200 mOsmol/kg to about 5000 mOsmol/kg. In one embodiment, the osmolality is from about 250 mOsmol/kg to about 4000 mOsmol/kg. In another embodiment, the osmolality is from about 500 mOsmol/kg to about 3000 mOsmol/kg. In another embodiment, the osmolality is from about 500 mOsmol/kg to about 2000 mOsmol/kg. In another embodiment, the osmolality is from about 500 mOsmol/kg to about 1000 mOsmol/kg. In another embodiment the osmolality is from about 100 mOsmol/kg to about 3600 mOsmol/kg. In other embodiments the osmolality is from about 100, 150, 200, 250, 300, 350, 400, 450, 500, 550 or 600 mOsmol/kg to about 2000, 2250, 2500, 2750, 3000, 3250, 3500 or 3600 mOsmol/kg. With respect to osmolality, and also elsewhere in the present application, “about” when used to refer to a quantitative value (other than in the context of pH, where as described in greater detail below with regard to buffers, the meaning of “about” a specified pH is provided) means that a specified quantity may be greater than or less than the indicated amount by 1, 2, 3, 4, 5, 6, 7, 8, 9, 10, 11, 12, 13, 14, 15, 16, 17, 18, 19 or percent of the stated numerical value.

In other embodiments, a pharmaceutical composition is provided that in certain further embodiments comprises a simple liquid solution of a nitrite anion or a salt thereof, and in certain other distinct embodiments comprises a nitrite- or nitric oxide-donating compound formulation, wherein these pharmaceutical compositions may have a permeant ion concentration of from about 30 mM to about 300 mM and preferably from about 50 mM to about 200 mM. In certain such embodiments, one or more permeant ions in the composition are selected from the group consisting of chloride and bromide.

In other embodiments, a pharmaceutical composition is provided that in certain further embodiments comprises a complex liquid comprising a nitrite anion or a salt thereof encapsulated or complexed with water soluble excipients such as lipids, liposomes, cyclodextrins, microencapsulations, and emulsions, and in certain other distinct embodiments comprises a complex liquid comprising a nitrite- or nitric oxide-donating compound formulation (e.g., nitrite- or nitric oxide-donating compound) encapsulated or complexed with water soluble excipients such as lipids, liposomes, cyclodextrins, microencapsuations, and emulsions, said complex liquid pharmaceutical compositions having a solution osmolality from about 200 mOsmol/kg to about 5000 mOsmol/kg. In one embodiment, the osmolality is from about 250 mOsmol/kg to about 4000 mOsmol/kg. In another embodiment, the osmolality is from about 500 mOsmol/kg to about 3000 mOsmol/kg. In another embodiment, the osmolality is from about 500 mOsmol/kg to about 2000 mOsmol/kg. In another embodiment, the osmolality is from about 500 mOsmol/kg to about 1000 mOsmol/kg. In another embodiment, the osmolality is from about 100 mOsmol/kg to about 1000 mOsmol/kg. In another embodiment, the osmolality is from about 100 mOsmol/kg to about 500 mOsmol/kg. In another embodiment, the osmolality is from about 100 mOsmol/kg to about 300 mOsmol/kg. In another embodiment the osmolality is from about 100 mOsmol/kg to about 3600 mOsmol/kg. In other embodiments the osmolality is from about 100, 150, 200, 250, 300, 350, 400, 450, 500, 550 or 600 mOsmol/kg to about 2000, 2250, 2500, 2750, 3000, 3250, 3500 or 3600 mOsmol/kg.

In certain other embodiments, a pharmaceutical composition is provided that includes a complex liquid nitrite compound (e.g., nitrite anion or salt thereof), or in related but distinct embodiments a nitrite-, or nitric oxide-donating compound (e.g., nitrite- or nitric oxide-donating compound), wherein the compound is present as a low water-soluble stable nanosuspension alone or in co-crystal/co-precipitate complexes, or mixtures with low solubility lipids, such as lipid nanosuspensions.) Preferably the pharmaceutical composition of these embodiments will have a solution osmolality from about 200 mOsmol/kg to about 5000 mOsmol/kg. In one embodiment, the osmolality is from about 250 mOsmol/kg to about 4000 mOsmol/kg. In another embodiment, the osmolality is from about 500 mOsmol/kg to about 3000 mOsmol/kg. In another embodiment the osmolality is from about 500 mOsmol/kg to about 2000 mOsmol/kg. In another embodiment, the osmolality is from about 500 mOsmol/kg to about 1000 mOsmol/kg. In another embodiment, the osmolality is from about 100 mOsmol/kg to about 1000 mOsmol/kg. In another embodiment, the osmolality is from about 100 mOsmol/kg to about 500 mOsmol/kg. In another embodiment, the osmolality is from about 100 mOsmol/kg to about 300 mOsmol/kg. In another embodiment the osmolality is from about 100 mOsmol/kg to about 3600 mOsmol/kg. In other embodiments the osmolality is from about 100, 150, 200, 250, 300, 350, 400, 450, 500, 550 or 600 mOsmol/kg to about 2000, 2250, 2500, 2750, 3000, 3250, 3500 or 3600 mOsmol/kg.

In other embodiments, a pharmaceutical composition such as any of those just described is provided that includes a complex liquid nitrite compound formulation, or in a related but distinct embodiment a nitrite- or nitric oxide-donating compound formulation, said formulations having a permeant ion concentration from about 30 mM to about 300 mM, or from about 50 mM to about 200 mM. In certain of such embodiments, one or more permeant ions in the composition are selected from the group consisting of chloride and bromide.

In other embodiments including certain preferred embodiments disclosed herein, a nitrite compound formulation as provided herein, or a pharmaceutical composition as provided herein, includes a taste-masking agent. As non-limiting examples, a taste-masking agent may include a sugar, saccharin (e.g., sodium saccharin [Na Saccharin]), sweetener or other compound or agent that beneficially affects taste, after-taste, perceived unpleasant saltiness, sourness or bitterness, or that reduces the tendency of an oral or inhaled formulation to irritate a recipient (e.g., by causing coughing or sore throat or other undesired side effect, such as may reduce the delivered dose or adversely influence patient compliance with a prescribed therapeutic regimen). Certain taste-masking agents may form complexes with a nitrite compound (e.g., nitrite anion or a salt thereof such as sodium nitrite), or in related embodiments, with a nitrite- or nitric oxide-donating compound. In certain related embodiments, the taste-masking agent has a high potency, e.g. greater sweetening or taste-masking capacity at lower concentrations when compared to sugar. Without limitation, such high potency agents include aspartame, saccharin, sucralose or neotame.

In certain preferred embodiments that relate to the nitrite compound formulations disclosed herein, the formulation comprises a nitrite compound and a taste-masking agent and may be optimized with respect to a desired osmolality, and/or an optimized permeant ion concentration. In certain such embodiments, the taste-masking agent comprises saccharin (e.g., sodium saccharin), which according to non-limiting theory affords certain advantages associated with the ability of this taste-masking agent to provide desirable taste effects even when present in extremely low concentrations, such as may have little or no effect on the detectable osmolality of a solution, thereby permitting the herein described formulations to deliver aqueous solutions containing effective concentrations of liquid-dissolved nitrite anion and/or liquid-dissolved NO (i.e., NO at concentrations that can be retained in solution and so does not evolve as readily visible gas bubbles). Non-limiting examples of these and related embodiments include a nitrite compound formulation for pulmonary delivery as described herein that comprises an aqueous solution having a pH of from about 4.7 to about 6.5 and an osmolality of from about 100 to about 3600 mOsm/kg, the solution comprising sodium nitrite and sodium saccharin at a sodium nitrite:sodium saccharin molar ratio of from about 1.3×10³:1 to about 4.4×10³:1. A related non-limiting example further comprises citrate (e.g., citric acid) in the aqueous solution at a sodium nitrite:citrate molar ratio of from about 2.0×10²:1 to about 6.9×10²:1.

Similarly, in certain preferred embodiments that relate to the nitrite compound formulations disclosed herein (including in some embodiments certain contemplated nitrite compound formulation compositions), the formulation comprises a nitrite compound and a taste-masking agent and may be optimized with respect to a desired osmolality, and/or an optimized permeant ion concentration. In certain such embodiments, the taste-masking agent comprises saccharin (e.g., sodium saccharin), which provides desirable taste effects even when present in extremely low concentrations, such as may have little or no effect on the detectable osmolality of a solution, thereby permitting delivery of the herein described formulations with a pH range of about 7.0 to about 9.0. Nonlimiting examples of these and related embodiments include a nitrite compound formulation for pulmonary delivery as described herein that comprises an aqueous solution containing nitrite at about 0.667 mg/mL to about 100 mg/mL, having a pH of from about 7.0 to about 9.0, an osmolality of from about 300 to about 3600 mOsm/kg, and sodium saccharin where sodium saccharin is present, between from about 0.1 mM to 2.0 mM, and sodium phosphate buffer where sodium phosphate is present between from about 0.1 mM to 5.0 mM.

Similarly, in certain preferred embodiments that relate to the nitrate compound formulations disclosed herein (including in some embodiments certain contemplated nitrite compound formulation compositions), the formulation comprises a nitrite compound and a taste-masking agent and may be optimized with respect to a desired osmolality, and/or an optimized permeant ion concentration. In certain such embodiments, the taste-masking agent comprises saccharin (e.g., sodium saccharin), which provides desirable taste effects even when present in extremely low concentrations, such as may have little or no effect on the detectable osmolality of a solution, thereby permitting delivery of the herein described formulations with a pH range of about 7.0 to about 9.0. Non-limiting examples of these and related embodiments include a nitrite compound formulation for pulmonary delivery as described herein that comprises an aqueous solution containing sodium nitrite at about 10 mg/mL to about 100 mg/mL, having a pH of from about 7.0 to about 9.0, an osmolality of from about 300 to about 3600 mOsm/kg, and sodium saccharin where sodium saccharin is present between from about 0.1 mM to 2.0 mM, and sodium phosphate buffer where sodium phosphate is present between from about 0.1 mM to 5.0 mM.

In another embodiment, a pharmaceutical composition is provided that includes an agent that reduces nitrite anion, or in distinct but related embodiments that reacts with a nitrite- or nitric oxide-donating compound, to produce nitric oxide in the nitrite compound formulation (or in the nitrite- or nitric oxide-donating compound formulation) prior to administration. Such agents may include, for example, reducing acids such as ascorbic acid, or reducing sugars such as dextrose co-formulated or vialed separately for admixture, prior to administration, with the nitrite compound (e.g., nitrite anion or salt thereof), or with the nitrite- or nitric oxide-donating compound, such that the resulting admixture may be optimized for a desired osmolality as described herein, and/or for an optimized permeant ion concentration.

In certain other embodiments, a pharmaceutical composition is provided that comprises a formulation which includes an agent that lowers (e.g., decreases in a detectable and statistically significant manner) the solution pH such that nitrite anion or a salt thereof, or in related but distinct embodiments a nitrite- or nitric oxide-donating compound, can produce nitric oxide in the formulation prior to administration. By non-limiting example such agents may include organic buffers such as citric acid. The resulting pH following formulation or admixture of such agents with a nitrite anion or salt thereof, or with a nitrite- or nitric oxide-donating compound, to obtain a desired osmolality, and/or an desired permeant ion concentration such as those disclosed herein, may be from about pH 4.0 to about pH 8.5, more preferably from about pH 4.7 to about pH 7.5, more preferably from about pH 4.7 to about pH 6.5, or more preferably from about pH 5.0 to about pH 6.0.

In another embodiment, a pharmaceutical composition is provided to produce a neutral pH formulation prior to administration. By non-limiting example such agents may include organic buffers such as citric acid or an inorganic buffer such as phosphate. The formulation may in certain embodiments be prepared without a pH buffer, as nitrite anion and nitrite salts are neutral by nature. However, inclusion of a buffer may usefully promote pH stability, in these and related embodiments, including those which are formulated to obtain a desired osmolality and/or an desired permeant ion concentration such as those disclosed herein, the resulting pH of the nitrite compound aqueous solution may be from about pH 6.0 to bout pH 9.0, more preferably from about pH 6.5 to about pH 8.0, or more preferably from about pH 7.0 to about pH 8.0.

In other embodiments, pharmaceutical compositions are provided that include a simple dry powder formulation comprising a nitrite compound, or a nitrite- or nitric oxide-donating compound, alone in dry powder form or with a blending agent such as lactose. In other embodiments, the pharmaceutical composition used in a liquid, dry powder or meter-dose inhalation device is provided such that the nitrite salt is sodium, magnesium, potassium, lithium or calcium. In other embodiments, a pharmaceutical composition is provided that includes a complex dry powder nitrite anion, nitrite salt, or nitrite- or nitric oxide-donating compound formulation (e.g., nitrite, nitrite salt, or nitrite or nitric oxide-donating compound in co-crystal/co-precipitate/spray dried complex or mixture with low water soluble excipients/salts in dry powder form with or without a blending agent such as lactose).

In other embodiments, a system is provided for administering a nitrite compound, or in distinct embodiments a nitrite- or nitric oxide-donating compound, that includes a container comprising a solution of the nitrite compound or the nitrite- or nitric oxide-donating compound formulation, and a liquid nebulizer physically coupled or co-packaged with the container and adapted to produce an aerosol of the solution having a particle size from about 0.1 microns to about 5 microns volumetric mean, or from about 2 to about 5 microns mean mass aerodynamic diameter and a particle size geometric standard deviation of less than or equal to about 2.5 microns mean mass aerodynamic diameter. In one embodiment, the particle size geometric standard deviation is less than or equal to about 3.0 microns. In one embodiment, the particle size geometric standard deviation is less than or equal to about 2.0 microns.

In other embodiments, a system is provided for administering a nitrite compound, or a nitrite- or nitric oxide-donating compound, that includes a container comprising a dry powder of a nitrite compound, or of a nitrite- or nitric oxide-donating compound, and a dry powder inhaler coupled to the container and adapted to produce a dispersed dry powder aerosol having a particle size from about 2 microns to about 5 microns mean mass aerodynamic and a particle size standard deviation of less than or equal to about 3.0 microns. In one embodiment, the particle size standard deviation is less than or equal to about 2.5 microns. In one embodiment, the particle size standard deviation is less than or equal to about 2.0 microns.

In another embodiment, a kit is provided that includes a container comprising a pharmaceutical formulation comprising a nitrite compound (e.g., a nitrite anion or a nitrite salt thereof, such as sodium nitrite, potassium nitrite or magnesium nitrite), or in an alternative distinct embodiment, a nitrite or nitric oxide-donating compound, and an aerosolizer adapted to aerosolize the pharmaceutical formulation (e.g., in certain preferred embodiments, a liquid nebulizer) and deliver it to the lower respiratory tract, for instance, to a pulmonary compartment such as alveoli, alveolar ducts and/or bronchioles, following intraoral and/or intranasal administration. The formulation may also be delivered as a dry powder or through a metered-dose inhaler.

In another embodiment, a kit is provided that includes a container comprising a pharmaceutical formulation comprising a nitrite compound (e.g. a nitrite anion or a nitrite salt thereof, such as sodium nitrite, potassium nitrite or magnesium nitrite), or in an alternative distinct embodiment, a nitrite- or nitric oxide-donating compound, and an aerosolizer adapted to aerosolize the pharmaceutical formulation (e.g., in certain preferred embodiments, a liquid nebulizer) and deliver it to a nasal cavity, and/or to one or more other respiratory tract compartments (e.g., pharyngeal, tracheal, laryngeal, bronchial, bronchiolar, pulmonary, etc.) following intranasal and/or, intraoral administration. The formulation may also be delivered as a dry powder or through a metered-dose inhaler.

In another embodiment, methods, formulations and devices are that result in delivery of nitrite resulting in a plasma Cₘₐₓ of ˜10 μM and range down to a Cₘₐₓ of ˜0.1 μM. For example, a liquid nitrite salt solution administered by inhalation following nebulization from a device providing a fine particle dose percent (FPD %) of ˜25%: 1 mg (˜0.25 mg FPD) to 360 mg (˜90 mg FPD) device-loaded sodium nitrite provides human plasma nitrite levels between ˜0.1 μM and ˜10 μM; and dry powder sodium nitrite administered by inhalation following dispersion in a device providing a FPD % of ˜50%: 0.35 mg (˜0.18 mg FPD) to 35 mg (˜18 mg FPD) device-loaded dry powder sodium nitrite provides human plasma nitrite levels between ˜0.1 μM and ˜10 μM.

It is to be understood that both the foregoing general description and the following detailed description are exemplary and explanatory only and are not restrictive of the invention, as claimed.

### DEFINITIONS

The terms “administration” or “administering” and “delivery” or “delivering” refer to a method of giving to a vertebrate, or in the case of transplant, giving to an isolated tissue or organ, a dosage of a therapeutic or prophylactic formulation, such as a nitrite compound formulation described herein, for example as an anti-hypertensive, or to counter ischemia-reperfusion injury, or as an antimicrobial pharmaceutical (composition, or for other purposes. The preferred delivery method or method of administration can vary depending on various factors, e.g., the components of the pharmaceutical composition, the desired site at which the formulation is to be introduced, delivered or administered, the site where therapeutic benefit is sought, the site of a potential or actual microbial (e.g., bacterial, fungal, parasitic, viral, etc.) infection, the particular microbe involved, and/or the severity of an actual microbial infection.

A “carrier” or “excipient” is a compound or material used to facilitate administration of the compound, for example, to increase the solubility of the compound. Solid carriers include, e.g., starch, lactose, dicalcium phosphate, sucrose, and kaolin. Liquid carriers include, e.g., sterile water, saline, buffers, non-ionic surfactants, and edible oils such as oil, peanut and sesame oils. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

A “diagnostic” as used herein is a compound, method, system, or device that assists in the identification and characterization of a health or disease state. The diagnostic can be used in standard assays as is known in the art.

The term “mammal” is used in its usual biological sense. Thus, it specifically includes humans, cattle, horses, dogs, and cats, but also includes many other species.

The term “microbial infection” refers to the undesired proliferation or presence of invasion of pathogenic microbes (e.g., bacteria, fungi, viruses, microbial parasites including protozoa, etc.) in a host organism. This includes the excessive growth of microbes that are normally present in or on the body of a mammal or other organism. More generally, a microbial infection can be any situation in which the presence of a microbial population(s) is damaging to a host mammal. Thus, a microbial infection exists when excessive numbers of a microbial population are present in or on a mammal's body, or when the effects of the presence of a microbial population(s) is damaging the cells or other tissue of a mammal.

The term “pulmonary arterial hypertension” (PAH) refers to symptomatic presentation of exertional dyspnea, which is indicative of an inability to increase pulmonary blood flow with exercise. Exertional chest pain, syncope, and edema are indications of more severely impaired right heart function. Diagnosis of PAH is often made by echocardiography, which demonstrates evidence of right ventricular volume and pressure overload. Catheterization measuring arterial pressures may also be used in diagnosis.

The term “ischemic reperfusion injury” refers to damage to tissue caused when blood supply returns to the tissue after a period of ischemia. The absence of oxygen and nutrients from blood creates a condition in which the restoration of circulation results in inflammation and oxidative damage through the induction of oxidative stress rather than restoration of normal function

The term “transplant” refers to the moving of a whole or partial organ from one body to another (or from a donor site on the patient's own body), for the purpose of replacing the recipient's damaged or failing organ with a working one from the donor site.

The term “stroke” refers to the clinical designation for a rapidly developing loss of brain function due to an interruption in the blood supply to all or part of the brain.

The term “catheterization” refers to the process of inserting a tube (catheter) into a body cavity, duct or vessel. Catheters thereby allow drainage or injection of fluids or access by surgical instruments.

The term “ischemia” or “ischemic episode” refers to an inadequate flow of blood to a part of the body, tissue or organ, caused by constriction or blockage of the blood vessels supplying it, or in the case of transplantation, the lack of blood flow to a donor tissue/organ during the transplantion process. The result of decreased blood flow is inadequate oxygenation of tissue or organ.

The term “flushate” refers to a solution or formulation used to wash or bathe a tissue, organ or other mass.

The term “perfusate” refers to a solution or formulation administered ex vivo to a tissue or organ when systemic, blood low is not available, e.g., as in the case of a donor tissue or organ during the transplantation process, prior to recipient insertion and vascular connection.

The term “ex vivo” refers to experimentation or manipulation done in or on living tissue in an artificial environment outside the organism.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable salt” refers to salts that retain the biological effectiveness and properties of the compounds of this invention and, which are not biologically or otherwise undesirable. In many cases, the compounds of this invention are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, naphtoic acid, oleic acid, palmitic acid, pamoic (emboic) acid, stearic acid, glycolic acid, pyruvic acid, oxalic acid, maleic acid, malonic acid, succinic acid, fumaric acid, tartaric acid, citric acid, ascorbic acid, glucoheptonic acid, glucuronic acid, lactic acid, lactobioic acid, tartaric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, histidine, arginine, lysine, benethamine, N-methyl-glucamine, and ethanolamine. Other acids include dodecylsulfuric acid, naphthalene-1,5-disulfonic acid, naphthalene-2-sulfonic acid, and saccharin.

The term “nitrite, nitrite salt, or nitrite- or nitric oxide-donating compound” refers to nitrite anion-containing compounds and salt forms thereof that retain the biological effectiveness and properties of the nitrite anion as disclosed herein and that are not biologically or otherwise undesirable, and to other compounds that act as sources of nitrite as may be chemically and/or enzymatically converted to NO, or as donors of NO such as the compositions disclosed herein and salt forms thereof, and which are not biologically or otherwise undesirable. As noted above, in certain particularly preferred embodiments disclosed herein a nitrite compound comprises nitrite anion or a salt thereof, such as sodium nitrite, potassium nitrite or magnesium nitrite. Such species are those that upon reduction, oxidation, hydrolysis, or other chemical or biological process including enzymatic catalysis, produce or release nitric oxide for therapeutic or prophylactic purposes. Nitric oxide may be detected by any of a number of methodologies with which persons skilled in the art will be familiar, for example, using a NO Nanosensor as described in US 2005/0036949.

The term “fine particle dose (FPD)” means the amount of inhaled drug present in particles less than or equal to 5 microns in diameter (that which is expected to deposit in the lung following inhalation). Fine particle dose percent (FPD %) is the FPD expressed as percent of nominal dose.

Accordingly, in particularly preferred embodiments disclosed herein, a nitrite compound such as nitrite anion or a salt thereof, may be provided as sodium nitrite, potassium nitrite or magnesium nitrite, and may act as a therapeutic or prophylactic agent.

In certain other distinct embodiments, another nitrite- or nitric oxide-donating compound may serve directly as a therapeutic or prophylactic agent.

Whereas the preferred embodiments disclosed herein contemplate a nitrite compound that comprises nitrite anion or a salt thereof, such as sodium nitrite, potassium nitrite, pot nitrite or magnesium nitrite, other embodiments are not intended to be so limited such that a “nitrite- or nitric oxide-donating compound” may include, without limitation, one or more species such as nitrate, nitrogen dioxide, nitric oxide (gas) itself, nitrous acid, arginine, nitrosothiols, nitroglycerine, glutamine, lysine, asparagine, amyl nitrite, nitric oxide-donating aspirin, NG-nitro-L-arginine methylester, nitroprusside, nitrosobenzene, nitrosyl chloride, O-nitrosoethanol, ethyl nitrite, ethyl nitrate, S-nitrosoglutathione, Ruthenium(III) nitrosyl chloride, Nitrosyl tetrafluoroborate, Potassium pentachloronitrosylruthenate(II), Ruthenium(III) nitrosyl nitrate, 1-Nitroso-2-naphthol, 1-Nitroso-2-naphthol-3,6-disulfonic acid, 2-Methyl-2-nitrosopropane, 2-Nitroso-1-naphtho, 3-(3-Hydroxy-4-nitroso-N-propylanilino)propanesulfonic acid, 3-Hydroxy-4-nitroso-2,7-naphthalenedisulfonic acid, 6-Nitroso-1,2-benzopyrone, Cupferron, N-Benzyl-N-nitroso-p-toluenesulfonamide, N,N-Dimethyl-4-nitrosoaniline, N-Nitroso-N-ethylbutylamine, N-Nitroso-N-ethylurea, N-Nitroso-N-methylbutylamine, N-Nitroso-N-methylurea, N-Nitrosodiphenylamine, S-Nitroso-N-acetyl-DL-penicillamine, 1,3,5-Tri-tert-butyl-2-nitrosobenzene, 4-Hydroxy-3-nitroso-1-naphthalenesulfonic acid, Diazald®, N,N-Diethyl-4-nitrosoaniline, N-Nitrosxodiphenylamine, N-Nitrosodiphenylamine, N-Nitrosodiphenylamine solution, Dephostatin, Diazald®-N-methyl, PAPA NONOate, 6-Amino-1-methyl-5-nitrosouracil, Diazald®-N-methyl-N-methyl, 1,3-difluoro-2-nitroso-benzene, 1,8-dihydroxy-2-nitroso-3,6-naphthalenedisulfonic acid, copper complex, 1-ethyl-3-nitroso-2-phenylindole, 1-ethyl-3-nitroso-piperazine, 17-alpha-chloro-1-beta-nitroso-5-alpha-androstane, 2,6-diamino-5-nitroso-4-pyrimidinol, 2-nitro-1-nitroso-1-phenylcyclohexane, 2-nitroso-1,2-dihydroharmaline, 2-nitroso-1-naphtho-3,6-disulfonic acid, 2-nitroso-4,7,7-trimethyl-2-azabicyclo(2.2.1)heptan-3-one, 2-tert-butyl-6-methyl-4-nitroso-phenol, 3,5-dimethyl-4-nitroso-1H-pyrazole-3-alpha-chloro-3-beta-nitroso-5-alpha-cholestane, 3-alpha-chloro-3-beta-nitroso-5-alpha-cholestane, 3-cloro-3-nitroso-5-beta-cholestane, 3-nitro-1-nitroso-1-octylguanidine, 3-nitroso-1-oxa-3-azaspiro(4,5)decan-2-one, 3-nitroso-2,4,6-triacetamidopyridine, 3-nitroso-2-phenylimidazo[1,2-A]pyrimidine, 4-alpha-chloro-4-beta, nitroso-5-alpha-cholestane, 4-hydroxy-3-nitroso-1-naphthalene-sulfonic acid, 4-hydroxy-3-nitroso-1-naphthalene-sulfonic acid, 5-(3,5-di-tert-butylphenyl)-3-nitroso-2-oxazolidinone, 5-nitroso-quinolin-8-ol, 6-amino-5-nitroso-2-thiouracil, 7-alpha-chloro-7-beta-nitroso-5-alpha-cholestane, 7-methyl-3-nitroso-2-phenylimidazo[1,2-A]pyridine, diethyl-(3-nitroso-phenyl)-amine, 1N-(2-ethoxy-Ph)-2-(1-nitroso-3-oxo-1,2,3,4-tetrahydroquinozalin-2-yl)-acetamine-(4-bromo-phenyl)-5-nitroso-pyrimidine-2,4,6-triamine, N-methyl-N-nitroso-3-tetrahydrothiophen amine-1,1-dioxide, N—(N′-methyl-N′-nitroso-amino-methyl)-benzamide, N-nitroso-N-(2-pyridyl)-3-(trifluoromethyl)amine, N-nitroso-N-(trimethylsilylmethyl)-P-toluenesulfonamine, S-(9-nitrosos-9H-purin-6-yl)-2-chloroethylthiocarbamate, 2-Nitrosotoluene, 4-Nitrosodiphenylamine, N-Nitrosodiethylamine, Nitrosobenzene, Semustine, Butyl nitrite, Calcium nitrite, Dicyclohexylamine nitrite, Dicyclohexylammonium nitrite, Ethyl nitrite, Isoamyl nitrite, Isobutyl nitrite, Isopentyl nitrite, Potassium nitrite, Silver nitrite, Sodium nitrite, tert-Butyl nitrite, Tetrabutylammonium nitrite, Bis(triphenyl phosphoranylidene)ammonium nitrite, 2-Ethylhexyl nitrate, Isobutyl nitrate, Isopropyl nitrate, and magnesium nitrite.

The term “reducing acid” refers to acids that retain the biological effectiveness and properties of the compounds of this invention and, which are not biologically or otherwise undesirable. In many cases, the compounds of this invention are capable of reducing nitrite, nitrite salt, or nitrite- or nitric oxide-donating compound to produce or release nitric oxide. Pharmaceutically acceptable reducing acids include, for example, organic acids such as acetic acid, propionic acid, naphtoic acid, oleic acid, palmitic acid, pamoic (emboic) acid, stearic acid, glycolic acid, pyruvic acid, oxalic acid, maleic acid, malonic acid, succinic acid, fumaric acid, tartaric acid, citric acid, ascorbic acid, glucoheptonic acid, glucuronic acid, lactic acid, lactobioic acid, tartaric acid, benzoic acid, cinnamic acid, mandelic acid, methanesulfonic acid, ethanesulfonic acid, p-toluenesulfonic acid, salicylic acid, and the like.

The term “pH-reducing acid” refers to acids that retain the biological effectiveness and properties of the compounds of this invention and, which are not biologically or otherwise undesirable. In many cases, the compounds of certain embodiments are capable of reducing nitrite anion or a salt thereof, or a nitrite- or nitric oxide-donating compound, to produce or release nitric oxide. Pharmaceutically acceptable reducing acids include, for example, inorganic acids such as, e.g., hydrochloric acid, hydrobromic acid, sulfuric acid, nitric acid, phosphoric acid, and the like. Also by nonlimiting example, pH-reducing acids may also include organic acids such as citric acid, acetic acid, propionic acid, naphtoic acid, oleic acid, palmitic acid, pamoic (emboic) acid, stearic acid, glycolic acid, pyruvic acid, oxalic acid, maleic acid, malonic acid, succinic acid, fumaric acid, tartaric acid, citric acid, ascorbic acid, glucoheptonic acid, glucuronic acid, lactic acid, lactobioic acid, tartaric acid, benzoic acid, cinnamic acid, mandelic acid, methanesulfonic acid, ethanesulfonic acid, p-toluenesulfonic acid, salicylic acid, and the like.

According to certain herein disclosed embodiments a nitrite compound formulation may comprise an “acidic excipient” that is typically present as an acidic excipient aqueous solution. An “acidic excipient” refers to a non-reducing acid and as used herein expressly excludes, e.g., ascorbic acid or other acids that are capable of inducing a reaction with a nitrite compound at a pH of from about 4.7 to about 7.4 that could undesirably lead to detectable generation of nitrogen dioxide, such as detectable evolution of visible nitrogen dioxide 9 as bubbles from solution, or generation of deleterious levels of nitrogen dioxide in solution as assessed by standard cytotoxicity or toxicology assays. An acid that is “non-reducing” means a compound whose standard redox potential at 25° C. (relative to a hydrogen electrode) is greater than 0 volts. Examples of non-reducing acid salts include phosphate, sulphate, nitrate, acetate, formate, citrate, tartrate, propionate and sorbate. Non-reducing organic acids include carboxylic acids, sulfonic acids, phosphonic acids, phosphinic acids, phosphoric monoesters, and phosphoric diesters, and/or other organic acids that contain from 1 to 12 carbon atoms. Examples of non-reducing organic acids include citric acid, acetic acid, formic acid, propionic acid, butyric acid, benzoic acid, mono-, di-, and trichloroacetic acid, salicylic acid, trifluoroacetic acid, benzenesulfonic acid, toluenesulfonic acid, methylphosphonic acid, methylphosphinic acid, dimethylphosphinic acid, and phosphonic acid monobutyl ester.

A “buffer” refers to a compound that functions as a pH buffer. In certain related embodiments the pH buffer is present under conditions and in sufficient quantity to maintain a pH that is “about” a recited pH value. “About” such a pH refers to the functional presence of that buffer, which, as is known in the art, may be a consequence of a variety of factors including pKa value(s) of the buffer, buffer concentration, working temperature, effects of other components of the composition on pKa (i.e., the pH at which the buffer is at equilibrium between protonated and deprotonated forms, typically the center of the effective buffering range of pH values), and other factors.

Hence, “about” in the context of pH may be understood to represent a quantitative variation in pH that may be more or less than the recited value by no more than 0.5 pH units, more preferably no more than 0.4 pH units, more preferably no more than 0.3 pH units, still more preferably no more than 0.2 pH units, and most preferably no more than 0.1-0.15 pH units, (As also noted above, “about” when used to refer to a quantitative value other than in the context of pH, means that a specified quantity may be greater than or less than the indicated amount by 1, 2, 3, 4, 5, 6, 7, 8, 9, 10, 11, 12, 13, 14, 15, 16, 17, 18, 19 or 20 percent of the stated numerical value.)

As also noted above, in certain embodiments a substantially constant pH (e.g., a pH that is maintained within the recited range for an extended time period) may be from about pH 4.7 to about pH 7, from about pH 4.8 to about pH 6.9, from about pH 4.9 to about pH 6.8, from about pH 5.0 to about pH 6.7, from about pH 5.1 to about pH 6.6, or from about pH 5.2 to about pH 6.5, or any other pH or pH range as described herein, which in preferred embodiments may be from about pH 4.7 to about pH 6.5 for a nitrite compound formulation, and greater than about pH 7.0 for a nitrite compound aqueous solution. Maintenance of a substantially constant pH preferably includes an ability to regulate the pH of the composition or formulation so that it remains at “about” a recited pH for a lengthy period of time, typically on the order of at least 0.25, 0.5, 0.75, 1.0 or more hours.

Therefore the pH buffer typically may comprise a composition that, when present under appropriate conditions and in sufficient quantity, is capable of maintaining a desired pH level as may be selected by those familiar with the art, for example, buffers comprising citrate, malate, pyridine, piperazine, succinate, histidine, maleate, bis-Tris, pyrophosphate, PIPES, ACES, histidine, MES, cacodylic acid, H₂CO₃/NaHCO₃ and N-(2-Acetamido)-2-iminodiacetic acid (ADA) or other buffers for maintaining, preserving, enhancing, protecting or otherwise promoting desired biological or pharmacological activity of a nitrite compound, based on the disclosure herein. Suitable buffers may include those in Table 1 or known to the art (see, e.g., Calbiochem® Biochemicals & Immunochemicals Catalog 2004/2005, pp. 68-69 and catalog pages cited therein, EMD Biosciences, La Jolla, Calif.).

Non-limiting examples of buffers that may be used according to certain embodiments disclosed herein as may relate to a nitrite compound formulation that comprises in pertinent part a buffer that has a pKa between 5.1 and 6.8 and that is present at a concentration sufficient to maintain a pH from about 4.7 to about 6.5 for a time period of at least one hour at 23° C. are shown, with their pKa values, in Table 1:

TABLE 1
Exemplary Buffers and Relevant pKa

| | |
|---|---|
| Citric acid | 4.76 |
| Malate | 5.13 |
| Pyridine | 5.23 |
| Piperazine | 5.33 |
| Succinate | 5.64 |
| Histidine | 6.04 |
| Maleate | 6.24 |
| Citric acid | 6.40 |
| Bis-Tris | 6.46 |
| Pyrophosphate | 6.70 |
| PIPES | 6.76 |
| ACES | 6.78 |
| Histidine | 6.80 |
| MES | 6.15 |
| Cacodylic acid | 6.27 |
| H₂CO₃/NaHCO₃ | 6.37 |
| ADA | 6.60 |

Key:
ACES: N-(2-acetamido)-2-aminoethanesulfonic acid
ADA: N-(2-ametamino)iminodiacetic acid
BIS-TRIS: Bis(2-hydroxytheyl(amino-tris(hydroxymethyl)methane
MES: 4-morpholineethanesulfonic acid
PIPES: Piperazine-N,N′-bis(2-ethanesulfonic acid)

Non-limiting examples of buffers that may be used according to certain embodiments disclosed herein as may relate to a nitrite compound formulation that comprises a buffer that has a pKa between 6.5 and 9.3 and that is present at a concentration sufficient to maintain a pH from about 7.0 to about 9.0, with their pKa values, are presented in Table 2:

TABLE 2
Exemplary Buffers and Relevant pKa

| | |
|---|---|
| 2-amino-2-methyl-1,3-propanediol | 8.8 |
| ACES | 6.8 |
| ADA | 6.6 |
| AMPSO | 9.0 |
| BES | 7.1 |
| BICINE | 8.3 |
| BIS-TRIS | 6.5 |
| BIS-TRIS Propane | 6.8 |
| CHES | 9.3 |
| DIPSO | 7.6 |
| EPPS | 8.0 |
| Diglycine | 8.2 |
| HEPBS | 8.3 |
| HEPES | 7.5 |
| MOPS | 7.2 |
| MOPSO | 6.9 |
| PIPES | 6.8 |
| POPSO | 7.8 |
| Sodium phosphate dibasic | 6.8 |
| Sodium phosphate monobasic | 6.8 |
| Potassium phosphate dibasic | 6.8 |
| Potassium phosphate monobasic | 6.8 |
| TAPS | 8.4 |
| TAPSO | 7.6 |
| TES | 7.5 |
| Tricine | 8.1 |
| TRIZMA | 8.1 |

Key:
ACES: N-(2-acetamido)-2-aminoethanesulfonic acid
ADA: N-(2-ametamino)iminodiacetic acid
AMPSO: N-(1,1-dimethyl-2-hydroxyethyl)-3-amino-2-hydroxypropane-sulfonic acid
BES: N,N-Bis(2-hydroxyethyl)-2-aminoethanesulfonic acid
BICINE: N,N-Bis(2-hydroxyethyl)glycine
BIS-TRIS: Bis(2-hydroxytheyl(amino-tris(hydroxymethyl)methane
BIS-TRIS Propane: 1,3-Bis[tris(hydroxymethyl)methylamino]propane
CHES: 2-(cyclohexylamino)ethanesulfonic acid
DIPSO: 3-(N,N-Bis[2-hydroxyethyl]amino)-2-hydroxypropanesulfonic acid
EPPS: N-(2-hydroxyethyl)piperazine-N′-(3-propanesulfonic acid)
HEPBS: Diglycine, N-(2-hydroxyethyl)piperazine-N′-(4-butanesulfonic acid)
HEPES: N-(2-hydroxyethyl)piperazine-N′-(2-ethanesulfonic acid)
MOPS: 4-morpholinepropanesulfonic acid
MOPSO: beta-hydroxy-4-morpholinepropanesulfonic acid
PIPES: Piperazine-N,N′-bis(2-ethanesulfonic acid)
POPSO: Piperazine-N,N′-bis(2-hydroxypropanesulfonic acid)
TAPS: [(2-hydroxy-1,1-bis(hydroxymethyl)ethyl)amino]-1-propane-sulfonic acid
TAPSO: 2-hydroxy-3-[tris(hydroxymethyl)methylamino]-1-propanesulfonic acid
TES: N-[tris(hydroxymethyl)methyl]-2-aminoethanesulfonic acid
TRIZMA: 2-amino-2-(hydroxymehtyl)-1,3-propanediol

“Solvate” refers to the compound formed by the interaction of a solvent and nitrite, or nitrite- or nitric oxide-donating compound, antimicrobial, a metabolite, or salt thereof. Suitable solvates are pharmaceutically acceptable solvates including hydrates.

In the context of the response of a microbe, such as a bacterium, to an antimicrobial agent, the term “susceptibility” refers to the sensitivity of the microbe for the presence of the antimicrobial agent. So, to increase the susceptibility means that the microbe will be inhibited by a lower concentration of the antimicrobial agent in the medium surrounding the microbial cells. This is equivalent to saying that the microbe is more sensitive to the antimicrobial agent. In most cases the minimum inhibitory concentration (MIC) of that antimicrobial agent will have been reduced.

By “therapeutically effective amount” or “pharmaceutically effective amount” is meant a nitrite, nitrite salt, or nitrite- or nitric oxide-donating compound, as disclosed for this invention, which has a therapeutic effect. The doses of nitrite, nitrite salt, or nitrite- or nitric oxide-donating compound which are useful in treatment are therapeutically effective amounts. Thus, as used herein, a therapeutically effective amount means those amounts of nitrite, nitrite salt, or nitrite- or nitric oxide-donating compound which produce the desired therapeutic effect as judged by clinical trial results and/or model animal pulmonary hypertension, reperfusion and/or transplant studies. In particular embodiments, the nitrite, nitrite salt, or nitrite- or nitric oxide-donating compound are administered in a pre-determined dose, and thus a therapeutically effective amount would be an amount of the dose administered. This amount and the amount of the nitrite, nitrite salt, or nitrite- or nitric oxide-donating compound can be routinely determined by one of skill in the art, and will vary, depending on several factors, such as the particular microbial strain where a infection is applicable, or a therapeutic or prophylactic effect for pulmonary hypertension or reperfusion injury occurs, and how distant that disease site is from the initial respiratory location receiving the initial inhaled aerosol dose. This amount can further depend upon the patient's height, weight, sex, age and medical history. For prophylactic treatments, a therapeutically effective amount is that amount which would be effective to prevent a microbial infection, pulmonary hypertension or reperfusion injury.

A “therapeutic effect” relieves, to some extent and in a manner having clinical significance according to accepted parameters as may be known and applied by the art to a given indication, disease, disorder or clinical condition, one or more of the symptoms of infection, pulmonary hypertension, or ischemic effects or sequelae in an organ subjected to reperfusion or transplant. This effect includes curing such disease or disorder, slowing the progression of, or preventing infection in, pulmonary hypertension or reperfusion injury, or reducing (e.g., decreasing in a statistically significant manner) the severity of same. “Curing” means that the symptoms of disease are eliminated, or at a point below the threshold of detection by traditional measurements. However, certain long-term or permanent effects of the disease, disorder or condition may exist even after a cure is obtained (such as extensive tissue damage). As used herein, for infection, a “therapeutic effect” is defined as a statistically significant reduction in microbial (e.g., bacterial, fungal, viral, parasitic such as, e.g., protozoan parasite, etc.) load in a host, emergence of resistance, or improvement in infection symptoms as measured by human clinical results or animal studies.

For pulmonary hypertension, a “therapeutic effect” is defined as a statistically significant reduction in pulmonary arterial pressures and/or increase in exercise performance. For myocardial ischemic reperfusion injury, a “therapeutic effect” is defined as a statistically significant improvement in post-ischemic cardiac output and/or cardiac rhythm and/or cardiac electrical conduction. For cerebral ischemic reperfusion injury, a “therapeutic effect” is defined as a statistically significant decrease in post-ischemic infarct, size and/or decrease in cerebral edema and/or improvement in neurologic function. For ischemic reperfusion injury associated with lung transplant, a “therapeutic effect” is defined as a statistically significant improvement in pulmonary gas exchange and/or pulmonary radiographic infiltrates and/or duration of mechanical ventilation post-transplantation. For ischemic reperfusion injury associated with heart transplant, a “therapeutic effect” is defined as a statistically significant improvement in cardiac output and/or cardiac rhythm and/or cardiac electrical conduction. For ischemic reperfusion injury associated with kidney transplant, a “therapeutic effect” is defined as a statistically significant improvement in renal function (if want to define more tightly: electrolyte status and/or acid base status and/or intra and extravascular fluid status). For ischemic reperfusion injury associated with liver transplant, a “therapeutic effect” is defined as a statistically significant improvement in post-transplant hepatic synthetic function and/or hepatic metabolic function.

“Treat,” “treatment,” or “treating,” as used herein refers to administering a pharmaceutical composition for prophylactic and/or therapeutic purposes. The term “prophylactic treatment” refers to treating a patient who is not yet diseased, but who is susceptible to, or otherwise at risk of, a particular disease. The term “therapeutic treatment” refers to administering treatment to a patient already suffering from a disease. Thus, in preferred embodiments, treating is the administration to a mammal (either for therapeutic or prophylactic purposes) of therapeutically effective amounts of a nitrite, nitrite salt, or nitrite- or nitric oxide-donating compound.

Pharmacokinetics (PK) is concerned with the time course of nitrite, or nitrite- or nitric oxide-donating compound concentration in the body. Pharmacodynamics (PD) is concerned with the relationship between pharmacokinetics and efficacy in vivo. PK/PD parameters correlate nitrite, or nitrite- or nitric oxide-donating compound exposure with efficacious activity. Accordingly, to predict the therapeutic efficacy of nitrite, nitrite salts, or nitrite- or nitric oxide-donating compound with diverse mechanisms of action different PK/PD parameters may be used.

The term “dosing interval” refers to the time between administrations of the two sequential doses of a pharmaceutical during multiple dosing regimens.

As used herein, the “peak period” of a pharmaceutical's in vivo concentration is defined as that time of the pharmaceutical dosing interval when the pharmaceutical concentration is not less than 50% of its maximum plasma or site-of-disease concentration. In some embodiments, “peak period” is used to describe an interval of nitrite, or nitrite- or nitric oxide-donating compound dosing.

The “respirable delivered dose” is the amount of aerosolized drug-containing particles inhaled during the inspiratory phase of the breath simulator that is equal to or less than 5 microns using a simulator programmed to the European Standard pattern of 15 breaths per minute, with an inspiration to expiration ratio of 1:1 or following single or multiple inhalations of a dry powder or meter-dose inhalation device.

### Advantages of Inhaled Aerosol and Topical (Non-Oral) Drug Delivery

Inhalation therapy of aerosolized nitrite, or nitrite- or nitric oxide-donating compound enables direct deposition of the sustained-release or active substance in the respiratory tract (be that intra-nasal or pulmonary) for therapeutic action at that site of deposition or systemic absorption to regions immediately down stream of the vascular absorption site. In the case of pulmonary, or intra-nasal or sinus infections, intra-nasal inhalation aerosol delivery deposits nitrite, or nitrite- or nitric oxide-donating compound directly to that site of nasal infection or provides direct access through the ostia of the sinus for potential sinus infection therapy. Similarly, a pulmonary infection can be treated or prevented by oral inhalation and/or nasal inhalation of aerosol therapy to the lung.

Therapeutic and/or prophylactic activity against pulmonary arterial hypertension by administration of inhaled aerosol nitrite compound, or in distinct embodiments of inhaled aerosol nitrite- or nitric oxide-donating compound, appears to depend upon exposure of the nitrite compound (or NO-donating compound to the reductive and/or acid environment of the pulmonary lining fluid, and/or exposure to the pulmonary vasculature. These interactions then liberate nitric oxide which in turn serves as a vasodilator and/or agent that halts and/or reverses diseased vascular remodeling associated with this disease.

Similar to the intra-nasal and pulmonary applications described above, treatment or prevention of ischemic reperfusion injury to organs outside the respiratory tract involves absorption to the systemic vascular compartment for transport of prodrug or drug (e.g., nitrite compound) to these extra-respiratory sites. In the case of treating or preventing ischemic reperfusion injury in either the myocardium or cerebrum, deposition of drug in the respiratory tract, more specifically the deep lung, will enable direct access to these organs through the left atrium to either the carotid arteries or coronary arteries. This direct delivery will permit direct dosing of a high concentration of nitrite compound (or in distinct embodiments of nitrite or nitric oxide-donating compound) while avoiding general systemic exposure. Similarly, this route permits titration of the dose to a level that is appropriate for these indications. This rationale also applies to presently disclosed embodiments that are directed to organ transplant recipients, specifically, for example, organs that are immediately downstream of the left ventrical (by way of illustration and not limitation, the heart, liver and kidney). Pulmonary transplants are dosed directly through pulmonary absorption.

To test the hypothesis that inhaled sodium nitrite delivered directly to the lung could serve as a nitric oxide donor and elicit a reduction in pulmonary arterial hypertension, 300 mg sodium nitrite in 5 mL was administered via aerosol to newborn lambs subjected to antecedent hypoxia to induce pulmonary hypertension. Hypoxia was associated with a rapid rise in pulmonary arterial pressure (PAP) from 21±1 to 34±2 mmHg, a 20% rise in pulmonary vascular resistance and a modest 20% decrease in systemic vascular resistance. Inhaled nitrite at a dose of 15 mg/minute elicited a rapid and sustained reduction (approximately 65%) in hypoxia-induced pulmonary hypertension compared with saline nebulization. Minimal effective doses of sodium nitrite were 1.5 mg/min in these lambs (Hunter, et al., 2004).

The magnitude of inhaled nitrite effect approached that of 20 ppm inhaled NO gas. Interestingly, this reduction in PAP was maintained for at least 1 hour after cessation of inhalation of the nebulized nitrite compared to nitric oxide gas wherein efficacy was lost within minutes following discontinuation of NO treatment. Nitrite-induced reduction of PAP was associated with the immediate appearance of NO in expired air, peaking at approximately 15 ppb after 20 minutes inhalation. Pulmonary vasodilation elicited by aerosolized nitrite was deoxyhemoglobin- and pH-dependent and was associated with increased blood levels of iron-nitrosyl-hemoglobin. Notably, from a therapeutic standpoint, short-term delivery of nitrite dissolved in saline through nebulization produced selective, sustained pulmonary vasodilation with no clinically significant increase in blood methemoglobin levels, rising from a basal level of 2% to a peak level of 3% 30 minutes following nebulization. Plasma nitrite concentration increased from a basal level of approximately 2 μmol/L pre-nebulization to a peak of 30 μmol/L after 20 minutes sodium nitrite nebulization. Plasma nitrite levels dropped rapidly upon cessation of inhalation, approaching basal levels at 90 minutes following discontinuation of nebulization.

### Pharmaceutical Compositions

For purposes of the methods described herein according to certain embodiments, a nitrite compound (e.g., nitrite anion or a salt thereof, preferably sodium nitrite, magnesium nitrite or potassium nitrite), or in distinct embodiments a nitrite- or nitric oxide-donating compound, may be administered using a liquid nebulization, dry powder or metered-dose inhaler. In some embodiments, a nitrite, nitrite salt, or nitrite- or nitric oxide-donating compound disclosed herein is produced as a pharmaceutical composition suitable for aerosol formation, dose for indication, deposition location, pulmonary or intra-nasal delivery for pulmonary, intranasal/sinus, or extra-respiratory therapeutic action, good taste, manufacturing and storage stability, and patient safety and tolerability.

In some embodiments, the isoform content of the manufactured nitrite, nitrite salt, or nitrite- or nitric oxide-donating compound, most preferably sodium nitrite or other nitrite salt form may be optimized for drug substance and dr-g product stability, dissolution (in the case of dry powder or suspension formulations) in the nose and/or lung, tolerability, antimicrobial activity and site of action (be that lung, nasal/sinus, or systemic).

### Administration

The nitrite, nitrite salt, or nitrite- or nitric oxide-donating compound, most preferably sodium nitrite or other nitrite salt form disclosed herein can be administered at a therapeutically effective dosage, e.g., a dosage sufficient to provide treatment for the disease states previously described. Generally, for example, a daily aerosol dose of nitrite compound (e.g., nitrite anion) in a nitrite compound formula ion may be from about 0.01 to 10.0 mg, nitrite anion/kg of body weight, preferably about 0.05 to 8.0 mg/kg of body weight, and more preferably about 0.1 to 5.0 mg/kg of body weight. Thus, for administration to a 70 kg person, the dosage range would be about 0.7 to 700.0 mg nitrite anion per day, preferably about 3.5 to 560.0 mg per day, and more preferably about 7.0 to 350.0 mg per day. The amount of active compound administered will, of course, be dependent on the subject and disease state being treated, the severity of the affliction, the manner and schedule of administration, the location of the disease (e.g., whether it is desired to effect intra-nasal or upper airway delivery, pharyngeal or laryngeal delivery, bronchial delivery, pulmonary delivery and/or pulmonary delivery with subsequent systemic absorption), and the judgment of the prescribing physician; for example, a likely dose range for aerosol administration of nitrite anion in preferred embodiments, or in other embodiments of nitrite or nitric oxide-donating compound, would be about 7.0 to 350.0 mg per day.

Administration of the nitrite compound (e.g., nitrite anion or salt thereof), or of a nitrite- or nitric oxide-donating compound, preferably sodium nitrite or another nitrite salt form as disclosed herein, such as a pharmaceutically acceptable salt thereof, can be via any of the accepted modes of administration for agents that serve similar utilities including, but not limited to, aerosol inhalation such as nasal and/or oral inhalation of a mist or spray containing liquid particles, for example, as delivered by a nebulizer.

Pharmaceutically acceptable compositions thus may include solid, semi-solid, liquid and aerosol dosage forms, such as, e.g., powders, liquids, suspensions, complexations, liposomes, particulates, or the like. Preferably, the compositions are provided in unit dosage forms suitable for single administration of a precise dose. The unit dosage form can also be assembled and packaged together to provide a patient with a weekly or monthly supply and can also incorporate other compounds such as saline, taste masking agents, pharmaceutical excipients, and other active ingredients or carriers.

The nitrite compound (e.g., nitrite anion or a salt thereof), or nitrite- or nitric oxide-donating compound, preferably sodium nitrite or other nitrite salt form, can be administered either alone or more typically in combination with a conventional pharmaceutical carrier, excipient or the like (e.g., mannitol, lactose, starch, magnesium stearate, sodium saccharin (which as disclosed herein may also be present in certain preferred embodiments as a taste-masking agent, including at a range of specified molar ratios relative to sodium nitrite), talcum, cellulose, sodium crosscarmellose, glucose, gelatin, sucrose, magnesium carbonate, magnesium chloride, magnesium sulfate, calcium chloride, lactose, sucrose, glucose and the like). If desired, the pharmaceutical composition can also contain minor amounts of nontoxic auxiliary substances such as wetting agents, emulsifying agents, solubilizing agents, pH buffering agents and the like (e.g., citric acid, ascorbic acid, sodium phosphate, potassium phosphate, sodium acetate, sodium citrate, cyclodextrin derivatives, sorbitan monolaurate, triethanolamine acetate, triethanolamine oleate, and the like). Generally, depending on the intended mode of administration, the pharmaceutical formulation will contain about 0.005% to 95%, preferably about 0.5% to 50% by weight of a compound of the invention. Actual methods of preparing such dosage forms are known, or will be apparent, to those skilled in this art; for example, see Remington's Pharmaceutical Sciences, Mack Publishing Company, Easton, Pa.

In one preferred embodiment, the compositions will take the form of a unit dosage form such as vial containing a liquid, solid to be suspended, dry powder, lyophilisate, or other composition and thus the composition may contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like.

Liquid pharmaceutically administrable compositions can, for example, be prepared by dissolving, dispersing, etc. an active compound as defined above and optional pharmaceutical adjuvants in a carrier (e.g., water, saline, aqueous dextrose, glycerol, glycols, ethanol or the like) to form a solution or suspension. Solutions to be aerosolized can be prepared in conventional forms, either as liquid solutions or suspensions, as emulsions, or in solid forms suitable for dissolution or suspension in liquid prior to aerosol production and inhalation. The percentage of active compound contained in such aerosol compositions is highly dependent on the specific nature thereof, as well as the activity of the compound and the needs of the subject. However, percentages of active ingredient of 0.01% to 90% in solution are employable, and will be higher if the composition is a solid, which will be subsequently diluted to the above percentages. In some embodiments, the composition will comprise 1.0%-50.0% of the active agent in solution.

Nitrite, nitrite salt, or nitrite- or nitric oxide-donating compound formulations can be separated into two groups; those of simple formulation and complex formulations providing taste-masking for improved tolerability, pH-optimized properties for nitric oxide formation and/or release, and/or area-under-the-curve (AUC) shape-enhancing properties. Simple formulations can be further separated into three groups. 1. Simple formulations may include water-based liquid formulations for nebulization. By non-limiting example water-based liquid formulations may consist of the nitrite, nitrite salt, or nitrite- or nitric oxide-donating compound alone or with non-encapsulating water soluble excipients. 2. Simple formulations may also include organic-based liquid formulations for nebulization or meter-dose inhaler. By non-limiting example organic-based liquid formulations may consist of the nitrite, nitrite salt, or nitrite- or nitric oxide-donating compound or with non-encapsulating organic soluble excipients. 3. Simple formulations may also include dry powder formulations for administration with a dry powder inhaler. By non-limiting example dry powder formulations may consist of the nitrite, nitrite salt, or nitrite- or nitric oxide-donating compound alone or with either water soluble or organic soluble non-encapsulating excipients with or without a blending agent such as lactose. Complex formulations can be further separated into five groups. 1. Complex formulations may include water-based liquid formulations for nebulization. By non-limiting example water-based liquid complex formulations may consist of the nitrite, nitrite salt, or nitrite- or nitric oxide-donating compound encapsulated or complexed with water-soluble excipients such as lipids, liposomes cyclodextrins, microencapsulations, and emulsions. 2. Complex formulations may also include organic-based liquid formulations for nebulization or meter-dose inhaler. By non-limiting example organic-based liquid complex formulations may consist of the nitrite, nitrite salt, or nitrite- or nitric oxide-donating compound encapsulated or complexed with organic-soluble excipients such as lipids, microencapsulations, and reverse-phase water-based emulsions. 3. Complex formulations may also include low-solubility, water-based liquid formulations for nebulization. By non-limiting example low-solubility, water-based liquid complex formulations may consist of the nitrite, nitrite salt, or nitrite- or nitric oxide-donating compound as a low-water soluble, stable nanosuspension alone or in co-crystal/co-precipitate excipient complexes, or mixtures with low solubility lipids, such as lipid nanosuspensions. 4. Complex formulations may also include low-solubility, organic-based liquid formulations for nebulization or meter-dose inhaler. By non-limiting example low-solubility, organic-based liquid complex formulations may consist of the nitrite, nitrite salt, or nitrite- or nitric oxide-donating compound as a low-organic soluble, stable nanosuspension alone or in co-crystal/co-precipitate excipient complexes, or mixtures with low solubility lipids, such as lipid nanosuspensions. 5. Complex formulations may also include dry powder formulations for administration using a dry powder inhaler. By non-limiting example, complex dry powder formulations may consist of the nitrite, nitrite salt, or nitrite- or nitric oxide-donating compound in co-crystal/co-precipitate/spray dried complex or mixture with low-water soluble excipients/salts in dry powder form with or without a blending agent such as lactose. Specific methods for simple and complex formulation preparation are described herein.

### Aerosol Delivery

Nitrite, nitrite salt, or nitrite- or nitric oxide-donating compound as described herein, are preferably directly administered as an aerosol to a site of pulmonary pathology including pulmonary hypertension, pulmonary transplant or pulmonary infection. The aerosol may also be delivered to the pulmonary compartment for absorption into the pulmonary vasculature for therapy or prophylaxis of extra-pulmonary pathologies such as myocardial and cerebral reperfusion injury following, by non-limiting example myocardial infarction or stroke, respectively. Extrapulmonary pathologies may also include kidney, liver, and heart transplants and their associated potential for ischemic reperfusion injury. Pulmonary transplant is also recognized as a pathology. In some embodiments, aerosol delivery is used to treat an infection in the lungs, such as a Pseudomonas lung infection.

Several device technologies exist to deliver either dry powder or liquid aerosolized products. Dry powder formulations generally require less time for drug administration, yet longer and more expensive development efforts. Conversely, liquid formulations have historically suffered from longer administration times, yet have the advantage of shorter and less expensive development efforts. The nitrite, nitrite salt, or nitrite- or nitric oxide-donating compound disclosed herein range in solubility, are generally stable and have a range of tastes. In one such embodiment, the nitrite, nitrite salt, or nitrite- or nitric oxide-donating compounds are water soluble at neutral pH, is stable in aqueous solution and have limited to no taste. Such salts include sodium nitrite and magnesium nitrite.

Accordingly, in one embodiment, a particular formulation of the nitrite, nitrite salt, or nitrite- or nitric oxide-donating compound disclosed herein is combined with a particular aerosolizing device to provide an aerosol for inhalation that is optimized for maximum drug deposition at a site of infection, pulmonary arterial hypertension, pulmonary or intra-nasal site for systemic absorption for extra-nasal and/or extra-pulmonary indications, and maximal tolerability. Factors that can be optimized include solution or solid particle formulation, rate of delivery, and particle size and distribution produced by the aerosolizing device.

### Particle Size and Distribution

Generally, inhaled particles are subject to deposition by one of two mechanisms: impaction, which usually predominates for larger particles, and sedimentation, which is prevalent for smaller particles. Impaction occurs when the momentum of an inhaled particle is large enough that the particle does not follow the air stream and encounters a physiological surface. In contrast, sedimentation occurs primarily in the deep lung when very small particles which have traveled with the inhaled air stream encounter physiological surfaces as a result of random diffusion within the air stream.

For pulmonary administration, the upper airways are avoided in favor of the middle and lower airways. Pulmonary drug delivery may be accomplished by inhalation of an aerosol through the mouth and throat. Particles having a mass median aerodynamic diameter (MMAD) of greater than about 5 microns generally do not reach the lung; instead, they tend to impact the back of the throat and are swallowed and possibly orally absorbed. Particles having diameters of about 2 to about 5 microns are small enough to reach the upper- to mid-pulmonary region (conducting airways), but are too large to reach the alveoli. Smaller particles, i.e., about 0.5 to about 2 microns, are capable of reaching the alveolar region. Particles having diameters smaller than about 0.5 microns can also be deposited in the aiveolar region by sedimentation, although very small particles may be exhaled. Measures of particle size can be referred to as volumetric mean diameter (VMD), mass median diameter (MMD), or MMAD. These measurements may be made by impaction (MMD and MMAD) or by laser (VMD). For liquid particles, VMD, MMD and MMAD may be the same if environmental conditions are maintained, e.g., standard humidity. However, if humidity is not maintained, MMD and MMAD determinations will be smaller than VMD due to dehydration during impactor measurements. For the purposes of this description, VMD, MMD and MMAD measurements are considered to be under standard conditions such that descriptions of VMD, MMD and MMAD will be comparable. Similarly, dry powder particle size determinations in MMD, and MMAD are also considered comparable.

In some embodiments, the particle size of the aerosol is optimized to maximize the nitrite compound (or in distinct embodiments, the nitrite- or nitric oxide-donating compound) deposition at the site of pulmonary pathology, respiratory infection and/or extra-pulmonary, systemic distribution, and to maximize tolerability (or in the later case, systemic absorption). Aerosol particle size may be expressed in terms of the mass median aerodynamic diameter (MMAD). Large particles (e.g., MMAD>5 μm) may deposit in the upper airway because they are too large to navigate the curvature of the upper airway. Small particles (e.g., MMAD<2 μm) may be poorly deposited in the lower airways and thus become exhaled, providing additional opportunity for upper airway deposition. Hence, intolerability (e.g., cough and bronchospasm) may occur from upper airway deposition from both inhalation impaction of large particles and settling of small particles during repeated inhalation and expiration. Thus, in one embodiment, an optimum particle size is used (e.g., MMAD=2-5 μm) in order to maximize deposition at a mid-lung site of infection and to minimize intolerability associated with upper airway deposition. Moreover, generation of a defined particle size with limited geometric standard deviation (GSD) may optimize deposition and tolerability. Narrow GSD limits the number of particles outside the desired MMAD size range. In one embodiment, an aerosol containing one or more compounds disclosed herein is provided having a MMAD from about 2 microns to about 5 microns with a GSD of less than or equal to about 2.5 microns. In another embodiment, an aerosol having an MMAD from about 2.8 microns to about 4.3 microns with a GSD less than or equal to 2 microns is provided. In another embodiment, an aerosol having an MMAD from about 2.5 microns to about 4.5 microns with a GSD less than or equal to 1.8 microns is provided. In certain other preferred embodiments there is provided one or a plurality of liquid particles of about 0.1 to 5.0 microns VMD, the particle comprising a nitrite compound formulation as described herein.

The nitrite compound (e.g., nitrite anion or salt thereof, such as sodium nitrite, magnesium nitrite or potassium nitrite) according to preferred embodiments or, in separate but related embodiments, the nitrite- or nitric oxide-donating compound, as disclosed herein and intended for respiratory delivery (for either systemic or local distribution) can be administered as aqueous formulations, as suspensions or solutions in halogenated hydrocarbon propellants, or as dry powders. Aqueous formulations may be aerosolized by liquid nebulizers employing either hydraulic or ultrasonic atomization. Propellant-based systems may use suitable pressurized metered-dose inhalers (pMDIs). Dry powders may use dry powder inhaler devices (DPIs), which are capable of dispersing the drug substance effectively. A desired particle size and distribution may be obtained by choosing an appropriate device.

### Liquid Nebulizer

In one embodiment, a nebulizer is selected on the basis of allowing the formation of an aerosol of a nitrite, nitrite salt, or nitrite- or nitric oxide-donating compound disclosed herein having an MMAD predominantly between about 2 to about 5 microns. In one embodiment, the delivered amount of nitrite, nitrite salt, or nitrite- or nitric oxide-donating compound provides a therapeutic effect for pulmonary pathology, respiratory infections and/or extra-pulmonary, systemic distribution.

Previously, two types of nebulizers, jet and ultrasonic, have been shown to be able to produce and deliver aerosol particles having sizes between 2 and 4 um. These particle sizes have been shown as being optimal for middle airway deposition and hence, treatment of pulmonary bacterial infections caused by gram-negative bacteria such as Pseudomonas aeruginosa, Escherichia coli, Enterobacter species, Klebsiella pneumoniae, K. oxytoca, Proteus mirabilis, Pseudomonas aeruginosa, Serratia marcescens, Haemophilus influenzae, Burkholderia cepacia, Stenotrophomonas maltophilia, Alcaligenes xylosoxidans, and multidrug resistant Pseudomonas aeruginosa. However, unless a specially formulated solution is used, these nebulizers typically need larger volumes to administer sufficient amount of drug to obtain a therapeutic effect. A jet nebulizer utilizes air pressure breakage of an aqueous solution into aerosol droplets. An ultrasonic nebulizer utilizes shearing of the aqueous solution by a piezoelectric crystal. Typically, however, the jet nebulizers are only about 10% efficient under clinical conditions, while the ultrasonic nebulizer is only about 5% efficient. The amount of pharmaceutical deposited and absorbed in the lungs is thus a fraction of the 10% in spite of the large amounts of the drug placed in the nebulizer. Smaller particle sizes or slow inhalation rates permit deep lung deposition. Both middle-lung and alveolar deposition may be desired for this invention depending on the indication, e.g., middle airway deposition for antimicrobial activity, or middle and/or aiveolar deposition for pulmonary arterial hypertension and systemic delivery. Exemplary disclosure of compositions and methods for formulation delivery using nebulizers can be found in, e.g., US 2006/0276483, including descriptions of techniques, protocols and characterization of aerosolized mist delivery using a vibrating mesh nebulizer.

Accordingly, in one embodiment, a vibrating mesh nebulizer is used to deliver in preferred embodiments an aerosol of the nitrite compound as disclosed herein (e.g., nitrite anion or salt thereof), or in other embodiments, a nitrite- or nitric oxide-donating compound as disclosed herein. A vibrating mesh nebulizer comprises a liquid storage container in fluid contact with a diaphragm and inhalation and exhalation valves, in one embodiment, about 1 to about 5 mL of the nitrite compound formulation (or in another related embodiment, of a nitrite- or NO-donating compound formulation) is placed in the storage container and the aerosol generator is engaged producing atomized aerosol of particle sizes selectively between about 1 and about 5 μm volumetric mean diameter.

Thus, for example, in preferred embodiments a nitrite compound formulation as provided herein, or in alternative embodiments a nitrite- or nitric oxide-producing compound formulation as disclosed herein, is placed in a liquid nebulization inhaler and prepared in dosages to deliver from about 7 to about 700 mg from a dosing solution of about 1 to about 5 mL, preferably from about 17.5 to about 700 mg in about 1 to about 5 mL, more preferably from about 175 to about 350 mg in about 1 to about 5 mL, preferably about 0.1 to about 300 mg in about 1 to about 5 mL and more preferable 0.25 to about 90 mg in about 1 to about 5 mL with volumetric mean diameter particles sizes between about 1 to about 5 μm being produced.

By non-limiting example, a nebulized nitrite, nitrite salt, or nitrite- or nitric oxide-donating compound may be administered in the described respirable delivered dose in less than about 20 min, preferably less than about 10 min, more preferably less than about 7 min, more preferably less than about 5 min, more preferably less than about 3 min, and in some cases most preferable if less than about 2 min.

By non-limiting example, in other circumstances, a nebulized nitrite, nitrite salt, or nitrite- or nitric oxide-donating compound may achieve improved tolerability and/or exhibit an area-under-the-curve (AUC) shape-enhancing characteristic when administered over longer periods of time. Under these conditions, the described respirable delivered dose in more than about 2 min, preferably more than about 3 min, more preferably more than about 5 min, more preferably more than about 7 min, more preferably more than about 10 min, and in some cases most preferable from about 10 to about 20 min.

As disclosed herein, there is provided an exemplary nitrite compound formulation composition comprising (i) a nitrite compound aqueous solution having a pH greater than 7.0; and (ii) an acidic excipient aqueous solution. In certain embodiments the nitrite compound formulation composition is provided in the form of at least the two separate liquid solution components (i) and (ii) which can be admixed to form a nitrite compound formulation, such as may be used to load a nebulizer for delivery to a human patient or a veterinary subject. As also noted above, certain surprising advantages of the herein disclosed embodiments derive from the selection of the components for (i) and (ii) such that upon admixture to form the nitrite compound formulation, the nitrite compound is present at a concentration of from about 14.5 mM to about 2.174 M nitrite anion, the nitrite compound formulation has a pH of from about 4.7 to about 6.5, and nitric oxide bubbles are not visually detectable for at least 15, 30, 45 or 60 minutes following admixture. “Visually detectable” refers to bubbles that would be readily discernible in a standard clear laboratory glass vessel by the unaided human eye of an individual having normal vision. In certain other embodiments the nitrite compound formulation is provided as an aqueous solution having a pH of from about 4.7 to about 6.5, the solution comprising a nitrite compound at a concentration of from about 14.5 mM to 2.174 M nitrite anion; and citric acid at a concentration of from about 0.021 mM to about 3.2 mM. In certain other embodiments the nitrite compound formulation is provided as an aqueous solution having a pH of from about 4.7 to about 6.5, the solution comprising a nitrite compound at a concentration of from about 14.5 mM to 2.174 M nitrite anion; and a buffer that has a pKa between 5.1 and 6.8 and that is present at a concentration sufficient to maintain a pH from about 4.7 to about 6.5 for a time period of at least one hour at 23° C.

In particular, and as described herein, selection of the nitrite compound formulation according to these and related embodiments provides a formulation in which NO that is formed remains in solution as a dissolved solute; the rate of NO formation, according to non-limiting theory, is not sufficient to result in visually detectable NO bubbles as would result in loss of NO to the atmosphere. The absence of such NO gas evolution surprisingly permits the nitrite compound formulation to be administered using a vibrating mesh nebulizer to form an aerosol comprising liquid particles of about 0.1 to about 5.0 microns volumetric mean diameter and 12-1800 parts per billion (ppb) NO, an unexpected advantage for such a formulation insofar as previously described acidified nitrite solutions are characterized by NO gas evolution that would cause gas bubbles to block the mesh of a vibrating mesh nebulizer. By contrast, the presently disclosed nitrite compound formulation does not detectably impair the vibrating mesh nebulizer, as can be assessed by comparing (i) the time-to-dryness of nebulizing a known volume of the nitrite compound formulation and (ii) the time-to-dryness of nebulizing an equivalent volume of the nitrite compound aqueous solution (which contains nitrite but has a pH greater than 7 and so would not be a source of appreciable NO generation).

By way of elaboration, according to this criterion, elapsed nebulizer running times are determined, in separate runs, for complete discharge from the nebulizer reservoir of equal fluid volumes of the formulation (i) and the solution (ii). Comparable times-to-dryness indicate that the two liquid preparations are dispensed by the nebulizer with equal efficiency, signifying that in the formulation (i) no gas bubble formation can be detected, as would otherwise decrease the discharge rate and lead to an increased time-to-dryness, i.e., a longer elapsed time before the fluid reservoir has been discernibly emptied as a result of nebulized liquid discharge from the device.

For aqueous and other non-pressurized liquid systems, a variety of nebulizers (including small volume nebulizers) are available to aerosolize the formulations. Compressor-driven nebulizers incorporate jet technology and use compressed air to generate the liquid aerosol. Such devices are commercially available from, for example, Healthdyne Technologies, Inc.; Invacare, Inc.; Mountain Medical Equipment Inc.; Pari Respiratory, Inc. (Midiothian, Va.); Mada Medical, Inc.; Puritan-Bennet; Schuco, Inc., DeVilbiss Health Care, Inc.; and Hospitak, Inc. Ultrasonic nebulizers rely on mechanical energy in the form of vibration of a piezoelectric crystal to generate respirable liquid droplets and are commercially available from, for example, Omron Heathcare, Inc. and DeVilbiss Health Care, Inc. Vibrating mesh nebulizers rely upon either piezoelectric or mechanical pulses to respirable liquid droplets generate. Other examples of nebulizers for use with nitrite, nitrite salt, or nitrite or nitric oxide-donating compound described herein are described in U.S. Pat. Nos. 4,268,460; 4,253,468; 4,046,146; 3,826,255; 4,649,911; 4,510,929; 4,624,251; 5,164,740; 5,586,550; 5,758,637; 8,644,304; 6,338,443; 5,906,202; 5,934,272; 5,960,792; 5,971,951; 6,070,575; 6,192,876; 6,230,706; 6,349,719; 6,367,470; 6,543,442; 6,584,971; 6,601,581; 4,263,907; 5,709,202; 5,823,179; 6,192,876; 6,644,304; 5,549,102; 6,083,922; 6,161,536; 6,264,922; 6,557,549; and 6,612,303 all of which are hereby incorporated by reference in their entireties. Commercial examples of nebulizers that can be used with the nitrite, nitrite salt, or nitrite- or nitric oxide-donating compound described herein include Respirgard II®, Aeroneb®, Aeroneb® Pro, and Aeroneb® Go produced by Aerogen (Aerogen, Inc., Galway, Ireland); AERx® and AERx Essence™ produced by Aradigm; Porta-Neb®, Freeway Freedom™, Sidestream, Ventstream and I-neb produced by Respironics, Inc. (Murrysville, Pa.); and PARI LC-Plus®, PARI LC-Star®, and e-Flow™ produced by PARI, GmbH (PARI Respiratory Equipment, Inc., Midiothian, Va.; PARI GmbH, Starnberg, Germany). By further non-limiting example, U.S. Pat. No. 6,196,219, is hereby incorporated by reference in its entirety.

In some embodiments, the drug solution is formed prior to use of the nebulizer by a patient. In other embodiments, the drug is stored in the nebulizer in solid form. In this case, the solution is mixed upon activation of the nebulizer, such as described in U.S. Pat. No. 6,427,682 and PCT Publication No. WO 03/035030, both of which are hereby incorporated by reference in their entireties. In these nebulizers, the solid drug, optionally combined with excipients to form a solid composition, is stored in a separate compartment from a liquid solvent.

The liquid solvent is capable of dissolving the solid composition to form a liquid composition, which can be aerosolized and inhaled. Such capability is, among other factors, a function of the selected amount and, potentially, the composition of the liquid. To allow easy handling and reproducible dosing, the sterile aqueous liquid may be able to dissolve the solid composition within a short period of time, possibly under gentle shaking. In some embodiments, the final liquid is ready to use after no longer than about 30 seconds. In some cases, the solid composition is dissolved within about 20 seconds, and advantageously, within about 10 seconds. As used herein, the terms “dissolve(d)”, “dissolving”, and “dissolution” refer to the disintegration of the solid composition and the release, i.e., the dissolution, of the active compound. As a result of dissolving the solid composition with the liquid solvent a liquid composition is formed in which the active compound is contained in the dissolved state. As used herein, the active compound is in the dissolved state when at least about 90 wt.-% are dissolved, and more preferably when at least about 95 wt.-% are dissolved.

With regard to basic separated-compartment nebulizer design, it primarily depends on the specific application whether it is more useful to accommodate the aqueous liquid and the solid composition within separate chambers of the same container or primary package, or whether they should be provided in separate containers. If separate containers are used, these are provided as a set within the same secondary package. The use of separate containers is especially preferred for nebulizers containing two or more doses of the active compound. There is no limit to the total number of containers provided in a multi-dose kit. In one embodiment, the solid composition is provided as unit doses within multiple containers or within multiple chambers of a container, whereas the liquid solvent is provided within one chamber or container. In this case, a favorable design provides the liquid in a metered-dose dispenser, which may consist of a glass or plastic bottle closed with a dispensing device, such as a mechanical pump for metering the liquid. For instance, one actuation of the pumping mechanism may dispense the exact amount of liquid for dissolving one dose unit of the solid composition.

In another embodiment for multiple-dose separated-compartment nebulizers, both the solid composition and the liquid solvent are provided as matched unit doses within multiple containers or within multiple chambers of a container. For instance, two-chambered containers can be used to hold one unit of the solid composition in one of the chambers and one unit of liquid in the other. As used herein, one unit is defined by the amount of drug present in the solid composition, which is one unit dose. Such two-chambered containers may, however, also be used advantageously for nebulizers containing only one single drug dose.

In one embodiment of a separated-compartment nebulizer, a blister pack having two blisters is used, the blisters representing the chambers for containing the solid composition and the liquid solvent in matched quantities for preparing a dose unit of the final liquid composition. As used herein, a blister pack represents a thermoformed or pressure-formed primary packaging unit, most likely comprising a polymeric packaging material that optionally includes a metal foil, such as aluminum. The blister pack may be shaped to allow easy dispensing of the contents. For instance, one side of the pack may be tapered or have a tapered portion or region through which the content is dispensable into another vessel upon opening the blister pack at the tapered end. The tapered end may represent a tip.

In some embodiments, the two chambers of the blister pack are connected by a channel, the channel being adapted to direct fluid from the blister containing the liquid solvent to the blister containing the solid composition. During storage, the channel is closed with a seal. In this sense, a sea is any structure that prevents the liquid solvent from contacting the solid composition. The seal is preferably breakable or removable; breaking or removing the seal when the nebulizer is to be used will allow the liquid solvent to enter the other chamber and dissolve the solid composition. The dissolution process may be improved by shaking the blister pack. Thus, the final liquid composition for inhalation is obtained, the liquid being present in one or both of the chambers of the pack connected by the channel, depending on how the pack is held.

According to another embodiment, one of the chambers, preferably the one that is closer to the tapered portion of the blister pack, communicates with a second channel, the channel extending from the chamber to a distal position of the tapered portion. During storage, this second channel does not communicate with the outside of the pack but is closed in an air-tight fashion. Optionally, the distal end of the second channel is closed by a breakable or removable cap or closure, which may e.g., be a twist-off cap, a break-off cap, or a cut-off cap.

In one embodiment, a vial or container having two compartments is used, the compartment representing the chambers for containing the solid composition and the liquid solvent in matched quantities for preparing a dose unit of the final liquid composition. The liquid composition and a second liquid solvent may be contained in matched quantities for preparing a dose unit of the final liquid composition (by non-limiting example in cases where two soluble excipients or the nitrite, nitrite salt, or nitrite- or nitric oxide-donating compound and excipient are unstable for storage, yet desired in the same mixture for administration.

In some embodiments, the two compartments are physically separated but in fluid communication such as when so the vial or container are connected by a channel or breakable barrier, the channel or breakable barrier being adapted to direct fluid between the two compartments to enable mixing prior to administration. During storage, the channel is closed with a seal or the breakable barrier intact. In this sense, a seat is any structure that prevents mixing of contents in the two compartments. The seal is preferably breakable or removable; breaking or removing the seal when the nebulizer is to be used will allow the liquid solvent to enter the other chamber and dissolve the solid composition or in the case of two liquids permit mixing. The dissolution or mixing process may be improved by shaking the container. Thus, the final liquid composition for inhalation is obtained, the liquid being present in one or both of the chambers of the pack connected by the channel or breakable barrier, depending on how the pack is held.

The solid composition itself can be provided in various different types of dosage forms, depending on the physicochemical properties of the drug, the desired dissolution rate, cost considerations, and other criteria. In one of the embodiments, the solid composition is a single unit. This implies that one unit dose of the drug is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In a preferred embodiment, the solid composition is a highly porous lyophilized form. Such lyophilizates, sometimes also called wafers or lyophilized tablets, are particularly useful for their rapid disintegration, which also enables the rapid dissolution of the active compound.

On the other hand, for some applications the solid composition may also be formed as a multiple unit dosage form as defined above. Examples of multiple units are powders, granules, microparticles, pellets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution.

Another type of multiparticulate system which is also capable of achieving rapid drug dissolution is that of powders, granules, or pellets from water-soluble excipients which are coated with the drug, so that the drug is located at the outer surface of the individual particles. In this type of system, the water-soluble low molecular weight excipient is useful for preparing the cores of such coated particles, which can be subsequently coated with a coating composition comprising the drug and, preferably, one or more additional excipients, such as a binder, a pore former, a saccharide, a sugar alcohol, a film-forming polymer, a plasticizer, or other excipients used in pharmaceutical coating compositions.

In another embodiment, the solid composition resembles a coating layer that is coated on multiple units made of insoluble material. Examples of insoluble units include beads made of glass, polymers, metals, and mineral salts. Again, the desired effect is primarily rapid disintegration of the coating layer and quick drug dissolution, which is achieved by providing the solid composition in a physical form that has a particularly high surface-to-volume ratio. Typically, the coating composition will, in addition to the drug and the water-soluble low molecular weight excipient, comprise one or more excipients, such as those mentioned above for coating soluble particles, or any other excipient known to be useful in pharmaceutical coating compositions.

To achieve the desired effects, it may be useful to incorporate more than one water-soluble low molecular weight excipient into the solid composition. For instance, one excipient may be selected for its drug carrier and diluent capability, while another excipient may be selected to adjust the pH. If the final liquid composition needs to be buffered, two excipients that together form a buffer system may be selected.

In one embodiment, the liquid to be used in a separated-compartment nebulizer is an aqueous liquid, which is herein defined as a liquid whose major component is water. The liquid does not necessarily consist of water only; however, in one embodiment it is purified water. In another embodiment, the liquid contains other components or substances, preferably other liquid components, but possibly also dissolved solids. Liquid components other than water which may be useful include propylene glycol, glycerol, and polyethylene glycol. One of the reasons to incorporate a solid compound as a solute is that such a compound is desirable in the final liquid composition, but is incompatible with the solid composition or with a component thereof, such as the active ingredient.

Another desirable characteristic for the liquid solvent is that it is sterile. An aqueous liquid would be subject to the risk of considerable microbiological contamination and growth if no measures were taken to ensure sterility, in order to provide a substantially sterile liquid, an effective amount of an acceptable antimicrobial agent or preservative can be incorporated or the liquid can be sterilized prior to providing it and to seal it with an air-tight seal in one embodiment, the liquid is a sterilized liquid free of preservatives and provided in an appropriate air-tight container. However, according to another embodiment in which the nebulizer contains multiple doses of the active compound, the liquid may be supplied in a multiple-dose container, such as a metered-dose dispenser, and may require a preservative to prevent microbial contamination after the first use.

### Meier Dose Inhaler (MDI)

A propellant driven inhaler (pMDI) releases a metered dose of medicine upon each actuation. The medicine is formulated as a suspension or solution of a drug substance in a suitable propellant such as a halogenated hydrocarbon, pMDIs are described in, for example, Newman, S. P., Aerosols and the Lung, Clarke et al., eds., pp. 197-224 (Butterworths, London, England, 1984).

In some embodiments, the particle size of the drug substance in an MDI may be optimally chosen. In some embodiments, the particles of active ingredient have diameters of less than about 50 microns, in some embodiments, the particles have diameters of less than about 10 microns. In some embodiments, the particles have diameters of from about 1 micron to about 5 microns. In some embodiments, the particles have diameters of less than about 1 micron. In one advantageous embodiment, the particles have diameters of from about 2 microns to about 5 microns.

By non-limiting example, metered-dose inhalers (MDI), the nitrite, nitrite salt, or nitrite- or nitric oxide-donating compound disclosed herein are prepared in dosages to deliver from about 7 to about 700 mg from a formulation meeting the requirements of the MDI, preferably from about 17.5 to 700 mg in an MDI formulation, and more preferably from about 17.5 to 700 mg from an MDI formulation. The nitrite, nitrite salt, or nitrite- or nitric oxide-donating compound disclosed herein may be soluble in the propellant, soluble in the propellant plus a co-solvent (by non-limiting example ethanol), soluble in the propellant plus an additional moiety promoting increased solubility (by non-limiting example glycerol or phospholipid), or as a stable suspension or micronized, spray-dried or nanosuspension.

By non-limiting example, a metered-dose nitrite, nitrite salt, or nitrite- or nitric oxide-donating compound may be administered in the described respirable delivered dose in 10 or fewer inhalation breaths, more preferably in 8 or fewer inhalation breaths, more preferably in 6 or fewer inhalation breaths, more preferably in 8 or fewer inhalation breaths, more preferably in 4 or fewer inhalation breaths, more preferably in 2 or fewer inhalation breaths.

The propellants for use with the MDIs may be any propellants known in the art. Examples of propellants include chlorofluorocarbons (CFCs) such as dichlorodifluoromethane, trichlorofluoromethane, and dichorotetrafluoroethane; hydrofluoroalkanes (HFAs); and carbon dioxide. It may be advantageous to use HFAs instead of CFCs due to the environmental concerns associated with the use of CFCs. Examples of medicinal aerosol preparations containing HFAs are presented in U.S. Pat. Nos. 6,585,958; 2,868,691 and 3,014,844, all of which are hereby incorporated by reference in their entireties. In some embodiments, a co-solvent is mixed with the propellant to facilitate dissolution or suspension of the drug substance.

In some embodiments, the propellant and active ingredient are contained in separate containers, such as described in U.S. Pat. No. 4,534,345, which is hereby incorporated by reference in its entirety.

In some embodiments, the MDI used herein is activated by a patient pushing a lever, button, or other actuator. In other embodiments, the release of the aerosol is breath activated such that, after initially arming the unit, the active compound aerosol is released once the patient begins to inhale, such as described in U.S. Pat. Nos. 6,672,304; 5,404,871; 5,347,998; 5,284,133; 5,217,004; 5,119,806; 5,060,643; 4,664,107; 4,648,393; 3,789,843; 3,732,864; 3,636,949; 3,598,294; 3,565,070; 3,456,646; 3,456,645; and 3,456,644, each of which is hereby incorporated by reference in its entirety. Such a system enables more of the active compound to get into the lungs of the patient. Another mechanism to help a patient get adequate dosage with the active ingredient may include a valve mechanism that allows a patient to use more than one breath to inhale the drug, such as described in U.S. Pat. Nos. 4,470,412 and 5,385,140, both of which are hereby incorporated by reference in their entireties.

Additional examples of MDIs known in the art and suitable for use herein include U.S. Pat. Nos. 6,435,177; 6,585,958; 5,642,730; 6,223,746; 4,955,371; 5,404,871; 5,364,838; and 6,523,536, all of which are hereby incorporated by reference in their entireties,

### Dry Powder Inhaler (DPI)

There are two major designs of dry powder inhalers. One design is the metering device in which a reservoir for the drug is placed within the device and the patient adds a dose of the drug into the inhalation chamber. The second is a factory-metered device in which each individual dose has been manufactured in a separate container. Both systems depend upon the formulation of drug into small particles of mass median diameters from about 1 to about 5 μm, and usually involve co-formulation with larger excipient particles (typically 100 μm diameter lactose particles), Drug powder is placed into the inhalation chamber (either by device metering or by breakage of a factory-metered dosage) and the inspiratory flow of the patient accelerates the powder out of the device and into the oral cavity. Non-laminar flow characteristics of the powder path cause the excipient-drug aggregates to decompose, and the mass of the large excipient particles causes their impaction at the back of the throat, while the smaller drug particles are deposited deep in the lungs.

As with liquid nebulization and MDIs, particle size of the nitrite, nitrite salt, or nitrite- or nitric oxide-donating compound aerosol formulation may be optimized. If the particle size is larger than about 5 μm MMAD then the particles are deposited in upper airways. If the particle size of the aerosol is smaller than about 1 μm then it is delivered into the alveoli and may get transferred into the systemic blood circulation.

By non-limiting example, in dry powder inhalers, the nitrite, nitrite salt, or nitrite- or nitric oxide-producing compound disclosed herein are prepared in dosages to deliver from about 5 to about 750 mg from a dry powder formulation, preferably from about 5 to 100 mg from a dry powder formulation, preferably from about 5 to 50 mg, preferably from about 0.1 to 35 mg and more preferably about 0.18 to about 18 mg from a dispersed and delivered.

By non-limiting example, a dry powder nitrite, nitrite salt, or nitrite- or nitric oxide-donating compound may be administered in the described respirable delivered dose in 10 or fewer inhalation breaths, more preferably in 8 or fewer inhalation breaths, more preferably in 6 or fewer inhalation breaths, more preferably in 8 or fewer inhalation breaths, more preferably in 4 or fewer inhalation breaths, more preferably in 2 or fewer inhalation breaths.

In some embodiments, a dry powder inhaler (DPI) is used to dispense the nitrite, nitrite salt, or nitrite- or nitric oxide-donating compound described herein. DPIs contain the drug substance in fine dry particle form. Typically, inhalation by a patient causes the dry particles to form an aerosol cloud that is drawn into the patient's lungs. The fine dry drug particles may be produced by any technique known in the art. Some well-known techniques include use of a jet mill or other comminution equipment, precipitation from saturated or super saturated solutions, spray drying, in situ micronization (Hovione), or supercritical fluid methods. Typical powder formulations include production of spherical pellets or adhesive mixtures. In adhesive mixtures, the drug particles are attached to larger carrier particles, such as lactose monohydrate of size about 50 to about 100 microns in diameter. The larger carrier particles increase the aerodynamic forces on the carrier/drug agglomerates to improve aerosol formation. Turbulence and/or mechanical devices break the agglomerates into their constituent parts. The smaller drug particles are then drawn into the lungs while the larger carrier particles deposit in the mouth or throat. Some examples of adhesive mixtures are described in U.S. Pat. No. 5,478,578 and PCT Publication Nos. WO 95/11666, WO 87/05213, WO 96/23485, and WO 97/03649, all of which are incorporated by reference in their entireties. Additional excipients may also be included with the drug substance.

There are three common types of DPIs, all of which may be used with the nitrite, nitrite salt, or nitrite- or nitric oxide-donating compounds described herein. In a single-dose DPI, a capsule containing one dose of dry drug substance/excipients is loaded into the inhaler. Upon activation, the capsule is breached, allowing the dry powder to be dispersed and inhaled using a dry powder inhaler. To dispense additional doses, the old capsule must be removed and an additional capsule loaded. Examples of single-dose DPIs are described in U.S. Pat. Nos. 3,807,400; 3,906,950; 3,991,761; and 4,013,075, all of which are hereby incorporated by reference in their entireties. In a multiple unit dose DPI, a package containing multiple single dose compartments is provided. For example, the package may comprise a blister pack, where each blister compartment contains one dose. Each dose can be dispensed upon breach of a blister compartment. Any of several arrangements of compartments in the package can be used. For example, rotary or strip arrangements are common. Examples of multiple unit does DPIs are described in EPO Patent Application Publication Nos. 0211595A2, 0455463A1, and 0467172A1, all of which are hereby incorporated by reference in their entireties. In a multi-dose DPI, a single reservoir of dry powder is used. Mechanisms are provided that measure out single dose amounts from the reservoir to be aerosolized and inhaled, such as described in U.S. Pat. Nos. 5,829,434; 5,437,270; 2,587,215; 5,113,855; 5,840,279; 4,688,218; 4,667,668; 5,033,463; and 4,805,811 and PCT Publication No. WO 92/09322, all of which are hereby incorporated by reference in their entireties.

In some embodiments, auxiliary energy in addition to or other than a patient's inhalation may be provided to facilitate operation of a DPI. For example, pressurized air may be provided to aid in powder de-agglomeration, such as described in U.S. Pat. Nos. 3,906,950; 5,113,855; 5,388,572; 6,029,662 and PCT Publication Nos. WO 93/12831, WO 90/07351, and WO 99/62495, all of which are hereby incorporated by reference in their entireties. Electrically driven impellers may also be provided, such as described in U.S. Pat. Nos. 3,948,264; 3,971,377; 4,147,166; 6,006,747 and PCT Publication No. WO 98/03217, all of which are hereby incorporated by reference in their entireties. Another mechanism is an electrically powered tapping piston, such as described in PCT Publication No. WO 90/13327, which is hereby incorporated by reference in its entirety. Other DPIs use a vibrator, such as described in U.S. Pat. Nos. 5,694,920 and 6,026,809, both of which are hereby incorporated by reference in their entireties. Finally, a scraper system may be employed, such as described in PCT Publication No. WO 93/24165, which is hereby incorporated by reference in its entirety.

Additional examples of DPIs for use herein are described in U.S. Pat. Nos. 4,811,731; 5,113,855; 5,840,279; 3,507,277; 3,669,113; 3,635,219; 3,991,761; 4,353,365; 4,889,144, 4,907,538; 5,829,434; 6,681,768; 6,561,186; 5,918,594; 6,003,512; 5,775,320; 5,740,794; and 6,626,173, all of which are hereby incorporated by reference in their entireties.

In some embodiments, a spacer or chamber may be used with any of the inhalers described herein to increase the amount of drug substance that gets absorbed by the patient, such as is described in U.S. Pat. Nos. 4,470,412; 4,790,305; 4,926,852; 5,012,803; 5,040,527; 5,024,467; 5,816,240; 5,027,806; and 6,026,807, all of which are hereby incorporated by reference in their entireties. For example, a spacer may delay the time from aerosol production to the time when the aerosol enters a patient's mouth. Such a delay may improve synchronization between the patient's inhalation and the aerosol production. A mask may also be incorporated for infants or other patients that have difficulty using the traditional mouthpiece, such as is described in U.S. Pat. Nos. 4,809,692; 4,832,015; 5,012,804; 5,427,089; 5,645,049; and 5,988,160, all of which are hereby incorporated by reference in their entireties.

Dry powder inhalers (DPIs), which involve deaggregation and aerosolization of dry powders, normally rely upon a burst of inspired air that is drawn through the unit to deliver a drug dosage. Such devices are described in, for example, U.S. Pat. No. 4,807,814, which is directed to a pneumatic powder ejector having a suction stage and an injection stage; SU 628930 (Abstract), describing a hand-held powder disperser having an axial air flow tube; Fox et al., Powder and Bulk Engineering, pages 33-36 (March 1988), describing a venturi eductor having an axial air inlet tube upstream of a venturi restriction; EP 347 779, describing a hand-held powder disperser having a collapsible expansion chamber, and U.S. Pat. No. 5,785,049, directed to dry powder delivery devices for drugs.

### Solution/Dispersion Formulations

In one embodiment, aqueous formulations containing soluble or nanoparticulate drug particles are provided. For aqueous aerosol formulations, the drug may be present at a concentration of about 0.67 mg/mL up to about 700 mg/mL; in certain preferred embodiments the nitrite compound is present at a concentration of from about 0.667 mg nitrite anion per mL to about 100 mg nitrite anion per mL. Such formulations provide effective delivery to appropriate areas of the lung, with the more concentrated aerosol formulations having the additional advantage of enabling large quantities of drug substance to be delivered to the lung in a very short period of time. In one embodiment, a formulation is optimized to provide a well tolerated formulation. Accordingly, certain preferred embodiments comprise a nitrite compound (e.g., nitrite anion or a salt thereof, such as sodium nitrite, potassium nitrite or magnesium nitrite) and are formulated to have good taste, pH from about 4.7 to about 6.5, osmolarity from about 100 to about 3600 mOsmol/kg, and optionally in certain further embodiments, a permeant ion (e.g., chloride, bromide) concentration from about 30 to about 300 mM.

In one embodiment, the solution or diluent used for preparation of aerosol formulations has a pH range from about 4.5 to about 9.0, preferably from about 4.7 to about 6.5 (e.g., as an acidic admixture), or from about 7.0 to about 9.0 as a single vial configuration. This pH range improves tolerability, as does the inclusion of a taste-masking agent according to certain embodiments as described elsewhere herein. When the aerosol is either acidic or basic, it can cause bronchospasm and cough. Although the safe range of pH is relative and some patients may tolerate a mildly acidic aerosol, while others will experience bronchospasm. Any aerosol with a pH of less than about 4.5 typically induces bronchospasm. Aerosols with a pH from about 4.5 to about 5.5 will cause bronchospasm occasionally. Any aerosol having pH greater than about 8 may have low tolerability because body tissues are generally unable to buffer alkaline aerosols. Aerosols with controlled pH below about 4.5 and over about 8.0 typically result in lung irritation accompanied by severe bronchospasm cough and inflammatory reactions. For these reasons as well as for the avoidance of bronchospasm, cough or inflammation in patients, the optimum pH for the aerosol formulation was determined to be between about pH 5.5 to about pH 8.0. Consequently, in one embodiment, aerosol formulations for use as described herein are adjusted to pH between about 4.5 and about 7.5 with the most preferred pH range for the acidic admixture from about 4.7 to about 6.5, and the most preferred pH range for the single vial configuration from about 7.0 to about 8.0.

By non-limiting example, compositions may according to certain embodiments disclosed herein also include a pH buffer or a pH adjusting agent, typically a salt prepared from an organic acid or base, and in preferred embodiments an acidic excipient as described herein (e.g., a non-reducing acid such as citric acid or a citrate salt, such as sodium citrate) or a buffer such as citrate or other buffers described above and with reference to Table 1. These and other representative buffers thus may include organic acid salts of citric acid, ascorbic acid, gluconic acid, carbonic acid, tartaric acid, succinic acid, acetic acid, or phthalic acid, Tris, tromethamine, hydrochloride, or phosphate buffers.

Many patients have increased sensitivity to various chemical tastes, including bitter, salt, sweet, metallic sensations. To create well-tolerated drug products, by non-limiting example taste masking may be accomplished through the addition of taste-masking agents and excipients, adjusted osmolality, and sweeteners.

Many patients have increased sensitivity to various chemical agents and have high incidence of bronchospastic, asthmatic or other coughing incidents. Their airways are particularly sensitive to hypotonic or hypertonic and acidic or alkaline conditions and to the presence of any permanent ion, such as chloride. Any imbalance in these conditions or a presence of chloride above a certain concentration value leads to bronchospastic or inflammatory events and/or cough which greatly impair treatment with inhalable formulations. Both of these conditions may prevent efficient delivery of aerosolized drugs into the endobronchial space, absent the advantageous uses of regulated pH, osmolality and taste-masking agent according to certain embodiments disclosed herein.

In some embodiments, the osmolality of aqueous solutions of the nitrite compound (or in distinct embodiments of the nitrite- or nitric oxide-donating compound) disclosed herein are adjusted by providing excipients. In some cases, a certain amount of a permeant ion, such as chloride, bromide or another anion, may promote successful and efficacious delivery of aerosolized nitrite compound or nitrite- or nitric oxide-donating compound. However, it has been discovered that for the nitrite compound formulations disclosed herein, the amounts of such permeant ions may be lower than the amounts that are typically used for aerosolized administration of other drug compounds.

Bronchospasm or cough reflexes may not in all cases be ameliorated by the use of a diluent for aerosolization having a given osmolality. However, these reflexes often can be sufficiently controlled and/or suppressed when the osmolality of the diluent is within a certain range. A preferred solution for aerosolization of therapeutic compounds which is safe and tolerated has a total osmolality from about 100 to about 3600 mOsmol/kg with a range of chloride concentration of from about 30 mM to about 300 mM and preferably from about 50 mM to about 150 mM. This osmolality controls bronchospasm, and the chloride concentration, as a permeant anion, controls cough. Because they are both permeant ions, bromine or iodine anions may be substituted for chloride. In addition, bicarbonate may substituted for chloride ion.

By non-limiting example, the formulation according to certain preferred embodiments for an aerosol nitrite compound (or in distinct embodiments for a nitrite- or nitric oxide-donating compound) may comprise from about 0.667 mg nitrite anion per mL to about 100 mg nitrite anion per mL, and in certain other embodiments may comprise from about 0.7 to about 700 mg, from about 3.5 to about 560 mg, or from about 7.0 to about 350 mg nitrite compound (or in distinct embodiments, nitrite- or nitric oxide-donating compound) per about 1 to about 5 mL water or dilute saline (e.g., dilutions of between 1/10 to 1/1 normal saline, i.e., 145 mM NaCl). Accordingly, the solution concentration of a nitrite compound (e.g., nitrite anion or a salt thereof, such as sodium nitrite, potassium nitrite or magnesium nitrite) in such embodiments (or in distinct embodiments of a nitrite- or nitric oxide-donating compound) may be greater than about 5 mg/mL, greater than about 10 mg/mL, greater than about 25 mg/mL, greater than about 50 mg/mL, greater than about 75 mg/mL, greater than about 90 mg/mL, or greater than about 100 mg/mL.

In certain embodiments, solution osmolality is from about 100 mOsmol/kg to about 3600 mOsmol/kg. In various other embodiments, the solution osmolality is from about 300 mOsmol/kg to about 3000 mOsmol/kg; from about 400 mOsmol/kg to about 2500 mOsmol/kg; and from about 500 mOsmol/kg to about 2000 mOsmol/kg. In certain embodiments, permeant ion concentration is from about 25 mM to about 400 mM. In various other embodiments, permeant ion concentration is from about 30 mM to about 300 mM; from about 40 mM to about 200 mM; and from about 50 mM to about 150 mM.

### Solid Particle Formulations

In some embodiments, solid drug nanoparticles are provided for use in generating dry aerosols or for generating nanoparticles in liquid suspension. Powders comprising nanoparticulate drug can be made by spray-drying aqueous dispersions of a nanoparticulate drug and a surface modifier to form a dry powder which consists of aggregated drug nanoparticles. In one embodiment, the aggregates can have a size of about 1 to about 2 microns which is suitable for deep lung delivery. The aggregate particle size can be increased to target alternative delivery sites, such as the upper bronchial region or nasal mucosa by increasing the concentration of drug in the spray-dried dispersion or by increasing the droplet size generated by the spray dryer.

Alternatively, an aqueous dispersion of drug and surface modifier can contain a dissolved diluent such as lactose or mannitol which, when spray dried, forms respirable diluent particles, each of which contains at least one embedded drug nanoparticle and surface modifier. The diluent particles with embedded drug can have a particle size of about 1 to about 2 microns, suitable for deep lung delivery. In addition, the diluent particle size can be increased to target alternate delivery sites, such as the upper bronchial region or nasal mucosa by increasing the concentration of dissolved diluent in the aqueous dispersion prior to spray drying, or by increasing the droplet size generated by the spray dryer.

Spray-dried powders can be used in DPIs or pMDIs, either alone or combined with freeze-dried nanoparticulate powder. In addition, spray-dried powders containing drug nanoparticles can be reconstituted and used in either jet or ultrasonic nebulizers to generate aqueous dispersions having respirable droplet sizes, where each droplet contains at least one drug nanoparticle. Concentrated nanoparticulate dispersions may also be used in these embodiments of the invention.

Nanoparticulate drug dispersions can also be freeze-dried to obtain powders suitable for nasal or pulmonary delivery. Such powders may contain aggregated nanoparticulate drug particles having a surface modifier. Such aggregates may have sizes within a respirable range, e.g., about 2 to about 5 microns MMAD.

Freeze dried powders of the appropriate particle size can also be obtained by freeze drying aqueous dispersions of drug and surface modifier, which additionally contain a dissolved diluent such as lactose or mannitol. In these instances the freeze dried powders consist of respirable particles of diluent, each of which contains at least one embedded drug nanoparticle.

Freeze-dried powders can be used in DPIs or pMDIs, either alone or combined with spray-dried nanoparticulate powder. In addition, freeze-dried powders containing drug nanoparticles can be reconstituted and used in either jet or ultrasonic nebulizers to generate aqueous dispersions that have respirable droplet sizes, where each droplet contains at least one drug nanoparticle.

One embodiment of the invention is directed to a process and composition for propellant-based systems comprising nanoparticulate drug particles and a surface modifier. Such formulations may be prepared by wet milling the coarse drug substance and surface modifier in liquid propellant, either at ambient pressure or under high pressure conditions. Alternatively, dry powders containing drug nanoparticles may be prepared by spray-drying or freeze-drying aqueous dispersions of drug nanoparticles and the resultant powders dispersed into suitable propellants for use in conventional pMDIs. Such nanoparticulate pMDI formulations can be used for either nasal or pulmonary delivery. For pulmonary administration, such formulations afford increased delivery to the deep lung regions because of the small (e.g., about 1 to about 2 microns MMAD) particle sizes available from these methods. Concentrated aerosol formulations can also be employed in pMDIs.

Another embodiment is directed to dry powders which contain nanoparticulate compositions for pulmonary or nasal delivery. The powders may consist of respirable aggregates of nanoparticulate drug particles, or of respirable particles of a diluent which contains at least one embedded drug nanoparticle. Powders containing nanoparticulate drug particles can be prepared from aqueous dispersions of nanoparticles by removing the water via spray-drying or lyophilization (freeze drying). Spray-drying is less time consuming and less expensive than freeze-drying, and therefore more cost-effective. However, certain drugs, such as biologicals benefit from lyophilization rather than spray-drying in making dry powder formulations.

Conventional micronized drug particles used in dry powder aerosol delivery having particle diameters of from about 2 to about 5 microns MMAD are often difficult to meter and disperse in small quantities because of the electrostatic cohesive forces inherent in such powders. These difficulties can lead to loss of drug substance to the delivery device as well as incomplete powder dispersion and sub-optimal delivery to the lung. Many drug compounds, particularly proteins and peptides, are intended for deep lung delivery and systemic absorption. Since the average particle sizes of conventionally prepared dry powders are usually in the range of from about 2 to about 5 microns MMAD, the fraction of material which actually reaches the alveolar region may be quite small. Thus, delivery of micronized dry powders to the lung, especially the alveolar region, is generally very inefficient because of the properties of the powders themselves.

The dry powder aerosols which contain nanoparticulate drugs can be made smaller than comparable micronized drug substance and, therefore, are appropriate for efficient delivery to the deep lung. Moreover, aggregates of nanoparticulate drugs are spherical in geometry and have good flow properties, thereby aiding in dose metering and deposition of the administered composition in the lung or nasal cavities.

Dry nanoparticulate compositions can be used in both DPIs and pMDIs. As used herein, “dry” refers to a composition having less than about 5% water.

In one embodiment, compositions are provided containing nanoparticles which have an effective average particle size of less than about 1000 nm, more preferably less than about 400 nm, less than about 300 nm, less than about 250 nm, or less than about 200 nm, as measured by light-scattering methods. By “an effective average particle size of less than about 1000 nm” it is meant that at least 50% of the drug particles have a weight average particle size of less than about 1000 nm when measured by light scattering techniques. Preferably, at least 70% of the drug particles have an average particle size of less than about 1000 nm, more preferably at least 90% of the drug particles have an average particle size of less than about 1000 nm, and even more preferably at least about 95% of the particles have a weight average particle size of less than about 1000 nm.

For aqueous aerosol formulations, the nanoparticulate agent may be present at a concentration of about may comprise from about 0.667 mg nitrite anion per mL to about 100 mg nitrite anion per mL, and in certain other embodiments may comprise from about 0.7 to about 700 mg, from about 3.5 to about 560 mg, or from about 7.0 to about 350 mg nitrite compound (or in distinct embodiments, nitrite- or nitric oxide-donating compound) per about 1 to about 5 mL water or dilute saline (e.g., dilutions of between 1/10 to 1/1 normal saline, i.e., 145 mM NaCl). Accordingly, the solution concentration of a nitrite compound (e.g., nitrite anion or a salt thereof, such as sodium nitrite, potassium nitrite or magnesium nitrite) in such embodiments (or in distinct embodiments of a nitrite- or nitric oxide-donating compound) may be greater than about 5 mg/mL, greater than about 10 mg/mL, greater than about 25 mg/mL, greater than about 50 mg/mL, greater than about 75 mg/mL, greater than about 90 mg/mL, or greater than about 100 mg/mL for aqueous aerosol formulations, and about 0.1 mg up to about 50 mg nitrite anion or about 5.0 mg/g up to about 1000 mg/g for dry powder aerosol formulations, are specifically provided. Such formulations provide effective delivery to appropriate areas of the lung or nasal cavities in short administration times, i.e., single breath, double breath, triple breath or multiple breaths in less than about 3-15 seconds per dose as compared to administration times of up to 4 to 20 minutes as found in conventional pulmonary nebulizer therapies.

Nanoparticulate drug compositions for aerosol administration can be made by, for example, (1) nebulizing a dispersion of a nanoparticulate drug, obtained by either grinding or precipitation; (2) aerosolizing a dry powder of aggregates of nanoparticulate drug and surface modifier (the aerosolized composition may additionally contain a diluent); or (3) aerosolizing a suspension of nanoparticulate drug or drug aggregates in a non-aqueous propellant. The aggregates of nanoparticulate drug and surface modifier, which may additionally contain a diluent, can be made in a non-pressurized or a pressurized non-aqueous system. Concentrated aerosol formulations may also be made via such methods.

Milling of aqueous drug to obtain nanoparticulate drug may be performed by dispersing drug particles in a liquid dispersion medium and applying mechanical means in the presence of grinding media to reduce the particle size of the drug to the desired effective average particle size. The particles can be reduced in size in the presence of one or more surface modifiers. Alternatively, the particles can be contacted with one or more surface modifiers after attrition. Other compounds, such as a diluent, can be added to the drug/surface modifier composition during the size reduction process. Dispersions can be manufactured continuously or in a batch mode.

Another method of forming nanoparticle dispersion is by microprecipitation. This is a method of preparing stable dispersions of drugs in the presence of one or more surface modifiers and one or more colloid stability enhancing surface active agents free of any trace toxic solvents or solubilized heavy metal impurities. Such a method comprises, for example, (1) dissolving the drug in a suitable solvent with mixing; (2) adding the formulation from step (1) with mixing to a solution comprising at least one surface modifier to form a clear solution; and (3) precipitating the formulation from step (2) with mixing using an appropriate nonsolvent. The method can be followed by removal of any formed salt, if present, by dialysis or diafiltration and concentration of the dispersion by conventional means. The resultant nanoparticulate drug dispersion can be utilized in liquid nebulizers or processed to form a dry powder for use in a DPI or pMDI.

In a non-aqueous, non-pressurized milling system, a non-aqueous liquid having a vapor pressure of about 1 atm or less at room temperature and in which the drug substance is essentially insoluble may be used as a wet milling medium to make a nanoparticulate drug composition. In such a process, a slurry of drug and surface modifier may be milled in the non-aqueous medium to generate nanoparticulate drug particles. Examples of suitable non-aqueous media include ethanol, trichloromonofluoromethane, (CFC-11), and dichlorotetafluoroethane (CFC-114). An advantage of using CFC-11 is that it can be handled at only marginally cool room temperatures, whereas CFC-114 requires more controlled conditions to avoid evaporation. Upon completion of milling the liquid medium may be removed and recovered under vacuum or heating, resulting in a dry nanoparticulate composition. The dry composition may then be filled into a suitable container and charged with a final propellant. Exemplary final product propellants, which ideally do not contain chlorinated hydrocarbons, include HFA-134a (tetrafluoroethane) and HFA-227 (heptafluoropropane). While non-chlorinated propellants may be preferred for environmental reasons, chlorinated propellants may also be used in this embodiment of the invention.

In a non-aqueous, pressurized milling system, a non-aqueous liquid medium having a vapor pressure significantly greater than 1 atm at room temperature may be used in the milling process to make nanoparticulate drug compositions. If the milling medium is a suitable halogenated hydrocarbon propellant, the resultant dispersion may be filled directly into a suitable pMDI container. Alternately, the milling medium can be removed and recovered under vacuum or heating to yield a dry nanoparticulate composition. This composition can then be filled into an appropriate container and charged with a suitable propellant for use in a pMDI.

Spray drying is a process used to obtain a powder containing nanoparticulate drug particles following particle size reduction of the drug in a liquid medium. In general, spray-drying may be used when the liquid medium has a vapor pressure of less than about 1 atm at room temperature. A spray-dryer is a device which allows for liquid evaporation and drug powder collection. A liquid sample, either a solution or suspension, is fed into a spray nozzle. The nozzle generates droplets of the sample within a range of about 20 to about 100 μm in diameter which are then transported by a carrier gas into a drying chamber. The carrier gas temperature is typically from about 80 to about 200° C. The droplets are subjected to rapid liquid evaporation, leaving behind dry particles which are collected in a special reservoir beneath a cyclone apparatus.

If the liquid sample consists of an aqueous dispersion of nanoparticles and surface modifier, the collected product will consist of spherical aggregates of the nanoparticulate drug particles. If the liquid sample consists of an aqueous dispersion of nanoparticles in which an inert diluent material was dissolved (such as lactose or mannitol), the collected product will consist of diluent (e.g., lactose or mannitol) particles which contain embedded nanoparticulate drug particles. The final size of the collected product can be controlled and depends on the concentration of nanoparticulate drug and/or diluent in the liquid sample, as well as the droplet size produced by the spray-dryer nozzle. Collected products may be used in conventional DPIs for pulmonary or nasal delivery, dispersed in propellants for use in pMDIs, or the particles may be reconstituted in water for use in nebulizers.

In some instances it may be desirable to add an inert carrier to the spray-dried material to improve the metering properties of the final product. This may especially be the case when the spray dried powder is very small (less than about 5 μm) or when the intended dose is extremely small, whereby dose metering becomes difficult. In general, such carrier particles (also known as bulking agents) are too large to be delivered to the lung and simply impact the mouth and throat and are swallowed. Such carriers typically consist of sugars such as lactose, mannitol, or trehalose. Other inert materials, including polysaccharides and cellulosics, may also be useful as carriers.

Spray-dried powders containing nanoparticulate drug particles may used in conventional DPIs, dispersed in propellants for use in pMDIs, or reconstituted in a liquid medium for use with nebulizers.

For compounds that are denatured or destabilized by heat, such as compounds having a low melting point (i.e., about 70 to about 150° C.), or for example, biologics, sublimation is preferred over evaporation to obtain a dry powder nanoparticulate drug composition. This is because sublimation avoids the high process temperatures associated with spray-drying. In addition, sublimation, also known as freeze-drying or lyophilization, can increase the shelf stability of drug compounds, particularly for biological products. Freeze-dried particles can also be reconstituted and used in nebulizers. Aggregates of freeze-dried nanoparticulate drug particles can be blended with either dry powder intermediates or used alone in DPIs and pMDIs for either nasal or pulmonary delivery.

Sublimation involves freezing the product and subjecting the sample to strong vacuum conditions. This allows for the formed ice to be transformed directly from a solid state to a vapor state. Such a process is highly efficient and, therefore, provides greater yields than spray-drying. The resultant freeze-dried product contains drug and modifier(s). The drug is typically present in an aggregated state and can be used for inhalation alone (either pulmonary or nasal), in conjunction with diluent materials (lactose, mannitol, etc.), in DPIs or pMDIs, or reconstituted for use in a nebulizer.

### Liposomal Compositions

In some embodiments, nitrite, nitrite salt, or nitrite- or nitric oxide-donating compounds disclosed herein may be formulated into liposome particles, which can then be aerosolized for inhaled delivery. Lipids which are useful in the present invention can be any of a variety of lipids including both neutral lipids and charged lipids. Carrier systems having desirable properties can be prepared using appropriate combinations of lipids, targeting groups and circulation enhancers. Additionally, the compositions provided herein can be in the form of liposomes or lipid particles, preferably lipid particles. As used herein, the term “lipid particle” refers to a lipid bilayer carrier which “coats” a nucleic acid and has little or no aqueous interior. More particularly, the term is used to describe a self-assembling lipid bilayer carrier in which a portion of the interior layer comprises cationic lipids which form ionic bonds or ion-pairs with negative charges on the nucleic acid (e.g., a plasmid phosphodiester backbone). The interior layer can also comprise neutral or fusogenic lipids and, in some embodiments, negatively charged lipids. The outer layer of the particle will typically comprise mixtures of lipids oriented in a tail-to-tail fashion (as in liposomes) with the hydrophobic tails of the interior layer. The polar head groups present on the lipids of the outer layer will form the external surface of the particle.

Liposomal bioactive agents can be designed to have a sustained therapeutic effect or lower toxicity allowing less frequent administration and an enhanced therapeutic index. Liposomes are composed of bilayers that entrap the desired pharmaceutical. These can be configured as multilamellar vesicles of concentric bilayers with the pharmaceutical trapped within either the lipid of the different layers or the aqueous space between the layers.

By non-limiting example, lipids used in the compositions may be synthetic, semi-synthetic or naturally-occurring lipids, including phospholipids, tocopherols, steroids, fatty acids, glycoproteins such as albumin, negatively-charged lipids and cationic lipids. Phospholipids include egg phosphatidylcholine (EPC), egg phosphatidylglycerol (EPG), egg phosphatidylinositol (EPI), egg phosphatidylserine (EPS), phosphatidylethanolamine (EPE), and egg phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid can be made up of fatty acids of different chain lengths and different degrees of unsaturation. In particular, the compositions of the formulations can include dipalmitoylphosphatidylcholine (DPPC), a major constituent of naturally-occurring lung surfactant as well as dioleoylphosphatidylcholine (DOPC) and dioleoylphosphatidylglycerol (DOPG). Other examples include dimyristoylphosphatidycholine (DMPC) and dimyristoylphosphatidylglycerol (DMPG) dipalmitoylphosphatidcholine (DPPC) and dipalmitoylphosphatidylglycerol (DPPG) distearoylphosphatidylcholine (DSPC) and distearoylphosphatidylglycerol (DSPG), dioleylphosphatidylethanolamine (DOPE) and mixed phospholipids like palmitoylstearoylphosphatidylcholine (PSPC) and palmitoylstearoylphosphatidylglycerol (PSPG), and single acylated phospholipids like mono-oleoyl-phosphatidylethanolamine (MOPE).

In a preferred embodiment, PEG-modified lipids are incorporated into the compositions of the present invention as the aggregation-preventing agent. The use of a PEG-modified lipid positions bulky PEG groups on the surface of the liposome or lipid carrier and prevents binding of DNA to the outside of the carrier (thereby inhibiting cross-linking and aggregation of the lipid carrier). The use of a PEG-ceramide is often preferred and has the additional advantages of stabilizing membrane bilayers and lengthening circulation lifetimes. Additionally, PEG-ceramides can be prepared with different lipid tail lengths to control the lifetime of the PEG-ceramide in the lipid bilayer. In this manner, “programmable” release can be accomplished which results in the control of lipid carrier fusion. For example, PEG-ceramides having C₂₀-acyl groups attached to the ceramide moiety will diffuse out of a lipid bilayer carrier with a half-life of 22 hours. PEG-ceramides having C₁₄- and C₈-acyl groups will diffuse out of the same carrier with half-lives of 10 minutes and less than 1 minute, respectively. As a result, selection of lipid tail length provides a composition in which the bilayer becomes destabilized (and thus fusogenic) at a known rate. Though less preferred, other PEG-lipids or lipid-polyoxyethylene conjugates are useful in the present compositions. Examples of suitable PEG-modified lipids include PEG-modified phosphatidylethanolamine and phosphatidic acid, PEG-modified diacylglycerols and dialkylglycerols, PEG-modified dialkylamines and PEG-modified 1,2-diacyloxypropan-3-amines. Particularly preferred are PEG-ceramide conjugates (e.g., PEG-Cer-C₈, PEG-Cer-C₁₄ or PEG-Cer-C₂₀) which are described in U.S. Pat. No. 5,820,873, incorporated herein by reference.

The compositions of the present invention can be prepared to provide liposome compositions which are about 50 nm to about 400 nm in diameter. One with skill in the art will understand that the size of the compositions can be larger or smaller depending upon the volume which is encapsulated. Thus, for larger volumes, the size distribution will typically be from about 80 nm to about 300 nm.

### Surface Modifiers

Nitrite compounds (e.g., nitrite anion or salts thereof), or in distinct embodiments, nitrite- or nitric oxide-donating compounds, as disclosed herein may be prepared in a pharmaceutical composition with suitable surface modifiers which may be selected from known organic and inorganic pharmaceutical excipients. Such excipients include low molecular weight oligomers, polymers, surfactants and natural products. Preferred surface modifiers include nonionic and ionic surfactants. Two or more surface modifiers can be used in combination.

Representative examples of surface modifiers include cetyl pyridinium chloride, gelatin, casein, lecithin (phosphatides), dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens™, such as e.g., Tween 20™, and Tween 80™, (ICI Specialty Chemicals)); polyethylene glycols (e.g., Carbowaxs 3350™, and 1450™, and Carbopol 934™, (Union Carbide)), dodecyl trimethyl ammonium bromide, polyoxyethylenestearates, colloidal silicon dioxide, phosphates, sodium dodecylsulfate, carboxymethylcellulose calcium, hydroxypropyl cellulose (HPC, HPC-SL, and HPC-L), hydroxypropyl methylcellulose (HPMC), carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), polyvinylpyrrolidone (PVP), 4-(1,1,3,3-tetaamethylbutyl)-phenol polymer with ethylene oxide and formaldehyde (also known as tyloxapol, superione, and triton), poloxamers (e.g., Pluronics F68™, and F108™, which are block copolymers of ethylene oxide and propylene oxide); poloxamines (e.g., Tetronic 908™, also known as Poloxamine 908™, which is a tetrafunctional block copolymer derived from sequential addition of propylene oxide and ethylene oxide to ethylenediamine (BASF Wyandotte Corporation, Parsippany, N.J.)); a charged phospholipid such as dimyristoyl phosphatidyl glycerol, dioctylsulfosuccinate (DOSS); Tetronic 1508™; (T-1508) (BASF Wyandotte Corporation), dialkylesters of sodium sulfosuccinic acid (e.g., Aerosol OT™, which is a dioctyl ester of sodium sulfosuccinic acid (American Cyanamid)); Duponol P™, which is a sodium lauryl sulfate (DuPont); Tritons X-200™, which is an alkyl aryl polyether sulfonate (Rohm and Haas); Crodestas F-110™, which is a mixture of sucrose stearate and sucrose distearate (Croda Inc.); p-isononylphenoxypoly-(glycidol), also known as Olin-Log™, or Surfactant 10-G™, (Olin Chemicals, Stamford, Conn.); Crodestas SL-40™, (Croda, Inc.); and SA9OHCO, which is C₁8H₃₇CH₂ (CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; and the like. Tyloxapol is a particularly preferred surface modifier for the pulmonary or intranasal delivery of steroids, even more so for nebulization therapies.

Examples of surfactants for use in the solutions disclosed herein include, but are not limited to, ammonium laureth sulfate, cetamine oxide, cetrimonium chloride, cetyl alcohol, cetyl myristate, cetyl palmitate, cocamide DEA, cocamidopropyl betaine, cocamidopropylamine oxide, cocamide MEA, DEA lauryl sulfate, di-stearyl phthalic acid amide, dicetyl dimethyl ammonium chloride, dipalmitoylethyl hydroxethylmonium, disodium laureth sulfosuccinate, di(hydrogenated) tallow phthalic acid, glyceryl dilaurate, glyceryl distearate, glyceryl oleate, glyceryl stearate, isopropyl myristate nf, isopropyl palmitate nf, lauramide DEA, lauramide MEA, lauramide oxide, myristamine oxide, octyl isononanoate, octyl palmitate, octyldodecyl neopentanoate, olealkonium chloride, PEG-2 stearate, PEG-32 glyceryl caprylate/caprate, PEG-32 glyceryl stearate, PEG-4 and PEG-150 stearate & distearate, PEG-4 to PEG-150 laurate & dilaurate, PEG-4 to PEG-150 oleate & dioleate, PEG-7 glyceryl cocoate, PEG-8 beeswax, propylene glycol stearate, sodium C14-16 olefin sulfonate, sodium lauryl sulfoacetate, sodium lauryl sulphate, sodium trideceth sulfate, stearalkonium chloride, stearamide oxide, TEA-dodecylbenzene sulfonate, TEA lauryl sulfate

Most of these surface modifiers are known pharmaceutical excipients and are described in detail in the Handbook of Pharmaceutical Excipients, published jointly by the American Pharmaceutical Association and The Pharmaceutical Society of Great Britain (The Pharmaceutical Press, 1986), specifically incorporated by reference. The surface modifiers are commercially available and/or can be prepared by techniques known in the art. The relative amount of drug and surface modifier can vary widely and the optimal amount of the surface modifier can depend upon, for example, the particular drug and surface modifier selected, the critical micelle concentration of the surface modifier if it forms micelles, the hydrophilic-lipophilic-balance (HLB) of the surface modifier, the melting point of the surface modifier, the water solubility of the surface modifier and/or drug, the surface tension of water solutions of the surface modifier, etc.

In certain related embodiments of the present invention, the optimal ratio of drug to surface modifier may be from about 0.1% to about 99.9% nitrite compound or (in distinct embodiments) nitrite- or nitric oxide-donating compound, more preferably from about 10% to about 90%.

### Microspheres

Microspheres can be used for pulmonary delivery of nitrite, nitrite salt, or nitrite- or nitric oxide-donating compounds by first adding an appropriate amount of drug compound to be solubilzed in water. For example, in certain embodiments an aqueous solution comprising a nitrite compound (e.g., nitrite anion or a salt thereof), or in certain distinct embodiments a nitrite- or nitric oxide-donating compound, may be dispersed in methylene chloride containing a predetermined amount (e.g., 0.1-1% w/v) of poly(DL-lactide-co-glycolide) (PLGA) by probe sonication for 1-3 min on an ice bath. Separately, the nitrite compound (or in distinct embodiments, the nitrite- or nitric oxide-donating compound) is solubilized in methylene chloride containing PLGA (0.1-1% w/v). The resulting water-in-oil primary emulsion or the polymer/drug solution may be dispersed in an aqueous continuous phase consisting of 1-2% polyvinyl alcohol (previously cooled to 4° C.) by probe sonication for 3-5 min on an ice bath. The resulting emulsion is stirred continuously for 2-4 hours at room temperature to evaporate methylene chloride. Microparticles thus formed are separated from the continuous phase by centrifuging at 8,000-10,000 rpm for 5-10 min. Sedimented particles will be washed thrice with distilled water and freeze dried. Freeze-dried nitrite compound, or nitrite- or nitric oxide-donating compound, microparticles will be stored at −20° C.

By non-limiting example, a spray drying approach will be employed to prepare nitrite compound microspheres (or in distinct embodiments, nitrite- or NO-donating compound microspheres). An appropriate amount of nitrite compound or nitrite- or nitric oxide-donating compound may be solubilized in methylene chloride containing PLGA (0.1-1%). This solution will be spray dried to obtain the microspheres.

By non-limiting example, nitrite compound microparticles, or in distinct embodiments nitrite- or nitric oxide-donating compound microparticles, will be characterized for size distribution (in preferred embodiments: 90%<5 μm, 95%<10 μm), shape, drug loading efficiency and drug release using appropriate techniques and methods.

By non-limiting example, this approach may also be used to sequester and improve the water solubility of solid, area-under-the-curve (AUC) shape-enhancing formulations, such as low-solubility nitrite compound, or nitrite- or nitric oxide-donating compound, salt forms for nanoparticle-based formulations.

A certain amount of nitrite compound, or nitrite- or nitric oxide-donating compound, can be first dissolved in a minimal quantity of ethanol (e.g., 96%) as may maintain the compound in solution when diluted with water from about 96% to about 75% (v/v). This solution can then be diluted with water to obtain a 75% ethanol solution and then a certain amount of paracetamol can be added to obtain the following w/w drug/polymer ratios: 1:2, 1:1, 2:1, 3:1, 4:1, 6:1, 9:1, and 19:1. These final solutions are spray-dried under the following conditions: feed rate, 15 mL/min; inlet temperature, 110° C.; outlet temperature, 85° C.; pressure 4 bar and throughput of drying air, 35 m3/hr. Powder is then collected and stored under vacuum in a dessiccator.

### Solid Lipid Particles

Preparation according to certain embodiments of nitrite compound (e.g., nitrite anion or a salt thereof, such as sodium nitrite, potassium nitrite or magnesium nitrite) solid lipid particles, or in distinct embodiments of nitrite- or nitric oxide-donating compound solid lipid particles, may involve dissolving the drug in a lipid melt (phospholipids such as phosphatidyl choline and phosphatidyl serine) maintained at least at the melting temperature of the lipid, followed by dispersion of the drug-containing melt in a hot aqueous surfactant solution (typically 1-5% w/v) maintained at least at the melting temperature of the lipid. The coarse dispersion will be homogenized for 1-10 min using a Microfluidizer® to obtain a nanoemulsion. Cooling the nanoemulsion to a temperature between about 4-25° C. will re-solidify the lipid, leading to formation of solid lipid nanoparticles. Optimization of formulation parameters (type of lipid matrix, surfactant concentration and production parameters) will be performed so as to achieve a prolonged drug delivery. By non-limiting example, this approach may also be used to sequester and improve the water solubility of solid, AUC shape-enhancing formulations, such as low-solubility nitrite, nitrite salt, or nitrite- or nitric oxide-donating compound salt forms for nanoparticle-based formulations.

### Melt-Extrusion AUC Shape-Enhancing Formulation

Melt-Extrusion AUC shape-enhancing nitrite compound, or in distinct embodiments nitrite- or nitric oxide-donating compound, formulations may be preparation by dissolving the drugs in micelles by adding surfactants or preparing micro-emulsion, forming inclusion complexes with other molecules such as cyclodextrins, forming nanoparticles of the drugs, or embedding the amorphous drugs in a polymer matrix. Embedding the drug homogeneously in a polymer matrix produces a solid dispersion. Solid dispersions can be prepared in two ways: the solvent method and the hot melt method. The solvent method uses an organic solvent wherein the drug and appropriate polymer are dissolved and then (spray) dried. The major drawbacks of this method are the use of organic solvents and the batch mode production process. The hot melt method uses heat in order to disperse or dissolve the drug in an appropriate polymer. The melt-extrusion process is an optimized version of the hot melt method. The advantage of the melt-extrusion approach is lack of organic solvent and continuous production process. As the melt-extrusion is a novel pharmaceutical technique, the literature dealing with it is limited. The technical set-up involves a mixture and extrusion of the nitrite compound (e.g., nitrite anion or salt thereof such as sodium nitrite, potassium nitrite or magnesium nitrite), or in distinct embodiments of the nitrite- or nitric oxide-donating compound, hydroxypropyl-b-cyclodextrin (HP-b-CD), and hydroxypropylmethylcellulose (HPMC), in order to, by non-limiting example, create an AUC shape-enhancing formulation of nitrite compound (or nitrite- or nitric oxide-donating compound). Cyclodextrin is a toroidal-shaped molecule with hydroxyl groups on the outer surface and a cavity in the center. Cyclodextrin sequesters the drug by forming an inclusion complex. The complex formation between cyclodextrins and drugs has been investigated extensively. It is known that water-soluble polymer interacts with cyclodextrin and drug in the course of complex formation to form a stabilized complex of drug and cyclodextrin co-complexed with the polymer. This complex is more stable than the classic cyclodextrin-drug complex. As one example, HPMC is water soluble; hence using this polymer with HP-b-CD in the melt is expected to create an aqueous soluble AUC shape-enhancing formulation. By non-limiting example, this approach may also be used to sequester and improve the water solubility of solid, AUC shape-enhancing formulations, such as low-solubility nitrite compound, or nitrite- or nitric oxide-donating compound, salt forms for nanoparticle-based formulations.

### Co-Precipitates

Co-precipitate nitrite compound formulations, or in distinct embodiments nitrite- or nitric oxide-donating compound formulations, may be prepared by formation of co-precipitates with pharmacologically inert, polymeric materials. It has been demonstrated that the formation of molecular solid dispersions or co-precipitates to create an AUC shape-enhancing formulations with various water-soluble polymers can significantly slow their in vitro dissolution rates and/or in vivo absorption. In preparing powdered products, grinding is generally used for reducing particle size, since the dissolution rate is strongly affected by particle size. Moreover, a strong force (such as grinding) may increase the surface energy and cause distortion of the crystal lattice as well as reducing particle size. Co-grinding drug with hydroxypropylmethylcellulose, b-cyclodextrin, chitin and chitosan, crystalline cellulose, and gelatin, may enhance the dissolution properties such that AUC shape-enhancement is obtained for otherwise readily bioavailable nitrite compounds, or nitrite- or nitric oxide-donating compounds. By non-limiting example, this approach may also be used to sequester and improve the water solubility of solid, AUC shape-enhancing formulations, such as low-solubility nitrite, nitrite salt, or nitrite- or nitric oxide-donating compound salt forms for nanoparticle-based formulations.

### Dispersion-Enhancing Peptides

Compositions may include one or more di- or tripeptides containing two or more leucine residues. By further non-limiting example, U.S. Pat. No. 6,835,372 disclosing dispersion-enhancing peptides, is hereby incorporated by reference in its entirety. This patent describes the discovery that di-leucyl-containing dipeptides (e.g., dileucine) and tripeptides are superior in their ability to increase the dispersibility of powdered composition.

In another embodiment, highly dispersible particles including an amino acid are administered. Hydrophobic amino acids are preferred. Suitable amino acids include naturally occurring and non-naturally occurring hydrophobic amino acids. Some naturally occurring hydrophobic amino acids, include but are not limited to, non-naturally occurring amino acids include, for example, beta-amino acids. Both D, L and racemic configurations of hydrophobic amino acids can be employed. Suitable hydrophobic amino acids can also include amino acid analogs. As used herein, an amino acid analog includes the D or L configuration of an amino acid having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight-chained, branched or cyclic C₁-C₈ hydrocarbons which are completely saturated, which contain one or two heteroatoms such as nitrogen, oxygen or sulfur and/or which contain one or more units of desaturation.

Aromatic groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridinyl.

Suitable substituents on an aliphatic, aromatic or benzyl group include —OH, halogen (—Br, —Cl, —I and —F), —O(aliphatic, substituted aliphatic, benzyl, substituted benzyl, aryl or substituted aryl group), —CN, —NO₂, —COOH, —NH₂, —NH (aliphatic group, substituted aliphatic, benzyl, substituted benzyl, aryl or substituted aryl group), —N(aliphatic group, substituted aliphatic, benzyl, substituted benzyl, aryl or substituted aryl group)₂, —COO (aliphatic group, substituted aliphatic, benzyl, substituted benzyl, aryl or substituted aryl group), —CONH₂, —CONH (aliphatic, substituted aliphatic group, benzyl, substituted benzyl, aryl or substituted aryl group)), —SH, —S(aliphatic, substituted aliphatic, benzyl, substituted benzyl, aromatic or substituted aromatic group) and —NH—C(═NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more substituents. Modifying an amino acid substituent can increase, for example, the lipophilicity or hydrophobicity of natural amino acids which are hydrophilic.

A number of the suitable amino acids, amino acids analogs and salts thereof can be obtained commercially. Others can be synthesized by methods known in the art.

Hydrophobicity is generally defined with respect to the partition of an amino acid between a nonpolar solvent and water. Hydrophobic amino acids are those acids which show a preference for the nonpolar solvent. Relative hydrophobicity of amino acids can be expressed on a hydrophobicity scale on which glycine has the value 0.5. On such a scale, amino acids which have a preference for water have values below 0.5 and those that have a preference for nonpolar solvents have a value above 0.5. As used herein, the term hydrophobic amino acid refers to an amino acid that, on the hydrophobicity scale, has a value greater or equal to 0.5, in other words, has a tendency to partition in the nonpolar acid which is at least equal to that of glycine.

Examples of amino acids which can be employed include, but are not limited to: glycine, proline, alanine, cysteine, methionine, valine, leucine, tyosine, isoleucine, phenylalanine, tryptophan. Preferred hydrophobic amino acids include leucine, isoleucine, alanine, valine, phenylalanine and glycine. Combinations of hydrophobic amino acids can also be employed. Furthermore, combinations of hydrophobic and hydrophilic (preferentially partitioning in water) amino acids, where the overall combination is hydrophobic, can also be employed.

The amino acid can be present in the particles of the invention in an amount of at least 10 weight %. Preferably, the amino acid can be present in the particles in an amount ranging from about 20 to about 80 weight %. The salt of a hydrophobic amino acid can be present in the particles of the invention in an amount of at least 10 weight percent. Preferably, the amino acid salt is present in the particles in an amount ranging from about 20 to about 80 weight %. In preferred embodiments the particles have a tap density of less than about 0.4 g/cm³.

Methods of forming and delivering particles which include an amino acid are described in U.S. Pat. No. 6,586,008, entitled Use of Simple Amino Acids to Form Porous Particles During Spray Drying, the teachings of which are incorporated herein by reference in their entirety.

### Proteins/Amino Acids

Protein excipients may include albumins such as human serum albumin (HSA), recombinant human albumin (rHA), gelatin, casein, hemoglobin, and the like. Suitable amino acids (outside of dileucyl-peptides), which may also function in a buffering capacity, include alanine, glycine, arginine, betaine, histidine, glutamic acid, aspartic acid, cysteine, lysine, leucine, isoleucine, valine, methionine, phenylalanine, aspartame, tyrosine, tryptophan, and the like. Preferred are amino acids and polypeptides that function as dispersing agents. Amino acids falling into this category include hydrophobic amino acids such as leucine, valine, isoleucine, tryptophan, alanine, methionine, phenylalanine, tyrosine, histidine, and proline. Dispersibility-enhancing peptide excipients include dimers, trimers, tetramers, and pentamers comprising one or more hydrophobic amino acid components such as those described above.

### Carbohydrates

By non-limiting example, carbohydrate excipients may include monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), pyranosyl sorbitol, myoinositol, isomalt, trehalose and the like.

### Polymers

According to certain embodiments, compositions and formulations disclosed herein may also include, by way of non-limiting example, polymeric excipients/additives, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch, dextrates (by non-limiting example cyclodextrins may include, 2-hydroxypropyl-beta-cyclodextrin, 2-hydroxypropyl-gamma-cyclodextrin, randomly methylated beta-cyclodextrin, dimethyl-alpha-cyclodextrin, dimethyl-beta-cyclodextrin, maltosyl-alpha-cyclodextrin, glucosyl-1-alpha-cyclodextrin, glucosyl-2-alpha-cyclodextrin, alpha-cyclodextrin, beta-cyclodextrin, gamma-cyclodextrin, and sulfobutylether-beta-cyclodextrin), polyethylene glycols, and pectin may also be used.

Highly dispersible particles administered comprise a bioactive agent and a biocompatible, and preferably biodegradable polymer, copolymer, or blend. The polymers may be tailored to optimize different characteristics of the particle including: i) interactions between the agent to be delivered and the polymer to provide stabilization of the agent and retention of activity upon delivery; ii) rate of polymer degradation and, thereby, rate of drug release profiles; iii) surface characteristics and targeting capabilities via chemical modification; and iv) particle porosity.

Surface eroding polymers such as polyanhydrides may be used to form the particles. For example, polyanhydrides such as poly[(p-carboxyphenoxy)hexane anhydride](PCPH) may be used. Biodegradable polyanhydrides are described in U.S. Pat. No. 4,857,311. Bulk eroding polymers such as those based on polyesters including poly(hydroxy acids) also can be used. For example, polyglycolic acid (PGA), polylactic acid (PLA), or copolymers thereof may be used to form the particles. The polyester may also have a charged or functionalizable group, such as an amino acid. In a preferred embodiment, particles with controlled release properties can be formed of poly(D,L-lactic acid) and/or poly(DL-lactic-co-glycolic acid) (“PLGA”) which incorporate a surfactant such as dipalmitoyl phosphatidylcholine (DPPC).

Other polymers include polyamides, polycarbonates, polyalkylenes such as polyethylene, polypropylene, poly(ethylene glycol), poly(ethylene oxide), poly(ethylene terephthalate), poly vinyl compounds such as polyvinyl alcohols, polyvinyl ethers, and polyvinyl esters, polymers of acrylic and methacrylic acids, celluloses and other polysaccharides, and peptides or proteins, or copolymers or blends thereof. Polymers may be selected with or modified to have the appropriate stability and degradation rates in vivo for different controlled drug delivery applications.

Highly dispersible particles can be formed from functionalized polyester graft copolymers, as described in Hrkach et al., Macromolecules, 28: 4736-4739 (1995); and Hrkach et al., “Poly(L-Lactic acid-co-amino acid) Graft Copolymers: A Class of Functional Biodegradable Biomaterials” in Hydrogels and Biodegradable Polymers for Bioapplications, ACS Symposium Series No. 627, Raphael M, Ottenbrite et al., Eds., American Chemical Society, Chapter 8, pp. 93-101, 1996.

In a preferred embodiment of the invention, highly dispersible particles including a bioactive agent and a phospholipid are administered. Examples of suitable phospholipids include, among others, phosphatidylcholines, phosphatidylethanolamines, phosphatidylglycerols, phosphatidylserines, phosphatidylinositols and combinations thereof. Specific examples of phospholipids include but are not limited to phosphatidylcholines dipalmitoyl phosphatidylcholine (DPPC), dipalmitoyl phosphatidylethanolamine (DPPE), distearoyl phosphatidyicholine (DSPC), dipalmitoyl phosphatidyl glycerol (DPPG) or any combination thereof. Other phospholipids are known to those skilled in the art. In a preferred embodiment, the phospholipids are endogenous to the lung.

The phospholipid, can be present in the particles in an amount ranging from about 0 to about 90 weight %. More commonly it can be present in the particles in an amount ranging from about 10 to about 60 weight %.

In another embodiment, the phospholipids or combinations thereof are selected to impart controlled release properties to the highly dispersible particles. The phase transition temperature of a specific phospholipid can be below, around or above the physiological body temperature of a patient. Preferred phase transition temperatures range from 30 degrees C. to 50 degrees C. (e.g., within +/−10 degrees of the normal body temperature of patient). By selecting phospholipids or combinations of phospholipids according to their phase transition temperature, the particles can be tailored to have controlled release properties. For example, by administering particles which include a phospholipid or combination of phospholipids which have a phase transition temperature higher than the patient's body temperature, the release of dopamine precursor, agonist or any combination of precursors and/or agonists can be slowed down. On the other hand, rapid release can be obtained by including in the particles phospholipids having lower transition temperatures.

### Taste Masking, Flavor, Other

As also described above, nitrite compound formulations disclosed herein and related compositions, including nitrite- and NO-donating compound formulations, may further include one or more taste-masking agents such as flavoring agents, inorganic salts (e.g., sodium chloride), sweeteners, antioxidants, antistatic agents, surfactants (e.g., polysorbates such as “TWEEN 20” and “TWEEN 80”), sorbitan esters, saccharin (e.g., sodium saccharin or other saccharin forms, which as noted elsewhere herein may be present in certain embodiments at specific concentrations or at specific molar ratios relative to a nitrite compound such as sodium nitrite), bicarbonate, cyclodextrins, lipids (e.g., phospholipids such as lecithin and other phosphatidylcholines, phosphatidylethanolamines), fatty acids and fatty esters, steroids (e.g., cholesterol), and chelating agents (e.g., EDTA, zinc and other such suitable cations). Other pharmaceutical excipients and/or additives suitable for use in the compositions according to the invention are listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998).

By way of non-limiting example, taste-masking agents in nitrite compound formulations, or in nitrite- or nitric oxide-donating compound formulations, may include the use of one or more flavorings, sweeteners, and other various coating strategies, for instance, sugars such as sucrose, dextrose, and lactose, carboxylic acids, menthol, amino acids or amino acid derivatives such as arginine, lysine, and monosodium glutamate, and/or synthetic flavor oils and flavoring aromatics and/or natural oils, extracts from plants, leaves, flowers, fruits, etc. and combinations thereof. These may include cinnamon oils, oil of wintergreen, peppermint oils, clover oil, bay oil, anise oil, eucalyptus, vanilla, citrus oil such as lemon oil, orange oil, grape and grapefruit oil, fruit essences including apple, peach, pear, strawberry, raspberry, cherry, plum, pineapple, apricot, etc. Additional sweeteners include sucrose, dextrose, aspartame (Nutrasweet®), acesulfame-K, sucralose and saccharin (e.g., sodium saccharin or other saccharin forms, which as noted elsewhere herein may be present in certain embodiments at specific concentrations or at specific molar ratios relative to a nitrite compound such as sodium nitrite), organic acids (by non-limiting example citric acid and aspartic acid). Such flavors may be present at from about 0.05 to about 4 percent by weight, and may be present at lower or higher amounts as a factor of one or more of potency of the effect on flavor, solubility of the flavorant, effects of the flavorant on solubility or other physicochemical or pharmacokinetic properties of other formulation components, or other factors.

Another approach to improve or mask the unpleasant taste of an inhaled drug may be to decrease the drug's solubility, e.g., drugs must dissolve to interact with taste receptors. Hence, to deliver solid forms of the drug may avoid the taste response and result in the desired improved taste affect. Non-limiting methods to decrease solubility of a nitrite anion, nitrite salt thereof, or of a nitrite- or nitric oxide-donating compound solubility are described herein, for example, through the use in formulation of particular salt forms of nitrite anion, or of a nitrite- or nitric oxide-donating compound, such as complexation with xinafoic acid, oleic acid, stearic acid and/or pamoic acid. Additional co-precipitating agents include dihydropyridines and a polymer such as polyvinyl pyrrolidone.

Moreover, taste-masking may be accomplished by creation of lipophilic vesicles. Additional coating or capping agents include dextrates (by non-limiting example cyclodextrins may include, 2-hydroxypropyl-beta-cyclodextrin, 2-hydroxypropyl-gamma-cyclodextrin, randomly methylated beta-cyclodextrin, dimethyl-alpha-cyclodextrin, dimethyl-beta-cyclodextrin, maltosyl-alpha-cyclodextrin, glucosyl-1-alpha-cyclodextrin, glucosyl-2-alpha-cyclodextrin, alpha-cyclodextrin, beta-cyclodextrin, gamma-cyclodextrin, and sulfobutylether-beta-cyclodextrin), modified celluloses such as ethyl cellulose, methyl cellulose, hydroxypropyl cellulose, hydroxylpropyl methyl cellulose, polyalkylene glycols, polyalkylene oxides, sugars and sugar alcohols, waxes, shellacs, acrylics and mixtures thereof. By non-limiting example, other methods to deliver non-dissolved forms of a nitrite compound according to certain embodiments (e.g., nitrite anion or a salt thereof, such as sodium, magnesium or potassium nitrite), or, in other embodiments, non-dissolved forms of a nitrite- or nitric oxide-donating compound, are to administer the drug alone or in a simple, non-solubility affecting formulation, such as a crystalline micronized, dry powder, spray-dried, and/or nanosuspension formulation.

An alternative according to certain other preferred embodiments is to include taste-modifying agents in the nitrite compound formulation or, in certain other embodiments, in the nitrite- or NO-donating compound formulation. These embodiments contemplate including in the formulation a taste-masking substance that is mixed with, coated onto or otherwise combined with the active medicament nitrite anion or salt thereof, or the nitrite- or NO-donating compound. Inclusion of one or more such agents in these formulations may also serve to improve the taste of additional pharmacologically active compounds that are included in the formulations in addition to the nitrite compound or nitrite- or NO-donating compound, e.g., a mucolytic agent. Non-limiting examples of such taste-modifying substances include acid phospholipids, lysophospholipids, tocopherol polyethyleneglycol succinate, and embonic acid (pamoate). Many of these agents can be used alone or in combination with nitrite anion (or a salt thereof) or, in separate embodiments, with a nitrite- or nitric oxide-donating compound for aerosol administration.

### Mucolytic Agents

Methods to produce formulations that combine agents to reduce sputum viscosity during aerosol treatment with a nitrite compound as provided herein, or in distinct embodiments with a nitrite- or nitric oxide-donating compound as provided herein, include the following. These agents may be prepared in fixed combination, or may be administered in succession with, aerosolized nitrite compound therapy, or aerosolized nitrite- or nitric oxide-donating compound therapy.

The most commonly prescribed agent is N-acetylcysteine (NAC), which depolymerizes mucus in vitro by breaking disulphide bridges between macromolecules. It is assumed that such reduction of sputum tenacity facilitates its removal from the respiratory tract. In addition, NAC may act as an oxygen radical scavenger. NAC can be taken either orally or by inhalation. Differences between these two methods of administration have not been formally studied. After oral administration, NAC is reduced to cysteine, a precursor of the antioxidant glutathione, in the liver and intestine. The antioxidant properties could be useful in preventing decline of lung function in cystic fibrosis (CF). Nebulized NAC is commonly prescribed to patients with CF, in particular in continental Europe, in order to improve expectoration of sputum by reducing its tenacity. The ultimate goal of this approach is to slow down the decline of lung function in CF.

L-lysine-N-acetylcysteinate (ACC) or Nacystelyn (NAL) is a novel mucoactive agent possessing mucolytic, antioxidant, and anti-inflammatory properties. Chemically, it is a salt of ACC. This drug appears to present an activity superior to its parent molecule ACC because of a synergistic mucolytic activity of L-lysine and ACC. Furthermore, its almost neutral pH (6.2) allows its administration in the lungs with a very low incidence of bronchospasm, which is not the case for the acidic ACC (pH 2.2). NAL is difficult to formulate in an inhaled form because the required lung dose is very high (approximately 2 mg) and the micronized drug is sticky and cohesive and it is thus problematic to produce a redispersable formulation. NAL was first developed as a chlorofluorocarbon (CFC) containing metered-dose inhaler (MDI) because this form was the easiest and the fastest to develop to begin the preclinical and the first clinical studies. NAL MDI delivered 2 mg per puff, from which approximately 10% was able to reach the lungs in healthy volunteers. One major inconvenience of this formulation was patient compliance because as many as 12 puffs were necessary to obtain the required dose. Furthermore, the progressive removal of CFC gases from medicinal products combined with the problems of coordination met in a large proportion of the patient population (12) have led to the development of a new galenical form of NAL. A dry powder inhaler (DPI) formulation was chosen to resolve the problems of compliance with MDIs and to combine it with an optimal, reproducible, and comfortable way to administer the drug to the widest possible patient population, including young children.

The DPI formulation of NAL involved the use of a nonconventional lactose (usually reserved for direct compression of tablets), namely, a roller-dried (RD) anhydrous β-lactose. When tested in vitro with a monodose DPI device, this powder formulation produces a fine particle fraction (FPF) of at least 30% of the nominal dose, namely three times higher than that with MDIs. This approach may be used in combination with a nitrite compound as provided herein according to certain presently contemplated embodiments, or in distinct embodiments with a nitrite- or nitric oxide-donating compound as provided herein, for either co-administration or fixed combination therapy.

In addition to mucolytic activity, excessive neutrophil elastase activity within airways of cystic fibrosis (CF) patients results in progressive lung damage. Disruption of disulfide bonds on elastase by reducing agents may modify its enzymatic activity. Three naturally occurring dithiol reducing systems were examined for their effects on elastase activity: 1) Escherichia coli thioredoxin (Trx) system, 2) recombinant human thioredoxin (rhTrx) system, and 3) dihydrolipoic acid (DHLA). The Trx systems consisted of Trx, Trx reductase, and NADPH. As shown by spectrophotometric assay of elastase activity, the two Trx systems and DHLA inhibited purified human neutrophil elastase as well as the elastolytic activity present in the soluble phase (sol) of CF sputum. Removal of any of the three Trx system constituents prevented inhibition. Compared with the monothiols N-acetylcysteine and reduced glutathione, the dithiols displayed greater elastase inhibition. To streamline Trx as an investigational tool, a stable reduced form of rhTrx was synthesized and used as a single component. Reduced rhTrx inhibited purified elastase and CF sputum sol elastase without NADPH or Trx reductase. Because Trx and DHLA have mucolytic effects, we investigated changes in elastase activity after mucolytic treatment. Unprocessed CF sputum was directly treated with reduced rhTrx, the Trx system, DHLA, or DNase. The Trx system and DHLA did not increase elastase activity, whereas reduced rhTrx treatment increased sol elastase activity by 60%. By contrast, the elastase activity after DNase treatment increased by 190%. The ability of Trx and DHLA to limit elastase activity combined with their mucolytic effects makes these compounds potential therapies for CF.

In addition, bundles of F-actin and DNA present in the sputum of cystic fibrosis (CF) patients but absent from normal airway fluid contribute to the altered viscoelastic properties of sputum that inhibit clearance of infected airway fluid and exacerbate the pathology of CF. One approach to alter these adverse properties is to remove these filamentous aggregates using DNase to enzymatically depolymerize DNA to constituent monomers and gelsolin to sever F-actin to small fragments. The high densities of negative surface charge on DNA and F-actin suggest that the bundles of these filaments, which alone exhibit a strong electrostatic repulsion, may be stabilized by multivalent cations such as histones, antimicrobial peptides, and other positively charged molecules prevalent in airway fluid. Furthermore, it has been observed that bundles of DNA or F-actin formed after addition of histone H1 or lysozyme are efficiently dissolved by soluble multivalent anions such as polymeric aspartate or glutamate. Addition of poly-aspartate or poly-glutamate also disperses DNA and actin-containing bundles in CF sputum and lowers the elastic moduli of these samples to levels comparable to those obtained after treatment with DNase I or gelsolin. Addition of poly-aspartic acid also increased DNase activity when added to samples containing DNA bundles formed with histone H1. When added to CF sputum, poly-aspartic acid significantly reduced the growth of bacteria, suggesting activation of endogenous antibacterial factors. These findings suggest that soluble multivalent anions have potential alone or in combination with other mucolytic agents to selectively dissociate the large bundles of charged biopolymers that form in CF sputum.

Hence, NAC, unfractionated heparin, reduced glutathione, dithiols, Trx, DHLA, other monothiols, DNAse, dornase alfa, hypertonic formulations (e.g., osmolalities greater than about 350 mOsmol/kg), multivalent anions such as polymeric aspartate or glutamate, glycosidases and other examples listed above can be combined according to certain embodiments with a nitrite compound as provided herein (e.g, nitrite anion or a salt thereof such as sodium nitrite, magnesium nitrite or potassium nitrite), or in distinct embodiments with a nitrite- or nitric oxide-donating compound as provided herein, and optionally with one or more other mucolytic agents, for aerosol administration to improve biological activity such as antibacterial, vasodilatory, antihypertensive, anti-inflammatory or anti-proliferative activity through better distribution resulting from reduced sputum viscosity, and improved clinical outcome through improved pulmonary function (from improved sputum mobility and mucociliary clearance) and decreased lung tissue damage from the immune inflammatory response.

### OTHER DOCUMENTS

Chou S-H, Chai C-Y, Wu J-R, Tan M-S, Chiu C-C, Chen I-J, Jeng A Y, Chang C-I, Kwan A-L, Dai Z-K. The effects of debanding on the lung expression of ET-1, eNOS, and cGMP in rats with left ventricular pressure overload. Exp. Biol. Med. 2005. 231:954-9.
Gladwin M T, Raat M J, Shiva S, Dezfulian C, Hogg N, Kim-Shapiro D B, Patel R P. Nitrite as a vascular endocrine nitric oxide reservoir that contributes to hypoxic signaling, cytoprotection, and vasodilation. Am. J. Physiol. Heart Circ. Physiol. 2006. 291:H2026-35.
Hunter C J, Dejam A, Blood A B, Shield H, Kim-Shapiro D B, Machado R F, Tarekegn, Mulla N, Hopper A O, Schechter A N, Power G G, Gladwin M T. Inhaled nebulized nitrite is a hypoxia-sensitive NO-dependent selective pulmonary vasodilator. Nat. Med. 2004. 10:1122-1127.
Kurzyna M, Dabrowski M, Bielecki D, Fijalkowska A, Pruszczyk P, Opolski G, Burakowski J, Florczyk M, Tomkowski W Z, Wawrzynska L, Szturmowicz M, Torbicki A. Atrial septostomy in treatment of end-stage right heart failure in patients with pulmonary hypertension. Chest. 2007. 131:977-83.
Ozaki M, Kawashima S, Yamashita T, Ohashi Y, Rikitake Y, Inoue N, Hirata K I, Hayashi Y, Itoh H, Yokoyama M. Ozaki M, Kawashima S, Yamashita T, Ohashi Y, Rikitake Y, Inoue N, Hirata K I, Hayashi Y, Itoh H, Yokoyama M. Reduced hypoxic pulmonary vascular remodeling by nitric oxide from the endothelium. Hypertension. 2001. 37:322-7.
Rubin L J. 2006. Pulmonary arterial hypertension. Proc. Am. Thorac. Soc. 3:111-115.
Yamashita T, Yamamoto E, Kataoka K, Nakamura T, Matsuba S, Tokutomi Y, Dong Y F, Ichijo H, Ogawa H, Kim-Mitsuyama S. Apoptosis signal-regulating kinase-1 is involved in vascular endothelial and cardiac remodeling caused by nitric oxide deficiency. Hypertension. 2007. 50:519-24.
Yellon D. M. and Hausenloy D. J. 2007. Myocardial Reperfusion Injury N. Engl. J. Med. 357:1121-35.
Duranski M. R., Greer J. J. M., Dejam A., Jaganmohan S., Hogg N., Langston W., Patel R. P., Yet S-F., Wang X., Kevil C. G., Gladwin M. T., and Lefer D. J. Cytoprotective effects of nitrite during in vivo ischemia-reperfusion of the heart and liver. J. Clin. Invest. 2005. 115:1232-1240.
Jung K-H., Chu, K., Ko S-Y., Lee S-T., Sinn D-I., Park D-K., Kim J-M., Song E-C., Kim M., and Roh J-K. Early intravenous infusion of sodium nitrite protects brain against in vivo ischemia-reperfusion injury. Stroke. 2006. 37:2744-2750.
de Perrot M., Liu M., Waddell T. K., and Keshavjee S. Ischemia-Reperfusion-induced Lung Injury. Am. J. Respir. Crit. Care Med. 2003. 167:490-511.
Esme H., Fidan H., Solak O., Dilek F. H., Demirel R., and Unlu M. Beneficial Effects of Supplemental Nitric Oxide Donor Given during Reperfusion Period in Reperfusion-Induced Lung Injury. Thorac. Cardiovasc. Surg. 2006. 54:477-483.
Neto J. S., Nakao A., Kimizuka k., Romanosky A. J., Stolz D. B., Uchiyama T., Nalesnik M. A., Otterbein L. E., and Murase N. Protection of transplant-induced renal ischemia-reperfusion injury with carbon monoxide. Am. J. Physiol. Renal. Physiol. 2004. 287: F979-F989.
Third World Health Conference on Pulmonary Hypertension, 2003, Venice.
Rich S. ed. Executive Summary from the World Symposium on Primary Pulmonary Hypertension, 1998, Evian, France.

### EXAMPLES

The following examples serve to more fully describe the manner of using the above-described invention, as well as to set forth the best modes contemplated for carrying out various embodiments of the invention. It is understood that these examples in no way serve to limit the true scope of this invention, but rather are presented for illustrative purposes. All references cited herein are incorporated by reference in their entireties to the extent they are not inconsistent with the disclosure herein.

### Example 1

### Pharmaceutical Development

Development activities were undertaken to obtain the following two formulation characteristics: 1. Two-vial admixture configuration: improve taste/decrease saltiness; optimize stability; final admixture pH from about 4.7 to about 6.5, preferably between 5 and 6 (facilitates generation of dissolved nitric oxide in the pre-nebulization admixed dosing solution and maintains nitric oxide in the dissolved state through nebulization and inhalation); optimize nebulization device performance (particle size and output rate); and enable flexibility in admixing the desired dose level. From these efforts it was determined that the addition of saccharin significantly reduced the salty taste associated with sodium nitrite. This improvement in taste enabled an increase in sodium nitrite concentration while in its absence sodium nitrite solution admixtures would be unpalatable. 2. Single-vial configuration: improve taste/decrease saltiness; final pH from about 7.0 to about 9.0, preferably between 7 and 8 (facilitates nitrite stability upon storage); and optimize nebulization device performance (particle size and output rate). From these efforts it was determined that the addition of saccharin significantly reduced the salty taste associated with sodium nitrite. This improvement in taste enabled an increase in sodium nitrite concentration while in its absence sodium nitrite solutions would be unpalatable.

To initiate the physico-chemical analysis of sodium nitrite in formulation, the relative solubility and pH of sodium nitrite in water was determined (Table 3).

TABLE 3
Solubility and pH of sodium nitrite in water

pH

| NaNO2 (M) | NaNO2 (mg/mL) | Initial | Stable |
|---|---|---|---|
| 0.73 | 50 | 6.6 | 7.5 |
| 1.45 | 100 | 7.2 | 8.2 |
| 2.90 | 200 | 8.0 | 8.7 |
| 5.80 | 400 | 8.2 | 8.9 |

From these results it appears that sodium nitrite is readily soluble in water to at least 400 mg/mL with a final stable pH of 8.9; the higher the concentration, the higher the pH. Also, in the absence of additional buffering capacity, sodium nitrite pH drifts upwards from that obtained initially (when sodium nitrite is first observed as solubilized) and where the pH becomes stable (within 30 min).

It may be desirable to create a formulation where the final pH is varied. To do this, citric acid was used as a pharmaceutically-acceptable excipient to titrate the pH of various sodium nitrite solutions prepared in water (Table 4).

TABLE 4
Citric acid pH titration and osmolality
of sodium nitrite solution in water

| | | | | Observations | |
|---|---|---|---|---|---|
| NaNO2 | Citric | pH | Osmolality | | Visible |
| (mg/mL) | Acid (mM) | (Stable) | (mOsm/Kg) | Soluble? | Gas? |
| 25 | 1.250 | 5.57 | ND | Yes | No |
| 25 | 0.156 | 6.22 | 648 | Yes | No |
| 25 | 0.078 | 6.40 | 648 | Yes | No |
| 25 | 0.039 | 6.52 | ND | Yes | No |
| 25 | 0.020 | 6.59 | ND | Yes | No |
| 50 | 0.313 | 6.16 | 1251 | Yes | No |
| 50 | 0.156 | 6.48 | 1249 | Yes | No |
| 50 | 0.078 | 6.73 | ND | Yes | No |
| 50 | 0.039 | 6.87 | ND | Yes | No |
| 50 | 0.000 | ND | 1282 | Yes | No |
| 75 | 1.875 | 5.36 | ND | Yes | No |
| 100 | 0.625 | 6.09 | 2383 | Yes | No |
| 100 | 0.313 | 6.45 | 2393 | Yes | No |
| 100 | 0.156 | 6.78 | ND | Yes | No |
| 100 | 0.078 | 7.12 | ND | Yes | No |
| 100 | 0.000 | ND | 2504 | Yes | No |
| 150 | 3.750 | 5.09 | ND | Yes | No |
| 150 | 0.234 | 6.47 | ND | Yes | No |
| 200 | 1.125 | 5.94 | ND* | Yes | No |
| 200 | 0.625 | 6.21 | ND | Yes | No |
| 200 | 0.313 | 6.78 | ND | Yes | No |
| 200 | 0.156 | 7.18 | ND | Yes | No |
| 200 | 0.000 | ND | ND* | Yes | No |
| 400 | 0.000 | ND | ND | Yes | No |

ND—Not determined
*Will not freeze (as required for osmolality determination)

These results suggest that the pH of sodium nitrite may be adjusted with addition of varying concentrations of citric acid over a range of sodium nitrite levels. This approach may be useful in designing aqueous pharmaceutical formulations of sodium nitrite. Moreover, the data in this table demonstrate that osmolality is approximately linear with sodium nitrite concentration, such that osmolality using sodium nitrite at 50 mg/mL is roughly one-half that observed at 100 mg/mL of the sodium nitrite. Similarly, osmolality for sodium nitrite at 25 mg/mL is roughly one-half that of 50 mg/mL. Hence, it can be extrapolated that 12.5 mg/mL sodium nitrite is roughly 300 mOsm/kg, and 6.25 mg/mL sodium nitrite is roughly 150 mOsm/kg.

When nitric oxide is delivered to various tissues it dilates the vasculature. By design, administration of nitrite to the lung or other tissues may be delivering either itself as the active pharmaceutical ingredient or serve as a sustained-release (or pro-drug) molecule that is converted to nitric oxide for therapeutic effect. Thus, if it was possible to create nitric oxide, most preferably dissolved nitric oxide (be that in the formulation solution or aerosolized particles), prior to or during aerosol administration, this may have an immediate and short-acting symptomatic and/or therapeutic effect by acutely reducing vascular pressures, e.g., aerosol delivery to the lung to provide sustained-release nitrite and acutely active dissolved nitric oxide. There are at least two methods to accomplish this formulation. One, is to lower the pH of the solution (e.g., by addition of citric acid) or, two, to include a reducing acid (e.g., ascorbic acid). Acidic pH is a more delicate method that easily prepares a solution with dissolved nitric oxide (Tables 3 and 4). To understand the amount of reducing acid required to produce formulation-dissolved nitric oxide ascorbic acid was titrated against several concentrations of sodium nitrite (Table 5).

TABLE 5
Ascorbic acid titration of sodium nitrite solution
in water: gas-evolution observations

| | | | NO₂⁻: | |
|---|---|---|---|---|
| | | | Ascorbic | |
| NaNO₂ | NaNO₂ | Ascorbic | Acid (Molar | |
| (M) | (mg/mL) | Acid (M) | ratio) | Observation |
| 0.37 | 25 | 0.64 | 1:1.73 | Highly effervescent |
| | | | | emitting yellow/brown |
| | | | | gas* |
| 0.73 | 50 | 0.64 | 1:0.88 | Highly effervescent |
| | | | | emitting yellow/brown |
| | | | | gas* |
| 1.45 | 100 | 0.64 | 1:0.44 | Highly effervescent |
| | | | | emitting yellow/brown |
| | | | | gas* |
| 2.90 | 200 | 0.64 | 1:0.22 | Highly effervescent |
| | | | | emitting yellow/brown |
| | | | | gas* |

*Color of gas suggests as nitrogen dioxide. Nitric oxide is also produced. Effervescence nearly overflowed the container at the higher molar ratios.

These results indicate that at high molar ratios of nitrite to ascorbic acid, solutions are unstable and produce a large amount of gas (both nitrogen dioxide and nitric oxide). From these observations it is clear that this solution is unstable and would not be easily nebulized. To identify an amount of ascorbic acid that would result in only dissolved nitric oxide, ascorbic acid was titrated against sodium nitrite (Table 6).

TABLE 6
Ascorbic acid titration of sodium nitrite solution in water: identification
of molar ratio providing dissolved-state nitric oxide gas

| | | | |
|---|---|---|---|
| Desired Ratio | Ascorbic | | |
| (NaNO₂:Ascorbic | Acid | Final | |
| Acid) | (mM) | pH | Observations |

75 mg/mL NaNO₂, 1.875 mM Citric Acid,
0.25 mM Na Saccharin, starting pH 5.41

| | | | |
|---|---|---|---|
| 16:1 | 64.7 | ND | Large visible bubbles upon mixing |
| 32:1 | 32.3 | ND | Small visible bubbles upon mixing |
| 64:1 | 16.2 | ND | Several small visible bubbles upon |
| | | | mixing |
| 128:1 | 8.1 | 5.84 | A few very small bubbles upon |
| | | | mixing |
| 256:1 | 4.0 | 5.80 | No visible bubbles, even after |
| | | | vortex mixing |
| 512:1 | 2.0 | 5.76 | No visible bubbles, even after |
| | | | vortex mixing |
| 1024:1 | 1.0 | 5.61 | No visible bubbles, even after |
| | | | vortex mixing |
| 2048:1 | 0.5 | ND | No visible bubbles, even after |
| | | | vortex mixing |
| 4096:1 | 0.3 | ND | No visible bubbles, even after |
| | | | vortex mixing |
| 8192:1 | 0.1 | ND | No visible bubbles, even after |
| | | | vortex mixing |

75 mg/mL NaNO₂, 0.117 mM Citric Acid,
0.25 mM Na Saccharin, starting pH 6.55

| | | | |
|---|---|---|---|
| 16:1 | 64.7 | ND | Large visible bubbles upon mixing |
| 32:1 | 32.3 | ND | Small visible bubbles upon mixing |
| 64:1 | 16.2 | ND | Several small visible bubbles upon |
| | | | mixing |
| 128:1 | 8.1 | 5.81 | A few very small bubbles upon |
| | | | mixing |
| 256:1 | 4.0 | 5.82 | No visible bubbles, even after |
| | | | vortex mixing |
| 512:1 | 2.0 | 5.81 | No visible bubbles, even after |
| | | | vortex mixing |
| 1024:1 | 1.0 | 5.96 | No visible bubbles, even after |
| | | | vortex mixing |
| 2048:1 | 0.5 | ND | No visible bubbles, even after |
| | | | vortex mixing |
| 4096:1 | 0.3 | ND | No visible bubbles, even after |
| | | | vortex mixing |
| 8192:1 | 0.1 | ND | No visible bubbles, even after |
| | | | vortex mixing |

From these results it appears that a below or equal to a molar ratio of 256 parts nitrite to 1 part ascorbic acid results in not visible gas formation. From this one may infer that any gas formed would be in the dissolved state. Results indicate that this mixture (at 256:1) produces and releases ˜800 ppb nitric oxide upon vibrating mesh nebulization (using the Aeroneb Go Lab nebulization device, Aerogen, Inc., Galway, Ireland).

From these results it is apparent that the pH of sodium nitrite in aqueous solution may be titrated with citric acid to produce and release ˜200 parts per billion nitric oxide or mixed with ascorbic acid at a 256:1 molar ratio to produce additional dissolved nitric oxide. These results also show that sodium nitrite is very soluble at multiple pH levels, providing the opportunity to administer very high concentrations of sodium nitrite using liquid nebulization. High concentrations permit reduced administration times (important for patient compliance), but suffer in that their associated osmolality and intense taste may mitigate this advantage. To this end, several of the above and more focused liquid formulations of sodium nitrite were prepared, nebulized using both a high efficiency (HE) vibrating mesh nebulizer (particle MMAD ˜3-4 micron and output ˜0.55 mL/min) and lower efficiency Aerogen Aeroneb Go (GO) vibrating mesh nebulizer (Aerogen, Inc., Galway, Ireland) (particle MMAD ˜3-4 micron and output ˜0.22 mL/min). Nebulized solutions were inhaled to a shallow throat level and analyzed for: taste (saltiness), throat irritation, and sore throat. In this analysis, sodium nitrite water was compared to sodium nitrite containing a pH-adjusting reagent (citric acid), a taste-masking agent (sodium saccharin, lactose or sodium bicarbonate), and/or ascorbic acid to produce greater dissolved nitric oxide. Sodium chloride is also considered in the art as helpful to alleviate cough. Therefore, this was also tested. The results are shown in Table 7 below.

TABLE 7
Taste-Testing of Sodium Nitrite Formulations:
Broad-Range Screen with HE Device

| | | | HCO₃ | NaCl | Na Saccharin | Observation | | | |
|---|---|---|---|---|---|---|---|---|---|
| pH | NaNO₂⁻ (mg/mL) | Citric Acid (mM) | (mM) | (mM) | (mM) | Salty Taste | Cough Irritation | Sore Throat | Sweetness |
| 6.22 | 25 | 0.078 | — | — | — | 2 | 2 | 2 | — |
| 7.48 | 50 | — | — | — | — | 4 | 4 | 4 | — |
| 6.16 | 50 | 0.156 | — | — | — | 5 | 5 | 5 | — |
| 6.16 | 50 | 0.156 | — | — | 1.70 | 6 | 6 | 5 | 6 |
| 7.10 | 50 | — | 560 | — | — | 5 | 4 | 3 | — |
| 6.87 | 50 | 0.020 | — | — | — | 4 | 4 | 3 | — |

Saltiness: 1-2 = little salty taste/aftertaste; 3-4 = mild-moderate salty taste/aftertaste; 5-6 = moderate to strong salty taste/aftertaste; and 7-8 = intolerable salty taste/aftertaste.
Cough Irritation: 1-2 = little cough/irritation; 3-4 = mild-moderate cough/irritation; 5-6 = moderate to strong cough/irritation; 7-8 = intolerable cough/irritation.
Sore throat: 1-2 = little sore throat; 3-4 = mild-moderate sore throat; 5-6 = moderate to strong sore throat; 7-8 = intolerable sore throat.
Sweetness: 1-2 = little sweetness; 3-4 = mild-moderate sweetness; 5-6 = moderate to strong sweetness; 7-8 = very strong sweetness.

From these observations, it is clear that all concentrations tested have at least some salty taste, cough irritation and lingerings of a sore throat. To relate this to a potential dose administration, by example, if one desired to deposit mg sodium nitrite in the lung, assuming use of the HE device and this device has an efficiency of theoretical deposition of 35%, at 0.55 mL/min, it would require 71.4 mg sodium nitrite to be loaded into the device. Using the lowest dose tested above (25 mg/mL formulation), this would equate to 2.9 mL. At 0.55 mL/min, administration of 2.9 mL formulation would take ˜5.3 min (or 13.1 min using the GO device). Thus, a patient would need to dose for 5.3 minutes with a formulation which is fairly salty, has some cough irritation and leaves them with a mild sore throat. To decrease this time of administration would require increasing the concentration which, as noted above, results in worse tolerability. It should also be noted that each of these formulation had a pH in the range of ˜6-7.

To understand the taste of formulations in the pH range of 5-6, a more defined pH titration of sodium nitrite citric acid in the presence of different levels of sodium saccharin was performed. These results are shown in Table 8.

TABLE 8
Titration of Sodium Nitrite with Citric Acid in
the Presence of Varying Sodium Saccharin Level

| NaNO₂ | Citric Acid | Na Saccharin | |
|---|---|---|---|
| (mg/mL) | (mM) | (mM) | pH |
| 150 | 1.875 | 0 | 5.03 |
| 150 | 0.117 | 0 | 6.37 |
| 150 | 1.875 | 1.00 | 5.07 |
| 150 | 0.117 | 1.00 | 6.43 |
| 150 | 1.875 | 5.00 | 5.09 |
| 150 | 0.117 | 5.00 | 6.47 |
| 100 | 1.250 | 0 | 5.13 |
| 100 | 0.078 | 0 | 6.49 |
| 100 | 1.250 | 1.00 | 5.13 |
| 100 | 0.078 | 1.00 | 6.52 |
| 100 | 1.250 | 5.00 | 5.15 |
| 100 | 0.078 | 5.00 | 6.57 |
| 75 | 0.938 | 0 | 5.35 |
| 75 | 0.059 | 0 | 6.72 |
| 75 | 0.938 | 1.00 | 5.35 |
| 75 | 0.059 | 1.00 | 6.79 |
| 75 | 0.938 | 5.00 | 5.36 |
| 75 | 0.059 | 5.00 | 6.77 |
| 50 | 0.625 | 0 | 5.55 |
| 50 | 0.039 | 0 | 6.85 |
| 50 | 0.625 | 1.00 | 5.56 |
| 50 | 0.039 | 1.00 | 6.92 |
| 50 | 0.625 | 5.00 | 5.57 |
| 50 | 0.039 | 5.00 | 6.92 |

Using the results in Table 6 above, additional formulations were prepared and taste-tested. These results are shown in Table 9.

TABLE 9
Taste-Testing of Sodium Nitrite Formulations:
Narrow-Range Screen with HE Device.

| | | | Na Saccharin | Lactose | Observation | | | |
|---|---|---|---|---|---|---|---|---|
| pH | NaNO₂ (mg/mL) | Citric Acid (mM) | (mM) | (mM) | Salty Taste | Cough Irritation | Sore Throat | Sweetness |
| 5.57 | 50 | 1.250 | — | — | 5 | 5 | 5 | — |
| 6.42 | 50 | 0.078 | — | — | 4 | 4 | 3 | — |
| 5.09 | 150 | 3.75 | — | — | 6 | 6 | 5 | — |
| 5.09 | 150 | 3.75 | 1.00 | — | 7 | 7 | 5 | 4 |
| 6.47 | 150 | 0.234 | — | — | 5 | 5 | 5 | — |
| 6.47 | 150 | 0.234 | 1.00 | — | 6 | 6 | 5 | 4 |
| 6.47 | 150 | 0.234 | 5.00 | — | 8 | 8 | 7 | 5 |
| 5.36 | 75 | 1.875 | — | — | 6 | 5 | 5 | — |
| 5.36 | 75 | 1.875 | 0.25 | — | 3 | 3 | 2 | 2 |
| 6.70 | 75 | 0.170 | 0.25 | — | 2 | 2 | 2 | 2 |
| 6.70 | 75 | 0.170 | 0.60 | — | 3 | 3 | 2 | 2 |
| 6.50 | 75 | 0.170 | — | 50.00 | 3 | 2 | 2 | 1 |

Saltiness: 1-2 = little salty taste/aftertaste; 3-4 = mild-moderate salty taste/aftertaste; 5-6 = moderate to strong salty taste/aftertaste; and 7-8 = intolerable salty taste/aftertaste.
Cough Irritation: 1-2 = little cough/irritation; 3-4 = mild-moderate cough/irritation; 5-6 = moderate to strong cough/irritation; 7-8 = intolerable cough/irritation.
Sore throat: 1-2 = little sore throat; 3-4 = mild-moderate sore throat; 5-6 = moderate to strong sore throat; 7-8 = intolerable sore throat.
Sweetness: 1-2 = little sweetness; 3-4 = mild-moderate sweetness; 5-6 = moderate to strong sweetness; 7-8 = very strong sweetness.

From these results it appears that less sodium saccharin is better than more, e.g., 0.25 mM improves the taste and tolerability of 75 mg/mL sodium nitrite, while concentrations such as 0.6 mM, 1.0 mM and 5.0 mM have lesser, to a worsening effect, respectively. From this data, the pH 6.7, 75 mg/mL sodium nitrite formulation with 0.25 mM has an improved tolerability over a similar pH, 50 mg/mL sodium nitrite formulation without sodium saccharin. Moreover, although making the formulation more acidic correlates with decreased tolerability, the pH 5.36, 75 mg/mL sodium nitrite formulation with 0.25 mM sodium saccharin also has an improved tolerability over this lower concentration. Hence, as an example using the earlier calculation and HE device, if one desired to deposit 25 mg sodium nitrite in the lung, it would require 71.4 mg sodium nitrite to be loaded into the device. Using this 75 mg/mL formulation (acidic or not), this would equate to 0.95 mL loaded into the device. At 0.55 mL/min, administration of 0.95 mL formulation would take ˜1.7 min (or 4.3 min using the GO device). Thus, although this formulation is slightly less tolerable than the 25 mg/mL formulation, it is administered in significantly less time (1.7 min or 4.3 min compared to 5.3 min and 13.1 min, respectively).

Given the information above, it was next hypothesized that slowing the rate of administration may also improve tolerability. To test this hypothesis, both the HE and GO devices were tested with similar formulations. In addition, as a comparison, the tolerability of ascorbic acid was also tested. The concentration of ascorbic acid used was that which gave the highest concentration of ascorbic acid without forming visible gas bubbles (256:1 sodium nitrite to ascorbic acid). The results are shown in Table 10.

TABLE 10
Taste-Testing of Sodium Nitrite Formulations:
Device Screen with HE and GO Devices

| | | | | | | Observation | | | |
|---|---|---|---|---|---|---|---|---|---|
| | | | Citric Acid | Na Saccharin | Ascorbic Acid | | Cough | | |
| Device | pH | NaNO₂ (mg/mL) | (mM) | (mM) | (mM) | Salty Taste | Irritation | Sore Throat | Sweetness |
| HE | 6.70 | 75 | 0.12 | 0.25 | — | 2 | 2 | 2 | 2 |
| GO | 6.70 | 75 | 0.12 | 0.25 | — | 1 | 1 | 1 | 1 |
| HE | 6.70 | 75 | 0.12 | 0.25 | 4.25 | 8 | 8 | 7 | 1 |
| GO | 6.70 | 75 | 0.12 | 0.25 | 4.25 | 4 | 4 | 3 | 2 |
| HE | 6.45 | 75 | 0.10 | 0.25 | — | 2 | 2 | 2 | 2 |
| GO | 6.45 | 75 | 0.10 | 0.25 | — | 1 | 1 | 1 | 2 |
| HE | 5.41 | 75 | 1.56 | 0.25 | — | 3 | 3 | 3 | 2 |
| GO | 5.41 | 75 | 1.56 | 0.25 | — | 2 | 1 | 1 | 2 |

Saltiness: 1-2 = little salty taste/aftertaste; 3-4 = mild-moderate salty taste/aftertaste; 5-6 = moderate to strong salty taste/aftertaste; and 7-8 = intolerable salty taste/aftertaste.
Cough Irritation: 1-2 = little cough/irritation; 3-4 = mild-moderate cough/irritation; 5-6 = moderate to strong cough/irritation; 7-8 = intolerable cough/irritation.
Sore throat: 1-2 = little sore throat; 3-4 = mild-moderate sore throat; 5-6 = moderate to strong sore throat; 7-8 = intolerable sore throat.
Sweetness: 1-2 = little sweetness; 3-4 = mild-moderate sweetness; 5-6 = moderate to strong sweetness; 7-8 = very strong sweetness.

These results indicate that the 75 mg/mL sodium nitrite formulation is fairly well tolerated with the addition of sodium saccharin. It appears that the amount of sodium saccharin is also important, such that too much is detrimental to tolerability. However, this ratio of sodium nitrite to sodium saccharin may translate improved tolerability to even higher sodium nitrite concentrations, e.g., 100 mg/mL or 150 mg/mL. Further, slowing the administration of these formulations further improves tolerability. Similarly, these higher sodium nitrite concentrations may also be better tolerated with slower administration.

As discussed herein, sodium nitrite in solution, stored under acidic conditions, is unstable. Therefore, to enable stability the two-vial admixture configuration was created to separate sodium nitrite from citric acid (or other acidifying agent) until admixture and administration. To further stabilize the sodium nitrite solution vial, sodium phosphate buffer was included in Vial 1 (sodium nitrite and sodium phosphate). However, it was important to carefully titrate the amount of phosphate buffer so that the pH of Vial 1 remained above pH 7; and, so that this level of phosphate buffer did not dominate the desired final admixture pH level. Thus, the amount of phosphate buffer to enable stability of the sodium nitrite vial, but in an amount that wouldn't elevate the final admixture pH above desired levels, was determined. Results are shown in Table 11.

TABLE 11
Phosphate Buffer Admixture Titration

| Sodium Phosphate | Citric Acid | Sodium Nitrite | Final |
|---|---|---|---|
| (mM)¹ | (mM) | (mg/mL) | pH |
| 0 | 3.20 | 12.6 | 4.6 |
| 1.0 | 3.19 | 12.6 | 4.7 |
| 2.5 | 3.18 | 12.5 | 4.8 |
| 4.0 | 3.17 | 12.5 | 5.0 |
| 6.4 | 3.16 | 12.4 | 5.2 |
| 7.9 | 3.15 | 12.4 | 5.4 |
| 9.3 | 3.14 | 12.4 | 5.6 |
| 11.7 | 3.13 | 12.3 | 5.9 |
| 14.1 | 3.11 | 12.2 | 6.2 |
| 16.4 | 3.09 | 12.2 | 6.4 |
| 18.3 | 3.08 | 12.1 | 6.5 |
| 20.6 | 3.07 | 12.1 | 6.6 |
| 22.9 | 3.05 | 12.0 | 6.6 |
| 25.2 | 3.04 | 12.0 | 6.7 |

¹The 500 mM sodium phosphate stock buffer solution was made up with 27.6 mg/mL sodium phosphate monobasic monohydrate and 53.6 mg/mL sodium phosphate dibasic heptahydrate.

As a result of the studies performed above, clinical trial materials were produced under cGMP-compliant conditions in 3 formulation/vialing configurations. The three admixture clinical trial vial formulations were:

Vial 1, Sodium Nitrite Solution, Sterile

Vial 2, Excipient Solution, Sterile

Vial 3, Placebo/Diluent Solution, Sterile

Vial 1 contains 300 mg/mL sodium nitrite and 0.1 mmol/L sodium phosphate, filled at a volume of 4 mL. Vial 2 contains 1.0 mmol/L sodium saccharin as a taste-masking agent and 6.4 mmol/L citric acid (pH 3.0) to moderate pH of the final admixture solution, filled at a volume of 3 mL. Vial 3 contains 0.1 mmol/L sodium phosphate alone to be used as placebo substituted for Vial 1 or diluent to allow further dilution of the Vial 1/Vial 2 admixture as needed to achieve the various AIR001 Inhalation Solution dosing configurations required for Phase 1 administration. The clinical trial vial formulations were put on a GMP stability program, and following 6 months of storage at 40° C. and 75% relative humidity and 9 months of storage at 25° C. and 60% relative there are no discernable changes in attributes of the 3 formulation/vialing configuration.

In addition to a two-vial admixture, single-vial sodium nitrite formulations were created containing varying amounts of sodium nitrite and different ratios of sodium saccharin. It is hypothesized that this single-vial configuration will be stable at room temperature and provide a range of well-tolerated sodium nitrite formulations. Each formulation was nebulized and assessed for taste and irritability. The concentration of sodium saccharin used was between 0 mM to about 2.0 mM. The results are shown in Table 12.

TABLE 12
Taste-Testing of Sodium Nitrite Formulations:
A single-Vial Configuration

| | | | | Sodium | Observation | | | |
|---|---|---|---|---|---|---|---|---|
| | | | Na Saccharin | Phosphate | | Cough | | |
| Device | pH | NaNO₂ (mg/mL) | (mM) | Buffer | Salty Taste | Irritation | Sore Throat | Sweetness |
| GO | 7.6 | 75.0 | 0.39 | 1.00 | 1.0 | 2.0 | 1.0 | 1.0 |
| GO | — | 90.0 | 0.95 | 1.0 | 1.0 | 2.0 | 0.7 | 2.7 |
| GO | — | 100.0 | 0.33 | 0.1 | 1.25 | 3.5 | 1.25 | 1.5 |
| GO | 7.4 | 100.0 | 0 | 5.0 | 3.0 | 1.5 | 1.0 | 0.25 |
| GO | — | 100.0 | 0.33 | 5.0 | 3.0 | 2.0 | 1.0 | 1.25 |
| GO | — | 100.0 | 0.17 | 1.0 | 3.0 | 2.3 | 1.0 | 1.3 |
| GO | — | 100.0 | 1.05 | 1.0 | 1.3 | 3.3 | 0.7 | 4.3 |
| GO | 7.3 | 100.9 | 2.03 | 5.0 | 1.0 | 3.7 | 1.3 | 5.0 |
| GO | 7.5 | 90 | 0.1 | 2.5 | 1.5 | 2.5 | 1.0 | 1.0 |
| GO | 7.5 | 90 | 0.3 | 2.5 | 2.0 | 1.5 | 1.0 | 1.25 |
| GO | — | 90 | 1.0 | 2.5 | 2.5 | 1.5 | 1.0 | 1.75 |
| GO | 7.4 | 90 | 0.5 | 2.5 | 2.25 | 1.75 | 2.0 | 1.75 |
| GO | 7.3 | 90 | 0.5 | 1.0 | 2.75 | 2.25 | 1.5 | 1.25 |
| GO | 7.4 | 60 | 0.3 | 2.5 | 1.25 | 1.25 | 1.0 | 1.25 |
| GO | 7.4 | 30 | 0.3 | 2.5 | 0.75 | 0.75 | 0.5 | 1.25 |
| GO | 7.3 | 10 | 0.3 | 2.5 | 0.75 | 0.0 | 0.0 | 2.5 |
| GO | — | 10 | 0.15 | 2.5 | 0.75 | 0.0 | 0.0 | 0.75 |
| GO | 7.4 | 90 | 0 | 2.5 | 3.25 | 3.0 | 1.25 | 0.5 |

Saltiness: 1-2 = little salty taste/aftertaste; 3-4 = mild-moderate salty taste/aftertaste; 5-6 = moderate to strong salty taste/aftertaste; and 7-8 = intolerable salty taste/aftertaste.
Cough Irritation: 1-2 = little cough/irritation; 3-4 = mild-moderate cough/irritation; 5-6 = moderate to strong cough/irritation; 7-8 = intolerable cough/irritation.
Sore throat: 1-2 = little sore throat; 3-4 = mild-moderate sore throat; 5-6 = moderate to strong sore throat; 7-8 = intolerable sore throat.
Sweetness: 1-2 = little sweetness; 3-4 = mild-moderate sweetness; 5-6 = moderate to strong sweetness; 7-8 = very strong sweetness.

These results indicate that formulations containing 100 mg/mL sodium nitrite were moderately well tolerated (with the addition of sodium saccharin). However, formulations containing 90 mg/mL or less sodium nitrite were better tolerated (with the addition of sodium saccharin). It appears that the amount of sodium saccharin was also important; such that too much or too little was detrimental to tolerability, while the range of 0.15 mM to about 1.0 mM appeared to be preferred for certain embodiments. Similarly, the 5 mM sodium phosphate buffer was less well tolerated than both 2.5 mM and 1.0 mM. However, lower sodium nitrite levels permitted greater sodium phosphate concentrations.

As a result of the studies performed above, formulation prototypes were manufactured to evaluate compatibility/stability of a single-vial system. Vial 1 contains 10 mg/mL sodium nitrite, 0.3 mmol/L sodium saccharin and 2.5 mmol/L sodium phosphate, at pH 7.5 and filled at a volume of 8 mL. Vial 2 contains 10 mg/mL sodium nitrite, 0.3 mmol/L sodium saccharin and 2.5 mmol/L sodium phosphate, at pH 7.3 and filled at a volume of 8 mL. Vial 3 contains 90 mg/mL sodium nitrite and 2.5 mmol/L sodium phosphate, at pH 7.3 and filled at a volume of 8 mL. The prototype formulations were put on a 6 month stability program to evaluate compatibility/stability. Results for samples stored for 1 month at 40° C. are shown in Table 13.

TABLE 13
Single-Vial Formulations and Stability

| | Vial 1 (10 mg/mL NaNO₂, 0.3 mM Na | |
|---|---|---|
| | saccharin, and 2.5 mM sodium phosphate buffer) | |
| Attribute | Initial | 1 Month* |
| pH | 7.5 | 7.6 |
| Potency (% of Label | 97 | 96 |
| Claim) | | |
| Impurities (%) | 0.05 | 0.03 |
| 1. RRT = 0.63 | | |
| Saccharin Assay (mM) | 2.7 | 2.7 |
| | Vial 2 (90 mg/mL NaNO₂, 0.3 mM Na | |
| | saccharin, and 2.5 mM sodium phosphate buffer) | |
| Attribute | Initial | 1 Month* |
| pH | 7.3 | 7.3 |
| Potency (% of Label | 95.3 | 95.8 |
| Claim) | | |
| Impurities (%) | 0.03 | 0.01 |
| 1. RRT = 0.63 | | |
| Saccharin Assay (mM) | NA | NA |

Vial 3 (90 mg/mL NaNO₂ and 2.5 mM
sodium phosphate buffer)

| Attribute | Initial | 1 Month* |
|---|---|---|
| pH | 7.3 | 7.4 |
| Potency (% of Label | 96.3 | 96.8 |
| Claim) | | |
| Impurities (%) | 0.01 | 0.02 |
| 1. RRT = 0.63 | | |
| Saccharin Assay (mM) | 0 | 0 |

*Storage at 40° C. and 75% relative humidity
NA = Not available

Based on the results presented in Table 13, the formulation prototypes can be considered to be stable following one month of accelerated storage (40° C. and 75% relative humidity).

Summary.

The addition of citric acid during the admixture step catalyzed the pH-dependent formation of a small amount of dissolved nitric oxide that is predicted to provide mild acute arteriodilation and potentially enhanced and immediate acute symptomatic relief of dyspnea in the PAH patients with elevated pulmonary arterial pressures. Combined with sustained deoxyhemoglobin-catalyzed generation of nitric oxide in vivo from the delivered nitrite compound, both acute relief and sustained-symptomatic relief are anticipated. The level of nitric oxide produced from the 75 mg/mL sodium nitrite, 1.56 mM citric acid, 0.25 mM sodium saccharin, pH 5.41 admixed dosing solution was ˜200 parts per billion compared to ˜150 parts per billion in the absence of citric acid. (Similarly, the same formulation with ascorbic acid produced ˜800 parts per billion. However, although in the dissolved state, it is predicted that this reagent may produce adverse levels of nitrogen dioxide which is toxic to the lung.) These results are in comparison to 5-80 parts per million administered using inhaled nitric oxide (Ehrenkranz, et al. 1997). Finally, because viscosity and surface tension can be important contributors to optimal I-neb (Respironics, Inc., Murrysville, Pa.), Aerogen Aeroneb Go (Aerogen, Inc., Galway, Ireland) or other vibrating mesh nebulizer performance, potential formulations were screened in these devices to measure the effect of formulation on device output rate and aerosol particle size. The solution formulation described here meets the above criteria to produce a nebulized aerosol that is projected to be well-tolerated and enables the broad-range dose-titration enabling moderation of dose levels as desired to achieve optimal intra-nasal, pulmonary, alveolar, and or blood levels for a given indication described herein.

The following desired two-vial admixture aqueous solution formulation for nebulization parameters were defined:
Sodium Nitrite:Sodium Saccharin molar ratio from about 1.3×10³:1 to about 4.4×10³:1;
Sodium Nitrite:Citric Acid molar ratio from about 2.0×10²:1 to about 6.9×10²:1;
Sodium Nitrite:Phosphate buffer molar ratio less than or equal to about 15:1 to about 180:1;
Admixed solution pH from about 4.7 to about 6.5, more preferably from about pH 5.0 to about pH 6.0

Sodium saccharin and citric acid are generally regarded as safe (GRAS) when administered via the pulmonary route. Aerosol administration of phosphate as an excipient to patients with asthma has been reported using millimolar doses of sodium phosphate. Gaston et al., 2006, concluded that this route of administration for sodium phosphate-containing formulations was safe and no serious adverse effects were noted.

In addition to the two-vial admixture formulation, several single-vial product configurations were also created and assessed. In these studies it was found that formulations containing sodium nitrite alone were poorly tolerated (salty taste, irritation and cough). However, addition of sodium saccharin with a molar ratio of between 0.1 mM to about 2.0 mM considerably improved tolerability (reduced throat irritation, reduced propensity to cough, and reduced salty taste). As with the two-vial configuration, because viscosity and surface tension can be important contributors to optimal I-neb, Aerogen Aeroneb Go or other vibrating mesh nebulizer performance, potential formulations were screened in these devices to measure the effect of formulation on device output rate and aerosol particle size. Therefore, like the two-vial configuration, this single-vial system also meets the criteria to produce a nebulized aerosol that is projected to be well-tolerated and allows the broad-range dose-titration permitting moderation of dose levels as desired to achieve optimal intra-nasal, pulmonary, alveolar, and or blood levels for a given indication described herein.

The following desired single-vial aqueous solution formulation for nebulization parameters were defined:
Sodium Nitrite less or equal to 100 mg/mL, more preferably less than or equal to 90 mg/mL;
Sodium Saccharin between about 0.1 mM and about 2.0 mM, more preferably from about 0.15 mM to about 1.0 mM;
Phosphate buffer between about 1.0 mM and about 5.0 mM, more preferably from about 1.0 mM to about 2.5 mM;
Solution pH from about 7.0 to about 9.0, more preferably from about 7.0 to about 8.0

Prototype Formulations.

Several prototype sodium nitrite formulations were created and characterized in the presence and absence of sodium phosphate, citric acid and sodium saccharin. Selecting from sodium nitrite concentrations ranging from 25-400 mg/mL, it was determined that two-vial admixture and single-vial formulation attributes listed above were optimal for achieving a palatable, tolerated, and stable formulation which generated dissolved-state nitric oxide. For the two-vial configuration, citric acid content was optimized to create dissolved nitric oxide. Because gaseous nitric oxide in the formulation solution interferes with nebulizer performance, this state was avoided in the described “Formulation I” Inhalation Solution formulation.) From these studies, it was determined that sodium nitrite was less stable at pH-levels below 7. Because an acidic dosing solution is desired to provide low-level (solution-dissolved) nitric oxide formation to promote immediate, mild acute symptomatic relief of dyspnea in PAH patients, it was essential to create a two-vial, admixture system where sodium nitrite would remain at the stability-enabling pH of greater than 7 until use. As noted above, the ratio of sodium nitrite to both sodium saccharin (for palatability) and citric acid (for pH adjustment) are important. It was also suggested as best to manufacture these two excipients together in a set ratio (Vial 2: 1.35 mg/mL citric acid, 0.24 mg/mL sodium saccharin dihydrate, pH 3.0±0.5). Because the pH of aqueous sodium nitrite tends to drift in the absence of a buffer, it was determined that a small amount of sodium phosphate should be included in Vial 1 to stabilize the pH above 7 (Vial 1: 300 mg/mL sodium nitrite, 0.1 mM sodium phosphate, pH 8.0±0.5).

At the highest sodium nitrite dosing admixture target concentration (150 mg/mL), an equal volume of Vial 1 and Vial 2 are admixed to produce 150 mg/mL sodium nitrite at the target optimized concentrations of sodium saccharin and citric acid. A third formulation may be produced to contain sodium phosphate buffer only (Vial 3). This formulation will substitute for Vial 1 in Placebo administrations, or will be used to dilute Vial 1 and Vial 2 admixtures to achieve lower sodium nitrite dose solution concentrations.

As for the two-vial admixture, the single-vial configuration was also optimized for taste and tolerability. However, in these studies because sodium nitrite is unstable under acidic pH, citric acid was not included. Thus, various sodium nitrite concentrations were assessed in the presence and absence of the sodium saccharin taste-masking agent to obtain an optimum ratio of active ingredient to excipient(s) for this formulation configuration. From these studies, it was determined that sodium saccharin was required for taste and tolerability at an optimum ratio of about 0.1 mM and about 2.0 mM. Further, from the phosphate buffer titrations, it was determined that sodium phosphate may be included between from about 1.0 to about 5 mM. Moreover, the single vial configuration appears stable for at least one month under accelerated conditions.

### Example 2

### Aqueous Sodium Nitrite Admix Formulation for Liquid Nebulization Administration

### Batches & Vial Configurations

TABLE 14
Sodium Nitrite Solution, pH 8.0 (Vial
1), 4 mL fill with argon overlay

| | | | |
|---|---|---|---|
| Chemical | MW | Vial Conc. | Amount/Vial |

| | | | | | |
|---|---|---|---|---|---|
| Sodium Nitrite | 69.00 | 300 | mg/mL | 1200 | mg |
| NaH₂PO₄—H₂O | 137.99 | 6.9 | μg/mL | 0.028 | mg |
| Na₂HPO₄—7 H₂O | 268.07 | 13.4 | μg/mL | 0.054 | mg |

| | | | | |
|---|---|---|---|---|
| SWFI (final vol) | — | — | 4 | mL |

1. To 50% total volume sterile water for injection (SWFI), add and dissolve:
Monobasic sodium phosphate (NaH₂PO₄)
Dibasic sodium phosphate (Na₂HPO₄)

2. After phosphates are dissolved in 50% total volume SWFI, add and dissolve:
Sodium nitrite

3. Measure and record pH (preliminary spec 8.0+/−0.5)

4. Adjust volume to 100% with SWFI

5. Re-measure and record pH

6. Pass entire formulation through two 0.22 μm Millipore PVDF filters in series, taking samples before and after filtration for sterility testing and nitrite quantification

7. Co-fill vials with sterile-filtered formulation and argon gas

8. Over-lay fills with argon gas just prior to inserting stoppers

TABLE 15
Excipient Solution, pH 3.0 (Vial 2) 3 mL fill

| Chemical | MW | Vial Conc | Amount/Vial | |
|---|---|---|---|---|
| Sodium Saccharin - 2H₂O | 241.19 | 1.0 mM | 0.724 | mg |
| | | (0.241 mg/mL) | | |
| Citric Acid - H₂O | 210.14 | 6.4 mM | 4.035 | mg |
| | | (1.345 mg/mL) | | |
| SWFI (q.s.) | — | — | 3 | mL |

1. To 70% total volume SWFI, add and dissolve:
Sodium Saccharin (dihydrate)

2. After Saccharin is dissolved in 70% total volume SWFI, add and dissolve:
Citric Acid

3. Measure and record pH (preliminary spec 3.0+/−0.5)

4. Adjust volume to 100% with SWFI

5. Re-measure and record pH

6. Pass entire formulation through two 0.22 μm Millipore PVDF filters in series taking samples before and after filtration for sterility testing

7. Fill vials with sterile-filtered formulation

8. Stopper vials

TABLE 16
Placebo/Diluent Solution, pH 8.0 (Vial 3) 4 mL fill

| Chemical | MW | Vial Conc. | Amount/Vial | |
|---|---|---|---|---|
| NaH₂PO₄—H₂O | 137.99 | 6.9 μg/mL | 0.028 | mg |
| Na₂HPO₄—7 H₂O | 268.07 | 13.4 μg/mL | 0.054 | mg |
| SWFI (final vol) | — | — | 4 | mL |

1. To 70% total volume SWFI, add and dissolve:
Monobasic sodium phosphate (NaH₂PO₄)
Dibasic sodium phosphate (Na₂HPO₄)

2. Measure pH (preliminary spec 8.0+/−0.5)

3. Adjust volume to 100% with SWFI

4. Pass entire formulation through two 0.22 μm Millipore PVDF filters in series taking samples before and after filtration for sterility testing

5. Fill vials with sterile-filtered formulation

6. Stopper vials

Vial Configurations (all have 8.4 mL fill capacity);

Vial 1—Sodium Nitrite Solution (4 mL)

Vial 2—Excipient Solution (3 mL)

Vial 3—Placebo/Diluent Solution (4 mL)

Vial 4—Empty Mixing Vial

Vials 1, 2, and 3 may be diluted to achieve dosing solutions for the proposed Phase 1 studies as described above. Table 14 is an exemplary listing of mixing instructions to prepare the highest (150 mg/mL sodium nitrite formulation) through potential lower sodium nitrite admixed dosing solutions.

As outlined in Table 17, the high concentration dosing solution is first prepared by adding 3 mL of Vial 1 to the 3 mL present in Vial 2 to create a 150 mg/mL sodium nitrite solution. This mixture may be used directly to administer a 150 mg/mL sodium nitrite dosing solution. By example, to create a 125 mg/mL sodium nitrite dosing solution, combine 5 mL of this 150 mg/mL sodium nitrite dosing solution with 1 mL Placebo/Diluent Solution (Vial 3) into the empty Vial 4. Following this scheme, several dilutions may be prepared. By example, Table 17 shows dilutions creating dosing solutions down to 0.75 mg/mL sodium nitrite.

TABLE 17
Formulation I Inhalation Solution:
Representative Dilutions and Dose-Level Concentrations

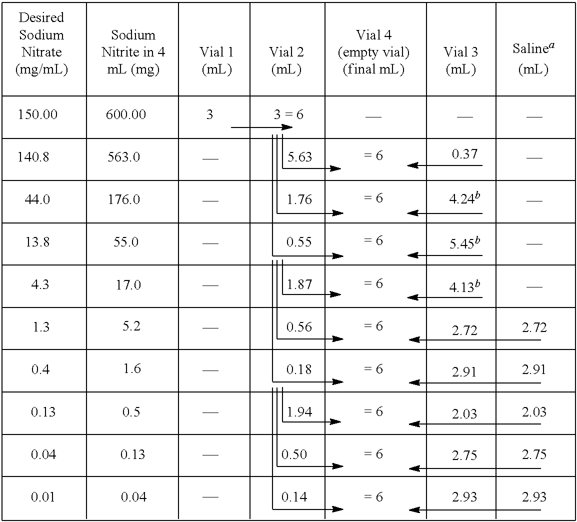

^{a}Sodium Chloride Injection, USP. Sodium chloride addition adjusts tonicity to enhance acute tolerability during inhalation of these lower sodium nitrite dosing solutions.

Table 18 shows the relative stability for the two-vial drug product following admixture over an 8 hour period. As predicted, nitrite assay decreases over this period.

TABLE 18
Admixture Characterization

| Admixture (NaNO2) | 25° C. Incubation (hr) | | | |
|---|---|---|---|---|
| Concentration | 0 | | | 8 |
| (mg/mL) | pH | 1 | 4 | pH |
| 150 | 5.20 | ND | ND | 5.25 |
| 120 | 5.92 | ND | ND | ND |
| 100 | 5.61 | ND | ND | ND |
| 75 | 5.35 | ND | ND | ND |
| 50 | 5.26 | ND | ND | ND |
| 30 | 5.12 | ND | ND | ND |
| 15 | 4.97 | ND | ND | 5.00 |

| | % Label Claim | % Label Claim | % Label Claim | % Label Claim |
|---|---|---|---|---|
| Concentration | (Sodium | (Sodium | (Sodium | (Sodium |
| (mg/mL) | Nitrite) | Nitrite) | Nitrite) | Nitrite) |
| 150 | 101.7 | 100 | 99 | 98 |
| 15 | 99.0 | 99 | 100 | 99 |

| | Impurity | Impurity | Impurity | Impurity |
|---|---|---|---|---|
| Concentration | (Nitrate) | (Nitrate) | (Nitrate) | (Nitrate) |
| (mg/mL) | mg/mL | mg/mL | mg/mL | mg/mL |
| 150 | 0.33 | 0.41 | 0.60 | 0.89 |
| 15 | 0.04 | 0.10 | 0.19 | 0.32 |
| | Impurity | Impurity | Impurity | Impurity |
| Concentration | (Peak 1) | (Peak 1) | (Peak 1) | (Peak 1) |
| (mg/mL) | % | % | % | % |
| 150 | 0.01 | 0.02 | 0.02 | 0.18 |
| 15 | 0.01 | 0.19 | 0.19 | 0.19 |
| | Total | Total | Total | Total |
| Concentration | Impurities | Impurities | Impurities | Impurities |
| (mg/mL) | (%) | (%) | (%) | (%) |
| 150 | 0.32 | 0.38 | 0.54 | 0.93 |
| 15 | 0.37 | 0.93 | 1.55 | 2.40 |

### Example 3

### Effect of Degassing Solution and Overlay on Sodium Nitrite Solution Stability

It was predicted that the stability of aqueous solution sodium nitrite may benefit from manufacturing vials in the absence of oxygen. To assist in determining the best manufacturing process, three batches of the Vial 1 configuration were prepared and placed on ambient and accelerated stability for 2 months.

### Vial 1 Manufacturing Processes.

Process 1: 300 mg/mL sodium nitrite, 0.1 mM sodium phosphate, formulated in nitrogen-sparged sterile-water for injection (SWFI), then vialed and stoppered with an argon overlay.

Process 2: 300 mg/mL sodium nitrite, 0.1 mM sodium phosphate, formulated in SWFI, then vialed and stoppered with an argon overlay.
Process 3: 300 mg/mL sodium nitrite, 0.1 mM sodium phosphate, formulated in SWFI, then vialed and stoppered under ambient atmosphere.

Results from Table 19 demonstrate that each manufacturing process enables equivalent sodium nitrite solution stability for out to two months at 25° C. and 60° C. However, inclusion of an argon overlay to enable long-term solution stability may be a reasonable practice.

TABLE 19
Effect of Degassing and Inert Gas Overlay
on Sodium Nitrite Solution Stability

| Vial 1 (Sodium | | | | |
|---|---|---|---|---|
| Nitrite/Phosphate) | Measurement | 0 | 1 | 2 |
| | | 25° C. Stability (months) | | |
| Process 1 | pH | 8.28 | 8.26 | 8.31 |
| | Sodium Nitrite | 102.8 | 101.8 | 101.9 |
| | (% Label Claim) | | | |
| | Sodium Nitrate | 0.7 | 0.5 | 0.5 |
| | (mg/mL) | | | |
| Process 2 | pH | 8.43 | 8.36 | 8.40 |
| | Sodium Nitrite | 102.8 | 101.6 | 102.0 |
| | (% Label Claim) | | | |
| | Sodium Nitrate | 0.7 | 0.5 | 0.5 |
| | (mg/mL) | | | |
| Process 3 | pH | 8.14 | 8.13 | 8.18 |
| | Sodium Nitrite | 102.5 | 101.4 | 101.3 |
| | (% Label Claim) | | | |
| | Sodium Nitrate | 0.7 | 0.5 | 0.5 |
| | (mg/mL) | | | |
| | | 60° C. Stability (months) | | |
| Process 1 | pH | 8.28 | ND | 8.37 |
| | Sodium Nitrite | 102.8 | ND | 102.5 |
| | (% Label Claim) | | | |
| | Sodium Nitrate | 0.7 | ND | 0.6 |
| | (mg/mL) | | | |
| Process 2 | pH | 8.43 | ND | 8.43 |
| | Sodium Nitrite | 102.8 | ND | 102.8 |
| | (% Label Claim) | | | |
| | Sodium Nitrate | 0.7 | ND | 0.5 |
| | (mg/mL) | | | |
| Process 3 | pH | 8.14 | ND | 8.31 |
| | Sodium Nitrite | 102.5 | ND | 101.9 |
| | (% Label Claim) | | | |
| | Sodium Nitrate | 0.7 | ND | 0.6 |
| | (mg/mL) | | | |

### Example 4

### In Vitro Analysis of the Respironics I-neb, PARI LC STAR, And Aeroneb Go Nebulizers with Sodium Nitrite

The in vitro performance of the Respironics I-neb nebulizer (Respironics, Inc., Murrysville, Pa.), the PARI LC STAR nebulizer (PARI Respiratory Equipment, Inc., Midlothian, Va.; PARI GmbH, Stamberg, Germany), and the Aeroneb Go nebulizer (Aerogen, Inc., Galway, Ireland) were investigated for the delivery of a preliminary liquid formulation of sodium nitrite. The formulation comprised a solution of sodium nitrite (55.6 mg/mL), citric acid, and sodium saccharin. The Respironics I-neb (Respironics, Inc., Murrysville, Pa.) was studied with a 1.5 mL maximum fill volume (83.3 mg Na nitrite) medication chamber and a power 12 disc. It was evaluated in tidal breathing mode (TBM) only. The PARI LC STAR and Aeroneb Go nebulizers were studied with fill volumes of 5 mL (277.8 mg).

The output characteristics of the three nebulizers (one of each type) were measured to determine the particle size distribution, inspired dose, residual dose, nebulizer output and duration of nebulization. The nebulizer efficiency, defined as the inhaled fine particle dose as a percent of the total loaded dose, was also determined. Each nebulizer was studied in duplicate, for a total of 6 measures per device type.

### Materials:

1. Drug: sodium nitrite, 55.6 mg/mL.

2. Loading Dose:
a. Respironics I-neb
   1) 1.5 mL (83.3 mg)
b. PARI LC STAR and Aeroneb Go
   1) 5 mL (277.8 mg)

3. Devices and power source (n=3 each):
a. Respironics I-neb with 1.5 mL chamber
   1) Emergency Disc power level 12.
b. PARI LC STAR
   1) PARI Proneb Ultra compressor.
c. EVO Aeroneb Go
   1) AC power supply.

Particle Sizing.

Nebulizers were weighed dry, full, and at the end of each study to determine gravimetric output and residual volume. Nebulizers were connected to the inhalation cell of the Insitec Laser (Malvern Instruments Ltd, Malvern, Worcestershire, UK) using a flexible airtight connector. The nebulizer and inhalation cell were oriented in a horizontal position. The output end of the inhalation cell was connected to a vacuum generator providing a continuous flow of 20 LPM across the laser beam. For measurements made with the I-neb, the output end of the inhalation cell was connected to a breath simulator, which permitted cyclic particle size measurements using the following breathing pattern: Rate=15 bpm, Volume=500 mL, I.T=2.0 seconds. This is the breathing pattern used for the output studies as well.

Each study was timed from the beginning of nebulization and run for a total of 2 minutes. During the first minute, no measurements were made to allow for equilibration of the solution. Particle sizing was begun and analyzed continuously for the duration of the 2nd minute. All data points were averaged for each measure.

1. VMD: Volume Median Diameter

2. GSD: Geometric Standard Deviation

3. %<3μ: The percent of particles <3 microns

4. %<5μ: The percent of particles <5 microns

5. Duration: Two minute total cycle time

Drug Output.

Three devices were studied two times each. The devices were weighed dry, after the addition of drug, and at the conclusion of nebulization. An inspiratory filter was also weighed dry, prior to measurement and at the conclusion of each run to determine gravimetric change. The nebulizer was connected with its mouthpiece to an inspiratory filter and to a PARI Compass breath simulator (PARI Respiratory Equipment, Inc., Midlothian, Va.; PARI GmbH, Starnberg, Germany), programmed to develop the following breathing pattern: Rate=15 bpm, Volume=500 mL, I.T=2.0 seconds. Nebulization was begun and timed from the beginning until 1 minute past the onset of sputter (PARI STAR) The I neb was timed from the beginning of nebulization until automatic shut-off and the Aeroneb Go from the beginning until the loss of visible particle generation.

At the end of nebulization, the devices and filters were weighed to determine gravimetric change, and washed with distilled water to collect deposited drug. For the Aeroneb Go, drug remaining within the medication cup was assayed but not that within the nebulizer body. Each sample was evaluated for drug concentration with a spectrophotometer at 540λ.

Output Measurements Made were:

1. Duration of nebulization

2. Loading dose (LD): Total drug loaded within the nebulizer

3. Residual dose (RD): Total drug remaining in the nebulizer.

4. Inspired dose (ID): The predicted amount of ED deposited within the lung.

5. Expired Dose: Total drug on the expiratory filter. Collected only on the PARI STAR

6. Fine Particle Dose (FPD): The proportion of inspired dose with particles ≦5 microns.

7. Ultra-Fine Particle Dose (UFPD): The proportion of inspired dose with particles ≦3 microns.

8. Output (FPD per minute): The calculated FPD delivered per minute of nebulization

9. FPD %: The FPD expressed as percent of nominal dose

TABLE 20
Device Characterization Results (n = 6)

| | PARI STAR | I-neb | Aeroneb Go |
|---|---|---|---|
| VMD | 1.7 ± 0.1 | 4.8 ± 0.5 | 4.6 ± 0.1 |
| GSD | 2.8 ± 0.1 | 1.8 ± 0.1 | 2.1 ± 0.1 |
| Duration (minutes) | 19.5 ± 0.7 | 12.5 ± 1.0 | 6.2 ± 1.1 |
| Loading Dose (mg) | 277.8 | 83.3 | 277.8 |
| Residual Dose (mg) | 115.4 ± 14.6 | 5.4 ± 0.8 | 22.9 ± 4.8* |
| Inspired Dose (mg) | 106.3 ± 10.1 | 78.2 ± 3.0 | 74.6 ± 10.6 |
| Expired Dose (mg) | 49.5 ± 7.2 | NA** | NA** |
| Total Recovered (mg) | 271.2 ± 6.5 | 83.6 ± 2.7 | 97.5 ± 10.9 |
| Fine Particle Dose | 89.9 ± 7.8 | 40.7 ± 1.4 | 39.9 ± 5.2 |
| (mg) | | | |
| Ultra Fine Particle | 73.6 ± 7.0 | 21.5 ± 0.8 | 25.3 ± 3.6 |
| Dose (mg) | | | |
| Output (FPD/Min) | 4.6 ± 0.4 | 3.3 ± 0.2 | 6.5 ± 0.6 |
| FPD % | 32.4 ± 3.1 | 48.8 ± 1.9 | 14.4 ± 2.0 |

Mean ± sd
*Contains only drug within the medication cup.
**Exhaled drug not measured

When analyzing these data, one must keep in mind that the I-neb nominal dose was only 30% that of the other devices. The I-neb only nebulizes during a portion of inspiration, and one scintigraphy study showed that 63% of the emitted dose was deposited in the lung with this device in TBM operation. In this case using a starting dose of 83.3 mg, that would translate to an estimated lung dose of 49 mg in 12.5 minutes.

The estimated lung dose with the other devices is more difficult to predict, but some data suggests that for nebulizers, the 3 micron cutoff approximates lung dose fairly well in adults. Using that cutoff, the PARI LC STAR would give an estimated lung dose of 73.6 mg in 19.5 minutes, and the Aeroneb Go would give 25.3 mg in 6.2 minutes. Using this logic, all devices delivered about the same “estimated lung dose per minute” (3.8-4.1 mg/min).

To further differentiate the devices, one needs to consider the indication for the drug, the target in the lung, the acceptance of the device, and the device expense.

The I-neb (Respironics, Inc., Murrysville, Pa.) is a very complex electronic device that is already on the market for delivery of iloprost for pulmonary hypertension. One benefit is that drug is dosed during inhalation only, thus preventing contamination of the surroundings and/or caregivers. It is also already approved for a pulmonary hypertension product, and is battery operated (portable). If it were used in the Target Inhalation Mode, there is a good chance that delivery time can be reduced and that distal airway targeting would be enhanced.

The Aeroneb Go (Aerogen) is a portable electronic nebulizer with a vibrating mesh that was designed to be as efficient as the PARI LC PLUS (PARI Respiratory Equipment, Inc., Midlothian, Va.; PARI GmbH, Starnberg, Germany). The Aeroneb Go is intermediate in price, and is available as an open device. It also is more portable than a jet nebulizer, and has the advantage of silent operation. It is also the fastest at total drug output.

The PARI LC STAR (PARI Respiratory Equipment, Inc., Midlothian, Va.; PARI GmbH, Starnberg, Germany) powered by a standard compressor (PRONEB ULTRA) is widely available and widely used, and is the least expensive option. Downsides are that it is the least portable device and is noisy. It was the most time-consuming device for total drug delivery, but compensated for that by producing the smallest particles.

Conclusion: Each device has advantages and disadvantages, but the estimated lung dose delivery per unit time is likely very similar. Thus, it may be predicted that for the pulmonary hypertension and/or indications requiring systemic absorption for treatment of prevention of ischemic reperfusion injury indication, any of these devices may be selected.

### Example 5

### Ex Vivo Pharmacology

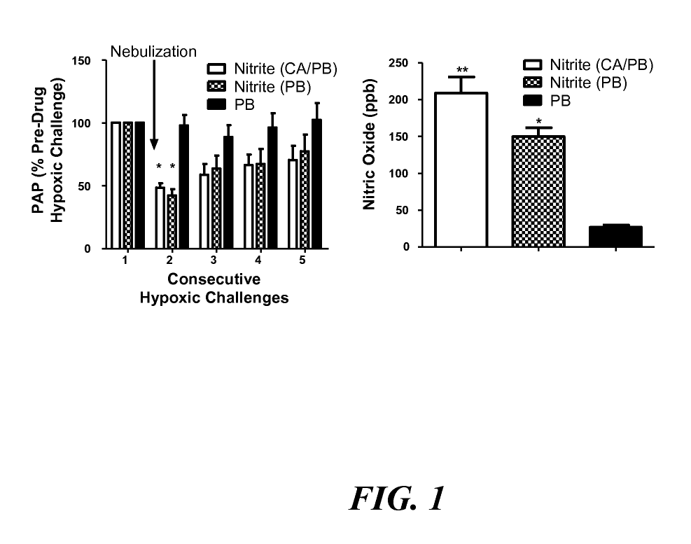

Preliminary work in an ex vivo rabbit model tested whether inhaled, nebulized sodium nitrite solution would reduce the pulmonary hypertension caused by reduced oxygen pressures. These experiments also assessed whether three different formulations of sodium nitrite altered its efficacy on pulmonary hypertension and nitric oxide production. Isolated rabbit lungs cannulated in the pulmonary artery were perfused with buffer containing a ˜12% hematocrit. Lungs were ventilated and pulmonary and arterial pressures were monitored by pressure transducers. After stabilization, hypoxic maneuvers were induced by lowering the oxygen content to 3% over 15 minute periods which resulted in increased pulmonary arterial pressure (PAP). Sodium nitrite (16.7 mg/mL) prepared in either phosphate buffer (pH 7.4), citric acid/saccharin/phosphate buffer (pH 5.5), or citric acid/ascorbic acid/saccharin/phosphate buffer (pH 5.5) was then administered via nebulization (5 min nebulization time) at the start of a single hypoxic challenge. Hypoxia-induced elevated PAP was significantly reduced by the sodium nitrite preparations in either phosphate buffer or phosphate/citric acid buffer (FIG. 1). Expired nitric oxide (measured via ventilator-inline Sievers 280 NOA nitric oxide analyzer) was higher in the citric acid/saccharin/phosphate and citric acid/ascorbic acid/saccharin/phosphate preparations. Lung weights, a measure of edema, were stable at doses up to 4.2 mg lung-delivered sodium nitrite (2.8 mg nitrite), while lung weight increased significantly at delivered doses ≧20.6 mg sodium nitrite (13.8 mg nitrite).

Isolated rabbit lungs were cannulated in the pulmonary artery and perfused with buffer containing ˜12% hematocrit. Lungs were ventilated as described by Weissmann et al 2001, and pulmonary/arterial pressures were monitored by pressure transducers. After system stabilization, hypoxic maneuvers were induced by lowering the oxygen content to 3% over 15 minute periods which resulted in increased PAP. The effect of sodium nitrite prepared in either phosphate buffer (PB) or citric acid (CA)/phosphate buffer (both at pH 5.5, n=5/6 per group) was then measured after administered via nebulization during the second hypoxic challenge. FIG. 1, Left panel: sodium nitrite in both buffer systems significantly decreased PAP (over 50%) compared with pre-drug hypoxic challenge (p<0.05). FIG. 1, Right panel: expired nitric oxide was significantly increased by both sodium nitrite preparations compared to control, but sodium nitrite prepared in citric acid produced significantly more nitric oxide prepared in phosphate buffer only (p<0.05). *Indicates significant difference from control, **indicates significant difference from nitrite in phosphate buffer.

Formulations containing citric acid, pH ˜5.5 and 1:256 molar ratio of ascorbic acid to nitrite produce ˜4-fold more nitric oxide than the same formulation lacking ascorbic acid. However, reduction of nitrite with ascorbic acid results in nitrogen dioxide gas formation (visualized as a brown gas). Nitrogen dioxide is considered a toxic substance when exposed to the lungs. These data indicated that while formulations with or without citric acid were efficacious (as measured by reduction of hypoxia-induced increases in PAP) the addition of ascorbic acid appears toxic. However, because the addition of citric acid produced more formulation-dissolved and expired nitric oxide than sodium nitrite formulations lacking citric acid, the inclusion of citric acid may enable immediate acute symptomatic relief upon inhalation of this nebulized formulation.

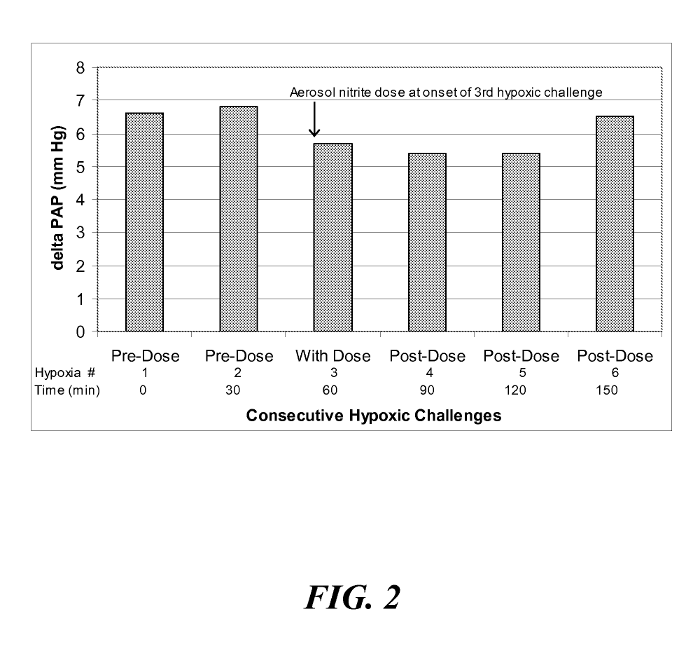

FIG. 2 shows the sustained-effect of administering sodium nitrite as a nebulized, inhaled solution using the procedure described above.

Isolated rabbit lungs were cannulated in the pulmonary artery and perfused as described in FIG. 1. After system stabilization, hypoxic maneuvers were induced by lowering the oxygen content to 3% over 15 minute periods which resulted in increased PAP. The effect of sodium nitrite prepared in phosphate buffer was then administered via nebulization during the third hypoxic challenge. The sustained effect is measured as a function of time to return to the same level of hypoxia-induced PAP as that measured prior to dosing. Half life is calculated as ˜10 min, with a sustained effect being ≧60 min.

The results in FIG. 2 indicate that nebulized aerosol administration of inhaled sodium nitrite results in a sustained effect lasting more than 60 min. This result can also be seen in comparison to inhaled nitric oxide gas where the effect of the inhaled gas is immediately lost upon termination of dosing (Hunter et al., 2004).

### Example 6

### 7-Day Inhalation Toxicology

This example summarizes the results from 7-day dose range finding studies in rat and dog administered inhaled sodium nitrite using a dose-ranging formulation composed of sodium nitrite, sodium phosphate, sodium saccharin, and citric acid, pH ˜5.5 (Formulation I Inhalation Solution).

TABLE 21
Experimental Design

| | | Dog | Rat |
|---|---|---|---|
| Group | Group | Target Dose Level | Target Dose Level |
| Number | Designation | (mg/kg/day) | (mg/kg/day) |
| 1 | Control | 0 | 0 |
| 2 | Low Dose | 2 | 4 |
| 3 | Mid Dose | 10 | 22 |
| 4 | High Dose | 44 | 97*/72** |

*The targeted dose level and the number of animals used in Group 4 on Day 1 only. The dose level for the high dose group was decreased due to the adverse clinical signs and deaths following Day 1 of exposure. Replacement animals were dosed for the next 6 days as originally scheduled.
**Target dose level and animal numbers from Days 2 to 7.

Formulations.

Aliqouts of the required volume of the test article formulations (final admixture) for Groups 2, 3 and 4 were prepared fresh each dosing day. The formulations defined below were titrated by nebulization time to achieve the dose levels define in Table 21.

0 mg/mL of Sodium Nitrite (Control: Group 1)

A. Vial 1:

0 mg/mL sodium nitrite

6.9 μg/mL monobasic sodium phosphate (NaH₂PO₄)

13.4 μg/mL dibasic sodium phosphate (Na₂HPO₄)

The solutions were mixed in sterile water for injection USP

The pH was recorded

B. Vial 2:

6.4 mM citric acid (monohydrate)

1.0 mM sodium saccharin (dihydrate)

The solutions were mixed in sterile water for injection USP

The solution was filtered with a 0.22 μm PVDF filter

The pH was recorded

C. Mix (for Final Formulation):

1. 1 part of Vial 1 was mixed with 1 part of Vial 2 to create the final formulation

2. The final formulation was filtered with a 0.22 μm PVDF filter

3. The pH was recorded pH once daily

12 mg/mL of Sodium Nitrite (Low Dose: Group 2)

A. Vial 1:

24 mg/mL sodium nitrite

6.9 μg/mL monobasic sodium phosphate (NaH₂PO₄)

13.4 μg/mL dibasic sodium phosphate (Na₂HPO₄)

The solutions were mixed in sterile water for injection USP

Once daily a representative formulation sample was collected

The pH was recorded

B. Vial 2:

0.5 mM citric acid (monohydrate)

0.1 mM sodium saccharin (dihydrate)

The solutions were mixed in sterile water for injection USP

The solution was filtered with a 0.22 μm PVDF filter

The pH was recorded

C. Mix (for Final Formulation):

1. 1 part of Vial 1 was mixed with 1 part of Vial 2 to create final the formulation

2. The final formulation was filtered with a 0.22 μm PVDF filter

3. A 1-mL representative formulation sample was collected (pre-filtration on Day 1 and post-filtration for Days 1 to 7) for each aliquot of the final mixture

4. The pH was recorded once daily from a representative formulation sample

60 mg/mL of Sodium Nitrite (Mid Dose: Group 3)

A. Vial 1:

120 mg/mL sodium nitrite

6.9 μg/mL monobasic sodium phosphate (NaH₂PO₄)

13.4 g/mL dibasic sodium phosphate (Na₂HPO₄)

The solutions were mixed in sterile water for injection USP

A representative formulation sample was collect once daily

The pH was recorded

B. Vial 2:

2.6 mM citric acid (monohydrate)

0.4 mM sodium saccharin (dihydrate)

The solutions were mixed in sterile water for injection USP

The solution was filtered with a 0.22 μm PVDF filter

The pH was recorded

C. Mix (for Final Formulation):

1. 1 part of Vial 1 was mixed with 1 part of Vial 2 to create the final formulation

2. The final formulation was filtered with a 0.22 μm PVDF filter

3. A 1-mL representative formulation sample was collected (pre-filtration on Day 1 and post-filtration for Days 1 to 7) for each aliquot of the final mixture

4. The pH was recorded once daily from a representative formulation sample

150 mg/mL of Sodium Nitrite (High Dose: Group 4)

A. Vial 1:

300 mg/mL sodium nitrite

6.9 μg/mL monobasic sodium phosphate (NaH₂PO₄.H₂O)

13.4 g/mL dibasic sodium phosphate (Na₂HPO₄.7H₂O)

The solutions were mixed in sterile water for injection USP

A representative formulation sample was collect once daily

The pH was recorded

B. Vial 2:

6.4 mM citric acid (monohydrate)

1.0 mM sodium saccharin (dihydrate)

The solutions were mixed in sterile water for injection USP

The solution was filtered with a 0.22 μm PVDF filter

The pH was recorded

C. Mix (for Final Formulation):

1. 1 part of Vial 1 was mixed with 1 part of Vial 2 to create the final formulation

2. The final formulation was filtered with a 0.22 μm PVDF filter

3. A 1-mL representative formulation sample was collected (pre-filtration on Day 1 and post-filtration for Days 1 to 7) for each aliquot of the final mixture

4. The pH was recorded once daily from a representative formulation sample

Results & Discussion (Rats).

A 7-Day range-finding study with nebulized Formulation I Inhalation Solution was performed via inhalation through the nose of male and female Sprague-Dawley rats. Rats were exposed to either vehicle (citric acid/saccharin/sodium phosphate buffer), or Formulation I Inhalation Solution prepared in a vehicle identical to control vehicle to achieve target doses of nitrite of 4, 22 or 97 mg/kg/day for 7 days (Table 17). Actual administered doses in the study were determined as 4, 18 and 101 mg/kg/day, respectively. Nebulization times and hence drug exposure times were 60-120 minutes, depending on treatment group with particle sizes as shown in Table 22.

TABLE 22
Particle size distribution measurements

| | | | Sodium Nitrite Particle Size Data | | |
|---|---|---|---|---|---|
| | Group | Group | | | % of particles |
| Species | Number | Designation | MMAD | σg | <3 μm |
| Rat | 2 | Low Dose | 1.0 | 1.85 | 97.6 |
| | 3 | Mid Dose | 1.4 | 1.87 | 87.6 |
| | 4 | High Dose | 1.7 | 1.93 | 78.5 |
| Dog | 2 | Low Dose | 1.2 | 2.53 | 86.2 |
| | 3 | Mid Dose | 1.7 | 2.39 | 73.9 |
| | 4 | High Dose | 1.9 | 1.97 | 73.3 |

MMAD = Mass median aerodynamic diameter (μm)
σg = Geometric standard deviation.

After completion of the first dose, high dose animals appeared cyanotic as evidenced by development of a bluish color at mucous membranes, eyes and feet. Thirty percent of the females died at the high dose level after receiving the first dose (6/20 rats). Subsequently, after day one, the high dose was lowered to an administered target dose of 72 mg/kg/day in both male and females. No remarkable clinical observations were noted in the controls and low or mid dose groups at day 1 and no remarkable clinical observations were noted throughout days 2-7 at any dose level. Methemoglobin levels in blood were increased in all dose groups and increased as a function of dose (Table 23) only increasing to ˜1% in the low dose and up to 4.5% in the mid dose group. Higher methemoglobin levels were observed in females at the middle and high dose level and correlated with Day 1 high dose group mortality. No cumulative effect of repetitive dosing on methemoglobin at these dose levels was observed.

TABLE 23
Mortality and Methemoglobin concentrations after a single dose and 7 days dosing
of an inhaled sodium nitrite solution

| | Dose: | Calculated | | | Peak Peak | | Peak Peak | |
|---|---|---|---|---|---|---|---|---|
| | mg/kg/day | deposited | Mortality | | MetHgb (%) | | MetHgb (%) | |
| | or | dose | (%) | | On Day 1 | | On Day 7 | |
| Species | (mg/M²) | (mg/kg)* | Male | Female | Male | Female | Male | Female |
| Rat | 0 (0) | 0 | 0 | 0 | 0.8 ± 0.1 | 0.7 ± 0.1 | 0.8 ± 0.0 | 0.9 ± 0.0 |
| | 4 (40) | 0.4 | 0 | 0 | 1.0 ± 0.0 | 1.0 ± 0.1 | 1.0 ± 0.0 | 1.3 ± 0.1 |
| | 18 (180) | 1.8 | 0 | 0 | 3.4 ± 0.3 | 4.5 ± 1.2 | 2.3 ± 0.2 | 3.8 ± 0.6 |
| | 101 | 10.1 (7.6) | 0 | 30 | 41 ± 3.3 | 50 ± 3.0 | 16 ± 4.7* | 36 ± 3.0* |
| | (76)**: | | | | | | | |
| | (1010 | | | | | | | |
| | (760)) | | | | | | | |
| Dog | 0 (0) | 0 | 0 | 0 | 0.0 ± 0.0 | 0.0 ± 0.0 | 0.0 ± 0.0 | 0.0 ± 0.0 |
| | 2 (50) | 0.5 | 0 | 0 | 0.2 ± 0.1 | 0.35 ± 0.4 | 0.2 ± 0.0 | 0.2 ± 0.0 |
| | 12 (300) | 3.0 | 0 | 0 | 2.6 ± 0.5 | 3.4 ± 1.3 | 2.1 ± 0.5 | 1.6 ± 0.2 |
| | 54 (1350) | 13.5 | 0 | 0 | 16 ± 5.2 | 19 ± 0.5 | 16 ± 3.2 | 17 ± 2.6 |

*Assumes: 1) average weight of rat = 0.250 kg and body surface area of 0.025 m²; and 2) and average weight of dog = 10 kg and body surface area of 0.4 m².
**Note dose was lowered to 72 mg/kg/day in the high dose group after day 1.

Gross pathology was in general unremarkable in all animals that were dosed 7 days with few small red areas in thymuses in both control and treated animals. In the animals that died after the first dose, gross pathology was noted in some but not all animals included mottled or non-collapsing or lungs. The observed changes in histopathology in the rats that died included mild lung edema, moderate congestion as well as moderate vacuolation of the vomeronasal organ. Among animals surviving the full treatment period, histopathology included minimal perivascular mixed cell infiltrates of the lung. This was seen in both the control and high dose treated animals only (no other groups were examined at this time). All other findings were considered incidental or procedure-related. Therefore, based mainly on the transient increases in methemoglobin, an NOAEL of 18 mg/kg/day was established.

Results & Discussion (Dogs).

A 7-Day dose-range-finding study with nebulized Formulation I Inhalation Solution was performed via inhalation through the nose and mouth of male and female beagle dogs. Dogs were exposed to either vehicle (citric acid/saccharin/sodium phosphate buffer), or Formulation I Inhalation Solution prepared in identical vehicle to achieve target doses of 2, 10 or 44 mg/kg/day for 7 days (Table 21). Administered doses were 2, 12 and 54 mg/kg/day. Nebulization time and hence drug exposure times were 60-120 minutes, depending on dose with particle sizes shown in Table 22 above. No remarkable clinical observations were noted in any treatment group over the 7-day period. Methemoglobin levels in blood increased appreciably in the high-dose group and minimally above basal levels in the mid-dose group (Table 23). Gross necropsies of the treated groups were in general similar to controls which included the presence of small red foci in the lung area, were few in nature and not associated with a dose responsive test-article relationship. Histopathology included mild focal pneumonia with minimal to mild focal peribronchiolar/perivascular mononuclear cell infiltrate and minimal to mild focal alveolar mixed cell infiltrate in the lungs of both the vehicle and high dose groups (low- and mid-dose groups not examined). M/E ratios were decreased in high-dose group males. However, there was no corresponding significant decrease in females and there were no morphological changes in any animals indicating that, for this study, the finding is of minimal toxicological significance. Therefore, inhalation of sodium nitrite at doses up to 54 mg/kg/day produced only mild and/or transient changes (e.g., M/E, methemoglobin). Therefore, based on the transient changes during this 7 day study, an NOAEL of 12 mg/kg/day was established.

### Example 7

### Micronization and Blending

To assess the ability to micronize sodium nitrite (NaNO₂) for inhalation delivery, micronization and blending experiments were performed. For animal pharmacology, NaCl was selected as the blending agent to maintain content uniformity by approximately matching particle densitys of both NaCl and NaNO₂.

Both sodium chloride (NaCl) and NaNO₂ salts were successfully micronized using a jet mill with compressed air supply. Particle size distributions (PSD) of micronized NaCl and NaNO₂ samples were determined in medium chain triglyceride oil using laser diffraction technique. Particle size distributions of both micronized materials were determined to be less than 10 microns at D₅₀ (median) as summarized in Table 24.

TABLE 24
Particle size distributions of micronized NaCl and NaNO₂ samples in
medium chain triglyceride oil using laser diffraction technique.

| | | | | |
|---|---|---|---|---|
| Reading No. | Obscuration | D(v, 0.1) | D(v, 0.5) | D(v, 0.9) |

Sodium Chloride (NaCl)

| | | | | |
|---|---|---|---|---|
| 1 | 18.8% | 3.90 | 7.74 | 17.14 |
| 2 | 17.9% | 3.93 | 7.15 | 18.47 |
| Average | — | 3.92 | 7.45 | 17.81 |

Sodium Nitrite (NaNO₂)

| | | | | |
|---|---|---|---|---|
| 1 | 18.5% | 4.37 | 9.53 | 23.35 |
| 2 | 18.8% | 4.36 | 9.50 | 23.15 |
| 3 | 18.8% | 4.35 | 9.46 | 22.89 |
| Average | — | 4.36 | 9.50 | 23.13 |

D(v, 0.1) = 10% of the mean particle size distribution
D(v, 0.5) = 50% of the mean particle size distribution
D(v, 0.9) = 90% of the mean particle size distribution

Particle sizes of NaCl and NaNO₂ samples were also confirmed under a light microscope. Four blends of NaCl and NaNO₂ mixture at various ratios were manufactured using a geometric dilution technique. Each micronized material was de-lumped by passing through a 70-mesh sieve prior to blending. Each blend was mixed stepwise using the vortexer for 1 minute between each mixing step as shown in Table 25.

TABLE 25
Blending of micronized NaCl and NaNO₂.

Weight (mg)

| Ingredient | Blend-1 | Blend-2 | Blend-3 | Blend-4 |
|---|---|---|---|---|
| Micronized NaNO₂ | 25.0 | 75.0 | 250.0 | 0 |
| Micronized NaCl | 1975.0 | 1925.0 | 1750.0 | 2000.0 |
| Total | 2000.0 | 2000.0 | 2000.0 | 2000.0 |

Summary:

Sodium chloride and sodium nitrite salts were successfully micronized using jet pulverization mill with compressed air supply. Particle size distribution of both micronized samples were determined in medium chain triglyceride oil using laser diffraction to be <10 microns at D50 (median). Four mixture blends were prepared successfully using geometric dilution technique.

### Example 8

### In Vivo Pharmacokinetics

The pharmacokinetics of sodium nitrite was assessed after intratracheal administration when prepared as a dry powder, as a nebulized solution prepared in phosphate buffer or after IV administration in phosphate buffer. Male Sprague-Dawley rats (˜280-300 g) were purchased with an indwelling catheter in the jugular vein and the catheter was flushed with sterile saline containing 10 U/mL of heparin prior to dosing. For intratracheal administration of dry powder, animals were anesthetized with isoflorane and using a Penn-Century insufflator (model DP-4), powdered sodium nitrite was insufflated just above the first bifurcation of the trachea. Exact dose was determined gravimetrically. For intratracheal administration of sodium nitrite in phosphate buffer (100 μl, 30 mg/mL), a Penn-Century Microsprayer Aerosolizer (model IA-1C/FMJ 250; Philadelphia, Pa.) was used and dosing was performed in the same manner as above. IV administration of sodium nitrite (10 mg/kg) was delivered via the rat tail vein. Blood was collected in heparinized tubes 5, 15, 30, 60, 120 and 240 minutes after dosing, immediately put on ice and then centrifuged at 13,000 rpm in a microcentrifuge for 45 sec. Plasma was harvested and frozen at −80° C. until analysis. Sodium nitrite was analyzed by a commercially available kit (R&D Systems). Administration of IV administrated sodium nitrite resulted in rapid disappearance of nitrite in plasma with a t_{1/2} of 20 minutes (Table 26).

TABLE 26
Pharmacokinetics of Sodium Nitrite Following IV and IT (Both
Liquid Nebulized and Dry Insufflated) Administration.

| | | | IT (Dry powder, |
|---|---|---|---|
| | | IT (liquid, 10 | normalized to 10 |
| | IV (10 mg/kg) | mg/kg) | mg/kg) |
| AUC (ug*min/mL) | 487 | 169 | 256 |
| Cmax (μM) | 312 | 164 | 172 |
| Tmax (minutes) | 5 | 15 | 5 |
| T_{1/2} (minutes) | 20 | 10 | 19 |

Administration of sodium nitrite via intratracheal insufflation administration of either dry powder or liquid also resulted in rapid absorption (Cₘₐₓ of 5 and 15 minutes, respectively) and elimination (t_{1/2} of 19 and 10 minutes, respectively). These data indicate that sodium nitrite given as a dry powder has similar PK characteristics as IV administration. These results also indicate that plasma pharmacokinetics of sodium nitrite following intratracheal instillation/insufflation are similar, suggesting that the dissolution rate of micronized sodium nitrite is readily bioavailable to the pulmonary effect compartment and may provide a dose-equivalent efficacious response. However, a limitation of this study is that because the dry powder insufflation device requires 2-5 mg of material for proper function, administration of lower amounts of unblended sodium nitrite was not possible.

To study the pharmacokinetics of lower amounts of delivered sodium nitrite, sodium nitrite was blended with sodium chloride. Sodium chloride was selected to enable content uniformity as the density of other blending agents, such as lactose is roughly one-half that of sodium nitrite while sodium chloride is equivalent. Following blending and micronization, rats were administered blends targeting 1.0, 0.1 and 0.01 mg/kg of sodium nitrite. Rats (n=3-4/group, ˜400 gm) were anesthetized with Isoflorane and the dry powder was insufflated intratracheally using a Penn-Century Insufflator (model DP-4: Philadelphia, Pa.). Blood was collected in heparinized tubes just prior to dosing, 5, 15, 30 and 60 minutes after dosing, immediately put on wet ice and then centrifuged at 13,000 rpm in a microcentrifuge for 45 sec. Plasma was harvested and frozen at −80° C. until analysis. Analysis for nitrite was performed using an HPLC method with fluorescence detection as described (Li et al., J Chromatogr B Biomed Sci Appl. 2000 Sep. 15; 746(2):199-207). Results are shown in Table 27.

TABLE 27
Plasma Pharmacokinetics Following Intratracheal Insufflation
Administration of Dry Powder Sodium Nitrite to the Rat Lung.

| | Dry Powder Sodium Nitrite | | |
|---|---|---|---|
| μg/kg | 700 | 90 | 10 |
| AUC (μg*min/mL) | 17.5 | 0.95 | ND |
| Cmax (μm) | 25.8 | 1.8 | * |
| Tmax (minutes) | 5 | 5 | * |
| T_{1/2} (minutes) | 10 | 4 | * |

Not Detected
N = 3-4/group

The data demonstrate a dose-dependent increase in AUC and Cmax with no detection at the lowest dose of dry powder sodium nitrite (10 μg/kg). These data also show that sodium nitrite can be dosed as a blend to achieve similar pharmacokinetic properties as the unblended dosage form.

### Example 9

### In Vivo Pharmacology

Preliminary work in an in vivo rat model of monocrotaline-induced pulmonary hypertension tested whether inhaled, nebulized sodium nitrite solution would reduce the pulmonary hypertension as assessed by changes in right ventricle: left ventricle+septum ratios. Male Sprague-Dawley rats (n=8/group, ˜300 g) were injected subcutaneously with either saline vehicle (control group) or monocrotaline (MCT: 50 mg/kg, sc) prepared in saline and pulmonary hypertension was allowed to develop over a 3-week period prior to therapeutic dosing. At 3 weeks, groups of rats began treatment with inhaled nebulized solutions of either phosphate buffer saline (PBS), or sodium nitrite admixture (30 mg/5 mL), containing 0.13 mM citric acid, 0.02 mM sodium saccharin and 0.002 mM phosphate buffer (pH 5.5), or vehicle of the sodium nitrite admixture (citric acid, sodium saccharin and phosphate buffer alone) nebulized into a ventilated chamber for 20 minutes, 3 days a week for 3 additional weeks. Based on the exposure time, rat ventilation rate, and concentration of nebulized sodium nitrite in the dosing chamber, rats were exposed to approximately a 5 μg/kg dose every exposure period. After three weeks of treatment (6 weeks after MCT injection), rats were euthanized and hearts were removed: the right ventricle and left ventricle with septum were weighed and the ratio of weights were recorded (RV:LV+S) as an indicator of right heart hypertrophy resulting from pulmonary hypertension. Compared to vehicle treated controls, rats exposed to monocrotaline for a total of six weeks developed severe pulmonary hypertension as assessed by nearly 2.5-3-fold increases in RV:LV+S ratios (Table 28). When exposed to the sodium nitrite admixture, RV:LV+S ratios were significantly reduced by approximately 50%, demonstrating a benefit in this disease state.

TABLE 28
Inhaled Liquid Sodium Nitrite Therapy in Rat
Monocrotaline Model of Pulmonary Hypertension

| MCT | − | − | + | + |
|---|---|---|---|---|
| Treatment | PBS | Admixture | Admixture | Sodium Nitrite |
| | alone | Control | Control | Admixture |
| RV: LV + S | 0.234 ± | 0.218 ± | 0.644 ± | 0.421 ± |
| | 0.010 | 0.005 | 0.052 | 0.014* |

*Significantly different from MCT/Citric acid/saccharin/phosphate group (p < 0.05, one way ANOVA).

### Example 10

### In Vivo Pharmacology

To assess the efficacy of dry powder sodium nitrite in the treatment of pulmonary hypertension, the in vivo rat model of monocrotaline-induced pulmonary hypertension was tested as described herein. One week following MCT (50 mg/kg, sc) injection, rats were administered micronized sodium nitrite blended with sodium chloride or micronized sodium chloride alone (vehicle control) using a Penn-Century dry powder insufflator (model DP-4, Philadelphia, Pa.). Because the dry powder insufflation device requires 2-5 mg of material for proper function, administration of lower sodium nitrite levels predicted to be efficacious required blending. Sodium chloride was selected as the blending agent to enable content uniformity as the density of other blending agents, such as lactose is roughly one-half that of sodium nitrite while sodium chloride is equivalent. Following micronization and blending, animals received ˜1 μg of sodium nitrite/kg/dose or ˜10 μg sodium nitrite/kg/dose or equivalent sodium chloride blend alone. Intratracheal insufflation administration of sodium nitrite dry powder was initiated one week following MCT injection and occurred three times per week for four weeks. On the 32^{nd} day following MCT injection, rats were euthanized and hearts removed. The right ventricle and left ventricle with septum were weighed and the ratio of weights were recorded (RV:LV+S) as an indicator of right heart hypertrophy resulting from pulmonary hypertension. Results are shown in Table 29.

TABLE 29
Inhaled Dry Powder Sodium Nitrite Therapy in Rat
Monocrotaline Model of Pulmonary Hypertension

| MCT | − | + | + | + |
|---|---|---|---|---|
| Treatment | Sodium | Sodium | Sodium Nitrite | Sodium Nitrite |
| | Chloride | Chloride | 1 μg/kg | 10 μg/kg |
| | Alone | Alone | | |
| RV: LV ± S | 0.226. ± | 0.423 ± | 0.363 ± | 0.328 ± |
| | 0.007 | 0.032 | 0.029 | 0.025* |

*Significant by ANOVA/Bonferroni post-hoc test (p < 0.05).

MCT significantly increased RV:LV+S over untreated controls (from 0.226 to 0.443), while treatment with sodium nitrite dose-dependently decreased RV:LV+S up to 48% at the high dose (p<0.05). These results suggest that dry powder sodium nitrite is efficacious in the rat model of monocrotaline-induced pulmonary hypertension. PK analysis may be found in Example 8, Tables 27.

Combining these efficacy data (here and Example 9) with plasma pharmacokinetics (Example 8), it is observed that a dose of 90 μg/kg results in a plasma Cₘₐₓ of 1.8 μM while a dose of 700 μg/kg results in a plasma Cₘₐₓ of 25.8 μM showing both an approximate dose-proportionality and that these dose levels result in plasma levels known in the art to be related to efficacy. Further, by example and shown herein, 10 μg/kg dry powder aerosol resulted in efficacy with a Cₘₐₓ plasma nitrite concentration of ˜0.2 μM (Table 29), as did 5 μg/kg liquid aerosol with an extrapolated ˜0.1 μM plasma nitrite (Example 9, Table 28).

Extending this relationship to human exposure (Example 12), detectable plasma nitrite levels with an immediate post-dose Cₘₐₓ of 0.66 μM were observed following inhalation of a 1.6 mg aerosol dose or ˜23 μg/kg (assuming a 70 kg human) over a 10 min period. The lowest dose resulting in adverse systemic hypotension was 176 mg or ˜2,500 μg/kg administered over the same period (Cₘₐₓ of 11.57 μM). Following dose de-escalation, it was determined that a 125 mg inhaled aerosol dose was safe (˜1.79 mg/kg, administered over 10 min, resulting in a Cₘₐₓ of 9.23 μM). Taking together, it appears that doses resulting in less than or equal to ˜9 μM plasma nitrite are safe. Pharmacodynamically, it appears that inhaled doses resulting in as low as 0.1 μM plasma nitrite are efficacious. Assuming these efficacious doses in animals translate linearly into humans, these results suggest a maximum therapeutic index of ˜92 (9.23 μM/0.1 μM).

In another example, it was shown that intravenous administration of 6.2 μg/kg nitrite directly to a hypoxia-induced hypertensive human vasculature results in a plasma Cₘₐₓ of ˜10 μM nitrite and efficacy (˜20% systemic vasodilation) (Hypoxic modulation of exogenous nitrite-induced vasodilation in humans. Maher A R, Milsom A B, Gunaruwan P, Abozguia K, Ahmed I, Weaver R A, Thomas P, Ashrafian H, Born G V, James P E, Frenneaux M P. Circulation. 2008 Feb. 5; 117(5):670-7.). These results indicate that: 1. compared to the liquid and dry powder aerosol delivery directly to the lung described herein less intravenous drug results in higher plasma levels suggesting, amoung other scenarios, that nitrite delivered directly to the pulmonary compartment is slowly bioavailable to the vascular circulation; 2. this observation supports the safety conclusion that plasma nitrite levels greater than ˜9 μM result in the adverse event of systemic hypotension; and 3. taken together these observations support that less inhaled nitrite is required for pulmonary-related efficacy than that administered by the intravenous route. Thus, aerosol inhalation delivery for treatment of pulmonary disease requires less nitrite for efficacy than if delivered by parenteral routes. Moreover, the amount of parenteral nitrite required for pulmonary efficacy would prove unsafe in the clinical setting.

Combining these data, it appears that plasma nitrite levels above ˜10 μM are potentially unsafe in the human clinical setting. Further, animal and human efficacies were observed at plasma nitrite levels less than 10 μM, with animal data supporting down to 0.1 μM plasma nitrite. By non-limiting example, to achieve these doses the following may be adminstered (rationale compiled from Examples 1, 8, 9, 10, and 12):
Administration of nitrite such that the resultant plasma nitrite level exceeds a Cₘₐₓ of ˜10 M is potentially unsafe for human use;
Human and animal studies indicate observed efficacy with doses resulting in a plasma Cₘₐₓ of ˜10 μM and range down to a Cₘₐₓ of ˜0.1 μM;
Liquid nitrite salt solution administered by inhalation following nebulization from a device providing a FPD % of ˜25%: 1 mg (˜0.25 mg FPD) to 360 mg (˜90 mg FPD) device-loaded sodium nitrite provides human plasma nitrite levels between ˜0.1 μM and ˜10 μM; and
Dry powder sodium nitrite administered by inhalation following dispersion in a device providing a FPD % of ˜50%: 0.35 mg (˜0.18 mg FPD) to 35 mg (˜18 mg FPD) device-loaded dry powder sodium nitrite provides human plasma nitrite levels between ˜0.1 μM and ˜10 μM.
Using the same FPD % relationship to FPD, devices exhibiting a different FPD % will require a different device-loaded amount of either liquid or dry powder nitrite.

### Example 11

### Ex Vivo Pharmacology

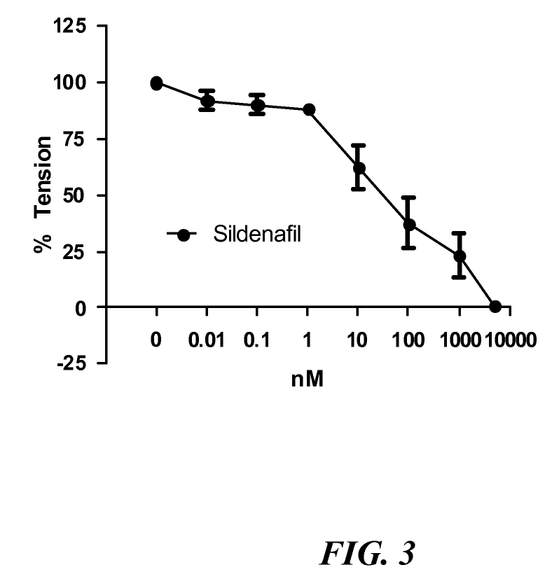

To assess the potentiation and/or synergy between the PDE5 inhibitor Sildenafil and sodium nitrite an isolated rat aortic ring model was employed. Specifically, this model was used to measure the ability of Sildenafil and/or sodium nitrite to reduce phenylephrine-induced contractions of aortic rings in vitro. In the first experiment, Sildenafil was titrate versus a contracted aortic ring to determine the dose where the drug was 50% effective (effective dose, ED₅₀). Briefly, a rat aorta was excised and cleansed of fat and adhering tissue. Vessels were then cut into individual ring segments (2-3 mm in width) and suspended from a force-displacement transducer in a tissue bath. Ring segments were bathed in a bicarbonate-buffered, Krebs-Henseleit (KH) solution of the following composition (mM): NaCl 118; KCl 4.6; NaHCO₃ 27.2; KH₂PO₄ 1.2; MgSO₄ 1.2; CaCl₂ 1.75; Na₂EDTA 0.03, and glucose 11.1. A passive load of 2 grams was applied to all ring segments and maintained at this level throughout the experiments. At the beginning of each experiment, indomethacin-treated ring segments were depolarized with KCl (70 mM) to determine the maximal contractile capacity of the vessel. Rings were then washed extensively and allowed to equilibrate. For subsequent experiments, vessels were submaximally contracted (50% of KCl response) with phenylephrine (PE) (3×10⁻⁸-10⁻⁷ M). The first set of studies defined the dose-dependent relaxation of aortic smooth rings in the presence of increasing concentrations of Sildenafil (FIG. 3).

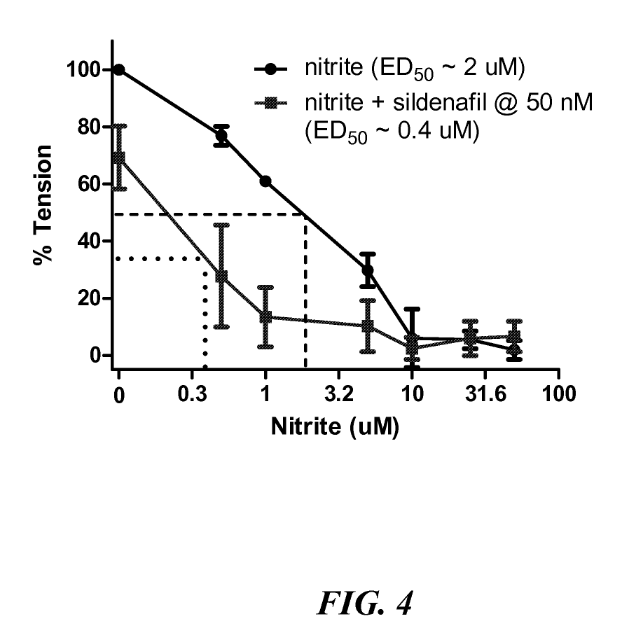

Results from FIG. 3 indicate an ED₅₀ of 50 nM for Sildenafil. To determine if sodium nitrite potentiates or acts synergistically with Sildenafil, two experiments were performed. The first experiment titrated sodium nitrite (as described above for Sildenafil alone), while the second performed the same sodium nitrite titration, but in the presence of ED₅₀ Sildenafil (50 nM). Briefly, aortic rings were first exposed to sildenafil at 50 mM to partially reduce aortic ring constriction. After equilibration, increasing amounts of sodium nitrite (500 nM-50 μM) were added to the buffer with tension measurements recorded after each addition. FIG. 4 demonstrates that sodium nitrite has an ED₅₀ of ˜2 μM in dialating contracted aortic rings. Further, in the presence of ED₅₀ Sildenafil, the ED₅₀ of sodium nitrite reduces to ˜0.4 μM. Thus, nitrite potentiates and/or acts synergistically with Sildenafil to further relax constricted rat aortic rings (leftward shift of the dose-response curve). It is noteworthy that these observed in vitro results further support in vivo results of efficacy shown in Examples 9 and 10.

### Example 12

### First-in-Man Dose Escalation Study to Maximum Tolerated Dose

This example summarizes the results from Protocol AIR001-CS01: A placebo-controlled, phase 1, dose escalation study to evaluate the safety, tolerability and pharmacokinetics of sodium nitrite inhalation solution (AIR001 Inhalation Solution) in normal, healthy volunteers.

### Experimental Design

Inhaled NO has been demonstrated to improve pulmonary hemodynamics acutely in patients with pulmonary hypertension. Inhaled nebulized sodium nitrite solution has been demonstrated to lower pulmonary arterial pressure acutely in preclinical models of pulmonary hypertension, putatively through a mechanism of sustained NO release. Repeat dosing of inhaled nebulized sodium nitrite solution has also been demonstrated to result in sustained improvement in pulmonary hemodynamics, right ventricular hypertrophy and in pulmonary vasculopathy in animal models of pulmonary hypertension.

AIR001 Inhalation Solution was studied as a treatment for pulmonary arterial hypertension. The current study was a first-in-man investigation undertaken to define the safety, tolerability, conversion of nitrite to NO and pharmacokinetic profile of inhaled nebulized AIR001 Inhalation Solution in normal male and female volunteers.

AIR001 Inhalation Solution was an admixture system prepared immediately prior to inhalation delivery to patients via electronic nebulization. The three AIR001 Inhalation Solution clinical trial formulations used in this study were as follows:

AIR001 Inhalation Solution Vial 1, Sodium Nitrite Solution

AIR001 Inhalation Solution Vial 2, Excipient Solution

AIR001 Inhalation Solution Vial 3, Placebo/Diluent Solution

Vial 1 contained 300 mg/mL sodium nitrite and 0.1 mM sodium phosphate buffer. Vial 2 contained 1.0 mM sodium saccharin as a taste-masking agent and 6.4 mM citric acid, to moderate pH of the final admixture solution. Vial 3 contained 0.1 mM sodium phosphate only. In the preliminary dose escalation to the maximum tolerated dose (MTD), immediately prior to administration, an equal portion of Vial 1 and Vial 2 were admixed and then diluted with Vial 3 contents as appropriate to achieve lower concentration dosing solutions for the dose-escalation protocol. Following establishment of the Vial 1+Vial 2 admixed test material MTD, additional dosing cohorts of 3 subjects were enrolled at the Vial 1+Vial 2 MTD, using Vial 3-diluted Vial 1 contents only.

### Results and Discussion

A total of 33 normal male and female subjects received a single dose of AIR001 Inhalation Solution via inhalation of an aerosol solution delivered by electronic nebulization. Each subject also received vehicle control. The nebulizer used for this study was the Aerogen Idehaler. The Aerogen Idehaler is a combination of two units. The nebulization head is the Aeroneb® Solo (Aerogen, Galway, Ireland) and the aerosol-reservoir attachment Idehaler™ (Diffusion Technique Francais, Saint Etienne, France). The Aeroneb Solo is 510K-cleared while the Idehaler reservoir attachment is CE-marked. These two units are supplied together from Aerogen. Together they create a silent, portable, high-efficiency electronic nebulizer that uses Aerogen's continuously vibrating mesh aerosol generation technology and the Idehaler reservoir to collect nebulized aerosol between inhalations. Together, this nebulizer allows high drug output and efficiency, minimal loss of drug to the environment between inhalations and a reproducible droplet size distribution for optimal delivery of drugs to the distal pulmonary tree. The performance of this and other nebulizers with AIR001 Inhalation Solution is shown in Tables 30 and 31. Measurements were obtained as outlined in Example 4.

Output Measurements Made were:

1. Duration of nebulization

2. Loading dose (LD): Total drug loaded within the nebulizer

3. Residual dose (RD): Total drug remaining in the nebulizer.

4. Inspired dose (ID): The predicted amount of ED deposited within the lung.

5. Expired Dose: Total drug on the expiratory filter. Collected only on the PARI STAR

6. Fine Particle Dose (FPD): The proportion of inspired dose with particles ≦5 microns.

7. Output (FPD per minute): The calculated FPD delivered per minute of nebulization

8. FPD %: The FPD expressed as percent of nominal dose

TABLE 30
AIR001 Inhalation Solution: Aerogen Idehaler Performance.

| | AIR001 Inhalation Solution | | | |
|---|---|---|---|---|
| Loaded Dose (mg) | 2.0 | 20.0 | 120.0 | 600.0 |
| Duration (minutes) | 12.3 ± 1.2 | 11.93 ± 1.34 | 12.1 ± 1.6 | 12.58 ± 1.34 |
| Loading Dose (mg) | 2.0 | 20 | 120 | 600 |
| Residual Dose (mg) * | 0.08 ± 0.02 | 0.65 ± 0.21 | 3.84 ± 1.15 | 16.16 ± 4.4 |
| Inspired Dose (mg) | 1.52 ± 0.16 | 17.18 ± 1.96 | 101.4 ± 6.86 | 463.24 ± 68.79 |
| Expired Dose (mg) | 0.10 ± 0.02 | 0.93 ± 0.29 | 6.3 ± 2.2 | 28.5 ± 4.24 |
| Total Recovered | 1.7 ± 0.18 | 18.76 ± 1.82 | 111.6 ± 8.2 | 507.92 ± 74.4 |
| (mg) | | | | |
| Fine Particle | 1.39 ± 0.13 | 14.69 ± 1.53 | 92.3 ± 5.7 | 370.60 ± 50.24 |
| Dose (mg) | | | | |
| Output (FPD/min) | 0.11 ± 0.02 | 1.25 ± 0.24 | 7.7 ± 0.87 | 29.91 ± 6.38 |
| FPD % | 69.3% ± 7.3 | 73.4 ± 8.4 | 76.9% ± 5.2 | 61.8 ± 9.2 |

* Contains only drug within the medication cup.
mean ± sd

TABLE 31
AIR001 Inhalation Solution: Aerogen Aeroneb Go Performance.

| | AIR001 Inhalation Solution | | | |
|---|---|---|---|---|
| Loaded Dose (mg) | 2.0 | 20.0 | 120.0 | 600.0 |
| Duration (minutes) | 7.03 ± 0.92 | 6.78 ± 0.96 | 6.43 ± 0.92 | 6.32 ± 0.81 |
| Loading Dose (mg) | 2.0 | 20.0 | 120 | 600 |
| Residual Dose (mg) * | 0.12 ± 0.02 | 1.12 ± 0.12 | 5.72 ± 0.93 | 31.49 ± 3.03 |
| Inspired Dose (mg) | 0.80 ± 0.07 | 6.41 ± 0.63 | 32.83 ± 3.87 | 192.41 ± 19.13 |
| Expired Dose (mg) | ND | ND | ND | ND |
| Total Recovered | 0.93 ± 0.05 | 7.52 ± 0.55 | 38.45 ± 3.68 | 223.9 ± 16.48 |
| (mg) | | | | |
| Fine Particle | 0.50 ± 0.04 | 4.25 ± 0.38 | 24.66 ± 2.65 | 133.34 ± 12.1 |
| Dose (mg) | | | | |
| Output (FPD/min) | 0.07 ± 0.01 | 0.63 ± 0.06 | 3.85 ± 0.28 | 21.18 ± 0.87 |
| FPD % | 25.2% ± 2.2 | 21.2% ± 2.1 | 20.5% ± 2.4 | 22.2% ± 2.2 |

* Contains only drug within the medication cup.
mean ± sd
ND = Not determined.

These in vitro results suggest that the Aerogen Idehaler delivers-3-fold more fine particle dose (mg inhaled mass in aerosol particles less than 4.7 microns, as determined by Andersen Cascade Impaction) than the Aerogen Aeroneb Go device. By example, and in relationship to recommended doses outlined in Example 10, to deliver a fine particle dose (FPD) of 0.25 mg (that which results in an ˜0.1 μM plasma nitrite concentration) sodium nitrite, the Aerogen Aeroneb Go (exhibiting an FPD % of ˜25%) would require a loaded dose (that placed into the nebulizer prior to nebulization and administration) of 1 mg sodium nitrite, while the Aerogen Idehaler (exhibiting an FPD % of ˜70%) would require a loaded dose of 0.36 mg. By further example, to deliver a FPD of 90 mg (that which results in an ˜10 M plasma nitrite concentration) sodium nitrite, the Aerogen Aeroneb Go would require a loaded dose of 360 mg sodium nitrite, while the Aerogen Idehaler would require a loaded dose of ˜129 mg. Following these FPD relationships to loaded dose, devices exhibiting different efficiencies of delivery (e.g. different FPD) will require different amounts of loaded drug.

In the human study using the Aerogen Idehaler nebulization device, the dose-limiting toxicity was symptomatic hypotension with a maximum observed tolerated dose of 125 mg (device-loaded sodium nitrite). An increase in heart rate was noted across all dose groups. An increase in methemoglobin level was identified to be dose-proportional with no subjects exceeding 2.9%. The AIR001 Inhalation Solution admixture was well tolerated while AIR001 Inhalation Solution lacking taste-masking excipient (Vial 3-diluted Vial 1 contents only) resulted in poor taste and cough.

Analysis of serum nitrite levels was performed. Pharmacokinetic analysis demonstrated a dose-proportional increase in the maximum serum nitrite concentration (Table 32) and further defined pharmacokinetic parameters (Table 33).

TABLE 32
Maximum plasma nitrite concentration following
aerosol dosing of AIR001 Inhalation Solution.

C_{Max} (μm)

| Dose | N | Mean | SD |
|---|---|---|---|
| 1.6 mg | 3 | 1.63 | 0 |
| 5.2 mg | 3 | 1.63 | 0 |
| 17 mg | 3 | 1.63 | 0 |
| 55 mg | 3 | 7.37 | 3.30 |
| 125 mg (with excipients) | 3 | 13.74 | 9.05 |
| 125 mg (without excipients) | 3 | 12.85 | 1.76 |

TABLE 33
Nitrite plasma pharmacokinetics following aerosol
dosing of AIR001 Inhalation Solution.

| | | | | Apparent | Baseline Weight |
|---|---|---|---|---|---|
| | | | Apparent Total | Volume of | Adjusted Volume |
| | | Half-Life | Clearance | Distribution | of Distribution^{b} |
| Statistic | T_{Max} (h) | (h) | (mL/min)^{b} | (L)^{b} | (L/kg) |
| N | 15 | 13 | 13 | 13 | 13 |
| Mean | 0.270 | 0.410 | 3691.367 | 160.387 | 2.160 |
| (SD)^{c} | (0.1550) | (0.2702) | (2364.6635) | (85.2046) | (1.0066) |
| Median | 0.200 | 0.548 | 2703.186 | 128.266 | 2.149 |
| Min, Max | 0.08, 0.52 | 0.17, 7.91 | 458.82, 9922.21 | 54.67, 342.60 | 0.78, 4.04 |

^{a}All pharmacokinetic parameters were derived using concentration results on or after the first inhalation of AIR001 Inhalation Solution.
^{b}Uses bioavailability equal to 0.70.
^{c}Harmonic mean used for half-life.

Bioconversion of nitrite to nitric oxide was demonstrated by dose-dependent increase in exhaled NO levels (Table 34). Exhaled nitric oxide was measured using the Niox Mino device (Aerocrine, Inc., USA, New Providence, N.J.).

TABLE 34
Exhaled NO following aerosol dosing
of AIR001 Inhalation Solution.

| | | Exhaled NO (ppb) |
|---|---|---|
| Dose | N | Change from Baseline |
| 0.04 mg | 3 | −6.0 |
| 0.13 mg | 3 | −0.3 |
| 0.5 mg | 3 | −2.3 |
| 1.6 mg | 3 | 1.7 |
| 5.2 mg | 3 | −2.7 |
| 17 mg | 3 | 31.7 |
| 55 mg | 3 | 45.7 |
| 125 mg (with excipients | 3 | 57.3 |
| 125 mg (without excipients) | 3 | 8.3 |

In summary, doses less than or equal to 125 mg (device loaded sodium nitrite) were well tolerated, demonstrated conversion of nitrite to NO, and resulted in plasma nitrite levels shown to be efficacious in animal models of pulmonary hypertension (See Examples 5 and 11 (ex vivo pharmacology), 9 and 10 (in vivo pharmacodynamics), and 8 (in vivo pharmacokinetics).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entireties to the extent they are not inconsistent with the disclosures herein. Aspects of the embodiments can be modified, if necessary to employ concepts of the various patents, applications and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-49. (canceled)

50. A method of treating pulmonary arterial hypertension or ischemic reperfusion injury comprising administering, via inhalation using a dry powder inhaler, to a subject in need thereof a therapeutically effective dose of a dry powder nitrite compound formulation composition wherein the dry powder inhaler delivers to the subject an aerosol containing about 0.18 to 18 mg sodium nitrite in particles of less than 5 microns volumetric mean diameter.

51. A dry powder inhaler for single or multiple dosing loaded with a dry powder sodium nitrite formulation so that the dry powder inhaler contains about 0.35 mg to about 35 mg per inhalation breath of sodium nitrite wherein said dry powder inhaler delivers to the subject an aerosol containing about 0.18 mg to about 18 mg sodium nitirite in particles of less than 5 microns mean diameter per inhalation breath.

52. The method according to claim 50 wherein the administration of the sodium nitrite results in about 0.1 μM to about 10 μM peak plasma nitrite.

53. The inhalation device of claim 51 wherein the delivery results in about 0.1 μM to about 10 μM peak plasma nitrite.

54-55. (canceled)
